(19) United States
(12) Patent Application Publication
(10) Pub. No.: US 2026/0366055 A1
(43) Pub. Date: Nov. 19, 2026

(54) METHODS FOR IMPROVED PRODUCTION OF REBAUDIOSIDE D AND REBAUDIOSIDE M

(71) Applicant: Danstar Ferment AG, Schweiz (CH)

(72) Inventors: Michael Dalgaard Mikkelsen, Vaerlose (DK); Jorgen Hansen, Frederiksberg (DK); Ernesto Simon, Copenhagen (DK); Federico Brianza, Riehen (CH); Angelika Semmler, Copenhagen (DK); Kim Olsson, Søborg (DK); Simon Carlsen, Copenhagen (DK); Louis Düring, Copenhagen (DK); Alexei Ouspenski, Mulhouse (FR); Paula Hicks, Bend, OR (US)

(21) Appl. No.: 19/743,949

(22) Filed: Jul. 16, 2026

Related U.S. Application Data
(63) Continuation of application No. 18/052,477, filed on Nov. 3, 2022, now Pat. No. 12,709,769.
Application No. 18/052,477 is a continuation of application No. 16/799,541, filed on Feb. 24, 2020, now Pat. No. 11,530,431.
Application No. 16/799,541 is a continuation of application No. 15/908,214, filed on Feb. 28, 2018, now Pat. No. 10,612,066.
Application No. 15/908,214 is a division of application No. 14/761,629, filed on Feb. 6, 2014, now Pat. No. 9,957,540.
(60) Provisional application No. 61/761,490, filed on Feb. 6, 2013.
(60) Provisional application No. 61/886,442, filed on Oct. 3, 2013.

(51) Int. Cl. C12P 19/56; A23L 2/60; A23L 27/30; C12N 9/02; C12N 9/10; C12N 9/90; C12N 15/81

(52) U.S. Cl. CPC C12P 19/56; A23L 2/60; A23L 27/36; C12N 9/0071; C12N 9/1051; C12N 9/1081; C12N 9/1085; C12N 9/90; C12N 15/81; C12Y 204/01; C12Y 204/99

(57) ABSTRACT

Methods for recombinant production of steviol glycoside and compositions containing steviol glycosides are provided by this invention.

### CROSS-REFERENCE TO RELATED APPLICATIONS

This application is a continuation of the U.S. application Ser. No. 18/052,477, filed on Nov. 3, 2022, which is a continuation of the U.S. application Ser. No. 16/799,541, filed on Feb. 24, 2020, and issued as U.S. Pat. No. 11,530,431 on Dec. 20, 2022, which is a continuation of the U.S. application Ser. No. 15/908,214, filed on Feb. 28, 2018, and issued as the U.S. Pat. No. 10,612,066 on Apr. 7, 2020, which is a divisional of the U.S. application Ser. No. 14/761,629, filed on Jul. 17, 2015, and issued as the U.S. Pat. No. 9,957,540 on May 1, 2018, which is the U.S. National Stage Application of International Application No. PCT/EP2014/052363, filed on Feb. 6, 2014, and claims the benefit of the U.S. Provisional Application No. 61/761,490, filed on Feb. 6, 2013 and the U.S. Provisional Application No. 61/886,442, filed on Oct. 3, 2013, the disclosures of each of which are explicitly incorporated by reference herein in their entirety.

### REFERENCE TO AN ELECTRONIC SEQUENCE LISTING

The instant application contains a Sequence Listing that has been submitted electronically and which is hereby incorporated by reference in its entirety. The Sequence Listing was created on Nov. 3, 2022, is named “13-1364-US-CON2.xml” and is 293,126 bytes in size.

### BACKGROUND OF THE INVENTION

### Field of the Invention

The invention disclosed herein relates generally to the field of recombinant production of steviol glycosides. Particularly, the invention provides methods for recombinant production of steviol glycoside and compositions containing steviol glycosides.

### Description of Related Art

Sweeteners are well known as ingredients used most commonly in the food, beverage, or confectionary industries. The sweetener can either be incorporated into a final food product during production or for stand-alone use, when appropriately diluted or as a tabletop sweetener. Sweeteners include natural sweeteners such as sucrose, high fructose corn syrup, molasses, maple syrup, and honey and artificial sweeteners such as aspartame, saccharine and sucralose. Stevia extract is a natural sweetener that can be isolated and extracted from a perennial shrub, Stevia rebaudiana. Stevia is commonly grown in South America and Asia for commercial production of Stevia extract. Stevia extract, purified to various degrees, is used commercially as a high intensity sweetener in foods and in blends or alone as a tabletop sweetener.

Extracts of the Stevia plant contain Rebaudiosides and other steviol glycosides that contribute to the sweet flavor, although the amount of each glycoside often varies among different production batches. Existing commercial products are predominantly Rebaudioside A with lesser amounts of other glycosides such as Rebaudioside C, D, and F. Stevia extracts can also contain contaminants such as plant-derived compounds that contribute to off-flavors or have other undesirable effects. These contaminants can be more or less problematic depending on the food system or application of choice. Potential contaminants include pigments, lipids, proteins, phenolics, saccharides, spathulenol and other sesquiterpenes, labdane diterpenes, monoterpenes, decanoic acid, 8,11,14-eicosatrienoic acid, 2-methyloctadecane, pentacosane, octacosane, tetracosane, octadecanol, stigmasterol, beta-sitosterol, alpha- and beta-amyrin, lupeol, beta-amryin acetate, pentacyclic triterpenes, centauredin, quercitin, epi-alpha-cadinol, carophyllenes and derivatives, beta-pinene, beta-sitosterol, and gibberellin.

As recovery and purification of steviol glycosides from the Stevia plant have proven to be labor intensive and inefficient, there remains a need for a recombinant production system that can produce high yields of desired steviol glycosides such as Rebaudioside D and Rebaudioside M with less plant-based contaminants, including but not limited to stevioside. Steviol glycoside-producing Saccharomyces cerevisiae strains as well as bio-conversion and biosynthesis in vitro are described in PCT Application Nos. PCT/US2012/050021 and PCT/US2011/038967, which are incorporated herein by reference in their entirety.

In nature, the Stevia uridine diphosphate dependent glycosyltransferase 76G1 (UGT76G1) catalyzes several glycosylation reactions on the steviol backbone, which leads to the production of steviol glycosides. Recently, it has been shown that UGT76G1 can convert 1,2-stevioside to Rebaudioside A and 1,2-bioside to Rebaudioside B (see Richman et al., 2005, The Plant Journal 41:56-67). Thus, there is a need in the art to identify reactions directed towards producing glycosylated Rebaudiosides by UGT76G1 or other UGT enzymes. Particularly, there is a need to explore or identify other reactions catalysed by UGT76G1 as well a need to increase UGT76G1's catalytic capability in order to produce higher yields of steviol glycosides such as Rebaudioside D and Rebaudioside M.

### SUMMARY OF THE INVENTION

It is against the above background that the present invention provides certain advantages and advancements over the prior art.

In particular, the invention is directed to biosynthesis of Rebaudioside D and Rebaudioside M and Rebaudioside D and Rebaudioside M preparations from genetically modified cells.

In particular embodiments, the invention is directed to Rebaudioside D and Rebaudioside M preparations from genetically modified cells having significantly improved biosynthesis rates and yields.

This disclosure relates to the production of steviol glycosides. In particular, this disclosure relates to the production of steviol glycosides including Rebaudioside M:
(2S,3R,4S,5R,6R)-5-hydroxy-6-(hydroxymethyl)-3,4-bis({[(2S,3R,4S,5S,6R)-3,4,5-trihydroxy-6-(hydroxymethyl)oxan-2-yl]oxy})oxan-2-yl(1R,5R,9S,13R)-13-{[(2S,3R,4S,5R,6R)-5-hydroxy-6-(hydroxymethyl)-3,4-bis({[(2S,3R,4S,5S,6R)-3,4,5-trihydroxy-6-(hydroxymethyl)oxan-2-yl]oxy})oxan-2-yl]oxy}-5,9-dimethyl-14-methylidenetetracyclo[11.2.1.0^{1,10}0.0^{4,9}]hexadecane-5-carboxylate
and Rebaudioside D:
4,5-dihydroxy-6-(hydroxymethyl)-3-{[3,4,5-trihydroxy-6-(hydroxymethyl)oxan-2-yl]oxy}oxan-2-yl 13-{[5-hydroxy-6-(hydroxymethyl)-3,4-bis({[3,4,5-trihydroxy-6-(hydroxymethyl) oxan-2-yl]oxy})oxan-2-yl]oxy}-5,9-dimethyl-14-methylidene tetracyclo[11.2.1.^{01,10}.0^{4,9}]hexadecane-5-carboxylate
   by means not limited to in recombinant hosts such as recombinant microorganisms, through bioconversion, and in vitro.

Thus, in one aspect, the disclosure provides a recombinant host, for example, a microorganism, comprising one or more biosynthetic genes, wherein the expression of one or more biosynthetic genes results in production of steviol glycosides including Rebaudioside M and Rebaudioside D.

In particular, expression of one or more uridine 5′-diphospho (UDP) glycosyl transferases described herein, such as EUGT11, UGT74G1, UGT76G1, UGT85C2, and UGT91D2, facilitate production and accumulation of Rebaudioside M or Rebaudioside D in recombinant hosts or certain in vitro systems.

Although this invention disclosed herein is not limited to specific advantages or functionality, the invention provides a composition comprising from about 1% to about 99% w/w of Rebaudioside M, wherein the composition has a reduced level of Stevia-derived contaminants relative to a Stevia extract, wherein at least one of said contaminants is a plant-derived compound. In certain instances, said plant-derived contaminating compound can, inter alia, contribute to off-flavors.

In some aspects, the composition comprising from about 1% to about 99% w/w of Rebaudioside M has less than 0.1% of Stevia-derived contaminants relative to a Stevia extract, wherein at least one of said contaminants is a plant-derived compound. In certain instances, said plant-derived contaminating compound can, inter alia, contribute to off-flavors.

The invention further provides a food product comprising the composition as described above.

In some aspects, the food product is a beverage or a beverage concentrate.

The invention further provides a recombinant host cell that expresses:
(a) a recombinant gene encoding a GGPPS;
(b) a recombinant gene encoding an ent-copalyl diphosphate synthase (CDPS) polypeptide;
(c) a recombinant gene encoding a kaurene oxidase (KO) polypeptide;
(d) a recombinant gene encoding a kaurene synthase (KS) polypeptide;
(e) a recombinant gene encoding a steviol synthase (KAH) polypeptide;
(f) a recombinant gene encoding a cytochrome P450 reductase (CPR) polypeptide;
(g) a recombinant gene encoding a UGT85C2 polypeptide;
(h) a recombinant gene encoding a UGT74G1 polypeptide;
(i) a recombinant gene encoding a UGT76G1 polypeptide;
(j) a recombinant gene encoding a UGT91d2 polypeptide; and
(k) a recombinant gene encoding a EUGT11 polypeptide;
wherein at least one of said genes is a recombinant gene and wherein the cell produces Rebaudioside D, Rebaudioside M, Rebaudioside Q, and/or Rebaudioside I.

The invention further provides a recombinant host cell comprising exogenous nucleic acids comprising:
(a) a recombinant gene encoding a GGPPS;
(b) a recombinant gene encoding an ent-copalyl diphosphate synthase (CDPS) polypeptide;
(c) a recombinant gene encoding a kaurene oxidase (KO) polypeptide;
(d) a recombinant gene encoding a kaurene synthase (KS) polypeptide;
(e) a recombinant gene encoding a steviol synthase (KAH) polypeptide;
(f) a recombinant gene encoding a cytochrome P450 reductase (CPR) polypeptide;
(g) a recombinant gene encoding a UGT85C2 polypeptide;
(h) a recombinant gene encoding a UGT74G1 polypeptide;
(i) a recombinant gene encoding a UGT76G1 polypeptide;
(j) a recombinant gene encoding a UGT91d2 polypeptide; and
(k) a recombinant gene encoding a EUGT11 polypeptide;
wherein the cell produces Rebaudioside D, Rebaudioside M, Rebaudioside Q, and/or Rebaudioside I.

The invention further provides a recombinant host cell that expresses a GGPPS, an ent-copalyl diphosphate synthase (CDPS) polypeptide, a kaurene oxidase (KO) polypeptide, a kaurene synthase (KS) polypeptide; a steviol synthase (KAH) polypeptide, a cytochrome P450 reductase (CPR) polypeptide, a UGT74G1 polypeptide, a UGT76G1 polypeptide, a UGT91d2 polypeptide, and a EUGT11 polypeptide, wherein at least one of said polypeptides is encoded by an exogenous or heterologous gene having been introduced into said cell;
wherein the cell produces a di-glycosylated steviol glycoside (13-hydroxy kaur-16-en-18-oic acid, [2-O-β-D-glucopyranosyl-β-D-glucopyranosyl]ester) or a tri-glycosylated steviol glycoside (13-hydroxy kaur-16-en-18-oic acid; [2-O-β-D-glucopyranosyl-3-O-β-D-glucopyranosyl-β-D-glucopyranosyl]ester).

In some embodiments, targeted production of individual Rebaudiosides can be accomplished by controlling the relative levels of UDP-glycosyl transferase activities (see FIG. 1).

In some aspects, targeted production of individual Rebaudiosides can be accomplished by differential copy numbers of the UGT-encoding genes (see FIG. 1) in the recombinant cell, differential promoter strengths, and/or by utilizing mutants with increased specificity/activity towards the product of interest. For example, low levels of Rebaudioside D, E, and M will be formed if EUGT11 is expressed at low levels in comparison to the other UGTs, which would favor Rebaudioside A formation. High levels of EUGT11 expression result in production of more 19-O 1,2 diglucoside that can serve as substrate for UGT76G1 to form Rebaudioside M. In certain advantageous embodiments, additional copies or mutant versions of UGT76G1 in recombinant cells of the invention can improve the rate of Rebaudioside M formation from Rebaudioside D.

In some embodiments, UGT76G1 catalyzes glycosylation of steviol and steviol glycosides at the 19-O position. Thus, in some embodiments, one or more of RebM, RebQ, RebI, di-glycosylated steviol glycoside (13-hydroxy kaur-16-en-18-oic acid, [2-O-β-D-glucopyranosyl-β-D-glucopyranosyl]ester), or tri-glycosylated steviol glycoside ((13-hydroxy kaur-16-en-18-oic acid; [2-O-β-D-glucopyranosyl-3-O-β-D-glucopyranosyl-β-D-glucopyranosyl]ester) are produced in a recombinant host expressing a recombinant gene encoding a UGT76G1 polypeptide, through bioconversion, or through catalysis by UGT76G1 in vitro. In some embodiments, UGT76G1 catalyzes the glycosylation of steviol and steviol glycosides at the 13-O position and preferentially glycosylates steviol glycoside substrates that are 1,2-di-glycosylated at the 13-O position or mono-glycosylated at the 13-O position. In some embodiments, UGT76G1 does not show a preference for the glycosylation state of the 19-0 position.

In some aspects, the GGPPS comprises Synechococcus sp. GGPPS set forth in SEQ ID NO:24.

In some aspects, the CDP polypeptide comprises a Z. mays CDPS polypeptide set forth in SEQ ID NO:13, wherein the polypeptide is lacking a chloroplast transit peptide.

In some aspects, the KO polypeptide comprises a KO polypeptide having 70% or greater identity to the amino acid sequence of the S. rebaudiana KO polypeptide set forth in SEQ ID NO:25.

In some aspects, the KS polypeptide comprises a KS polypeptide having 40% or greater identity to the amino acid sequence of the A. thaliana KS polypeptide set forth in SEQ ID NO:21.

In some aspects, the KAH polypeptide comprises a KAH polypeptide having 60% or greater identity to the S. rebaudiana KAH amino acid sequence set forth in SEQ ID NO:11.

In some aspects, the CPR polypeptide comprises a CPR polypeptide having 65% or greater identity to a S. rebaudiana CPR amino acid sequence set forth in SEQ ID NO:4, an A. thaliana CPR polypeptide of the amino acid sequence set forth in SEQ ID NO:9 or a combination thereof.

In some aspects, the UGT85C2 polypeptide comprises a UGT85C2 polypeptide having 55% or greater identity to the amino acid sequence set forth in SEQ ID NO:26.

In some aspects, the UGT74G1 polypeptide comprises a UGT74G1 polypeptide having 55% or greater identity to the amino acid sequence set forth in SEQ ID NO:19.

In some aspects, the UGT76G1 polypeptide comprises a UGT76G1 polypeptide having 50% or greater identity to the amino acid sequence set forth in SEQ ID NO:2.

In some aspects, the UGT91d2 polypeptide comprises a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15 or a combination thereof.

In some aspects, the EUGT11 polypeptide comprises a EUGT11 polypeptide having 65% or greater identity to the Os03g0702000 amino acid sequence set forth in SEQ ID NO:16.

In some aspects, the UGT76G1 polypeptide comprises one or more of the UGT76G1 polypeptide variants comprising: T55K, T55E, S56A, Y128S, Y128E, H155L, H155R, Q198R, S285R, S285T, S253W, S253G, T284R, T284G, S285G, K337E, K337P and L379V of SEQ ID NO:2.

In some aspects, the UGT76G1 polypeptide comprises one or more of the UGT76G1 polypeptide variants comprising: Q23G, Q23H, I26F, I26W, T146A, T146G, T146P, H155R, L257P, L257W, L257T, L257G, L257A, L257R, L257E, S283G and S283N of SEQ ID NO:2.

In some aspects, the recombinant host cell is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides the cell as disclosed herein that produces Rebaudioside D.

The invention further provides the cell as disclosed herein that produces Rebaudioside M, Rebaudioside Q or Rebaudioside I.

The invention further provides the cell as disclosed herein that produces the di-glycosylated steviol glycoside (13-hydroxy kaur-16-en-18-oic acid, [2-O-β-D-glucopyranosyl-β-D-glucopyranosyl]ester) or the tri-glycosylated steviol glycoside (13-hydroxy kaur-16-en-18-oic acid; [2-O-β-D-glucopyranosyl-3-O-β-D-glucopyranosyl-β-D-glucopyranosyl]ester).

In some aspects, the Rebaudioside D is produced in the cell as disclosed herein at a concentration of between about 1,000 mg/L and about 2,900 mg/L.

In some aspects, the Rebaudioside D and Rebaudioside M are produced in the cell as disclosed herein at a ratio of between about 1:1 to about 1.7:1.

In some aspects, the Rebaudioside M is produced in the cell as disclosed herein at a concentration of between about 600 mg/L and about 2,800 mg/L.

In some aspects, the Rebaudioside M and Rebaudioside D are produced in the cell as disclosed herein at a ratio of between about 0.6:1 to about 1.1:1.

The invention further provides a method of producing Rebaudioside D, Rebaudioside M, Rebaudioside Q, Rebaudioside I, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester), comprising:
(a) culturing a recombinant cell in a culture medium, under conditions wherein genes encoding a GGPPS; an ent-copalyl diphosphate synthase (CDPS) polypeptide; a kaurene oxidase (KO) polypeptide; a kaurene synthase (KS) polypeptide; a steviol synthase (KAH) polypeptide; a cytochrome P450 reductase (CPR) polypeptide; a UGT85C2 polypeptide; a UGT74G1 polypeptide; a UGT76G1 polypeptide; a UGT91d2 polypeptide; and a EUGT11 polypeptide are expressed, comprising inducing expression of said genes or constitutively expressing said genes; and
(b) synthesizing Rebaudioside D, Rebaudioside M, Rebaudioside Q, Rebaudioside 1, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester) in the cell; and optionally
(c) isolating Rebaudioside D, Rebaudioside M, Rebaudioside Q, Rebaudioside 1, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester).

In some aspects, Rebaudioside D is produced by a cell as disclosed herein.

In some aspects, Rebaudioside M, Rebaudioside Q or Rebaudioside I is produced by a cell as disclosed herein.

In some aspects, di-glycosylated steviol glycoside (13-hydroxy kaur-16-en-18-oic acid, [2-O-β-D-glucopyranosyl-β-D-glucopyranosyl]ester) or the tri-glycosylated steviol glycoside (13-hydroxy kaur-16-en-18-oic acid; [2-O-β-D-glucopyranosyl-3-O-β-D-glucopyranosyl-β-D-glucopyranosyl]ester) is produced by a cell as disclosed herein.

In some aspects, Rebaudioside D is produced at a concentration of between about 1,000 mg/L and about 2,900 mg/L.

In some aspects, Rebaudioside D and Rebaudioside M are produced at a ratio of between about 1:1 to about 1.7:1.

In some aspects, Rebaudioside M is produced at a concentration of between about 600 mg/L and about 2,800 mg/L.

In some aspects, Rebaudioside M and Rebaudioside D are produced at a ratio of between about 0.6:1 to about 1.1:1.

In some aspects, a cell for practicing the methods disclosed herein is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides methods for producing Rebaudioside D, Rebaudioside M, Rebaudioside Q, Rebaudioside I, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester) through in vitro bioconversion of plant-derived or synthetic steviol or steviol glycosides using one or more UGT polypeptides.

In some aspects, said methods for producing Rebaudioside D or Rebaudioside M comprise using at least one UGT polypeptide that is:
a UGT85C2 polypeptide comprising a UGT85C2 polypeptide having 55% or greater identity to the amino acid sequence set forth in SEQ ID NO:26;
a UGT74G1 polypeptide comprising a UGT74G1 polypeptide having 55% or greater identity to the amino acid sequence set forth in SEQ ID NO:19;
a UGT76G1 polypeptide comprising a UGT76G1 polypeptide having 50% or greater identity to the amino acid sequence set forth in SEQ ID NO:2;
a UGT91d2 polypeptide comprising a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15 or a combination thereof; or
a EUGT11 polypeptide comprising a EUGT11 polypeptide having 65% or greater identity to the Os03g0702000 amino acid sequence set forth in SEQ ID NO:16.

In some aspects, the steviol glycoside used for production of Rebaudioside D comprises stevioside, RebA, RebB, RebE or mixtures thereof.

In some aspects, the steviol glycoside used for production of Rebaudioside M comprises stevioside, RebA, RebB, RebE, RebD or mixtures thereof.

In some aspects, methods for producing Rebaudioside Q comprise using at least one UGT polypeptide that is:
a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; or a UGT76G1 polypeptide comprising a UGT76G1 polypeptide having 50% or greater identity to the amino acid sequence set forth in SEQ ID NO:2.

In some aspects, the steviol glycoside used for producing Rebaudioside Q comprises rubusoside, RebG or mixtures thereof.

In some aspects, methods for producing Rebaudioside I comprise using at least one UGT polypeptide that is:
a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; a UGT76G1 polypeptide comprising a UGT76G1 polypeptide having 50% or greater identity to the amino acid sequence set forth in SEQ ID NO:2; a UGT91d2 polypeptide comprising a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15; or a combination thereof.

In some aspects, the steviol glycoside used for producing Rebaudioside I comprises 1,2-stevioside, RebA, or mixtures thereof.

In some aspects, methods for producing di-glycosylated steviol glycoside (13-hydroxy kaur-16-en-18-oic acid, [2-O-β-D-glucopyranosyl-β-D-glucopyranosyl]ester) comprise using at least one or UGT polypeptide that is:
a UGT74G1 polypeptide comprising a UGT74G1 polypeptide having 55% or greater identity to the amino acid sequence set forth in SEQ ID NO:19; or a EUGT11 polypeptide comprising a EUGT11 polypeptide having 65% or greater identity to the Os03g0702000 amino acid sequence set forth in SEQ ID NO:16.

In some aspects, the steviol glycoside used for producing di-glycosylated steviol glycoside (13-hydroxy kaur-16-en-18-oic acid, [2-O-β-D-glucopyranosyl-β-D-glucopyranosyl]ester) comprises steviol-19-O-glucoside.

In some aspects, methods for producing tri-glycosylated steviol glycoside (13-hydroxy kaur-16-en-18-oic acid; [2-O-β-D-glucopyranosyl-3-O-β-D-glucopyranosyl-β-D-glucopyranosyl]ester) comprise using at least one UGT polypeptide that is:
a UGT76G1 polypeptide comprising a UGT76G1 polypeptide having 50% or greater identity to the amino acid sequence set forth in SEQ ID NO:2; a UGT74G1 polypeptide comprising a UGT74G1 polypeptide having 55% or greater identity to the amino acid sequence set forth in SEQ ID NO:19; or a EUGT11 polypeptide comprising a EUGT11 polypeptide having 65% or greater identity to the Os03g0702000 amino acid sequence set forth in SEQ ID NO:16.

In some aspects, the steviol glycoside used for producing tri-glycosylated steviol glycoside (13-hydroxy kaur-16-en-18-oic acid; [2-O-β-D-glucopyranosyl-3-O-β-D-glucopyranosyl-β-D-glucopyranosyl]ester) comprises di-glycosylated steviol glycoside (13-hydroxy kaur-16-en-18-oic acid, [2-O-β-D-glucopyranosyl-β-D-glucopyranosyl]ester), steviol-19-O-glucoside, or mixtures thereof.

In some aspects, bioconversion methods as disclosed herein comprise enzymatic bioconversion or whole cell bioconversion.

In some aspects, a cell for practicing the methods disclosed herein is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides a recombinant host cell comprising exogenous nucleic acids comprising:
(a) a recombinant gene encoding a GGPPS;
(b) a recombinant gene encoding an ent-copalyl diphosphate synthase (CDPS) polypeptide;
(c) a recombinant gene encoding a kaurene oxidase (KO) polypeptide;
(d) a recombinant gene encoding a kaurene synthase (KS) polypeptide;
(e) a recombinant gene encoding a steviol synthase (KAH) polypeptide;
(f) a recombinant gene encoding a cytochrome P450 reductase (CPR) polypeptide; and/or
(g) a one or more recombinant genes encoding a one or more UGT polypeptide;
wherein the cell produces Rebaudioside D, Rebaudioside M, Rebaudioside Q, or Rebaudioside I, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester).

In some aspects of said recombinant host cells, the GGPPS comprises Synechococcus sp. GGPPS set forth in SEQ ID NO:24; the CDP polypeptide comprises a Z. mays CDPS polypeptide set forth in SEQ ID NO:13, wherein the polypeptide is lacking a chloroplast transit peptide; the KO polypeptide comprises a KO polypeptide having 70% or greater identity to the amino acid sequence of the S. rebaudiana KO polypeptide set forth in SEQ ID NO:25; the KS polypeptide comprises a KS polypeptide having 40% or greater identity to the amino acid sequence of the A. thaliana KS polypeptide set forth in SEQ ID NO:21; the KAH polypeptide comprises a KAH polypeptide having 60% or greater identity to the S. rebaudiana KAH amino acid sequence set forth in SEQ ID NO:11; the CPR polypeptide comprises a CPR polypeptide having 65% or greater identity to a S. rebaudiana CPR amino acid sequence set forth in SEQ ID NO:4, an A. thaliana CPR polypeptide of the amino acid sequence set forth in SEQ ID NO:9 or a combination thereof.

In some aspects, the cell produces Rebaudioside D or Rebaudioside M, wherein the UGT polypeptide is at least one UGT polypeptide that is:
a UGT85C2 polypeptide comprising a UGT85C2 polypeptide having 55% or greater identity to the amino acid sequence set forth in SEQ ID NO:26;
a UGT74G1 polypeptide comprising a UGT74G1 polypeptide having 55% or greater identity to the amino acid sequence set forth in SEQ ID NO:19;
a UGT76G1 polypeptide comprising a UGT76G1 polypeptide having 50% or greater identity to the amino acid sequence set forth in SEQ ID NO:2;
a UGT91d2 polypeptide comprising a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15 or a combination thereof; or
a EUGT11 polypeptide comprising a EUGT11 polypeptide having 65% or greater identity to the Os03g0702000 amino acid sequence set forth in SEQ ID NO:16.

In some aspects, the cell produces Rebaudioside Q, wherein the UGT polypeptide is at least one UGT polypeptide that is:
a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; or a UGT76G1 polypeptide comprising a UGT76G1 polypeptide having 50% or greater identity to the amino acid sequence set forth in SEQ ID NO:2.

In some aspects, the cell produces Rebaudioside I, wherein the UGT polypeptide is at least one UGT polypeptide that is:
a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; a UGT76G1 polypeptide comprising a UGT76G1 polypeptide having 50% or greater identity to the amino acid sequence set forth in SEQ ID NO:2; a UGT91d2 polypeptide comprising a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15; or a combination thereof.

In some aspects, the cell produces di-glycosylated steviol glycoside (13-hydroxy kaur-16-en-18-oic acid, [2-O-β-D-glucopyranosyl-β-D-glucopyranosyl]ester), wherein the UGT polypeptide is at least one UGT polypeptide that is:
a UGT74G1 polypeptide comprising a UGT74G1 polypeptide having 55% or greater identity to the amino acid sequence set forth in SEQ ID NO:19; or a EUGT11 polypeptide comprising a EUGT11 polypeptide having 65% or greater identity to the Os03g0702000 amino acid sequence set forth in SEQ ID NO:16.

In some aspects, the cell produces tri-glycosylated steviol glycoside (13-hydroxy kaur-16-en-18-oic acid; [2-O-β-D-glucopyranosyl-3-O-β-D-glucopyranosyl-β-D-glucopyranosyl]ester), wherein the UGT polypeptide is at least one UGT polypeptide that is:
a UGT76G1 polypeptide comprising a UGT76G1 polypeptide having 50% or greater identity to the amino acid sequence set forth in SEQ ID NO:2; a UGT74G1 polypeptide comprising a UGT74G1 polypeptide having 55% or greater identity to the amino acid sequence set forth in SEQ ID NO:19; or a EUGT11 polypeptide comprising a EUGT11 polypeptide having 65% or greater identity to the Os03g0702000 amino acid sequence set forth in SEQ ID NO:16

In some aspects, the UGT76G1 polypeptide comprises one or more of the UGT76G1 polypeptide variants comprising: Q23G, Q23H, I26F, I26W, T146A, T146G, T146P, H155R, L257P, L257W, L257T, L257G, L257A, L257R, L257E, S283G and S283N of SEQ ID NO:2.

In some aspects, the UGT76G1 polypeptide comprises one or more of the UGT76G1 polypeptide variants comprising: T55K, T55E, S56A, Y128S, Y128E, H155L, H155R, Q198R, S285R, S285T, S253W, S253G, T284R, T284G, S285G, K337E, K337P and L379V of SEQ ID NO:2.

In some aspects, the recombinant host cell is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides methods for producing Rebaudioside D by fermentation using a recombinant cell as disclosed herein.

The invention further provides methods for producing Rebaudioside M by fermentation using a recombinant cell as disclosed herein.

The invention further provides methods for producing Rebaudioside Q by fermentation using a recombinant cell as disclosed herein.

The invention further provides methods for producing Rebaudioside I by fermentation using a recombinant cell as disclosed herein.

The invention further provides methods for producing di-glycosylated steviol glycoside (13-hydroxy kaur-16-en-18-oic acid, [2-O-β-D-glucopyranosyl-β-D-glucopyranosyl]ester) by fermentation using a recombinant cell as disclosed herein.

The invention further provides methods for producing tri-glycosylated steviol glycoside (13-hydroxy kaur-16-en-18-oic acid; [2-O-β-D-glucopyranosyl-3-O-β-D-glucopyranosyl-β-D-glucopyranosyl]ester) by fermentation using a recombinant cell as disclosed herein.

In some aspects, a cell for practicing the methods disclosed herein is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides in vitro methods for producing Rebaudioside D or Rebaudioside M, comprising:
(a) adding one or more of a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; a UGT76G1 polypeptide comprising a UGT76G1 polypeptide having 50% or greater identity to the amino acid sequence set forth in SEQ ID NO:2; a UGT91d2 polypeptide comprising a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15 or a combination thereof; a EUGT11 polypeptide comprising a EUGT11 polypeptide having 65% or greater identity to the Os03g0702000 amino acid sequence set forth in SEQ ID NO:16, and plant-derived or synthetic steviol or steviol glycosides to the reaction mixture; and
(b) synthesizing Rebaudioside D or Rebaudioside M in the reaction mixture; and optionally
(c) isolating Rebaudioside D or Rebaudioside M in the reaction mixture.

The invention further provides in vitro methods for producing Rebaudioside Q, comprising:
(a) adding one or more of a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; a UGT76G1 polypeptide comprising a UGT76G1 polypeptide having 50% or greater identity to the amino acid sequence set forth in SEQ ID NO:2, and plant-derived or synthetic steviol or steviol glycosides to the reaction mixture; and
(b) synthesizing Rebaudioside Q in the reaction mixture; and optionally
(c) isolating Rebaudioside Q in the reaction mixture.

The invention further provides in vitro methods for producing Rebaudioside I, comprising:
(a) adding one or more of a UGT85C2 polypeptide comprising a UGT85C2 polypeptide having 55% or greater identity to the amino acid sequence set forth in SEQ ID NO:26; a UGT74G1 polypeptide comprising a UGT74G1 polypeptide having 55% or greater identity to the amino acid sequence set forth in SEQ ID NO:19; a UGT76G1 polypeptide comprising a UGT76G1 polypeptide having 50% or greater identity to the amino acid sequence set forth in SEQ ID NO:2; a UGT91d2 polypeptide comprising a UGT91d2 polypeptide having 90% or greater identity to the amino acid sequence set forth in SEQ ID NO:26 or a functional homolog thereof, a UGT91d2e polypeptide having a substitution at residues 211 and 286 of SEQ ID NO:15 or a combination thereof, and plant-derived or synthetic steviol or steviol glycosides to the reaction mixture; and
(b) synthesizing Rebaudioside I in the reaction mixture; and optionally
(c) isolating Rebaudioside I in the reaction mixture.

The invention further provides in vitro methods for producing a di-glycosylated steviol glycoside (13-hydroxy kaur-16-en-18-oic acid, [2-O-β-D-glucopyranosyl-β-D-glucopyranosyl]ester), comprising:
(a) adding one or more of a a UGT74G1 polypeptide comprising a UGT74G1 polypeptide having 55% or greater identity to the amino acid sequence set forth in SEQ ID NO:19; a EUGT11 polypeptide comprising a EUGT11 polypeptide having 65% or greater identity to the Os03g0702000 amino acid sequence set forth in SEQ ID NO:16, and plant-derived or synthetic steviol or steviol glycosides to the reaction mixture; and
(b) synthesizing the di-glycosylated steviol glycoside (13-hydroxy kaur-16-en-18-oic acid, [2-O-β-D-glucopyranosyl-β-D-glucopyranosyl]ester) in the reaction mixture; and optionally (c) isolating di-glycosylated steviol glycoside (13-hydroxy kaur-16-en-18-oic acid, [2-O-β-D-glucopyranosyl-β-D-glucopyranosyl]ester) in the reaction mixture.

The invention further provides in vitro methods for producing a tri-glycosylated steviol glycoside (13-hydroxy kaur-16-en-18-oic acid; [2-O-β-D-glucopyranosyl-3-O-β-D-glucopyranosyl-β-D-glucopyranosyl]ester), comprising:
(a) adding one or more of a a UGT76G1 polypeptide comprising a UGT76G1 polypeptide having 50% or greater identity to the amino acid sequence set forth in SEQ ID NO:2; a UGT74G1 polypeptide comprising a UGT74G1 polypeptide having 55% or greater identity to the amino acid sequence set forth in SEQ ID NO:19; a EUGT11 polypeptide comprising a EUGT11 polypeptide having 65% or greater identity to the Os03g0702000 amino acid sequence set forth in SEQ ID NO:16, and plant-derived or synthetic steviol or steviol glycosides to the reaction mixture; and
(b) synthesizing tri-glycosylated steviol glycoside (13-hydroxy kaur-16-en-18-oic acid; [2-O-β-D-glucopyranosyl-3-O-β-D-glucopyranosyl-β-D-glucopyranosyl]ester) in the reaction mixture; and optionally
(c) isolating tri-glycosylated steviol glycoside (13-hydroxy kaur-16-en-18-oic acid; [2-O-β-D-glucopyranosyl-3-O-β-D-glucopyranosyl-β-D-glucopyranosyl]ester) in the reaction mixture.

In some aspects, the UGT76G1 polypeptide for producing Rebaudioside D comprises one or more of the UGT76G1 polypeptide variants selected from the group consisting of: Q23G, Q23H, I26F, I26W, T146A, T146G, T146P, H155R, L257P, L257W, L257T, L257G, L257A, L257R, L257E, S283G and S283N of SEQ ID NO:2.

In some aspects, the UGT76G1 polypeptide for producing Rebaudioside M, Rebaudioside Q, Rebaudioside 1, di-glycosylated steviol glycoside and tri-glycosylated steviol glycoside comprises one or more of the UGT76G1 polypeptide variants selected from the group consisting of: T55K, T55E, S56A, Y128S, Y128E, H155L, H155R, Q198R, S285R, S285T, S253W, S253G, T284R, T284G, S285G, K337E, K337P and L379V of SEQ ID NO:2.

In some aspects, the in vitro method disclosed is enzymatic in vitro method or whole cell in vitro method.

In some aspects, a cell for practicing the methods disclosed herein is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides Rebaudioside Q produced by the methods disclosed herein.

The invention further provides Rebaudioside I produced by the methods disclosed herein.

The invention further provides a di-glycosylated steviol glycoside (13-hydroxy kaur-16-en-18-oic acid, [2-O-β-D-glucopyranosyl-β-D-glucopyranosyl]ester) produced by the methods disclosed herein.

The invention further provides a tri-glycosylated steviol glycoside (13-hydroxy kaur-16-en-18-oic acid; [2-O-β-D-glucopyranosyl-3-O-β-D-glucopyranosyl-β-D-glucopyranosyl]ester) produced by the methods disclosed herein.

The invention further provides a UGT76G1 polypeptide for producing Rebaudioside M, Rebaudioside Q, Rebaudioside I, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester), wherein the UGT76G1 polypeptide comprises a UGT76G1 polypeptide of SEQ ID NO:2 or one or more of the UGT76G1 polypeptide variants comprising: T55K, T55E, S56A, Y128S, Y128E, H155L, H155R, Q198R, S285R, S285T, S253W, S253G, T284R, T284G, S285G, K337E, K337P and L379V of SEQ ID NO:2.

The invention further provides a UGT76G1 polypeptide for producing Rebaudioside D, wherein the UGT76G1 polypeptide comprises a UGT76G1 polypeptide of SEQ ID NO:2 or one or more of the UGT76G1 polypeptide variants comprising: Q23G, Q23H, I26F, I26W, T146A, T146G, T146P, H155R, L257P, L257W, L257T, L257G, L257A, L257R, L257E, S283G and S283N of SEQ ID NO:2.

The invention further provides recombinant host cell comprising a recombinant gene encoding a UGT76G1 polypeptide, wherein the UGT76G1 polypeptide comprises one or more of the UGT76G1 polypeptide variants comprising: T55K, T55E, S56A, Y128S, Y128E, H155L, H155R, Q198R, S285R, S285T, S253W, S253G, T284R, T284G, S285G, K337E, K337P and L379V of SEQ ID NO:2.

In some aspects, the recombinant host cell as disclosed herein produces Rebaudioside D.

The invention further provides a recombinant host cell comprising a recombinant gene encoding a UGT76G1 polypeptide, wherein the UGT76G1 polypeptide comprises one or more of the UGT76G1 polypeptide variants comprising: Q23G, Q23H, I26F, I26W, T146A, T146G, T146P, H155R, L257P, L257W, L257T, L257G, L257A, L257R, L257E, S283G and S283N of SEQ ID NO:2.

In some aspects, the recombinant host cell as disclosed herein produces Rebaudioside M, Rebaudioside Q, Rebaudioside I, di-glycosylated steviol glycoside (13-hydroxy kaur-16-en-18-oic acid, [2-O-β-D-glucopyranosyl-β-D-glucopyranosyl]ester) or tri-glycosylated steviol glycoside (13-hydroxy kaur-16-en-18-oic acid; [2-O-β-D-glucopyranosyl-3-O-β-D-glucopyranosyl-β-D-glucopyranosyl]ester).

In some aspects, the recombinant host cell is a yeast cell, a plant cell, a mammalian cell, an insect cell, a fungal cell or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous or Candida albicans species.

In some aspects, the yeast cell is a Saccharomycete.

In some aspects, the yeast cell is a cell from Saccharomyces cerevisiae species.

The invention further provides a composition comprising from about 1% to about 99% w/w of Rebaudioside D, wherein the composition has a reduced level of Stevia-derived contaminants relative to a Stevia extract. In certain instances, the at least one of said contaminants is a plant-derived compound that inter alia contributes to off-flavors in the steviol glycoside product.

In some aspects, the composition has less than 0.1% of Stevia-derived contaminants relative to a Stevia extract. In certain instances, the at least one of said contaminants is a plant-derived compound that inter alia contributes to off-flavors in the steviol glycoside product.

The invention further provides a food product comprising the composition as disclosed herein.

In some aspects, the food product is a beverage or a beverage concentrate.

Any of the hosts described herein can be a microorganism (e.g., a Saccharomycete such as Saccharomyces cerevisiae, or Escherichia coli), or a plant or plant cell (e.g., a Stevia such as a Stevia rebaudiana or Physcomitrella).

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows the steviol glycoside glycosylation reactions and the enzymes by which they are catalyzed.

FIG. 2 shows the chemical structure of Rebaudioside M (RebM).

FIG. 3 shows the chemical structure of Rebaudioside D (RebD).

FIG. 4 shows biochemical pathway for the production of steviol.

FIG. 5 is a representative chromatogram of Liquid Chromatography-Mass Spectrometry (LC-MS) analysis showing formation of a hexa-glycosylated steviol glycoside at 1.31 min retention time. The traces, from top to bottom, correspond to the m/z indicated in Table 12.

FIG. 6 is a schematic of the methods for isolating hexa-glycosylated steviol glycosides.

FIG. 7A is a representative chromatogram of the semi-purified hexa-glycosylated steviol glycoside after flash chromatography.

FIG. 7B is a representative mass spectra from a liquid chromatography-quadrupole time-of-flight (LC-QTOF) analysis of the semi-purified hexa-glycosylated steviol glycoside after flash chromatography.

FIG. 8A is a chromatogram indicating compounds produced by fermentation of yeast strain EFSC 3044.

FIG. 8B is the NMR structure of the indicated di-glycosylated steviol glycoside (13-hydroxy kaur-16-en-18-oic acid, [2-O-β-D-glucopyranosyl-□β-D-glucopyranosyl]ester), an analog of steviol-1,2-bioside. The IUPAC name for di-glycosylated steviol glycoside is (2S,3R,4S,5S,6R)-4,5-dihydroxy-6-(hydroxymethyl)-3-{[(2S,3R,4S,5S,6R)-3,4,5-trihydroxy-6-(hydroxymethyl)oxan-2-yl]oxy}oxan-2-yl(1R,4S,5R,9S,10R,13S)-13-hydroxy-5,9-dimethyl-14-methylidenetetracyclo[11.2.1.0\hat{ }{1,10}.0\hat{ }{4,9}]hexadecane-5-carboxylate.

FIG. 8C is the structure of the NMR structure of the indicated tri-glycosylated steviol glycoside (13-hydroxy kaur-16-en-18-oic acid; [2-O-β-D-glucopyranosyl-3-O-□β-D-glucopyranosyl-β-D-glucopyranosyl]ester, an isomer of RebB. The IUPAC name for tri-glycosylated steviol glycoside is (2S,3R,4S,5R,6R)-5-hydroxy-6-(hydroxymethyl)-3,4-bis({[(2S,3R,4S,5S,6R)-3,4,5-tri hydroxy-6-(hydroxymethyl)oxan-2-yl]oxy})oxan-2-yl (1R,4S,5R,9S,10R,13S)-13-hydroxy-5,9-dimethyl-14-methylidenetetracyclo[11.2.1.0\hat{ }{1,10}.0\hat{ }{4,9}]hexadecane-5-carboxylate.

FIG. 9 shows RebD production by the EFSC 3261 yeast strain. Four fermentations of the EFSC 3261 yeast strain in minimal medium (MM) are shown.

FIG. 10 shows RebD and RebM production by the EFSC 3297 yeast strain.

FIG. 11 shows RebD, RebM, and RebA production by the EFSC 3841 yeast strain.

FIG. 12 compares RebD/RebM produced with one or two copies of UGT76G1.

FIG. 13A shows the relative rates of consumption of RebD and production of RebM by UGT76G1.

FIG. 13B shows the relative rates of consumption of RebE and production of RebD and RebM by UGT76G1.

FIG. 14 shows the variance in the three homology models of UGT76G1.

FIG. 15A is a scatter-plot of production of RebD and RebM in 96 and 4×24 deep-well plates.

FIG. 15B is a box-plot of RebD and RebM production in 96 and 4×24 deep-well plates.

FIG. 15C is a box-plot of RebD/RebM production in 96 and 4×24 deep-well plates.

FIG. 16 shows all data points of the initial UGT76G1 site saturation screen with wild type production shown as black triangles.

FIG. 17 shows the top RebD and RebM producing colonies selected for further study.

FIG. 18 shows a rescreen of UGT76G1 RebD and RebM top producers (as shown in FIG. 17) run in triplicate showing the same trends as the initial screen.

FIG. 19A shows the relative rates of consumption of Rubusoside and production of 1,3-stevioside (RebG) and Rebaudioside Q (“RebQ”) by UGT76G1.

FIG. 19B shows the relative rates of consumption of 1,2-stevioside and production of RebA by UGT76G1.

FIG. 19C shows the relative rates of consumption of 1,2-bioside and production of RebB by UGT76G1.

FIG. 20 shows chromatograms of 1,2-stevioside and RebA with or without UGT76G1 peaks indicating production of RebI.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the term “and/or” is utilized to describe multiple components in combination or exclusive of one another. For example, “x, y, and/or z” can refer to “x” alone, “y” alone, “z” alone, “x, y, and z,” “(x and y) or z,” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to production of steviol glycosides, wherein one or more steviol glycosides selected from a group are produced. In some embodiments, “and/or” is used to refer to production of steviol glycosides, wherein one or more steviol glycosides are produced through one or more of the following steps: culturing a recombinant cell, synthesizing one or more steviol glycosides in a cell, and isolating one or more steviol glycosides.

Highly-glycosylated steviol glycosides can be present in trace amounts in the Stevia plant, but at levels so low that extraction from the plant is impractical for use of such glycosides in food and beverage systems. See, Hellfritsch et al., J. Agric. Food Chem. 60: 6782-6793 (2012); DuBois G E, Stephenson R A., J Med Chem. Jan; 28:93-98 (1985); and US Patent Publication 2011-0160311.

Typically, stevioside and Rebaudioside A are the primary compounds in commercially-produced Stevia extracts. Stevioside is reported to have a more bitter and less sweet taste than Rebaudioside A. The composition of Stevia extract can vary from lot to lot depending on the soil and climate in which the plants are grown. Depending upon the sourced plant, the climate conditions, and the extraction process, the amount of Rebaudioside A in commercial preparations is reported to vary from 20 to 97% of the total steviol glycoside content.

Other steviol glycosides are present in varying amounts in Stevia extracts. For example, Rebaudioside B is typically present at less than 1-2%, whereas Rebaudioside C can be present at levels as high as 7-15%. Rebaudioside D is typically present in levels of 2% or less, and Rebaudioside F is typically present in compositions at 3.5% or less of the total steviol glycosides. The amount of the minor steviol glycosides, including but not limited to Rebaudioside M, can affect the flavor profile of a Stevia extract.

In addition, Rebaudioside D and other higher glycosylated steviol glycosides are thought to be higher quality sweeteners than Rebaudioside A. As such, the recombinant hosts and methods described herein are particularly useful for producing steviol glycoside compositions having an increased amount of Rebaudioside D for use, for example, as a non-caloric sweetener with functional and sensory properties superior to those of many high-potency sweeteners.

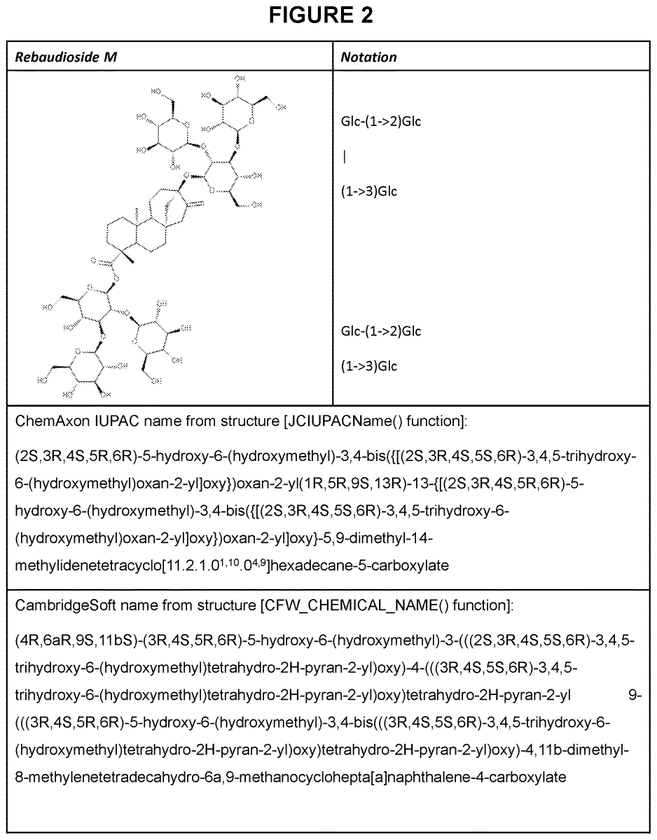

Rebaudioside M, a hexa-glycosylated steviol glycoside has been reported to be present in the Stevia plant and has an IUPAC name of (2S,3R,4S,5R,6R)-5-hydroxy-6-(hydroxymethyl)-3,4-bis({[(2S,3R,4S,5S,6R)-3,4,5-trihydroxy-6-(hydroxymethyl)oxan-2-yl]oxy})oxan-2-yl(1R,5R,9S,13R)-13-{[(2S,3R,4S,5R,6R)-5-hydroxy-6-(hydroxymethyl)-3,4-bis({[(2S,3R,4S,5S,6R)-3,4,5-tri hydroxy-6-(hydroxymethyl)oxan-2-yl]oxy})oxan-2-yl]oxy}-5,9-dimethyl-14-methylidenetetracyclo[11.2.1.0.^{01,10}.0^{4,9}]hexadecane-5-carboxylate. See, Ohta et al., MassBank record: FU000341, FU000342, FU000343 (2010) and Ohta et al (J. Applied Glycosides, 57(3):199-209, 2010). Rebaudioside M has been given a CAS number of 1220616-44-3. See FIG. 2 for the structure of Rebaudioside M.

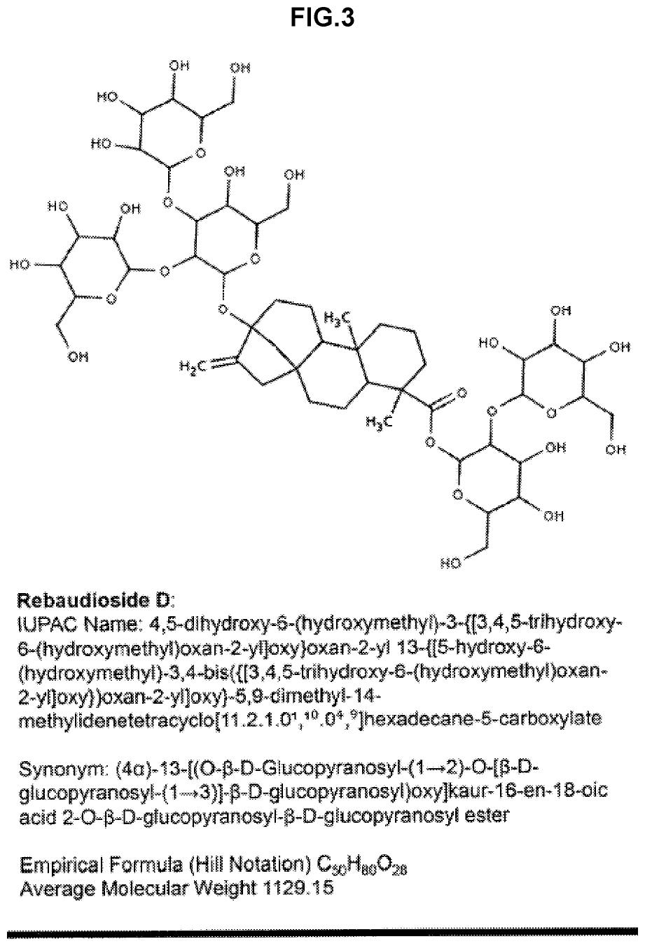

Rebaudioside D, a penta-glycosylated steviol glycoside, has also been reported to be present in the Stevia plant and has an IUPAC name of 4,5-dihydroxy-6-(hydroxymethyl)-3-{[3,4,5-trihydroxy-6-(hydroxymethyl)oxan-2-yl]oxy}oxan-2-yl 13-{[5-hydroxy-6-(hydroxymethyl)-3,4-bis({[3,4,5-trihydroxy-6-(hydroxymethyl)oxan-2-yl]oxy})oxan-2-yl]oxy}-5,9-dimethyl-14-methylidenetetracyclo[11.2.1.^{01,10}0.0^{4,9}]hexadecane-5-carboxylate. Rebaudioside D has been given a CAS number of 64849-39-4. See FIG. 3 for the structure of Rebaudioside D.

Provided herein are recombinant hosts such as microorganisms that express polypeptides useful for de novo biosynthesis of Rebaudioside M or Rebaurdioside D. Hosts described herein express one or more uridine 5′-diphospho (UDP) glycosyl transferases suitable for producing steviol glycosides. Expression of these biosynthetic polypeptides in various microbial systems allows steviol glycosides to be produced in a consistent, reproducible manner from energy and carbon sources such as sugars, glycerol, CO₂, H₂, and sunlight. The proportion of each steviol glycoside produced by a recombinant host can be tailored by incorporating preselected biosynthetic enzymes into the hosts and expressing them at appropriate levels, to produce a sweetener composition with a consistent taste profile. Furthermore, the concentrations of steviol glycosides produced by recombinant hosts are expected to be higher than the levels of steviol glycosides produced in the Stevia plant, which improves the efficiency of the downstream purification. Such sweetener compositions advantageously contain little or no plant based contaminants, relative to the amount of contaminants present in Stevia extracts.

The practice of the methods and recombinant host cells as disclosed are provided wherein at least one of the genes encoding a UGT85C2 polypeptide; a UGT74G1 polypeptide; a UGT76G1 polypeptide; or a UGT91d2 polypeptide is a recombinant gene, the particular recombinant gene(s) depending on the species or strain selected for use. Additional genes or biosynthetic modules can be included in order to increase steviol glycoside yield, improve efficiency with which energy and carbon sources are converted to steviol and its glycosides, and/or to enhance productivity from the cell culture. As used herein, “biosynthetic modules” refer to a collection of genes that are part of a common biosynthetic pathway and thus are often co-expressed in a recombinant organism. As used herein, such additional biosynthetic modules include genes involved in the synthesis of the terpenoid precursors, isopentenyl diphosphate and dimethylallyl diphosphate. Additional biosynthetic modules include terpene synthase and terpene cyclase genes, such as genes encoding geranylgeranyl diphosphate synthase and copalyl diphosphate synthase; these genes can be endogenous genes or recombinant genes.

### I. Steviol and Steviol Glycoside Biosynthesis Polypeptides

### A. Steviol Biosynthesis Polypeptides

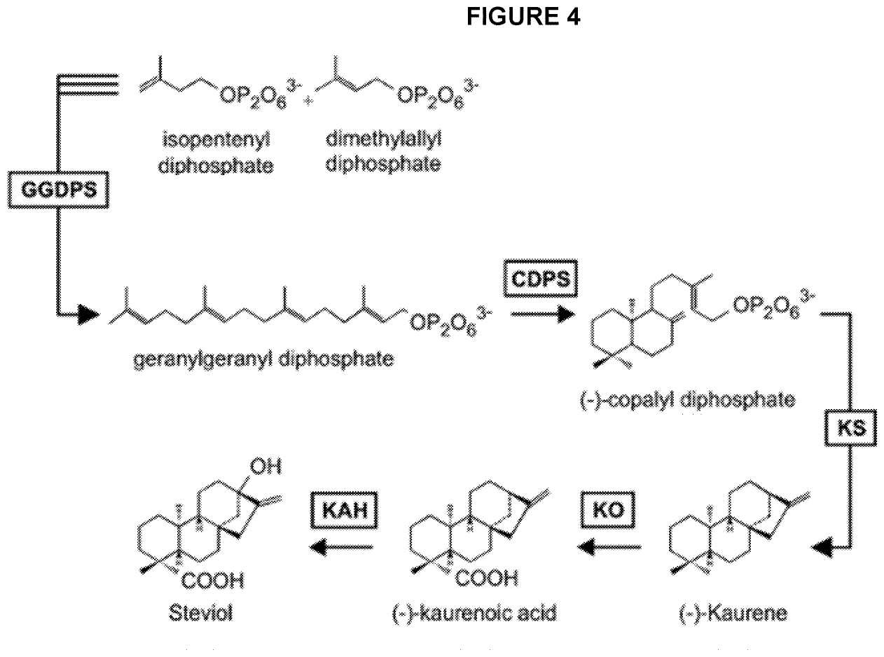

The biochemical pathway to produce steviol involves formation of the precursor, geranylgeranyl diphosphate (catalyzed by GGDPS), cyclization to (−) copalyl diphosphate (catalyzed by CDPS), followed by formation of (−)-kaurene (catalyzed by KS), followed by oxidation (catalyzed by KO), and hydroxylation (catalyzed by KAH) to form steviol. See FIG. 4. Thus, conversion of geranylgeranyl diphosphate to steviol in a recombinant microorganism involves the expression of a gene encoding a kaurene synthase (KS), a gene encoding a kaurene oxidase (KO), and a gene encoding a steviol synthetase (KAH). Steviol synthetase also is known as kaurenoic acid 13-hydroxylase.

Suitable KS polypeptides are known. For example, suitable KS enzymes include those made by Stevia rebaudiana, Zea mays, Populus trichocarpa, and Arabidopsis thaliana. See, Table 2 and PCT Application Nos. PCT/US2012/050021 and PCT/US2011/038967, which are incorporated herein by reference in their entirety. The nucleotide sequence encoding the A. thaliana KS polypeptide is set forth in SEQ ID NO:6, and the amino acid sequence of the A. thaliana KS polypeptide is set forth in SEQ ID NO:21.

TABLE 2
Kaurene synthase (KS) clones.

| Enzyme Source | | Accession | Construct | |
|---|---|---|---|---|
| Organism | gi Number | Number | Name | Length (nts) |
| Stevia | 4959241 | AAD34295 | MM-12 | 2355 |
| rebaudiana | | (amino acid SEQ | | (nt SEQ ID NO: 103) |
| | | ID NO: 27) | | |
| Stevia | 4959239 | AAD34294 | MM-13 | 2355 |
| rebaudiana | | (amino acid SEQ | | (nt SEQ ID NO: 104) |
| | | ID NO: 28) | | |
| Zea mays | 162458963 | NP_001105097 | MM-14 | 1773 |
| | | (amino acid SEQ | | (nt SEQ ID NO: 105) |
| | | ID NO: 29) | | |
| Populus | 224098838 | XP_002311286 | MM-15 | 2232 |
| trichocarpa | | (amino acid SEQ | | (nt SEQ ID NO: 106) |
| | | ID NO: 30) | | |
| Arabidopsis | 3056724 | AF034774 | EV-70 | 2358 |
| thaliana | | (amino acid SEQ | | (nt SEQ ID NO: 31) |
| | | ID NO: 32) | | |

Suitable KO polypeptides are known. For example, suitable KO enzymes include those made by Stevia rebaudiana, Arabidopsis thaliana, Gibberella fujikoroi and Trametes versicolor. See, e.g., Table 3 and POT Application Nos. PCT/US2012/050021 and PCT/US2011/038967, which are incorporated herein by reference in their entirety.

TABLE 3
Kaurene oxidase (KO) clones.

| Enzyme Source | | Accession | Construct | |
|---|---|---|---|---|
| Organism | gi Number | Number | Name | Length (nts) |
| Stevia | 76446107 | ABA42921 | MM-18 | 1542 |
| rebaudiana | | (amino acid SEQ | | (nt SEQ ID NO: 107) |
| | | ID NO: 33) | | |
| Arabidopsis | 3342249 | AAC39505 | MM-19 | 1530 |
| thaliana | | (amino acid SEQ | | (nt SEQ ID NO: 108) |
| | | ID NO: 34) | | |
| Gibberella | 4127832 | CAA76703 | MM-20 | 1578 |
| fujikoroi | | (amino acid SEQ | | (nt SEQ ID NO: 109) |
| | | ID NO: 35) | | |
| Trametes | 14278967 | BAB59027 | MM-21 | 1500 |
| versicolor | | (amino acid SEQ | | (nt SEQ ID NO: 110) |
| | | ID NO: 36) | | |

Suitable KAH polypeptides are known. For example, suitable KAH enzymes include those made by Stevia rebaudiana, Arabidopsis thaliana, Vitis vinifera and Medicago trunculata. See, e.g., Table 4, POT Application Nos. PCT/US2012/050021 and PCT/US2011/038967, U.S. Patent Publication Nos. 2008/0271205 and 2008/0064063, and Genbank Accession No. gi 189098312 (SEQ ID NO: 37) and GenBank Accession ABD60225; GI:89242710 (SEQ ID NO: 38), which are incorporated herein by reference in their entirety. The steviol synthetase from A. thaliana is classified as a CYP714A2.

TABLE 4
Steviol synthetase (KAH) clones.

| Enzyme Source | | Accession | Plasmid | Construct | |
|---|---|---|---|---|---|
| Organism | gi Number | Number | Name | Name | Length (nts) |
| Stevia | * | (amino acid SEQ | pMUS35 | MM-22 | 1578 |
| rebaudiana | | ID NO: 43) | | | (nt SEQ ID NO: 111) |
| Stevia | 189418962 | ACD93722 | pMUS36 | MM-23 | 1431 |
| rebaudiana | | (amino acid SEQ | | | (nt SEQ ID NO: 112) |
| | | ID NO: 39) | | | |
| Arabidopsis | 15238644 | NP_197872 | pMUS37 | MM-24 | 1578 |
| thaliana | | (amino acid SEQ | | | (nt SEQ ID NO: 113) |
| | | ID NO: 40) | | | |
| Vitis vinifera | 225458454 | XP_002282091 | pMUS38 | MM-25 | 1590 |
| | | (amino acid SEQ | | | (nt SEQ ID NO: 114) |
| | | ID NO: 41) | | | |
| Medicago | 84514135 | ABC59076 | pMUS39 | MM-26 | 1440 |
| trunculata | | (amino acid SEQ | | | (nt SEQ ID NO: 115) |
| | | ID NO: 42) | | | |

* = Sequence is identified with sequence identifier number 2 as shown in U.S. Patent Publication No. 2008-0064063.

In addition, a KAH polypeptide from Stevia rebaudiana that was identified as described in POT Application No. PCT/US2012/050021, which is incorporated herein by reference in its entirety, is particularly useful in a recombinant host. The nucleotide sequence encoding the S. rebaudiana KAH (SrKAHe1) is set forth in SEQ ID NO:91. A nucleotide sequence encoding the S. rebaudiana KAH that has been codon-optimized for expression in yeast is set forth in SEQ ID NO:8, and the amino acid sequence of the S. rebaudiana KAH polypeptide is set forth in SEQ ID NO:11. When expressed in S. cerevisiae, the S. rebaudiana KAH (SEQ ID NO:11) shows significantly higher steviol synthase activity as compared to the A. thaliana ent-kaurenoic acid hydroxylase described by Yamaguchi et al. (U.S. Patent Publication No. 2008/0271205 A1) and other S. rebaudiana KAH enzymes described in U.S. Patent Publication No. 2008/0064063 as well as the protein sequence deposited in GenBank as ACD93722. The S. rebaudiana KAH polypeptide (SEQ ID NO:11) has less than 20% identity to the KAH from U.S. Patent Publication No. 2008/0271205 and less than 35% identity to the KAH from U.S. Patent Publication No. 2008/0064063.

For example, the steviol synthase encoded by SrKAHe1 is activated by the S. cerevisiae CPR encoded by gene NCP1 (YHRO42W). Greater activation levels of the steviol synthase encoded by SrKAHe1 is observed when the A. thaliana CPR encoded by the gene ATR2 (SEQ ID NO:10) and the S. rebaudiana CPR encoded by the gene CPR8 (SEQ ID NO:5) are co-expressed. The amino acid sequence of the A. thaliana ATR2 is set forth in SEQ ID NO:9, and the amino acid sequence for S. rebaudiana CPR8 polypeptides is set forth in SEQ ID NO:4. In some embodiments, a recombinant microorganism contains a recombinant gene encoding a KO, KS, and a KAH polypeptide. Such microorganisms also typically contain a recombinant gene encoding a cytochrome P450 reductase (CPR) polypeptide, since certain combinations of KO and/or KAH polypeptides require expression of an exogenous CPR polypeptide. In particular, the activity of a KO and/or a KAH polypeptide of plant origin can be significantly increased by the inclusion of a recombinant gene encoding an exogenous CPR polypeptide. Suitable CPR polypeptides are known. For example, suitable CPR enzymes include those made by S. rebaudiana and A. thaliana. See, e.g., Table 5 and PCT Application Nos. PCT/US2012/050021 and PCT/US2011/038967, which are incorporated herein by reference in their entirety.

TABLE 5
Cytochrome P450 reductase (CPR) Clones.

| Enzyme Source | | Accession | Plasmid | Construct | |
|---|---|---|---|---|---|
| Organism | gi Number | Number | Name | Name | Length (nts) |
| Stevia | 93211213 | ABB88839 | pMUS40 | MM-27 | 2133 |
| rebaudiana | | (amino acid SEQ | | | (nt SEQ ID NO: 116) |
| | | ID NO: 44) | | | |
| Arabidopsis | 15233853 | NP_194183 | pMUS41 | MM-28 | 2079 |
| thaliana | | (amino acid SEQ | | | (nt SEQ ID NO: 117) |
| | | ID NO: 45) | | | |
| Giberella | 32562989 | CAE09055 | pMUS42 | MM-29 | 2142 |
| fujikuroi | | (amino acid SEQ | | | (nt SEQ ID NO: 118) |
| | | ID NO: 46) | | | |

The yeast gene DPP1 and/or the yeast gene LPP1 can reduce the yield of steviol by converting the GGPP and FPP precursors by these enzymes. These genes can be disrupted or deleted such that the degradation of farnesyl pyrophosphate (FPP) to farnesol is reduced and the degradation of geranylgeranylpyrophosphate (GGPP) to geranylgeraniol (GGOH) is reduced. Alternatively, the promoter or enhancer elements of an endogenous gene encoding a phosphatase can be altered such that the expression of their encoded proteins is altered. Homologous recombination can be used to disrupt an endogenous gene. For example, a “gene replacement” vector can be constructed in such a way to include a selectable marker gene. The selectable marker gene can be operably linked, at both 5′ and 3′ end, to portions of the gene of sufficient length to mediate homologous recombination using methods known to those skilled in the art.

A selectable marker can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, Southern blot analysis. Subsequent to its use in selection, a selectable marker can be removed from the genome of the host cell by, e.g., Cre-IoxP systems (see, e.g., Gossen et al., 2002, Ann. Rev. Genetics 36:153-173 and U.S. Application Publication No. 20060014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

An “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

Expression in a recombinant microorganism of these genes can result in the conversion of geranylgeranyl diphosphate to steviol.

### B. Steviol Glycoside Biosynthesis Polypeptides

Recombinant host cells are described herein that can convert steviol to a steviol glycoside. Such hosts (e.g., microorganisms) contains genes encoding one or more UDP Glycosyl Transferases, also known as UGTs. UGTs transfer a monosaccharide unit from an activated nucleotide sugar to an acceptor moiety, in this case, an —OH or —COOH moiety on steviol or steviol derivative or an —OH moiety on a glucose already attached to the steviol backbone. UGTs have been classified into families and subfamilies based on sequence homology. See Li et al., 2001, J. Biol. Chem. 276:4338-4343.

### Rubusoside Biosynthesis Polypeptides

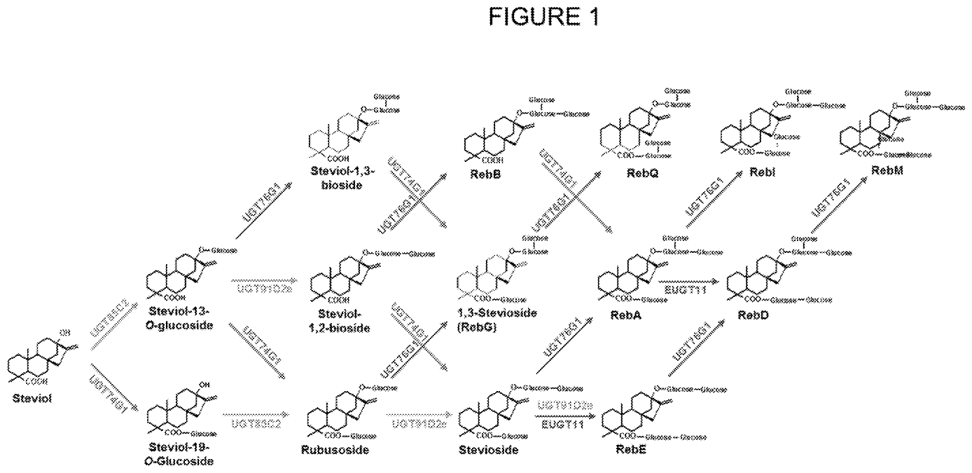

Biosynthesis of rubusoside involves glycosylation of the 13-OH and the 19-COOH of steviol. See FIG. 1. Conversion of steviol to rubusoside in a recombinant host such as a microorganism can be accomplished by expression of gene(s) encoding UGTs 85C2 and 74G1, which transfer a glucose unit to the 13-OH or the 19-COOH, respectively, of steviol.

A suitable UGT85C2 functions as a uridine 5′-diphosphoglucosyl:steviol 13-OH transferase, and a uridine 5′-diphosphoglucosyl:steviol-19-O-glucoside 13-OH transferase. Exemplary reactions for UGT85C2 include conversion of steviol and UDP-glucose to Steviol-13-O-glucoside or conversion of Steviol-19-O-glucoside and UDP-glucose to Rubusoside. See FIG. 1. Functional UGT85C2 polypeptides also can catalyze glucosyl transferase reactions that utilize steviol glycoside substrates other than steviol and steviol-19-O-glucoside.

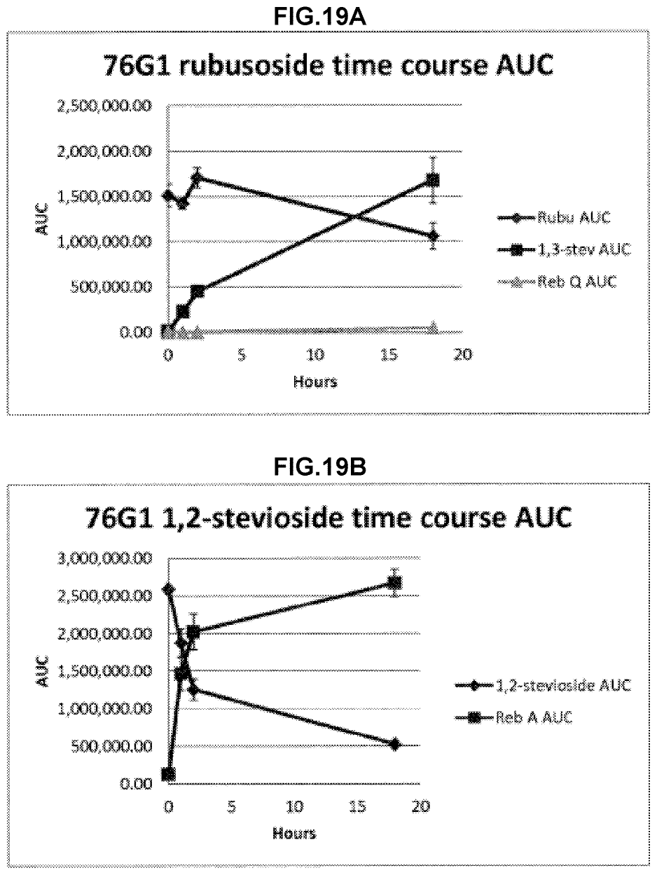

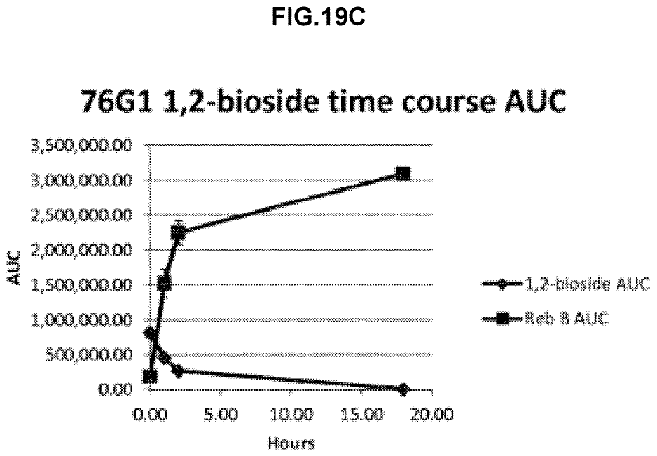

A suitable UGT74G1 polypeptide functions as a uridine 5′-diphospho glucosyl: steviol 19-COOH transferase and a uridine 5′-diphospho glucosyl: steviol-13-O-glucoside 19-COOH transferase. Exemplary reactions of 74G1 include conversion of steviol to steviol-19-O-glucoside and conversion of steviol-13-O-glucoside to Rubusoside. See FIG. 19 for these and other non-limiting examples of UGT74G1 reactions. Functional UGT74G1 polypeptides also can catalyze glycosyl transferase reactions that utilize steviol glycoside substrates other than steviol and steviol-13-O-glucoside, or that transfer sugar moieties from donors other than uridine diphosphate glucose.

A recombinant microorganism expressing a functional UGT74G1 and a functional UGT85C2 can make rubusoside and both steviol monosides (i.e., steviol 13-O-monoglucoside and steviol 19-O-monoglucoside) when steviol is used as a feedstock in the medium. Typically, however, genes encoding UGT74G1 and UGT85C2 are recombinant genes that have been transformed into a host (e.g., microorganism) that does not naturally possess them.

As used herein, the term “recombinant host” is intended to refer to (including but not limited to) a host cell, the genome of which has been augmented by at least one incorporated DNA sequence; extrachromosomal examples, like plasmids in bacteria and episomes comprising the 2-micron circle in yeast. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through the stable introduction of one or more recombinant genes. Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms, mammalian cells, insect cells, fungal cells, plant cells, and plants.

The term “recombinant gene” refers to a gene or DNA sequence that is introduced into a recipient host, regardless of whether the same or a similar gene or DNA sequence can already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified (including but not limited to regulated or inducible) expression of the gene product of that DNA.

Suitable UGT74G1 and UGT85C2 polypeptides include those made by S. rebaudiana. Genes encoding functional UGT74G1 and UGT85C2 polypeptides from Stevia are reported in Richman et al., 2005, Plant J. 41: 56-67. Amino acid sequences of S. rebaudiana UGT74G1 (SEQ ID NO: 19) and UGT85C2 (SEQ ID NO: 26) polypeptides are set forth in SEQ ID NOs: 1 and 3, respectively, of PCT Application No. PCT/US2012/050021, as are nucleotide sequences that encode UGT74G1 and UGT85C2 that have been optimized for expression in yeast and DNA 2.0 codon-optimized sequence for UGTs 85C2, 91D2e, 74G1 and 76G1. The Gene Art codon optimized nucleotide sequence encoding a S. rebaudiana UGT85C2 is set forth in SEQ ID NO:3. See also, the UGT85C2 and UGT74G1 variants described below in the “Functional Homolog” section. For example, a UGT85C2 polypeptide can contain substitutions at any one of the positions 65, 71, 270, 289, and 389 (e.g., A65S, E71Q, T270M, Q289H, and A389V) or a combination thereof.

In some embodiments, the recombinant host is a microorganism. Recombinant microorganism can be grown on media containing steviol in order to produce rubusoside. In other embodiments, however, the recombinant microorganism expresses genes involved in steviol biosynthesis, e.g., a CDPS gene, a KS gene, a KO gene, and a KAH gene. Suitable CDPS polypeptides are known. For example, suitable CDPS enzymes include those made by S. rebaudiana, Streptomyces clavuligerus, Bradyrhizobium japonicum, Zea mays, and Arabidopsis sp. See, e.g., Table 6 and PCT Application Nos. PCT/US2012/050021 and PCT/US2011/038967, which are incorporated herein by reference in their entirety.

In some embodiments, CDPS polypeptides that lack a chloroplast transit peptide at the amino terminus of the unmodified polypeptide can be used. For example, the first 150 nucleotides from the 5′ end of the Zea mays CDPS coding sequence (SEQ ID NO:12) can be removed, the truncated nucleotide sequence is shown in SEQ ID NO:133. Doing so removes the amino terminal 50 residues of the amino acid sequence shown in SEQ ID NO:13, which encode a chloroplast transit peptide; the truncated amino acid sequence is shown in SEQ ID NO:134. The truncated CDPS gene can be fitted with a new ATG translation start site and operably linked to a promoter, typically a constitutive or highly expressing promoter. When a plurality of copies, including but not limited to, one copy, two copies or three copies of the truncated coding sequence are introduced into a microorganism, expression of the CDPS polypeptide from the promoter results in an increased carbon flux towards ent-kaurene biosynthesis.

TABLE 6
CDPS Clones.

| Enzyme Source | | Accession | Plasmid | Construct | |
|---|---|---|---|---|---|
| Organism | gi Number | Number | Name | Name | Length (nts) |
| Stevia | 2642661 | AAB87091 | pMUS22 | MM-9 | 2364 |
| rebaudiana | | (amino acid SEQ | | | (nt SEQ ID NO: 119) |
| | | ID NO: 48) | | | |
| Streptomyces | 197705855 | EDY51667 | pMUS23 | MM-10 | 1584 |
| clavuligerus | | (amino acid SEQ | | | (nt SEQ ID NO: 120) |
| | | ID NO: 49) | | | |
| Bradyrhizobium | 529968 | AAC28895.1 | pMUS24 | MM-11 | 1551 |
| japonicum | | (amino acid SEQ | | | (nt SEQ ID NO: 121) |
| | | ID NO: 50) | | | |
| Zea mays | 50082774 | AY562490 | | EV65 | 2484 |
| | | (amino acid SEQ | | | (nt SEQ ID NO: 51) |
| | | ID NO: 52) | | | |
| Arabidopsis | 18412041 | NM_116512 | | EV64 | 2409 |
| thaliana | | (SEQ ID NO: 54) | | | (nt SEQ ID NO: 53) |

CDPS-KS bifunctional proteins also can be used. Nucleotide sequences encoding the COPS-KS bifunctional enzymes shown in Table 7 were modified for expression in yeast (see POT Application No. PCT/US2012/050021, which is incorporated herein by reference in its entirety). A bifunctional enzyme from Gibberella fujikuroi also can be used.

TABLE 7
CDPS-KS Clones.

| Enzyme Source | | Accession | Construct | |
|---|---|---|---|---|
| Organism | gi Number | Number | Name | Length (nts) |
| Phomopsis | 186704306 | BAG30962 | MM-16 | 2952 |
| amygdali | | (amino acid SEQ | | (nt SEQ ID NO: 122) |
| | | ID NO: 55) | | |
| Physcomitrella | 146325986 | BAF61135 | MM-17 | 2646 |
| patens | | (amino acid SEQ | | (nt SEQ ID NO: 123) |
| | | ID NO: 56) | | |
| Gibberella | 62900107 | Q9UVY5.1 | | 2859 |
| fujikuroi | | (amino acid SEQ | | (nt SEQ ID NO: 124) |
| | | ID NO: 57) | | |

Thus, a microorganism containing a COPS gene, a KS gene, a KO gene and a KAH gene in addition to a UGT74G1 and a UGT85C2 gene is capable of producing both steviol monosides and rubusoside without the necessity for using steviol as a feedstock.

In some embodiments, the recombinant microorganism further expresses a recombinant gene encoding a geranylgeranyl diphosphate synthase (GGPPS). Suitable GGPPS polypeptides are known. For example, suitable GGPPS enzymes include those made by S. rebaudiana, Gibberella fujikuroi, Mus musculus, Thalassiosira pseudonana, Streptomyces clavuligerus, Sulfulobus acidocaldarius, Synechococcus sp. and A. thaliana. See, Table 8 and PCT Application Nos. PCT/US2012/050021 and PCT/US2011/038967, which are incorporated herein by reference in their entirety.

TABLE 8
GGPPS Clones.

| Enzyme Source | | Accession | Plasmid | Construct | |
|---|---|---|---|---|---|
| Organism | gi Number | Number | Name | Name | Length (nts) |
| Stevia | 90289577 | ABD92926 | pMUS14 | MM-1 | 1086 |
| rebaudiana | | (amino acid SEQ | | | (nt SEQ ID NO: 125) |
| | | ID NO: 58) | | | |
| Gibberella | 3549881 | CAA75568 | pMUS15 | MM-2 | 1029 |
| fujikuroi | | (amino acid SEQ | | | (nt SEQ ID NO: 126) |
| | | ID NO: 59) | | | |
| Mus musculus | 47124116 | AAH69913 | pMUS16 | MM-3 | 903 |
| | | (amino acid SEQ | | | (nt SEQ ID NO: 127) |
| | | ID NO: 60) | | | |
| Thalassiosira | 223997332 | XP_002288339 | pMUS17 | MM-4 | 1020 |
| pseudonana | | (amino acid SEQ | | | (nt SEQ ID NO: 128) |
| | | ID NO: 61) | | | |
| Streptomyces | 254389342 | ZP_05004570 | pMUS18 | MM-5 | 1068 |
| clavuligerus | | (amino acid SEQ | | | (nt SEQ ID NO: 129) |
| | | ID NO: 62) | | | |
| Sulfulobus | 506371 | BAA43200 | pMUS19 | MM-6 | 993 |
| acidocaldarius | | (amino acid SEQ | | | (nt SEQ ID NO: 130) |
| | | ID NO: 63) | | | |
| Synechococcus | 86553638 | ABC98596 | pMUS20 | MM-7 | 894 |
| sp. | | (amino acid SEQ | | | (nt SEQ ID NO: 131) |
| | | ID NO: 64) | | | |
| Arabidopsis | 15234534 | NP_195399 | pMUS21 | MM-8 | 1113 |
| thaliana | | (amino acid SEQ | | | (nt SEQ ID NO: 132) |
| | | ID NO: 63) | | | |

In some aspects, the KAH gene encoding the KAH polypeptide set forth in SEQ ID NO: 11, comprising a recombinant cell of the invention is overexpressed. In some aspects, the KAH gene can be present in (including but not limited to) one, two or three copies. In some aspects, the KS gene encoding the KS polypeptide, set forth in SEQ ID NO:21, comprising a recombinant cell of the invention is overexpressed. In some aspects, the KS gene can be present in (including but not limited to) one, two or three copies.

In some embodiments, the recombinant microorganism further can express recombinant genes involved in diterpene biosynthesis or production of terpenoid precursors, e.g., genes in the methylerythritol 4-phosphate (MEP) pathway or genes in the mevalonate (MEV) pathway discussed below, have reduced phosphatase activity, and/or express a sucrose synthase (SUS) as discussed herein.

### Rebaudioside A, Rebaudioside D, and Rebaudioside E Biosynthesis Polypeptides

Biosynthesis of Rebaudioside A involves glucosylation of the aglycone steviol. Specifically, Rebaudioside A can be formed by glucosylation of the 13-OH of steviol which forms the 13-O-steviolmonoside, glucosylation of the C-2′ of the 13-O-glucose of steviolmonoside which forms steviol-1,2-bioside, glucosylation of the C-19 carboxyl of steviol-1,2-bioside which forms stevioside, and glucosylation of the C-3′ of the C-13-O-glucose of stevioside to produce Reb A. The order in which each glucosylation reaction occurs can vary. See FIG. 1.

Biosynthesis of Rebaudioside E and/or Rebaudioside D involves glucosylation of the aglycone steviol. Specifically, Rebaudioside E can be formed by glucosylation of the 13-OH of steviol which forms steviol-13-O-glucoside, glucosylation of the C-2′ of the 13-O-glucose of steviol-13-O-glucoside which forms the steviol-1,2-bioside, glucosylation of the C-19 carboxyl of the 1,2-bioside to form 1,2-stevioside, and glucosylation of the C-2′ of the 19-O-glucose of the 1,2-stevioside to form Rebaudioside E. Rebaudioside D can be formed by glucosylation of the C-3′ of the C-13-O-glucose of Rebaudioside E. The order in which each glycosylation reaction occurs can vary. For example, the glucosylation of the C-2′ of the 19-O-glucose can be the last step in the pathway, wherein Rebaudioside A is an intermediate in the pathway. See FIG. 1.

### Rebaudioside M Polypeptides

As provided herein, conversion of steviol to Rebaudioside M in a recombinant host can be accomplished by expressing combinations of the following functional UGTs: 91D2, EUGT11, 74G1, 85C2, and 76G1. See FIG. 1. It is particularly useful to express EUGT11 at high levels using a high copy number plasmid, or using a strong promoter, or multiple integrated copies of the gene, or episome under selection for high copy number of the gene. Thus, a recombinant microorganism expressing combinations of these UGTs can make Rebaudioside A (85C2; 76G1; 74G1; 91D2e), Rebaudioside D (85C2; 76G1; 74G1; 91D2e; EUGT11), Rebaudioside E (85C2; 74G1; 91D2e; EUGT11), or Rebaudioside M (85C2; 76G1; 74G1; 91D2e; EUGT11). See FIG. 1. Typically, one or more of these genes are recombinant genes that have been transformed into a microorganism that does not naturally possess them. It has also been discovered that UGTs designated herein as SM12UGT can be substituted for UGT91D2.

Targeted production of individual Rebaudiosides can be accomplished by differential copy numbers of the UGT-encoding genes (see FIG. 1) in the recombinant cell, differential promoter strengths, and/or by utilizing mutants with increased specificity/activity towards the product of interest. For example, low levels of Rebaudioside D, E, and M will be formed if EUGT11 is expressed at low levels in comparison to the other UGTs, which would favor Rebaudioside A formation. High levels of EUGT11 expression result in production of more 19-O 1,2 diglucoside that can serve as substrate for UGT76G1 to form Rebaudioside M. In certain advantageous embodiments, additional copies or mutant versions of UGT76G1 in recombinant cells of the invention can improve the rate of Rebaudioside M formation from Rebaudioside D.

In some embodiments, UGT76G1 catalyzes glycosylation of steviol and steviol glycosides at the 19-0 position. Thus, in some embodiments, one or more of RebM, RebQ, RebI, di-glycosylated steviol glycoside (13-hydroxy kaur-16-en-18-oic acid, [2-O-β-D-glucopyranosyl-β-D-glucopyranosyl]ester), or tri-glycosylated steviol glycoside ((13-hydroxy kaur-16-en-18-oic acid; [2-O-β-D-glucopyranosyl-3-O-β-D-glucopyranosyl-β-D-glucopyranosyl]ester) are produced in a recombinant host expressing a recombinant gene encoding a UGT76G1 polypeptide, through bioconversion, or through catalysis by UGT76G1 in vitro. In some embodiments, UGT76G1 catalyzes the glycosylation of steviol and steviol glycosides at the 13-O position and preferentially glycosylates steviol glycoside substrates that are 1,2-di-glycosylated at the 13-O position or mono-glycosylated at the 13-O position. In some embodiments, UGT76G1 does not show a preference for the glycosylation state of the 19-0 position.

In some aspects, a recombinant host cell of the invention comprises the gene encoding the UGT76G1 polypeptide set forth in SEQ ID NO:2. In some aspects, the gene encoding the UGT76G1 polypeptide set forth in SEQ ID NO:2, comprising a recombinant cell of the invention is overexpressed. In some aspects, the gene can be present in (including but not limited to) two or three copies.

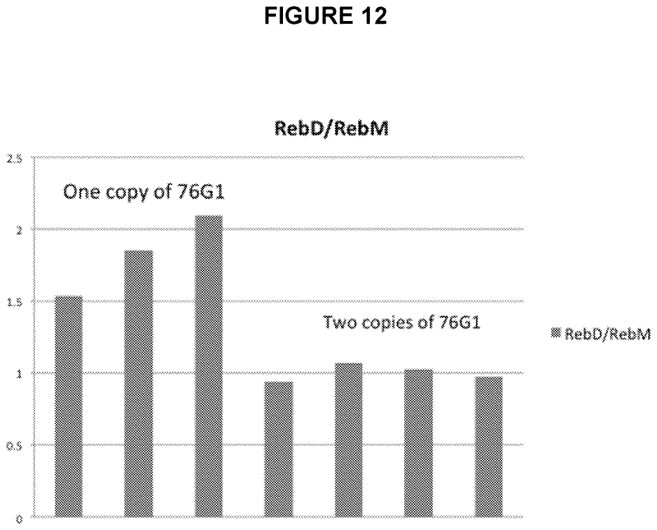

In some embodiments, the gene encoding the UGT76G1 polypeptide set forth in SEQ ID NO:2 is present in one copy. As shown in FIG. 12 (Example 9), a lower copy number (one copy) of the gene encoding the UGT76G1 polypeptide results in lower UGT76G1 expression and increases the Rebaudioside D/Rebaudioside M ratio.

In some embodiments, less than five (e.g., one, two, three, or four) UGTs are expressed in a host. For example, a recombinant microorganism expressing a functional EUGT11 can make Rebaudioside D when Rebaudioside A is used as a feedstock. A recombinant microorganism expressing a functional UGT76G1 can make Rebaudioside M when Rebaudioside D or Rebaudioside E is used as a feedstock. Rebaudioside M can be formed from either Rebaudioside D or Rebaudioside E by glucosylation of the C-3′ of the 19-O-glucose of Rebaudioside D or Rebuadioside E; in the case of Rebaudioside E a second glucosylation is required, of the 13-O-glucose to produce Rebaudioside M.

A recombinant microorganism expressing EUGT11, 74G1 or 76G1, and 91D2 can make Rebaudioside D or Rebaudioside M when rubusoside or 1,2-stevioside is used as a feedstock. As another alternative, a recombinant microorganism expressing EUGT11, 74G1, 76G1, and 91D2 can make Rebaudioside D or Rebaudioside M when the monoside, steviol-13-0-glucoside are added to the medium. Similarly, conversion of steviol-19-O-glucoside to Rebaudioside D in a recombinant microorganism can be accomplished by expressing in the cell genes encoding UGTs EUGT11, 85C2, 76G1, and 91D2e, when fed steviol-19-O-glucoside.

Suitable UGT74G1 and UGT85C2 polypeptides include those discussed above. A suitable UGT76G1 adds a glucose moiety to the C-3′ of the C-13-O-glucose of the acceptor molecule, a steviol 1,2 glycoside. UGT76G1 functions, for example, as a uridine 5′-diphospho glucosyl: steviol 13-O-1,2 glucoside C-3′ glucosyl transferase and a uridine 5′-diphospho glucosyl: steviol-19-O-glucose, 13-O-1,2 bioside C-3′ glucosyl transferase. Functional UGT76G1 polypeptides can also catalyze glucosyl transferase reactions that utilize steviol glycoside substrates that contain sugars other than glucose, e.g., steviol rhamnosides and steviol xylosides. See, FIG. 1. Suitable UGT76G1 polypeptides include those made by S. rebaudiana and reported in Richman et al., 2005, Plant J. 41: 56-67. A nucleotide sequence encoding the S. rebaudiana UGT76G1 polypeptide optimized for expression in yeast is set forth in SEQ ID NO:14. See also the UGT76G1 variants set forth in the “Functional Homolog” section.

A suitable EUGT11 or UGT91D2 polypeptide functions as a uridine 5′-diphospho glucosyl: steviol-13-O-glucoside transferase (also referred to as a steviol-13-monoglucoside 1,2-glucosylase), transferring a glucose moiety to the C-2′ of the 13-O-glucose of the acceptor molecule, steviol-13-O-glucoside.

A suitable EUGT11 or UGT91D2 polypeptide also functions as a uridine 5′-diphospho glucosyl: rubusoside transferase transferring a glucose moiety to the C-2′ of the 13-O-glucose of the acceptor molecule, rubusoside, to produce stevioside. EUGT11 polypeptides also can transfer a glucose moiety to the C-2′ of the 19-O-glucose of the acceptor molecule, rubusoside, to produce a 19-O-1,2-diglycosylated rubusoside (see FIG. 1).

Functional EUGT11 or UGT91D2 polypeptides also can catalyze reactions that utilize steviol glycoside substrates other than steviol-13-O-glucoside and rubusoside. For example, a functional EUGT11 polypeptide can utilize stevioside as a substrate, transferring a glucose moiety to the C-2′ of the 19-O-glucose residue to produce Rebaudioside E (see FIG. 1). Functional EUGT11 and UGT91D2 polypeptides can also utilize Rebaudioside A as a substrate, transferring a glucose moiety to the C-2′ of the 19-O-glucose residue of Rebaudioside A to produce Rebaudioside D. EUGT11 can convert Rebaudioside A to Rebaudioside D at a rate that is least 20 times faster (e.g., as least 25 times or at least 30 times faster) than the corresponding rate of wildtype UGT91D2e (SEQ ID NO: 15) when the reactions are performed under similar conditions, i.e., similar time, temperature, purity, and substrate concentration. As such, EUGT11 produces greater amounts of RebD than UGT91D2e under similar conditions in cells or in vitro, under conditions where the temperature-sensitive EUGT11 is stable.

In addition, a functional EUGT11 exhibits significant C-2′ 19-O-diglycosylation activity with rubusoside or stevioside as substrates, whereas UGT91D2e has no detectable diglycosylation activity with these substrates under some conditions. Thus, a functional EUGT11 can be distinguished from UGT91D2e by the differences in steviol glycoside substrate-specificity.

A functional EUGT11 or UGT91D2 polypeptide does not transfer a glucose moiety to steviol compounds having a 1,3-bound glucose at the C-13 position, i.e., transfer of a glucose moiety to steviol-1,3-bioside and 1,3-stevioside (RebG) does not occur.

Functional EUGT11 and UGT91D2 polypeptides can transfer sugar moieties from donors other than uridine diphosphate glucose. For example, a functional EUGT11 or UGT91D2 polypeptide can act as a uridine 5′-diphospho D-xylosyl: steviol-13-O-glucoside transferase, transferring a xylose moiety to the C-2′ of the 13-O-glucose of the acceptor molecule, steviol-13-O-glucoside. As another example, a functional EUGT11 or UGT91D2 polypeptide can act as a uridine 5′-diphospho L-rhamnosyl: steviol-13-O-glucoside transferase, transferring a rhamnose moiety to the C-2′ of the 13-O-glucose of the acceptor molecule, steviol-13-O-glucoside.

Suitable EUGT11 polypeptides are described herein and can include the EUGT11 polypeptide from Oryza sativa (GenBank Accession No. AC133334; SEQ ID NO:16). For example, an EUGT11 polypeptide can have an amino acid sequence with at least 70% sequence identity (e.g., at least 75, 80, 85, 90, 95, 96, 97, 98, or 99% sequence identity) to the amino acid sequence set forth in SEQ ID NO:16. The nucleotide sequence encoding the amino acid sequence of EUGT11 also is set forth in SEQ ID NO:17, as is a codon optimized nucleotide sequence for expression in yeast (SEQ ID NO:18).

Suitable functional UGT91D2 polypeptides include, e.g., the polypeptides designated UGT91D2e and UGT91D2m and functional homologs as described herein. The amino acid sequence of an exemplary UGT91D2e polypeptide from S. rebaudiana is set forth in SEQ ID NO:15, as is the nucleotide sequence encoding the UGT91D2e polypeptide that has been codon optimized for expression in yeast (SEQ ID NO:89). The amino acid sequences of exemplary UGT91D2m (SEQ ID NO:86) polypeptides from S. rebaudiana are set forth as SEQ ID NO: 10 in PCT Application No. PCT/US2012/050021, which is incorporated herein by reference in its entirety. In addition, UGT91D2 variants containing a substitution at amino acid residues 206, 207, and 343 can be used. For example, the amino acid sequence having G206R, Y207C, and W343R mutations with respect to wild-type UGT91D2e can be used. In addition, a UGT91D2 variant containing substitutions at amino acid residues 211 and 286 can be used. For example, a UGT91D2 variant can include a substitution of a methionine for leucine at position 211 and a substitution of an alanine for valine at position 286 (referred to as UGT91D2e-b). These variants, L211M and V286A, are variants of SEQ ID NO: 5 from PCT/US2012/050021, which is disclosed herein as SEQ ID NO: 66. Additional variants can include variants (except T144S, M152L, L213F, S364P, and G384C variants) described in Table 12 and Example 11 of the PCT/US2012/050021, which is incorporated herein by reference in its entirety.

As indicated above, UGTs designated herein as SM12UGT can be substituted for UGT91D2. Suitable functional SM12UGT polypeptides include those made by Ipomoea purpurea (Japanese morning glory) and described in Morita et al., 2005, Plant J. 42, 353-363. The amino acid sequence encoding the I. purpurea IP3GGT (SEQ ID NO: 67) (which is set forth in PCT Application No. PCT/US2012/050021, which is incorporated herein by reference in its entirety) as a nucleotide sequence (SEQ ID NO: 68) that encodes the polypeptide and that has been codon optimized for expression in yeast. Another suitable SM12UGT polypeptide is a UGT94B1 polypeptide having an R25S mutation (Bp94B1 polypeptide). See Osmani et al., 2008, Plant Phys. 148: 1295-1308 and Sawada et al., 2005, J. Biol. Chem. 280:899-906. The amino acid sequence of the Bellis perennis (red daisy) UGT94B1 (SEQ ID NO: 69) and the nucleotide sequence that has been codon optimized for expression in yeast (SEQ ID NO: 70) are set forth in PCT Application No. PCT/US2012/050021, which is incorporated herein by reference in its entirety.

In some embodiments, the recombinant microorganism is grown on media containing steviol-13-O-glucoside or steviol-19-O-glucoside in order to produce Rebaudioside M. In such embodiments, the microorganism contains and expresses genes encoding a functional EUGT11, a functional UGT74G1, a functional UGT85C2, a functional UGT76G1, and a functional UGT91D2, and is capable of accumulating Rebaudioside A, Rebaudioside D, Rebaudioside M or a combination thereof, depending on the relative levels of UDP-glycosyl transferase activities, when steviol, one or both of the steviolmonosides, or rubusoside is used as feedstock.

In other embodiments, the recombinant microorganism is grown on media containing rubusoside in order to produce Rebaudioside A, D, or M. In such embodiments, the microorganism contains and expresses genes encoding a functional EUGT11, a functional UGT76G1, and a functional UGT91D2, and is capable of producing Rebaudioside A, D, M or a combination thereof, depending on the relative levels of UDP-glycosyl transferase activities, when rubusoside is used as feedstock.

In other embodiments the recombinant microorganism expresses genes involved in steviol biosynthesis, e.g., a CDPS gene, a KS gene, a KO gene and/or a KAH gene. Thus, for example, a microorganism containing a CDPS gene, a KS gene, a KO gene and a KAH gene, in addition to a EUGT11, a UGT74G1, a UGT85C2, a UGT76G1, and a functional UGT91D2 (e.g., UGT91D2e), is capable of producing Rebaudioside A, D, E, and/or M without the necessity of including steviol in the culture media.

In some embodiments, the recombinant host further contains and expresses a recombinant GGPPS gene in order to provide increased levels of the diterpene precursor geranylgeranyl diphosphate, for increased flux through the steviol biosynthetic pathway.

In some embodiments, the recombinant host further contains a construct to silence expression of non-steviol pathways consuming geranylgeranyl diphosphate, ent-Kaurenoic acid or farnesyl pyrophosphate, thereby providing increased flux through the steviol and steviol glycosides biosynthetic pathways. As discussed below, flux to sterol production pathways such as ergosterol can be reduced by downregulation of the ERG9 gene. In cells that produce gibberellins, gibberellin synthesis can be downregulated to increase flux of ent-kaurenoic acid to steviol. In carotenoid-producing organisms, flux to steviol can be increased by downregulation of one or more carotenoid biosynthetic genes. In some embodiments, the recombinant microorganism further can express recombinant genes involved in diterpene biosynthesis or production of terpenoid precursors, e.g., genes in the MEP or MEV pathways discussed below, have reduced phosphatase activity, and/or express a SUS as discussed herein.

One with skill in the art will recognize that by modulating relative expression levels of different UGT genes, a recombinant host can be tailored to specifically produce steviol glycoside products in a desired proportion. Transcriptional regulation of steviol biosynthesis genes and steviol glycoside biosynthesis genes can be achieved by a combination of transcriptional activation and repression using techniques known to those in the art. For in vitro reactions, one with skill in the art will recognize that addition of different levels of UGT enzymes in combination or under conditions which impact the relative activities of the different UGTS in combination will direct synthesis towards a desired proportion of each steviol glycoside. One with skill in the art will recognize that a higher proportion of Rebaudioside D or M, or more efficient conversion to Rebaudioside D or M can be obtained with a diglycosylation enzyme that has a higher activity for the 19-O-glucoside reaction as compared to the 13-O-glucoside reaction (substrates Rebaudioside A and stevioside).

In some embodiments, a recombinant host such as a microorganism produces Rebaudioside M-enriched steviol glycoside compositions that have greater than at least 3% Rebaudioside M by weight total steviol glycosides, e.g., at least 4% Rebaudioside M, at least 5% Rebaudioside M, at least 10-20% Rebaudioside M, at least 20-30% Rebaudioside M, at least 30-40% Rebaudioside M, at least 40-50% Rebaudioside M, at least 50-60% Rebaudioside M, at least 60-70% Rebaudioside M, or at least 70-80% Rebaudioside M. Other steviol glycosides present can include those depicted in FIG. 1 such as steviol monosides, steviol glucobiosides, Rebaudioside A, Rebaudioside D, Rebaudioside E, and stevioside. In some embodiments, the Rebaudioside M-enriched composition produced by the host (e.g., microorganism) can be further purified and the Rebaudioside M so purified can then be mixed with other steviol glycosides, flavors, or sweeteners to obtain a desired flavor system or sweetening composition. For instance, a Rebaudioside M-enriched composition produced by a recombinant host can be combined with a Rebaudioside A, C, D, or E-enriched composition produced by a different recombinant host, with Rebaudioside A, C, D, or E purified from a Stevia extract, or with Rebaudioside A, C, D, or E produced in vitro.

In some embodiments, Rebaudioside M can be produced using in vitro methods while supplying the appropriate UDP-sugar and/or a cell-free system for regeneration of UDP-sugars. In some embodiments, sucrose and a sucrose synthase can be provided in the reaction vessel in order to regenerate UDP-glucose from the UDP generated during glycosylation reactions. The sucrose synthase can be from any suitable organism. For example, a sucrose synthase coding sequence from A. thaliana, S. rebaudiana, or Coffea arabica can be cloned into an expression plasmid under control of a suitable promoter, and expressed in a host such as a microorganism or a plant.

Conversions requiring multiple reactions can be carried out together, or stepwise. For example, Rebaudioside M can be produced from Rebaudioside D or Rebaudioside E that is commercially available as an enriched extract or produced via biosynthesis, with the addition of stoichiometric or excess amounts of UDP-glucose and UGT76G1. As an alternative, Rebaudioside D and Rebaudioside M can be produced from steviol glycoside extracts that are enriched for stevioside and Rebaudioside A, using EUGT11 and a suitable UGT76G1 enzyme. In some embodiments, phosphatases are used to remove secondary products and improve reaction yields. UGTs and other enzymes for in vitro reactions can be provided in soluble forms or in immobilized forms.

In some embodiments, Rebaudioside M can be produced using whole cells that are fed raw materials that contain precursor molecules such as steviol and/or steviol glycosides, including mixtures of steviol glycosides derived from plant extracts. The raw materials can be fed during cell growth or after cell growth. The whole cells can be in suspension or immobilized. The whole cells can be entrapped in beads, for example calcium or sodium alginate beads. The whole cells can be linked to a hollow fiber tube reactor system. The whole cells can be concentrated and entrapped within a membrane reactor system. The whole cells can be in fermentation broth or in a reaction buffer. Similar methodology can be applied to fermentation of recombinant cells.

In some embodiments, a permeabilizing agent is utilized for efficient transfer of substrate into the cells. In some embodiments, the cells are permeabilized with a solvent such as toluene, or with a detergent such as Triton-X or Tween. In some embodiments, the cells are permeabilized with a surfactant, for example a cationic surfactant such as cetyltrimethylammonium bromide (CTAB). In some embodiments, the cells are permeabilized with periodic mechanical shock such as electroporation or a slight osmotic shock. The cells can contain one recombinant UGT or multiple recombinant UGTs. For example, the cells can contain UGT76G1, 91D2e, 85C2, 74G1 and EUGT11 such that mixtures of steviol and/or steviol glycosides are efficiently converted to Rebaudioside M. In some embodiments, the whole cells are the host cells described below. In some embodiments, the whole cells are a Gram-negative bacterium such as E. coli. In some embodiments, the whole cell is a Gram-positive bacterium such as Bacillus. In some embodiments, the whole cell is a fungal species such as Aspergillus, or yeast such as Saccharomyces. In some embodiments, the term “whole cell biocatalysis” is used to refer to the process in which the whole cells are grown as described above (e.g., in a medium and optionally permeabilized) and a substrate such as Rebaudioside D, Rebaudioside E, or stevioside is provided and converted to the end product using the enzymes from the cells. The cells can or cannot be viable, and can or cannot be growing during the bioconversion reactions. In contrast, in fermentation, the cells are cultured in a growth medium and fed a carbon and energy source such as glucose and the end product is produced with viable cells.

### C. Other Polypeptides

Genes for additional polypeptides whose expression facilitates more efficient or larger scale production of steviol or a steviol glycoside can also be introduced into a recombinant host. For example, a recombinant microorganism, plant, or plant cell can also contain one or more genes encoding a geranylgeranyl diphosphate synthase (GGPPS, also referred to as GGDPS). As another example, the recombinant host can contain one or more genes encoding a rhamnose synthetase, or one or more genes encoding a UDP-glucose dehydrogenase and/or a UDP-glucuronic acid decarboxylase. As another example, a recombinant host can also contain one or more genes encoding a cytochrome P450 reductase (CPR). Expression of a recombinant CPR facilitates the cycling of NADP+ to regenerate NADPH, which is utilized as a cofactor for terpenoid biosynthesis. Other methods can be used to regenerate NADHP levels as well. In circumstances where NADPH becomes limiting, for example, strains can be further modified to include exogenous transhydrogenase genes. See, e.g., Sauer et al., 2004, J. Biol. Chem. 279: 6613-6619. Other methods are known to those with skill in the art to reduce or otherwise modify the ratio of NADH/NADPH such that the desired cofactor level is increased.

As another example the recombinant host can contain one or more genes encoding a sucrose synthase, and additionally can contain sucrose uptake genes if desired. The sucrose synthase reaction can be used to increase the UDP-glucose pool in a fermentation host, or in a whole cell bioconversion process. This regenerates UDP-glucose from UDP produced during glycosylation and sucrose, allowing for efficient glycosylation. In some organisms, disruption of the endogenous invertase is advantageous to prevent degradation of sucrose. For example, the S. cerevisiae SUC2 invertase can be disrupted. The sucrose synthase (SUS) can be from any suitable organism. For example, a sucrose synthase coding sequence from, without limitation, A. thaliana, S. rebaudiana, or C. arabica can be cloned into an expression plasmid under control of a suitable promoter, and expressed in a host (e.g., a microorganism or a plant). The sucrose synthase can be expressed in such a strain in combination with a sucrose transporter (e.g., the A. thaliana SUC1 transporter or a functional homolog thereof) and one or more UGTs (e.g., UGT85C2, UGT74G1, UGT76G1, and UGT91D2e, EUGT11 or functional homologs thereof). Culturing the host in a medium that contains sucrose can promote production of UDP-glucose, as well as one or more glucosides (e.g., steviol glycosides).

Expression of the ERG9 gene, which encodes squalene synthase (SQS), also can be reduced in recombinant hosts such that there is a build-up of precursors to squalene synthase in the recombinant host. SQS is classified under EC 2.5.1.21 and is the first committed enzyme of the biosynthesis pathway that leads to the production of sterols. It catalyzes the synthesis of squalene from farnesyl pyrophosphate via the intermediate presqualene pyrophosphate, wherein two units of farnesyl pyrophosphate are converted into squalene. This enzyme is a branch point enzyme in the biosynthesis of terpenoids/isoprenoids and is thought to regulate the flux of isoprene intermediates through the sterol pathway. The enzyme is sometimes referred to as farnesyl-diphosphate farnesyltransferase (FDFT1). In addition, a recombinant host can have reduced phosphatase activity as discussed herein.

### MEP Biosynthesis Polypeptides

As another example, the recombinant host can contain one or more genes encoding one or more enzymes in the MEP pathway or the mevalonate pathway. Such genes are useful because they can increase the flux of carbon into the diterpene biosynthesis pathway, producing geranylgeranyl diphosphate from isopentenyl diphosphate and dimethylallyl diphosphate generated by the pathway. The geranylgeranyl diphosphate so produced can be directed towards steviol and steviol glycoside biosynthesis due to expression of steviol biosynthesis polypeptides and steviol glycoside biosynthesis polypeptides. See, e.g., Brandle et al., 2007, Phytochemistry 68:1855-1863.

In some embodiments, a recombinant host contains one or more genes encoding enzymes involved in the methylerythritol 4-phosphate (MEP) pathway for isoprenoid biosynthesis. Enzymes in the MEP pathway include deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS) and 1-hydroxy-2-methyl-2(E)-butenyl 4-diphosphate reductase (HDR). One or more DXS genes, DXR genes, CMS genes, CMK genes, MCS genes, HDS genes and/or HDR genes can be incorporated into a recombinant microorganism. See, Rodriguez-Concepción and Boronat, Plant Phys. 130: 1079-1089 (2002).

Suitable genes encoding DXS, DXR, CMS, CMK, MCS, HDS and/or HDR polypeptides include those made by E. coli, A. thaliana and Synechococcus leopoliensis. Nucleotide sequences encoding DXR polypeptides (e.g., SEQ ID NO: 71) are described, for example, in U.S. Pat. No. 7,335,815.

### Mevalonate Biosynthesis Polypeptides

S. cerevisiae contains endogenous genes encoding the enzymes of a functional mevalonate pathway for isoprenoid synthesis. In some embodiments, a recombinant host also contains one or more heterologous genes encoding enzymes involved in the mevalonate pathway. Genes suitable for transformation into a host encode enzymes in the mevalonate pathway such as a truncated 3-hydroxy-3-methyl-glutaryl (HMG)-CoA reductase (tHMG), and/or a gene encoding a mevalonate kinase (MK), and/or a gene encoding a phosphomevalonate kinase (PMK), and/or a gene encoding a mevalonate pyrophosphate decarboxylase (MPPD). Thus, one or more HMG-CoA reductase genes, MK genes, PMK genes, and/or MPPD genes can be incorporated into a recombinant host such as a microorganism.

Suitable genes encoding mevalonate pathway polypeptides are known. For example, suitable polypeptides include those made by E. coli, Paracoccus denitrificans, S. cerevisiae, A. thaliana, Kitasatospora griseola, Homo sapiens, Drosophila melanogaster, Gallus gallus, Streptomyces sp. KO-3988, Nicotiana attenuata, Kitasatospora griseola, Hevea brasiliensis, Enterococcus faecium and Haematococcus pluvialis. See, e.g., Table 9, U.S. Pat. Nos. 7,183,089, 5,460,949, and 5,306,862, and PCT Application Nos. PCT/US2012/050021 and PCT/US2011/038967, which are incorporated herein by reference in their entirety.

TABLE 9
Sources of HMG CoA Reductases and other Mevalonate Genes

| | | | | Gene |
|---|---|---|---|---|
| Accession# | Organism | Enzyme | Size (nt) | name |
| XM_001467423 | Leishmania | Acetyl-CoA | 1323 | MEV- |
| (amino acid SEQ | infantum | C-acetyl- | (nt SEQ | 4 |
| ID NO: 72) | | trans- | ID NO: 94) | |
| | | ferase | | |
| YML075C | Saccharomyces | Truncated | 1584 | tHMG1 |
| (amino acid SEQ | cerevisiae | HMG | (nt SEQ | |
| ID NO: 73) | | (tHMG1) | ID NO: 95) | |
| EU263989 | Ganoderma | 3-HMG-CoA | 3681 | MEV- |
| (amino acid SEQ | lucidum | reductase | (nt SEQ | 11 |
| ID NO: 74) | | | ID NO: 96) | |
| BC153262 | Bos taurus | 3-HMG-CoA | 2667 | MEV- |
| (amino acid SEQ | | reductase | (nt SEQ | 12 |
| ID NO: 75) | | | ID NO: 97) | |
| AAD47596 | Artemisia | 3-HMG-CoA | 1704 | MEV- |
| (amino acid SEQ | annua | reductase | (nt SEQ | 13 |
| ID NO: 76) | | | ID NO: 98) | |
| AAB62280 | Trypanosoma | 3-HMG-CoA | 1308 | MEV- |
| (amiono acid | cruzi | reductase | (nt SEQ | 14 |
| SEQ ID NO: 77) | | | ID NO: 99) | |
| CAG41604 | Staph aureus | 3-HMG-CoA | 1281 | MEV- |
| (amino acid SEQ | | reductase | (nt SEQ | 15 |
| ID NO: 78) | | | ID NO: | |
| | | | 100) | |
| DNA2.0 | Archaeoglobus | 3-HMG-CoA | 1311 | HMG |
| sequence | fulgidus | reductase | (nt SEQ | re- |
| (amino acid SEQ | | | ID NO: | ductase |
| ID NO: 92) | | | 101) | |
| DNA2.0 | Pseudomonas | 3-HMG-CoA | 1287 | HMG |
| sequence | mevalonii | reductase | (nt SEQ | re- |
| (amino acid SEQ | | | ID NO: | ductase |
| ID NO: 93) | | | 102) | |

### Sucrose Synthase Polypeptides

Sucrose synthase (SUS) can be used as a tool for generating UDP-sugar, in particular UDP-glucose. SUS (EC 2.4.1.13) catalyzes the formation of UDP-glucose and fructose from sucrose and UDP. UDP generated by the reaction of UGTs thus can be converted by SUS into UDP-glucose in the presence of sucrose. See, e.g., Chen et al., 2001, J. Am. Chem. Soc. 123:8866-8867; Shao et al., 2003, Appl. Env. Microbiol. 69:5238-5242; Masada et al., 2007, FEES Lett. 581:2562-2566; and Son et al., 2009, J. Microbiol. Biotechnol. 19:709-712.

Sucrose synthases can be used to generate UDP-glucose and remove UDP, facilitating efficient glycosylation of compounds in various systems. For example, yeast deficient in the ability to utilize sucrose can be made to grow on sucrose by introducing a sucrose transporter and a SUS. For example, S. cerevisiae does not have an efficient sucrose uptake system, and relies on extracellular SUC2 to utilize sucrose. The combination of disrupting the endogenous S. cerevisiae SUC2 invertase and expressing recombinant SUS resulted in a yeast strain that was able to metabolize intracellular but not extracellular sucrose (Riesmeier et al., 1992, EMBO J. 11:4705-4713). The strain was used to isolate sucrose transporters by transformation with a cDNA expression library and selection of transformants that had gained the ability to take up sucrose.

The combined expression of recombinant sucrose synthase and a sucrose transporter in vivo can lead to increased UDP-glucose availability and removal of unwanted UDP. For example, functional expression of a recombinant sucrose synthase, a sucrose transporter, and a glycosyltransferase, in combination with knockout of the natural sucrose degradation system (SUC2 in the case of S. cerevisiae) can be used to generate a cell that is capable of producing increased amounts of glycosylated compounds such as steviol glycosides. This higher glycosylation capability is due to at least (a) a higher capacity for producing UDP-glucose in a more energy efficient manner, and (b) removal of UDP from growth medium, as UDP can inhibit glycosylation reactions.

The sucrose synthase can be from any suitable organism. For example, a sucrose synthase coding sequence from, without limitation, A. thaliana (e.g. SEQ ID NO: 79 or 80), or C. arabica (e.g., SEQ ID NO: 81) (see e.g., SEQ ID NOs: 178, 179, and 180 of PCT/US2012/050021, which is incorporated herein by reference in its entirety) includes the amino acid sequence of the sucrose transporter SUC1 from A. thaliana (SEQ ID NO: 80), and the amino acid sequence of the sucrose synthase from coffee (SEQ ID NO: 81).

The sucrose synthase can be from any suitable organism. For example, a sucrose synthase coding sequence from, without limitation, A. thaliana, S. rebaudiana, or C. arabica (see e.g., SEQ ID NOs: 79-81) can be cloned into an expression plasmid under control of a suitable promoter, and expressed in a host (e.g., a microorganism or a plant). A SUS coding sequence can be expressed in a SUC2 (sucrose hydrolyzing enzyme) deficient S. cerevisiae strain, so as to avoid degradation of extracellular sucrose by the yeast.

The sucrose synthase can be expressed in such a strain in combination with a sucrose transporter (e.g., the A. thaliana SUC1 transporter or a functional homolog thereof) and one or more UGTs (e.g., UGT85C2, UGT74G1, UGT76G1, EUGT11, and UGT91D2e, or functional homologs thereof). Culturing the host in a medium that contains sucrose can promote production of UDP-glucose, as well as one or more glucosides (e.g., steviol glucoside). It is to be noted that in some cases, a sucrose synthase and a sucrose transporter can be expressed along with a UGT in a host cell that also is recombinant for production of a particular compound (e.g., steviol).

### Modulating Expression of ERG9 Gene

Expression of the endogenous ERG9 gene can be altered in a recombinant host described herein using a nucleic acid construct. The construct can include two regions that are homologous to parts of the genome sequence within the ERG9 promoter or 5′ end of the ERG9 open reading frame (ORF), respectively. The construct can further include a promoter, such as either the wild type ScKex2 or wild type ScCyc1, and the promoter further can include a heterologous insert such as a hairpin at its 3′-end. The polypeptide encoded by the ORF advantageously has at least 70% identity to a squalene synthase (EC 2.5.1.21) or a biologically active fragment thereof, said fragment having at least 70% sequence identity to said squalene synthase in a range of overlap of at least 100 amino acids. See, for example, PCT/US2012/050021 (incorporated herein for all purposes in its entirety).

The heterologous insert can adapt the secondary structure element of a hairpin with a hairpin loop. The heterologous insert sequence has the general formula (I):

-X1-X2-X3-X4-X5, wherein
X2 comprises at least 4 consecutive nucleotides being complementary to, and forming a hairpin secondary structure element with at least 4 consecutive nucleotides of X4, and
X3 is optional and if present comprises nucleotides involved in forming a hairpin loop between X2 and X4, and
X1 and X5 individually and optionally comprise one or more nucleotides, and
X2 and X4 can individually consist of any suitable number of nucleotides, so long as a consecutive sequence of at least 4 nucleotides of X2 is complementary to a consecutive sequence of at least 4 nucleotides of X4. In some embodiments, X2 and X4 consist of the same number of nucleotides.

The heterologous insert is long enough to allow a hairpin to be completed, but short enough to allow limited translation of an ORF that is present in-frame and immediately 3′ to the heterologous insert. Typically, the heterologous insert is from 10-50 nucleotides in length, e.g., 10-30 nucleotides, 15-25 nucleotides, 17-22 nucleotides, 18-21 nucleotides, 18-20 nucleotides, or 19 nucleotides in length. As provided herein:

X2 can for example consist of in the range of 4 to 25 nucleotides, such as in the range of 4 to 20, 4 to 15, 6 to 12, 8 to 12, or 9 to 11 nucleotides.

X4 can for example consist of in the range of 4 to 25 nucleotides, such as in the range of 4 to 20, 4 to 15, 6 to 12, 8 to 12, or 9 to 11 nucleotides.

In some embodiments, X2 consists of a nucleotide sequence that is complementary to the nucleotide sequence of X4, all nucleotides of X2 are complementary to the nucleotide sequence of X4.

X3 can be absent, i.e., X3 can consist of zero nucleotides. It is also possible that X3 consists of in the range of 1 to 5 nucleotides, such as in the range of 1 to 3 nucleotides.

X1 can be absent, i.e., X1 can consist of zero nucleotides. It is also possible that X1 consists of in the range of 1 to 25 nucleotides, such as in the range of 1 to 20, 1 to 15, 1 to 10, 1 to 5, or 1 to 3 nucleotides.

X5 can be absent, i.e., X5 can consist of zero nucleotides. It is also possible that X5 can consist of in the range 1 to 5 nucleotides, such as in the range of 1 to 3 nucleotides.

The heterologous insert can be any suitable sequence fulfilling the requirements defined herein. For example, the heterologous insert can comprise tgaattcgttaacgaattc (SEQ ID NO: 82), tgaattcgttaacgaactc (SEQ ID NO: 83), tgaattcgttaacgaagtc (SEQ ID NO: 84), or tgaattcgttaacgaaatt (SEQ ID NO: 85).

Without being bound to a particular mechanism, ERG9 expression can be decreased by at least partly, sterically hindering binding of the ribosome to the RNA thus reducing the translation of squalene synthase. Thus, the translation rate of a functional squalene synthase (EC 2.5.1.21) can be reduced, for example. Using such a construct also can decrease turnover of farnesyl-pyrophosphate to squalene and/or enhance accumulation of a compound selected from the group consisting of farnesyl-pyrophosphate, isopentenyl-pyrophosphate, dimethylallyl-pyrophosphate, geranyl-pyrophosphate and geranylgeranyl-pyrophosphate.

In some instances it can be advantageous to include a squalene synthase inhibitor when culturing recombinant hosts described herein. Chemical inhibition of squalene synthase, e.g., by lapaquistat, is known in the art. Other squalene synthase inhibitors include Zaragozic acid and RPR 107393. Thus, in one embodiment the culturing step of the method(s) defined herein are performed in the presence of a squalene synthase inhibitor.

In some embodiments, the recombinant hosts described herein contain a mutation in the ERG9 open reading frame.

In some embodiments, the recombinant hosts described herein contain an ERG9[Delta]::HIS3 deletion/insertion allele.

### D. Functional Homologs

Functional homologs of the polypeptides described above are also suitable for use in producing steviol or steviol glycosides in a recombinant host. A functional homolog is a polypeptide that has sequence similarity to a reference polypeptide, and that carries out one or more of the biochemical or physiological function(s) of the reference polypeptide. A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional UGT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide:polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of steviol or steviol glycoside biosynthesis polypeptides. Sequence analysis can involve BLAST, Reciprocal BLAST, or PSI-BLAST analysis of nonredundant databases using a GGPPS, a CDPS, a KS, a KO or a KAH amino acid sequence as the reference sequence. Amino acid sequence is, in some instances, deduced from the nucleotide sequence. Those polypeptides in the database that have greater than 40% sequence identity are candidates for further evaluation for suitability as a steviol or steviol glycoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in steviol biosynthesis polypeptides, e.g., conserved functional domains.

Conserved regions can be identified by locating a region within the primary amino acid sequence of a steviol or a steviol glycoside biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing steviol glycosides in a recombinant host include functional homologs of EUGT11 (SEQ ID NO: 16), UGT91D2e (SEQ ID NO: 15), UGT91D2m (SEQ ID NO: 86), UGT85C (SEQ ID NO: 26), and UGT76G (SEQ ID NO:2). Such homologs have greater than 90% (e.g., at least 95% or 99%) sequence identity to the amino acid sequence of EUGT11, UGT91D2e, UGT91D2m, UGT85C, or UGT76G disclosed herein or in PCT Application No. PCT/US2012/050021, which is incorporated herein by reference in its entirety. Variants of EUGT11, UGT91D2, UGT85C, and UGT76G polypeptides typically have 10 or fewer amino acid substitutions within the primary amino acid sequence, e.g., 7 or fewer amino acid substitutions, 5 or conservative amino acid substitutions, or between 1 and 5 substitutions. However, in some embodiments, variants of EUGT11, UGT91D2, UGT85C, and UGT76G polypeptides can have 10 or more amino acid substitutions (e.g., 10, 15, 20, 25, 30, 35, 10-20, 10-35, 20-30, or 25-35 amino acid substitutions). The substitutions can be conservative, or in some embodiments, non-conservative. Non-limiting examples of non-conservative changes in UGT91D2e polypeptides include glycine to arginine and tryptophan to arginine. Non-limiting examples of non-conservative substitutions in UGT76G polypeptides include valine to glutamic acid, glycine to glutamic acid, glutamine to alanine, and serine to proline. Non-limiting examples of changes to UGT85C polypeptides include histidine to aspartic acid, proline to serine, lysine to threonine, and threonine to arginine.

In some embodiments, a useful UGT91D2 homolog can have amino acid substitutions (e.g., conservative amino acid substitutions) in regions of the polypeptide that are outside of predicted loops, e.g., residues 20-26, 39-43, 88-95, 121-124, 142-158, 185-198, and 203-214 are predicted loops in the N-terminal domain and residues 381-386 are predicted loops in the C-terminal domain of 91D2e (see SEQ ID NO:15). For example, a useful UGT91D2 homolog can include at least one amino acid substitution at residues 1-19, 27-38, 44-87, 96-120, 125-141, 159-184, 199-202, 215-380, or 387-473. In some embodiments, a UGT91D2 homolog can have an amino acid substitution at one or more residues selected from the group consisting of residues 30, 93, 99, 122, 140, 142, 148, 153, 156, 195, 196, 199, 206, 207, 211, 221, 286, 343, 427, and 438. For example, a UGT91D2 functional homolog can have an amino acid substitution at one or more of residues 206, 207, and 343, such as an arginine at residue 206, a cysteine at residue 207, and an arginine at residue 343. Other functional homologs of UGT91D2 can have one or more of the following: a tyrosine or phenylalanine at residue 30, a proline or glutamine at residue 93, a serine or valine at residue 99, a tyrosine or a phenylalanine at residue 122, a histidine or tyrosine at residue 140, a serine or cysteine at residue 142, an alanine or threonine at residue 148, a methionine at residue 152, an alanine at residue 153, an alanine or serine at residue 156, a glycine at residue 162, a leucine or methionine at residue 195, a glutamic acid at residue 196, a lysine or glutamic acid at residue 199, a leucine or methionine at residue 211, a leucine at residue 213, a serine or phenylalanine at residue 221, a valine or isoleucine at residue 253, a valine or alanine at residue 286, a lysine or asparagine at residue 427, an alanine at residue 438, and either an alanine or threonine at residue 462. In another embodiment, a UGT91D2 functional homolog contains a methionine at residue 211 and an alanine at residue 286.

In some embodiments, a useful UGT85C homolog can have one or more amino acid substitutions at residues 9, 10, 13, 15, 21, 27, 60, 65, 71, 87, 91, 220, 243, 270, 289, 298, 334, 336, 350, 368, 389, 394, 397, 418, 420, 440, 441, 444, and 471. Non-limiting examples of useful UGT85C homologs include polypeptides having substitutions (with respect to SEQ ID NO: 26) at residue 65 (e.g., a serine at residue 65), at residue 65 in combination with residue 15 (a leucine at residue 15), 270 (e.g., a methionine, arginine, or alanine at residue 270), 418 (e.g., a valine at residue 418), 440 (e.g., an aspartic acid at residue at residue 440), or 441 (e.g., an asparagine at residue 441); residues 13 (e.g., a phenylalanine at residue 13), 15, 60 (e.g., an aspartic acid at residue 60), 270, 289 (e.g., a histidine at residue 289), and 418; substitutions at residues 13, 60, and 270; substitutions at residues 60 and 87 (e.g., a phenylalanine at residue 87); substitutions at residues 65, 71 (e.g., a glutamine at residue 71), 220 (e.g., a threonine at residue 220), 243 (e.g., a tryptophan at residue 243), and 270; substitutions at residues 65, 71, 220, 243, 270, and 441; substitutions at residues 65, 71, 220, 389 (e.g., a valine at residue 389), and 394 (e.g., a valine at residue 394); substitutions at residues 65, 71, 270, and 289; substitutions at residues 220, 243, 270, and 334 (e.g., a serine at residue 334); or substitutions at residues 270 and 289. The following amino acid mutations did not result in a loss of activity in 85C2 polypeptides: V13F, F15L, H60D, A65S, E71Q, 187F, K220T, R243W, T270M, T270R, Q289H, L334S, A389V, 1394V, P397S, E418V, G440D, and H441N. Additional mutations that were seen in active clones include K9E, K10R, Q21H, M27V, L91P, Y298C, K350T, H368R, G420R, L431P, R444G, and M471T. In some embodiments, an UGT85C2 contains substitutions at positions 65 (e.g., a serine), 71 (a glutamine), 270 (a methionine), 289 (a histidine), and 389 (a valine).

In some embodiments, a useful UGT76G1 homolog (SEQ ID NO: 2) can have one or more amino acid substitutions at residues 29, 74, 87, 91, 116, 123, 125, 126, 130, 145, 192, 193, 194, 196, 198, 199, 200, 203, 204, 205, 206, 207, 208, 266, 273, 274, 284, 285, 291, 330, 331, and 346 (see TABLE 10). Non-limiting examples of useful UGT76G1 homologs include polypeptides having substitutions at residues 74, 87, 91, 116, 123, 125, 126, 130, 145, 192, 193, 194, 196, 198, 199, 200, 203, 204, 205, 206, 207, 208, and 291; residues 74, 87, 91, 116, 123, 125, 126, 130, 145, 192, 193, 194, 196, 198, 199, 200, 203, 204, 205, 206, 207, 208, 266, 273, 274, 284, 285, and 291; or residues 74, 87, 91, 116, 123, 125, 126, 130, 145, 192, 193, 194,196, 198, 199, 200, 203, 204, 205, 206, 207, 208, 266, 273, 274, 284, 285, 291, 330, 331, and 346. See, Table 10.

TABLE 10

| Clone | Variants |
|---|---|
| 76G_G7 | M29I, V74E, V87G, L91P, G116E, A123T, Q125A, I126L, |
| | T130A, V145M, C192S, S193A, F194Y, M196N, K198Q, |
| | K199I, Y200L, Y203I, F204L, E205G, N206K, I207M, |
| | T208I, P266Q, S273P, R274S, G284T, T285S, 287-3 bp |
| | deletion, L330V, G331A, L346I |
| 76G_H12 | M29I, V74E, V87G, L91P, G116E, A123T, Q125A, I126L, |
| | T130A, V145M, C192S, S193A, F194Y, M196N, K198Q, |
| | K199I, Y200L, Y203I, F204L, E205G, N206K, I207M, |
| | T208I, P266Q, S273P, R274S, G284T, T285S, 287-3 bp |
| | deletion |
| 76G_C4 | M29I, V74E, V87G, L91P, G116E, A123T, Q125A, I126L, |
| | T130A, V145M, C192S, S193A, F194Y, M196N, K198Q, |
| | K199I, Y200L, Y203I, F204L, E205G, N206K, I207M, |
| | T208I |

Methods to modify the substrate specificity of, for example, EUGT11 or UGT91D2e, are known to those skilled in the art, and include without limitation site-directed/rational mutagenesis approaches, random directed evolution approaches and combinations in which random mutagenesis/saturation techniques are performed near the active site of the enzyme. For example see Sarah A. Osmani, et al., Phytochemistry 70 (2009) 325-347.

A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A functional homolog polypeptide typically has a length that is from 95 percent to 105 percent of the length of the reference sequence, e.g., 90, 93, 95, 97, 99, 100, 105, 110, 115, or 120 percent of the length of the reference sequence, or any range between. A percent identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that functional UGTs can include additional amino acids that are not involved in glucosylation or other enzymatic activities carried out by the enzyme, and thus such a polypeptide can be longer than would otherwise be the case. For example, a EUGT11 polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some embodiments, a EUGT11 polypeptide includes an amino acid sequence that functions as a reporter, e.g., a green fluorescent protein or yellow fluorescent protein.

### II. Steviol and Steviol Glycoside Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

One or more genes can be combined in a recombinant nucleic acid construct in “modules” useful for a discrete aspect of steviol and/or steviol glycoside production. Combining a plurality of genes in a module, particularly a polycistronic module, facilitates the use of the module in a variety of species. For example, a steviol biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for steviol or steviol glycoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards steviol or steviol glycoside biosynthesis. For example, it can be desirable to downregulate synthesis of sterols in a yeast strain in order to further increase steviol or steviol glycoside production, e.g., by downregulating squalene epoxidase. As another example, it can be desirable to inhibit degradative functions of certain endogenous gene products, e.g., glycohydrolases that remove glucose moieties from secondary metabolites or phosphatases as discussed herein. As another example, expression of membrane transporters involved in transport of steviol glycosides can be inhibited, such that secretion of glycosylated steviosides is inhibited. Such regulation can be beneficial in that secretion of steviol glycosides can be inhibited for a desired period of time during culture of the microorganism, thereby increasing the yield of glycoside product(s) at harvest. In such cases, a nucleic acid that inhibits expression of the polypeptide or gene product can be included in a recombinant construct that is transformed into the strain. Alternatively, mutagenesis can be used to generate mutants in genes for which it is desired to inhibit function.

### III. Hosts

### Microorganisms

Recombinant hosts can be used to express polypeptides for the production of steviol glycosides, including mammalian, insect, and plant cells. A number of prokaryotes and eukaryotes are also suitable for use in constructing the recombinant microorganisms described herein, e.g., gram-negative bacteria, yeast and fungi. A species and strain selected for use as a steviol or steviol glycoside production strain is first analyzed to determine which production genes are endogenous to the strain and which genes are not present. Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species can be suitable. For example, suitable species can be in a genus selected from the group consisting of Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis, Candida glabrata, Candida albicans, and Yarrowia lipolytica. In some embodiments, a microorganism can be an Ascomycete such as Gibberella fujikuroi, Kluyveromyces lactis, Schizosaccharomyces pombe, Aspergillus niger, Yarrowia lipolytica, Ashbya gossypii, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of steviol glycosides.

Saccharomyces cerevisiae

Saccharomyces cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

A steviol biosynthesis gene cluster can be expressed in yeast using any of a number of known promoters. Strains that overproduce terpenes are known and can be used to increase the amount of geranylgeranyl diphosphate available for steviol and steviol glycoside production.

### Aspergillus spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of food ingredients such as steviol and steviol glycosides.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete spp.

Agaricus, Gibberella, and Phanerochaete spp. can be useful because they are known to produce large amounts of gibberellin in culture. Thus, the terpene precursors for producing large amounts of steviol and steviol glycosides are already produced by endogenous genes. Thus, modules containing recombinant genes for steviol or steviol glycoside biosynthesis polypeptides can be introduced into species from such genera without the necessity of introducing mevalonate or MEP pathway genes.

Arxula adeninivorans (Blastobotrys adeninivorans)

Arxula adeninivorans is a dimorphic yeast (it grows as a budding yeast like the baker's yeast up to a temperature of 42 °C, above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is a dimorphic yeast (see Arxula adeninivorans) that can grow on a wide range of substrates. It has a high potential for industrial applications but there are no recombinant products commercially available yet.

### Rhodobacter spp.

Rhodobacter can be used as the recombinant microorganism platform. Similar to E. coli, there are libraries of mutants available as well as suitable plasmid vectors, allowing for rational design of various modules to enhance product yield. Isoprenoid pathways have been engineered in membraneous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

Candida boidinii

Candida boidinii is a methylotrophic yeast (it can grow on methanol). Like other methylotrophic species such as Hansenula polymorpha and Pichia pastoris, it provides an excellent platform for the production of heterologous proteins. Yields in a multigram range of a secreted foreign protein have been reported. A computational method, IPRO, recently predicted mutations that experimentally switched the cofactor specificity of Candida boidinii xylose reductase from NADPH to NADH.

Hansenula polymorpha (Pichia angusta)

Hansenula polymorpha is another methylotrophic yeast (see Candida boidinil). It can furthermore grow on a wide range of other substrates; it is thermo-tolerant and can assimilate nitrate (see also Kluyveromyces lactis). It has been applied to the production of hepatitis B vaccines, insulin and interferon alpha-2a for the treatment of hepatitis C, furthermore to a range of technical enzymes.

Kluyveromyces lactis

Kluyveromyces lactis is yeast regularly applied to the production of kefir. It can grow on several sugars, most importantly on lactose which is present in milk and whey. It has successfully been applied among others to the production of chymosin (an enzyme that is usually present in the stomach of calves) for the production of cheese. Production takes place in fermenters on a 40,000 L scale.

Pichia pastoris

Pichia pastoris is a methylotrophic yeast (see Candida boidinii and Hansenula polymorpha). It provides an efficient platform for the production of foreign proteins. Platform elements are available as a kit and it is worldwide used in academia for the production of proteins. Strains have been engineered that can produce complex human N-glycan (yeast glycans are similar but not identical to those found in humans).

### Physcomitrella spp.

Physcomitrella mosses, when grown in suspension culture, have characteristics similar to yeast or other fungal cultures. This genera is becoming an important type of cell for production of plant secondary metabolites, which can be difficult to produce in other types of cells.

### IV. Methods of Producing Steviol Glycosides

Recombinant hosts described herein can be used in methods to produce steviol glycosides such as Rebaudioside M. For example, if the recombinant host is a microorganism, the method can include growing the recombinant microorganism in a culture medium under conditions in which steviol and/or steviol glycoside biosynthesis genes are expressed. The recombinant microorganism can be grown in a fed batch or continuous process. Typically, the recombinant microorganism is grown in a fermentor at a defined temperature(s) for a desired period of time. In certain embodiments, microorganisms include, but are not limited to S. cerevisiae, A. niger, A. oryzae, E. coli, L. lactis and B. subtilis. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

Depending on the particular microorganism used in the method, other recombinant genes such as isopentenyl biosynthesis genes and terpene synthase and cyclase genes can also be present and expressed. Levels of substrates and intermediates, e.g., isopentenyl diphosphate, dimethylallyl diphosphate, geranylgeranyl diphosphate, kaurene and kaurenoic acid, can be determined by extracting samples from culture media for analysis according to published methods.

After the recombinant microorganism has been grown in culture for the desired period of time, steviol and/or one or more steviol glycosides can then be recovered from the culture using various techniques known in the art. In some embodiments, a permeabilizing agent can be added to aid the feedstock entering into the host and product getting out. If the recombinant host is a plant or plant cells, steviol or steviol glycosides can be extracted from the plant tissue using various techniques known in the art. For example, a crude lysate of the cultured microorganism or plant tissue can be centrifuged to obtain a supernatant. The resulting supernatant can then be applied to a chromatography column, e.g., a C18 column such as Aqua®C18 column from Phenomenex or a Synergi™ Hydro RP 80 Å column, and washed with water to remove hydrophilic compounds, followed by elution of the compound(s) of interest with a solvent such as acetonitrile or methanol. The compound(s) can then be further purified by preparative HPLC. See also WO 2009/140394.

The amount of steviol glycoside (e.g., Rebaudioside M) produced can be from about 1 mg/L to about 2800 mg/L, e.g., about 1 to about 10 mg/L, about 3 to about 10 mg/L, about 5 to about 20 mg/L, about 10 to about 50 mg/L, about 10 to about 100 mg/L, about 25 to about 500 mg/L, about 100 to about 1,500 mg/L, or about 200 to about 1,000 mg/L. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

It will be appreciated that the various genes and modules discussed herein can be present in two or more recombinant microorganisms rather than a single microorganism. When a plurality of recombinant microorganisms is used, they can be grown in a mixed culture to produce steviol and/or steviol glycosides. For example, a first microorganism can comprise one or more biosynthesis genes for producing steviol while a second microorganism comprises steviol glycoside biosynthesis genes. Alternatively, the two or more microorganisms each can be grown in a separate culture medium and the product of the first culture medium, e.g., steviol, can be introduced into second culture medium to be converted into a subsequent intermediate, or into an end product such as Rebaudioside A. The product produced by the second, or final microorganism is then recovered. It will also be appreciated that in some embodiments, a recombinant microorganism is grown using nutrient sources other than a culture medium and utilizing a system other than a fermentor.

Steviol glycosides do not necessarily have equivalent performance in different food systems. It is therefore desirable to have the ability to direct the synthesis to steviol glycoside compositions of choice. Recombinant hosts described herein can produce compositions that are selectively enriched for specific steviol glycosides (e.g., Rebaudioside M) and have a consistent taste profile. Thus, the recombinant microorganisms, plants, and plant cells described herein can facilitate the production of compositions that are tailored to meet the sweetening profile desired for a given food product and that have a proportion of each steviol glycoside that is consistent from batch to batch. Microorganisms described herein do not produce the undesired plant byproducts found in Stevia extracts. Thus, steviol glycoside compositions produced by the recombinant microorganisms described herein are distinguishable from compositions derived from Stevia plants.

### V. Food Products

The steviol glycosides obtained by the methods disclosed herein can be used to make food and beverage products, dietary supplements and sweetener compositions. For example, substantially pure steviol glycoside such as Rebaudioside M can be included in food products such as ice cream, carbonated beverages, fruit juices, yogurts, baked goods, chewing gums, hard and soft candies, and sauces. Substantially pure steviol glycoside also can be included in non-food products such as pharmaceutical products, medicinal products, dietary supplements and nutritional supplements. Substantially pure steviol glycosides can also be included in animal feed products for both the agriculture industry and the companion animal industry. Alternatively, a mixture of steviol glycosides can be made by culturing recombinant microorganisms separately or growing different plants/plant cells, each producing a specific steviol or steviol glycoside, recovering the steviol or steviol glycoside in substantially pure form from each microorganism or plant/plant cells and then combining the compounds to obtain a mixture containing each compound in the desired proportion (e.g., Rebaudioside M with one or more other steviol glycosides). The recombinant microorganisms, plants, and plant cells described herein permit more precise and consistent mixtures to be obtained compared to current Stevia products. In another alternative, a substantially pure steviol glycoside can be incorporated into a food product along with other sweeteners, e.g. saccharin, dextrose, sucrose, fructose, erythritol, aspartame, sucralose, monatin, or acesulfame potassium. The weight ratio of steviol glycoside relative to other sweeteners can be varied as desired to achieve a satisfactory taste in the final food product. See, e.g., U.S. Patent Publication No. 2007/0128311. In some embodiments, the steviol glycoside can be provided with a flavor (e.g., citrus) as a flavor modulator.

Compositions produced by a recombinant microorganism, plant, or plant cell described herein can be incorporated into food products. For example, a steviol glycoside composition produced by a recombinant microorganism, plant, or plant cell can be incorporated into a food product in an amount ranging from about 20 mg steviol glycoside/kg food product to about 1800 mg steviol glycoside/kg food product on a dry weight basis, depending on the type of steviol glycoside and food product. For example, a steviol glycoside composition produced by a recombinant microorganism, plant, or plant cell can be incorporated into a dessert, cold confectionary (e.g., ice cream), dairy product (e.g., yogurt), or beverage (e.g., a carbonated beverage) such that the food product has a maximum of 500 mg steviol glycoside/kg food on a dry weight basis. A steviol glycoside composition produced by a recombinant microorganism, plant, or plant cell can be incorporated into a baked good (e.g., a biscuit) such that the food product has a maximum of 300 mg steviol glycoside/kg food on a dry weight basis. A steviol glycoside composition produced by a recombinant microorganism, plant, or plant cell can be incorporated into a sauce (e.g., chocolate syrup) or vegetable product (e.g., pickles) such that the food product has a maximum of 1000 mg steviol glycoside/kg food on a dry weight basis. A steviol glycoside composition produced by a recombinant microorganism, plant, or plant cell can be incorporated into bread such that the food product has a maximum of 160 mg steviol glycoside/kg food on a dry weight basis. A steviol glycoside composition produced by a recombinant microorganism, plant, or plant cell can be incorporated into a hard or soft candy such that the food product has a maximum of 1600 mg steviol glycoside/kg food on a dry weight basis. A steviol glycoside composition produced by a recombinant microorganism, plant, or plant cell can be incorporated into a processed fruit product (e.g., fruit juices, fruit filling, jams, and jellies) such that the food product has a maximum of 1000 mg steviol glycoside/kg food on a dry weight basis.

In some embodiments, a substantially pure steviol or steviol glycoside is incorporated into a tabletop sweetener or “cup-for-cup” product. Such products typically are diluted to the appropriate sweetness level with one or more bulking agents, e.g., maltodextrins, known to those skilled in the art. Steviol glycoside compositions enriched for Rebaudioside M can be package in a sachet, for example, at from 10,000 to 30,000 mg steviol glycoside/kg product on a dry weight basis, for tabletop use.

The invention will be further described in the following examples, which do not limit the scope of the invention described in the claims.

### EXAMPLES

The Examples that follow are illustrative of specific embodiments of the invention and various uses thereof. They are set forth for explanatory purposes only and are not to be taken as limiting the invention.

### Example 1: Strain Engineering and Fermentation of EFSC 3044

Yeast strain EFSC 3044 was derived from a wild type Saccharomyces cerevisiae strain containing three auxotrophic modifications, namely the deletions of URA3, LEU2 and HIS3. The strain can be manipulated using standard genetic methods and can be used as a regular diploid or haploid yeast strain. The strain was converted to steviol glycosides-producing yeast by genomic-integration of five DNA constructs. Each construct contained multiple genes and was introduced into the yeast genome by homologous recombination. Furthermore, the first, second, and fifth construct were assembled by homologous recombination in yeast.

The first construct contained eight genes and was inserted in the DPP1 locus and disrupted and partially deleted DPP1 (phosphatase). The DNA inserted contained: the Ashbya gossypii TEF1 promoter expressing the natMX gene (selectable marker) followed by the TEF1 terminator from A. gossypii; Gene Art codon optimized Stevia rebaudiana UGT85C2 (GenBank AAR06916.1; SEQ ID NO:3) expressed from the native yeast GPD1 promoter and followed by the native yeast CYC1 terminator; S. rebaudiana CPR-8 (SEQ ID NO:5) expressed using the native yeast TPI1 promoter followed by the native yeast TDH1 terminator; Arabidopsis thaliana kaurene synthase (SEQ ID NO:6, similar to GenBank AEE36246.1) expressed from the native yeast PDC1 promoter and followed by the native yeast FBA1 terminator; synthetic Synechococcus sp. GGPPS (SEQ ID NO: 22, GenBank ABC98596.1) expressed using the native yeast TEF2 promoter and followed by the native yeast PGI1 terminator; DNA2.0 codon-optimized S. rebaudiana KAHe1 (SEQ ID NO:8) expressed from the native yeast TEF1 promoter and followed by the native yeast ENO2 terminator; synthetic S. rebaudiana KO-1 (SEQ ID NO: 23, GenBank ABA42921.1) expressed using the native yeast FBA1 promoter and followed by the native yeast TDH2 terminator; and Zea mays truncated CDPS (SEQ ID NO:133) expressed using the native yeast PGK1 promoter and followed by the native yeast ADH2 terminator.

The second construct was inserted at the YPRCΔ15 locus and contained: the TEF1 promoter from A. gossypii in front of the kanMX gene (selectable marker) followed by the TEF1 terminator from A. gossypii; the Gene Art codon optimized A. thaliana ATR2 (SEQ ID NO: 10) expressed from the native yeast PGK1 promoter followed by the native yeast ADH2 terminator; S. rebaudiana UGT74G1 (SEQ ID NO:135, GenBank AAR06920.1) expressed from the native yeast TP11 promoter followed by the native yeast TDH1 terminator; Gene Art codon-optimized S. rebaudiana UGT76G1 (SEQ ID NO:14, encodes GenBankAAR06912) expressed from the native yeast TEF1 promoter followed by the native yeast ENO2 terminator; and GeneArt codon-optimized sequence encoding a S. rebaudiana UGT91D2e-b with the amino acid modifications L211M and V286A (SEQ ID NO:15 for UGT91D2e amino acid sequence for the wild type sequence; codon optimized nucleotide sequence is set forth in SEQ ID NO:90) and expressed from the native yeast GPD1 promoter and followed by the native yeast CYC1 terminator. UGT91D2e-b is disclosed herein as SEQ ID NO: 66 with mutations at methionine residue at residue 211 and an alanine residue at residue 286.

The first and the second construct were combined in the same spore clone by mating and dissection. This yeast strain was subsequently transformed with construct three and four in two successive events.

Construct three was integrated between genes PRP5 and YBR238C and contained the Kluyveromyces lactis leu2 promoter expressing the K. lactis leu2 gene followed by the leu2 terminator from K. lactis, the native yeast GPD1 promoter expressing the DNA2.0-optimized S. rebaudiana KAHe1 (SEQ ID NO:8) followed by the native yeast CYC1 terminator, and the native yeast TP11 promoter expressing the Zea mays truncated CDPS (SEQ ID NO: 133) followed by the native yeast TP11 terminator.

Construct four was integrated in the genome between genes ECM3 and YOR093C with an expression cassette containing the TEF1 promoter from A. gossypii expressing the K. pneumoniae hphMX gene followed by the TEF1 terminator from A. gossypii, Synechococcus sp. GGPPS (SEQ ID NO: 22) expressed from the native yeast GPD1 promoter followed by the native yeast CYC1 terminator, and the native yeast TP11 promoter expressing the A. thaliana KS (SEQ ID NO: 6) followed by the native yeast TP11 terminator.

The four introduced selectable markers natMX, kanMX, K. lactis LEU2 and K. pneumoniae hphMX and the promoters preceding and terminators succeeding the selectable marker genes were then removed by recombination.

In this yeast strain, the fifth construct was inserted and assembled by yeast transformation and homologue recombination. The fifth construct contained seven genes and was inserted at the YORWΔ22 locus. The DNA inserted contained: the A. gossypii TEF1 promoter expressing the Schizosaccharomyces Pombe HIS5 gene (selectable marker) followed by the TEF1 terminator from A. gossypii; S. rebaudiana KO-1 (SEQ ID NO: 23, GenBank ABA42921.1) expressed from the native yeast GPD1 promoter and followed by the native yeast CYC1 terminator; S. rebaudiana CPR-8 (SEQ ID NO: 5) expressed using the native yeast TP11 promoter followed by the native yeast TDH1 terminator; Arabidopsis thaliana kaurene synthase (SEQ ID NO: 6, similar to GenBank AEE36246.1) expressed from the native yeast PDC1 promoter and followed by the native yeast FBA1 terminator; a codon optimized version of the rice gene Os03g0702000 (SEQ ID NO:18, encoding EUGT11) expressed using the native yeast TEF2 promoter and followed by the native yeast PG11 terminator; DNA2.0 codon-optimized S. rebaudiana KAHe1 (SEQ ID NO: 8) expressed from the native yeast TEF1 promoter and followed by the native yeast ENO2 terminator; and Zea mays truncated CDPS (SEQ ID NO:133) expressed using the native yeast PGK1 promoter and followed by the native yeast ADH2 terminator.

The described yeast strain was made prototrophic by introduction of the two plasmids, EPSC2182 and EPSC2308. EPSC2182 was derived from a p415TEF CEN/ARS shuttle plasmid with a LEU2 marker and contains another copy of S. rebaudiana KAHe1 expressed from the native yeast TEF1 promoter and succeeded by the native yeast CYC1 terminator. EPSC2308 was a p416TEF-based CEN/ARS shuttle plasmid with the URA3 marker wherein the EUGT11 gene was cloned and expressed from the native yeast TEF1 promoter and succeeded by the native yeast CYC1 terminator. This yeast strain was then designated EFSC 3044.

TABLE 11
List of Recombinant Genes in Strain EFSC 3044.

| | Yeast | Construct |
|---|---|---|
| Gene Designation | Location | No. |
| UGT85C2 | Genomic | 1 |
| S. rebaudiana CPR-8 | Genomic | 1 |
| A thaliana Kaurene synthase | Genomic | 1 |
| Synechococcus sp. GGPPS | Genomic | 1 |
| S. rebaudiana KAHe1 | Genomic | 1 |
| S. rebaudiana KO-1 | Genomic | 1 |
| Zea mays truncated CDPS | Genomic | 1 |
| A. thaliana ATR2 | Genomic | 2 |
| S. rebaudiana UGT74G1 | Genomic | 2 |
| S. rebaudiana UGT76G1 | Genomic | 2 |
| Stevia UGT91D2e-b altered | Genomic | 2 |
| S. rebaudiana KAHe1 | Genomic | 3 |
| Zea mays truncated CDPS | Genomic | 3 |
| Synechococcus sp. GGPPS | Genomic | 4 |
| A. thaliana Kaurene synthase | Genomic | 4 |
| S. rebaudiana KO-1 | Genomic | 5 |
| S. rebaudiana CPR-8 | Genomic | 5 |
| A thaliana Kaurene synthase | Genomic | 5 |
| Os03g0702000 (EUGT11) | Genomic | 5 |
| S. rebaudiana KAHe1 | Genomic | 5 |
| Zea mays truncated CDPS | Genomic | 5 |
| S. rebaudiana KAHe1 | Plasmid | 6 |
| EUGT11 | Plasmid | 7 |

Fed-batch fermentation was carried out aerobically in 2 L (working volume) fermenters which included a ~16 hour growth phase in the base medium (Synthetic Complete media) followed by ~100 hours of feeding with glucose utilized as the carbon and energy source combined with trace metals, vitamins, salts, and Yeast Nitrogen Base (YNB) and/or amino acid supplementation. The pH was kept near pH 5, and the temperature setpoint was 30 °C. The feed rate was controlled to prevent oxygen depletion and to minimize ethanol formation (glucose-limited conditions). Whole culture samples (without cell removal) were taken and boiled in an equal volume of DMSO for total glycosides levels.

The following methodology was used to analyze steviol glycosides and steviol pathway intermediates, unless otherwise indicated. LC-MS analyses were performed using an UltiMate® 3000 UPLC system (Dionex, Sunnyvale, CA) fitted with an Acquity UPLC® BEH C18 column (100×2.1 mm, 1.7 μm particles; Waters, Milford, MA) connected to a TSQ Quantum Access (ThermoFisher Scientific) triple quadropole mass spectrometer with a heated electrospray ion (HESI) source. Elution was carried out using a mobile phase of eluent B (MeCN with 0.1% Formic acid) and eluent A (water with 0.1% Formic acid) by increasing the gradient from 29->48% B from min 0.0 to 4.0, increasing 48->100% B in min 4.0 to 4.2, holding 100% B from min 4.2 to 6.2, and re-equilibrating with 29% eluent B. The flow rate was 0.4 ml/min and the column temperature was kept at 55 °C. Steviol glycosides were detected using SIM (Single Ion Monitoring) in positive mode with the following m/z-traces in Table 12.

TABLE 12
Summary of Analytical Compounds Detected by LC/MS.

| | Exact | | Compound |
|---|---|---|---|
| Description | Mass | m/z trace | (typical t_{R} in min) |
| Steviol + | [M + H]⁺ | 481.2 ± 0.5 | 19-SMG (4.15), |
| 1 Glucose | 481.2796 | | 13-SMG (4.38) |
| | [M + Na]⁺ | 503.1 ± 0.5 | |
| | 503.2615 | | |
| Steviol + | [M + Na]⁺ | 665 ± 0.5 | Rubusoside (3.04) |
| 2 Glucose | 665.3149 | | Steviol-1,2-bioside |
| | | | (3.48) |
| | | | Steviol-1,3-bioside |
| | | | (4.05) |
| Steviol + | [M + Na]⁺ | 827.4 ± 0.5 | 1,2-Stevioside (2.28) |
| 3 Glucose | 827.3677 | | 1,3-Stevioside (2.82) |
| | | | Rebaudioside B (3.9) |
| Steviol + | [M + Na]⁺ | 989.4 ± 0.5 | Rebaudioside A (2.23) |
| 4 Glucose | 989.4200 | | |
| Steviol + | [M + Na]⁺ | 1151.4 ± 0.5 | Rebaudioside D (1.19) |
| 5 Glucose | 1151.4728 | | |
| Steviol + | [M + Na]⁺ | 1313.5 ± 0.5 | Rebaudioside M (1.31) |
| 6 Glucose | 1313.5257 | | |

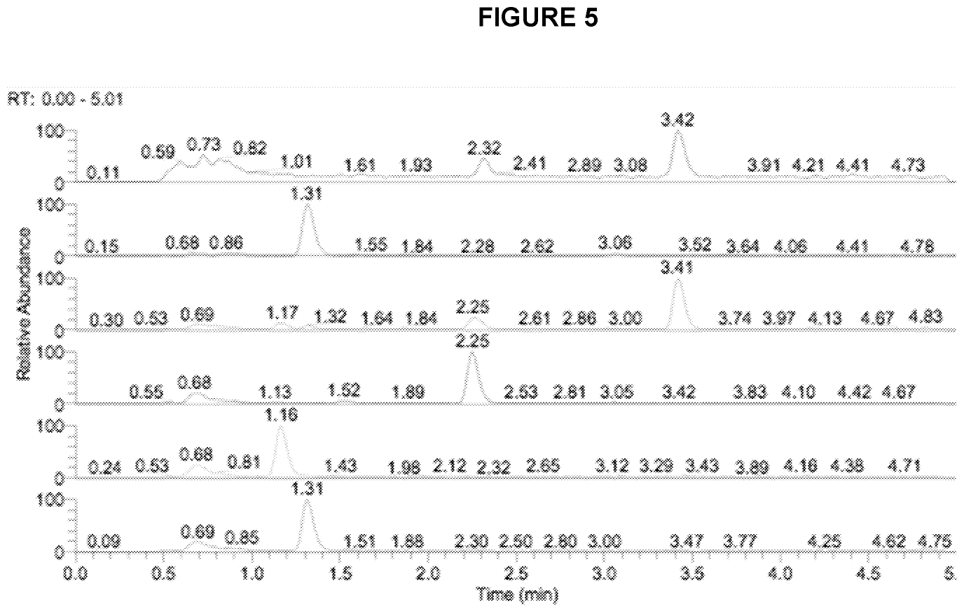

The level of steviol glycosides were quantified by comparing with calibration curves obtained with authentic standards from LGC Standards. For example, standard solutions of 0.5 to 100 μM Rebaudioside A (RebA) were typically utilized to construct a calibration curve. FIG. 5 contains representative mass spectra of fermentations that resulted in the formation of a hexaglycosylated steviol glycoside (retention time 1.31, mass traces corresponding to a hexa-glucose steviol glycoside and stevioside).

A modified LC-MS methodology (using a BEH RPshield C18 HPLC column (50×2.1 mm, 1.7 μm particles; Waters, Milford, MA) was used to analyze compounds described in Example 5 and in vitro experiment to determine relative rates for UGT76G1. The elution was carried out using a mobile phase of eluent B (MeCN with 0.1% formic acid) and eluent A (water with 0.1% formic acid) by increasing the gradient from 25->47% B from min 0.0 to 4.0, increasing 47->100% B in min 4.0 to 5.0, holding 100% B from min 5.0 to 6.5, and finally re-equilibrating with 25% B. The flow rate was 0.4 ml/min and the column temperature was kept at 35 °C. A modified LC-MS methodology resulted in shorter retention time for the compounds shown in Table 12. Typical retention times using the modified LC-MS methodology (t_{R} in min) were: 3.34 for 19-SMG; 3.54 for 13-SMG; 2.55 for Rubusoside; 2.95 for Steviol-1,2-bioside; 3.31 for Steviol-1,3-bioside; 2.04 for 1,2-Stevioside; 2.42 for 1,3-Stevioside; 2.91 for Rebaudioside B; 2.03 for Rebaudioside A; 1.1 for Rebaudioside D; and 1.32 for Rebaudioside M.

### Example 2: In Vitro Characterization of Reactions that Produce a Hexa-Glycosylated Steviol Glycoside

As described in Example 1, a hexa-glucosyl steviol glycoside was observed when EUGT11 was expressed at high levels in steviol-glycoside producing yeast strains. To characterize the reactions that were occurring to produce this molecule, further in vitro work was done with individual UGTs.

UGT76G1 (SEQ ID NO: 1) was cloned into the pET30a plasmid (EMD Millipore). The resulting vector was transformed into an appropriate DE3 E. coli strain and transformants were grown and induced according to manufacturer's protocols. The corresponding fusion protein (6×HIS-tagged) was purified by immobilized metal affinity chromatography using standard methods.

Approximately 0.08 μg of purified UGT76G1 per μL of reaction was incubated with 100 μM RebD, 300 μM UDP-glucose, and 10 U/mL Alkaline Phosphatase (Fermentas/Thermo Fisher, Waltham, MA). The reactions were performed at 30 °C in 20 mM Hepes-NaOH, pH 7.6, for 24 hours. Prior to LC-MS analysis, one volume of 100% DMSO was added to each reaction and vortexed, and samples were centrifuged at 16,000×g for 1 minute.

A new peak appeared during the reaction at a mass corresponding to steviol+6 glucose moieties, eluting at 1.31 min and corresponding to a trace of one of the hexaglucosyl steviol glycosides found upon the overexpression of EUGT11 in vivo. This result suggested that UGT76G1 can further glycosylate RebD, resulting in a hexaglycoside. It was hypothesized that UGT76G1, in addition to making a 1,3-glucose linkage with the primary glucose at C13 of the steviol backbone, has a secondary activity of adding a 1,3-bound glucose to the primary glucose at C19. It is likely that the only glycosylation site available in RebD available for UGT76G1 is the glucose at C19, which would result in the production of a hexaglycoside designated RebM. The hexaglycoside detected was isolated and determined to be Rebaudioside M, as shown in Examples 3 and 4.

### Example 3: Isolation of the Hexa-Qlycosylated Molecule

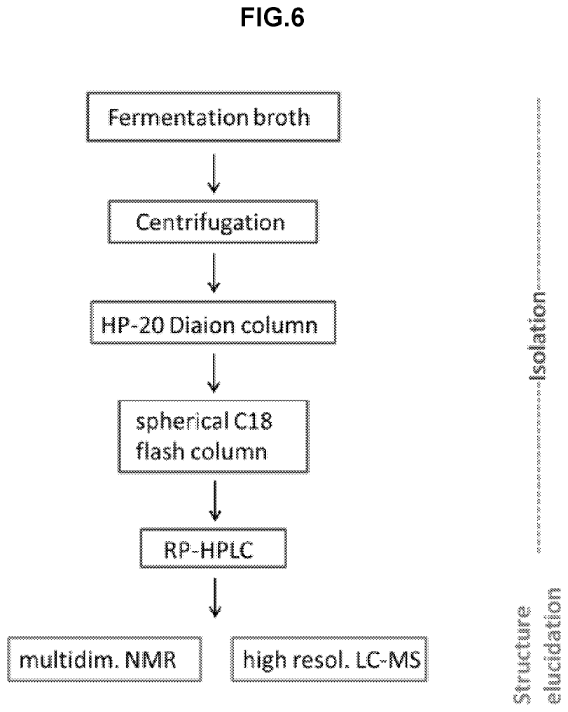

The hexa-glucosyl steviol glycoside product was isolated from a fermentation similar to that described in Example 1 for structural analysis following the scheme outlined in FIG. 6.

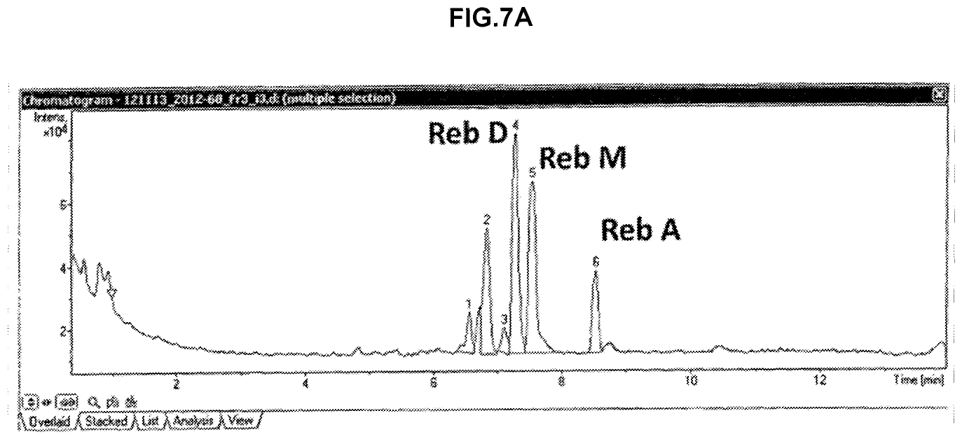

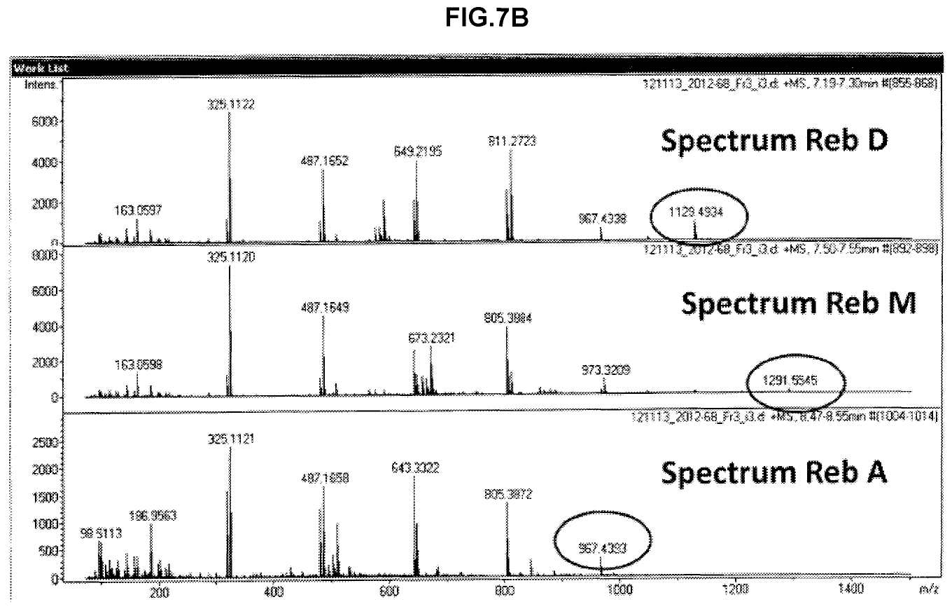

After the fermentation, the culture broth was centrifuged for 30 min at 7000 rpm at 4 °C and the supernatant was purified as follows: A glass column was filled with 150 mL HP20 Diaion® resin (Supelco), and an aliquot of 300 mL supernatant was loaded on to the column and washed with 2×250 mL MilliQ water. The glycoside product was eluted by stepwise incremental increases in the methanol concentration in MilliQ water (in 250 mL portions—starting with 0%→10%→40%→60%→80%→100% MeOH). The levels of steviol glycosides in each fraction were analyzed by LC-MS. The most promising fractions (60-80% MeOH) were combined and reduced to total of 10 mL using a vacuum evaporator. A glass column filled with 600 mL spherical C18 bonded flash silica gel (45-70 um, 70 Å/Supelco) was equilibrated with 5% aqueous acetonitrile (Acetronitrile: HPLC grade-Water: MilliQ). The concentrated residue from the HP20 purification was loaded on the column and eluted by stepwise increases in the acetonitrile contribution. The starting eluent was 5% acetonitrile in water. The level of acetonitrile was raised by 5% per step (each 400 mL). After reaching 50% acetonitrile 10% steps were made. All fractions were analyzed by LC-MS, pooled according to their steviol glycoside composition, and dried under vacuum. Table 13 contains a summary of the glycosides found in each of the fractions. FIG. 7 contains a chromatogram and mass spectra from LC-QTOF analysis of the semi-purified hexa-glycosylated compound after flash chromatography.

TABLE 13
Summary of Fractionation of Steviol Glycosides.

| mg | Fraction | Description |
|---|---|---|
| 321.1 | 2-11 | RebD and some 6X glycosylated steviol glycoside |
| 138.3 | 12-20 | RebD and some 6X glycosylated steviol glycoside |
| 357.4 | 21-27 | Bulk of 6X glycosylated steviol glycoside |
| 98.9 | 28-30 | RebA |
| 68.4 | 31-36 | Rubusoside, steviol-1,2-bioside |
| 14.8 | 34-45 | Mostly 13-SMG |
| 852.8 | Wash | Acetonitrile wash |

### Example 4: NMR Confirmation of Structure

To produce a pure sample for NMR, approximately 50 mg of the hexa-glycosylated enriched residue obtained in Example 3 were further purified on a semi-preparative HPLC system. The system was equipped with an Aqua® C18 column (Phenomenex: Dimension 250×21.2 mm, 5 micron). Elution was carried out using a mobile phase of eluent B (MeCN with 0.1% trifluoroacetic acid) and eluent A (water with 0.1% triflouroacidic acid) by increasing the gradient from 1%→50% B from min 0.0 to 21, 50->100% min 21.0 to 27.0 and finally washing with 100% B and re-equilibration. The flow rate was 14 mL/min at room temperature. The fractions were collected by time and analyzed by LC-QTOF-MS for the presence of steviol glycosides. The system used was a UPLC (Waters) coupled to a MicrOTOFII Mass Spectrometer (Bruker). The column used was Acquity UPLC® BEH C18, 100×2.1 mm, 1.7 μm (Waters). Mobile phases were A: 0.1% Formic Acid in water and B: 0.1% Formic Acid in Acetonitrile. The gradient applied was from 1% B to 50% B in 12 minutes and then to 100% B in 3 minutes. The flow rate was 0.4 ml/min.

Fraction 93 was utilized for NMR analysis. All NMR experiments were performed in DMSO-d6 at 25 C using a Bruker Avance III 600 MHz NMR spectrometer equipped with a 1.7 mm cryogenic TCI probe.

The structures were solved by means of standard homo- and heteronuclear multipulse NMR experiments, namely ¹H, ¹H—COSY, ¹H, ¹³C—HSQC and ¹H, ¹³C—HMBC experiments. The NMR data obtained was as follows:

1H NMR (600 MHz, DMSO-d6) δ ppm 0.78 (br. s., 1H) 0.83 (s, 3H) 0.92 (d, J=7.39 Hz, 2H) 0.97-1.04 (m, 1H) 1.17 (s, 3H) 1.30-1.54 (m, 6H) 1.67 (d, J=10.40 Hz, 1H) 1.72-1.86 (m, 4H) 1.92 (d, J=6.49 Hz, 1H) 1.96-2.05 (m, 2H) 2.08 (d, J=10.82 Hz, 1H) 2.32 (d, J=12.71 Hz, 1H) 2.91 (t, J=8.82 Hz, 1H) 2.95-3.01 (m, 1H) 3.02-3.27 (m, 14H) 3.31-3.55 (m, 10H) 3.57-3.86 (m, 10H) 4.47 (d, J=7.86 Hz, 1H) 4.51 (d, J=8.00 Hz, 1H) 4.53 (d, J=7.62 Hz, 1H) 4.66 (d, J=7.81 Hz, 1H) 4.73 (br. s., 1H) 4.80 (d, J=7.86 Hz, 1H) 5.11 (br. s., 1H) 5.53 (d, J=8.19 Hz, 1H)

13C NMR (150.91 MHz, DMSO-d6) δ ppm 16.4, 19.4, 19.9, 21.8, 28.3, 36.7, 37.2, 39.3, 40.3, 41.5, 41.6, 43.2, 43.7, 47.0, 47.2, 53.2, 57.0, 60.7, 61.2, 61.4, 61.8, 62.0, 62.1, 68.5, 68.9, 70.4 (3C), 71.2, 71.6, 74.1-74.3 (4C), 74.8, 75.8, 76.9-77.1 (6C), 77.6, 79.6, 85.8, 86.8, 87.1, 92.1, 96.2, 102.1, 102.8, 103.2, 103.3, 104.5, 153.1, 175.1

The confirmed structure of RebM (systematic name: 13-[β-D-glucopyranosyl-(1->3)-[β-D-glucopyranosyl-(1->2)]-□β-D-glucopyranosyl-1-oxy]kaur-16-en-18-oic acid, 18-[β-D-glucopyranosyl-(1->3)-[β-D-glucopyranosyl-(1->2))]-□β-D-glucopyranosyl-1-ester]) is shown in FIG. 2.

### Example 5: Isolation and Determination of Additional Fermentation Products of EFSC 3044

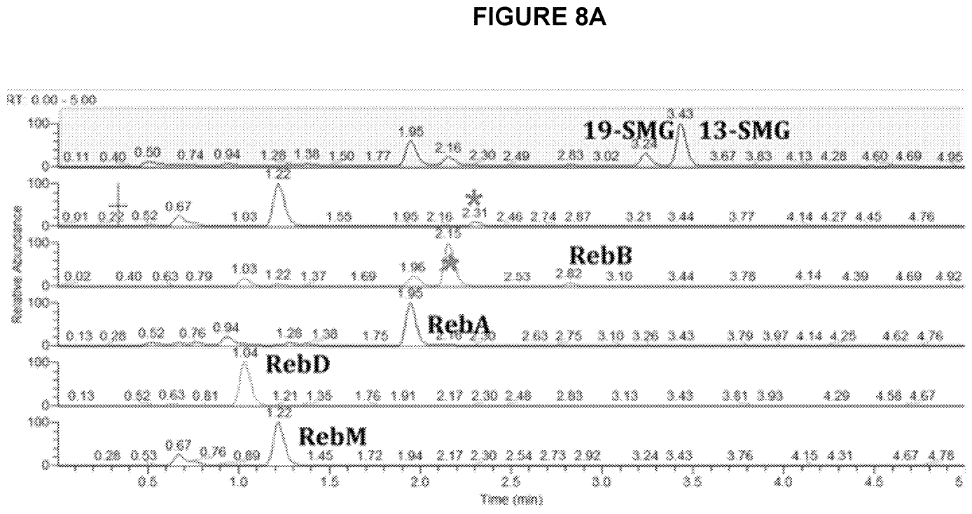

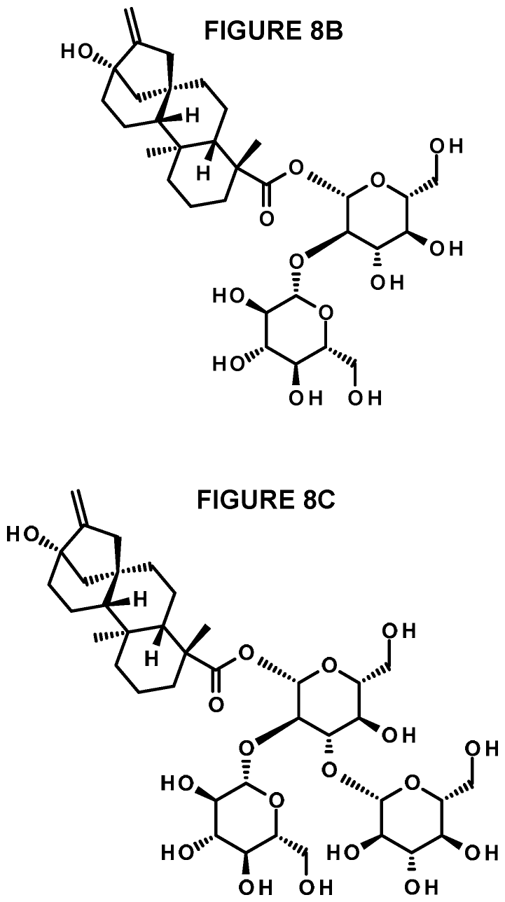

In addition to RebM, fermentation of EFSC 3044 resulted in formation of a di-glycosylated steviol glycoside (13-hydroxy kaur-16-en-18-oic acid, [2-O-β-D-glucopyranosyl-□β-D-glucopyranosyl]ester) with a retention time of 2.31 (FIG. 8B) and a tri-glycosylated steviol glycoside (13-hydroxy kaur-16-en-18-oic acid; [2-O-β-D-glucopyranosyl-3-O-□β-D-glucopyranosyl-β-D-glucopyranosyl]ester) with a retention time of 2.15 (FIG. 8C).

These compounds were isolated according to the following method. After the fermentation, the culture broth was centrifuged for 10 min at 5000 rpm at 4 °C and the supernatant was purified as follows: A glass column was filled with 300 mL HP20 Diaion® resin (Supelco), and an aliquot of 1700 mL supernatant was loaded on to the column and washed with 3.5 Litres of ddH2O. The compounds were eluted by using 2 L MeOH and fractions of 500 mL each collected. After LC-MS analysis, the fractions containing the majority of the target compounds were pooled and evaporated on a rotary evaporation system (Rotavap, Büchi, Switzerland) yielding 1.85 grams of dark grey material. The crude extract was re-dissolved in 3.5 mL of DMSO and injected in aliquots of 0.7 mL in a semi-preparative LC-MS for further purification. The column used was a XBridge C18, 19×250 mm, 5 um (Waters Corporation). Mobile phases were A: 0.1% TFA in water and B: 0.1% TFA in Acetonitrile. Elution was done by a linear gradient from 1% B to 60% B in 44 min. Fractions of 2.1 mL were continuously collected during the run. Fractions collected were analysed by LC-MS in order to evaluate the presence and purity of target analytes. Fractions containing compounds identified as ‘Peak 8’ and ‘Peak 9’ were neutralized by adding 0.8 mL of NH3 (aq.), pooled and dried by Genevac centrifugal evaporation system.

Structures of these compounds were determined by NMR with the method of Example 4.

The NMR data obtained for the di-glycosylated steviol glycoside are as follows:

1H NMR (600 MHz, DMSO-d₆) δ ppm 0.72-0.79 (m, 1H) 0.81 (s, 3H) 0.93 (d, J=8.07 Hz, 1H) 0.98-1.05 (m, 1H) 1.17 (s, 3H) 1.20 (d, J=11.37 Hz, 1H) 1.36 (d, J=4.03 Hz, 2H) 1.40-1.52 (m, 1H) 1.55-1.70 (m, 1H) 1.77 (d, J=9.54 Hz, 3H) 1.84-1.90 (m, 1H) 2.03 (d, J=8.07 Hz, 1H) 2.31-2.41 (m, 1H) 2.79-2.87 (m, 1H) 2.89-2.96 (m, 1H) 3.08 (s, 2H) 3.12-3.18 (m, 3H) 3.19-3.24 (m, 1H) 3.34 (d, J=4.77 Hz, 2H) 3.41-3.45 (m, 2H) 3.46-3.55 (m, 1H) 3.65 (d, J=11.37 Hz, 1H) 3.73 (dd, J=15.41, 8.44 Hz, 4H) 4.26-4.40 (m, 1H) 4.48 (d, J=7.70 Hz, 1H) 4.52-4.62 (m, 1H) 4.69 (br.s., 1H) 4.81 (d, J=7.70 Hz, 1H) 4.88 (br.s., 1H) 4.91-5.03 (m, 1H) 5.05-5.26 (m, 2H) 5.51 (d, J=7.70 Hz, 1H) 5.55 (br.s., 1H; the formula is C32H50O13; formula weight is 642.7316).

The NMR data obtained for the tri-glycosylated steviol glycoside are as follows:

1H NMR (600 MHz, DMSO-d₆) b ppm 0.73-0.79 (m, 1H) 0.81 (s, 3H) 0.89-0.97 (m, 2H) 0.99-1.05 (m, 1H) 1.17 (s, 3H) 1.19 (d, J=11.37 Hz, 1H) 1.24 (s, 2H) 1.31-1.40 (m, 4H) 1.40-1.51 (m, 3H) 1.55-1.63 (m, 1H) 1.67 (dd, J=14.12, 5.32 Hz, 1H) 1.71-1.82 (m, 5H) 1.88 (d, J=11.00 Hz, 1H) 1.98-2.08 (m, 2H) 2.23-2.30 (m, 1H) 2.94 (t, J=8.44 Hz, 1H) 3.01-3.11 (m, 3H) 3.12-3.17 (m, 1H) 3.19-3.28 (m, 3H) 3.44-3.52 (m, 5H) 3.54-3.60 (m, 1H) 3.61-3.71 (m, 3H) 4.36 (br. s., 1H) 4.49 (br. s., 1H) 4.55 (d, J=7.70 Hz, 1H) 4.69 (s, 1H) 4.73 (br. s., 1H) 4.88 (br. s., 1H) 4.91-5.05 (m, 2H) 5.17 (br.s., 1H) 5.31 (br.s., 1H) 5.44 (d, J=8.07 Hz, 1H) 5.55 (br. s., 1H; the formula is C38H60018; formula weight is 804.8722).

The di-glycosylated steviol glycoside ester was determined to be an analog of steviol-1,2-bioside (FIG. 8), and the tri-glycosylated steviol glycoside was determined to be an isomer of RebB, both of which are glycosylated at the 19-O position (FIG. 80) instead of the 13-O position of their respective isomers. This data suggests that these compounds form when the activity of UGT85C is low compared to the activity of EUGT11, UGT76G1, or UGT74G1.

### Example 6: Engineering and Fermentation of EFSC 3261

The wild type Saccharomyces strain utilized in Example 1 was modified to contain the heterologous genes in Table 14 involved in steviol glycoside production. The genes were all integrated into the chromosome of the host strain using similar methods described in Example 1.

TABLE 14
List of Recombinant Genes and Promoters Used in Strain EFSC 3261.

| | Number | |
|---|---|---|
| Heterologous pathway gene | of copies | Promoter(s) used |
| GGPPS7 (Synechococcus sp) | 2 | TEF2, GPD1 |
| synthetic gene | | |
| CDPS (truncated, Zea mays) | 3 | PGK1 (X2), TPI1 |
| native gene | | |
| KS5 (A. thaliana) native gene | 3 | TPI1, PDC1 (X2) |
| KO (S. rebaudiana KO1) | 2 | FBA1, GPD1 |
| synthetic gene | | |
| ATR2 synthetic gene | 1 | PGK1 |
| KAH (S. rebaudiana KAHe1) | 3 | GPD1, TEF1 (X2) |
| synthetic gene | | |
| S. rebaudiana CPR 8 native | 2 | TPI1 (X2) |
| gene | | |
| 85C2 (S. rebaudiana) | 1 | GPD1 |
| synthetic | | |
| 74G1 native (S. rebaudiana) | 1 | TPI1 |
| 76G1 synthetic (S. rebaudiana) | 1 | TEF1 |
| 91d2e-b 2X mutant from | 1 | GPD1 |
| S. rebaudiana | | |
| EUGT11 synthetic | 1 | TEF2 |
| (Oryza sativa) | | |

Fed-batch fermentation was carried out aerobically in 2 L (working volume) fermenters which included a ~16 hour growth phase in the base medium (minimal medium containing glucose, ammonium sulfate, trace metals, vitamins, salts, and buffer) followed by ~100 hours of feeding with a glucose-containing defined feed medium. Glucose was utilized as the carbon and energy source and combined with trace metals, vitamins, and salts. The pH was kept near pH 5 and the temperature setpoint was 30 °C. The feed rate was controlled to prevent oxygen depletion and to minimize ethanol formation (glucose-limited conditions). Whole culture samples (without cell removal) were taken and boiled in an equal volume of DMSO for total glycosides levels.

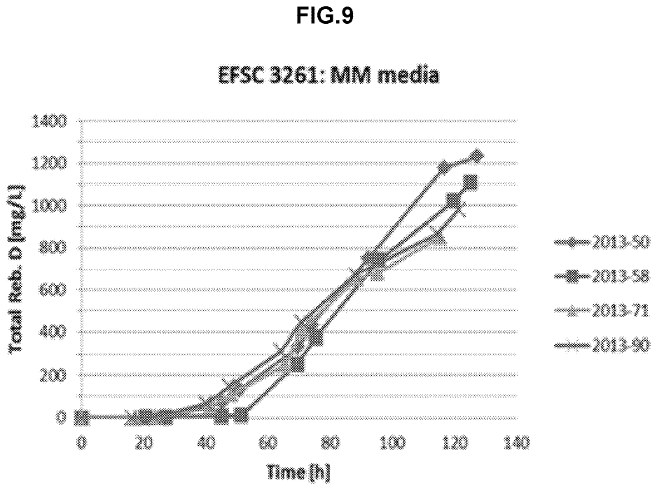

FIG. 9 shows production of RebD by EFSC 3261 in four separate trials. Total production (intracellular and extracellular combined) averaged titer were between 800-1200 mg/L. For fermentation run 58, the final total titer at 123 hours was 1109 mg/L RebD, 695 mg/L RebM; the ratio of D:M on a mass basis was 1.6. 394 mg/L RebA were also produced.

### Example 7: Strain Engineering and Fermentation of EFSC 3297 for Increased Production of RebD and RebM

The same wild type Saccharomyces strain utilized in Example 1 was modified to contain the heterologous genes in Table 15 involved in steviol glycoside production. The genes were all integrated into the chromosome of the host strain using similar methods described in Example 1. Although the genes used are identical to those in Example 1, increased copy numbers of bottleneck enzymes in the steviol pathway allowed for increased production of RebD and RebM. Fermentation of strain 3297 was carried out in a manner similar to that described above for strain 3261.

TABLE 15
List of Recombinant Genes and Promoters Used in Strain
EFSC 3297.

| | Number | |
|---|---|---|
| Heterologous pathway gene | of copies | Promoter(s) used |
| GGPPS7 (Synechococcus sp) | 3 | TEF2 (X2), GPD1 |
| synthetic gene | | |
| CDPS (truncated, Zea mays) | 4 | PGK1 (X3), TPI1 |
| native gene | | |
| KS5 (A. thaliana) native gene | 4 | TPI1, PDC1 (X3) |
| KO (S. rebaudiana KO1) | 2 | FBA1, GPD1 |
| synthetic gene | | |
| ATR2 synthetic gene | 1 | PGK1 |
| KAH (S. rebaudiana KAHe1) | 4 | GPD1 (X2), TEF1 |
| synthetic gene | | (X2) |
| S. rebaudiana CPR 8 native | 3 | TPI1 (X3) |
| gene | | |
| 85C2 (S. rebaudiana) | 1 | GPD1 |
| synthetic | | |
| 74G1 native (S. rebaudiana) | 1 | TPI1 |
| 76G1 synthetic (S. rebaudiana) | 1 | TEF1 |
| 91d2e-b 2X mutant from | 1 | GPD1 |
| S. rebaudiana | | |
| EUGT11 synthetic | 2 | TEF2 (X2) |
| (Oryza sativa) | | |

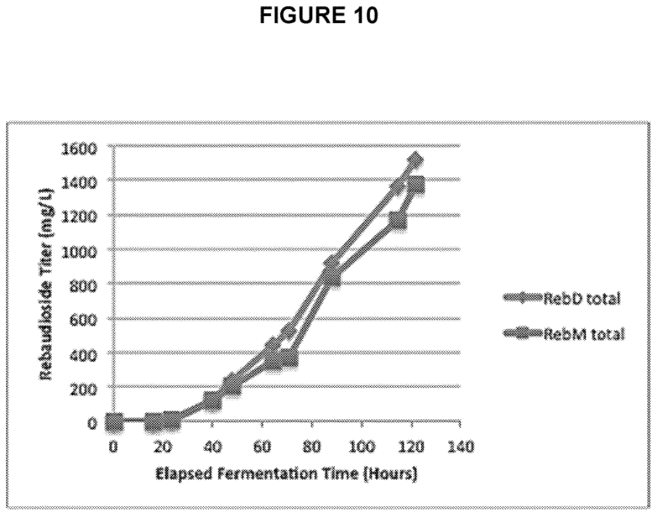

Production of RebD and RebM by EFSC 3297 is shown in FIG. 10. The ratio of D:M on a mass basis was 1.1. 1517 mg/L RebD was produced at the end of the fermentation (total, intracellular plus extracellular) and 1375 mg/L of RebM was produced.

### Example 8: Strain Engineering and Fermentation of EFSC 3841 with Two Copies of the UGT76G1 Gene

The wild type Saccharomyces strain utilized in Example 1 was modified to contain the heterologous genes in Table 16 involved in steviol glycoside production. The genes were all integrated into the chromosome of the host strain using similar methods described in Example 1. Fermentation conditions for 3841 were similar to those described above for strain 3261.

TABLE 16
List of Recombinant Genes and Promoters
Used in Strain EFSC 3841.

| | Number | |
|---|---|---|
| Heterologous pathway gene | of copies | Promoter(s) used |
| GGPPS7 (Synechococcus sp) | 3 | TEF2 (X3) |
| synthetic gene | | |
| CDPS (truncated, Zea mays) | 4 | PGK1 (X4) |
| native gene | | |
| KS5 (A. thaliana) native gene | 4 | PDC1 (X4) |
| KO (S. rebaudiana KO1) | 3 | FBA1, GPD1, TPI1 |
| synthetic gene | | |
| ATR2 synthetic gene | 2 | PGK1 (X2) |
| KAH (S. rebaudiana KAHe1) | 4 | GPD1, TEF1 (X3) |
| synthetic gene | | |
| S. rebaudiana CPR 8 native | 3 | TPI1 (X3) |
| gene | | |
| 85C2 (S. rebaudiana) | 2 | GPD1 (X2) |
| synthetic | | |
| 74G1 native (S. rebaudiana) | 2 | TPI1 (X2) |
| 76G1 synthetic (S. rebaudiana) | 2 | TEF1 (X2) |
| 91d2e-b 2X mutant from | 2 | GPD1 (X2) |
| S. rebaudiana | | |
| EUGT11 synthetic | 2 | TEF2, TEF1 |
| (Oryza sativa) | | |

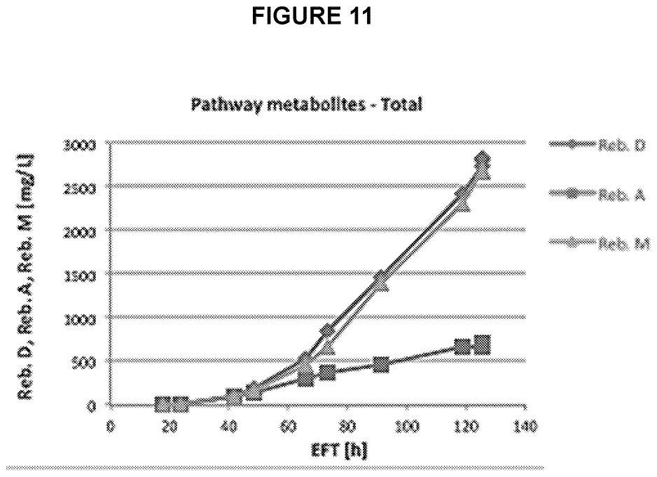

Production of RebD, RebM, and RebA by EFSC 3841 is shown in FIG. 11. Here, the total amount of RebD produced was 2786 mg/L, and the total amount of RebM produced was 2673 mg/L for a ratio of 1.04 D:M (g per g). 703.7 mg/L RebA was also produced.

### Example 9: Knockdown of One UGT76G1 Gene from EFSC 3841 and Decreased Production of RebD and RebM

An auxotrophic (leu2, ura3) version of strain EFSC 3841 described above, designated EFSC 3643, was further modified to delete one of the wild type 76G1 UGT genes. The performance of 3 colonies containing one copy of UGT 76G1 was tested versus 4 colonies of the unmodified strain which contains 2 copies of UGT 76G1. PCR was used to verify that the new strain only harbored one copy of the 76G1. Briefly, the disruption of one copy of UGT76G1 was verified by two PCR reactions amplifying a region upstream of the insertion site with part of the integration cassette and a region downstream of the insertion site with part of the integration cassette used for disruption. PCR primers designed for the wildtype 76G1 confirmed that wildtype 76G1 was still intact and present in the strain. The colonies were grown in 96 deep-well plates for 96 hours at 30 °C and 400 RPM. The total amounts of RebD and RebM were determined by LC/MS analysis.

From FIG. 12, it can be seen that the copy number of 76G1 significantly changes the RebD/RebM ratio. The ratio of RebD to RebM was plotted for the 3 colonies containing only one 76G1 copy (bars on the left-hand side of the graph), versus 4 colonies of the parent strain that contained 2 copies of the 76G1 UGT (right-hand bars of the graph).

### Example 10: Determination of Relative Rates of RebD and RebM Production

p416GPD containing WT-76G1 was expressed in the protease deficient yeast strain DSY6 for 48 h in SC-ura media. 100 μL of cells were then reinoculated in 3 mL of SC-ura media for 16 h. The cells were lysed with 200 μL CelLytic™ Y according to manufacturer's description. 6 μL of the lysate were added to 24 μL of the reaction mixture consisting of 20 mM Tris-buffer (pH 8.0), 0.3 μMUDPG, and 0.1 μM Reb D or Reb E. The reactions were incubated at 30 °C and stopped at 0, 1, 2, and 18 h by transferring 25 μL of the 30 μL reaction mixture to 25 μL DMSO. Amounts of RebD, RebE, and RebM were analyzed by LC-MS and assessed by peak integration during data processing as “area under the curve.”

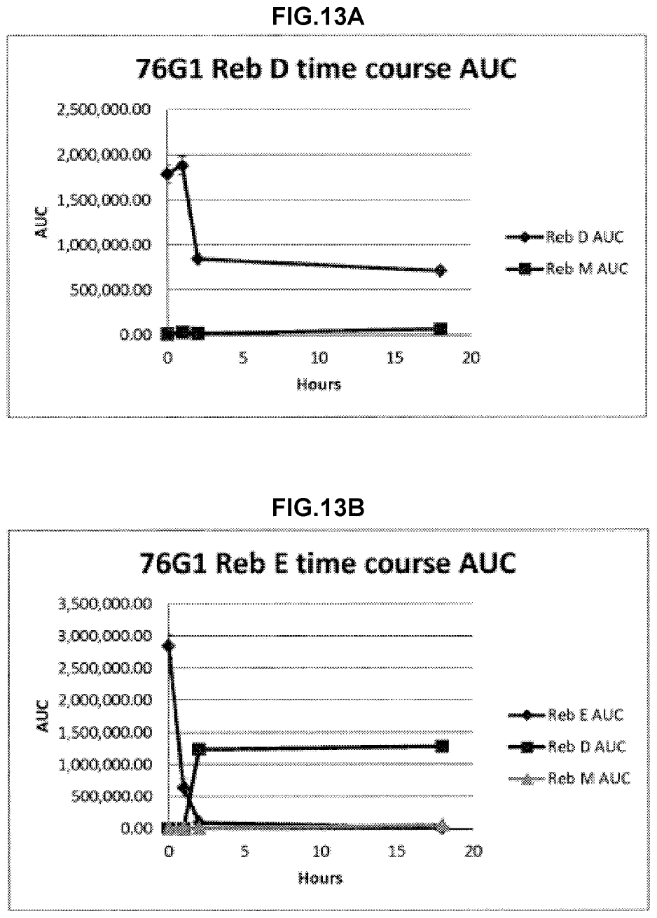

FIG. 13 shows that a large portion of RebD is consumed without generating a corresponding amount of RebM. It is also shown that RebE is consumed within 2 h and converted to RebD. This finding confirms an alternative glycosylation route from steviol to RebM through RebE instead of RebA is possible, which is first observed at the 18 h time point.

### Example 11: Prediction of Amino Acids Involved in RebM and RebD Binding in UGT76G1

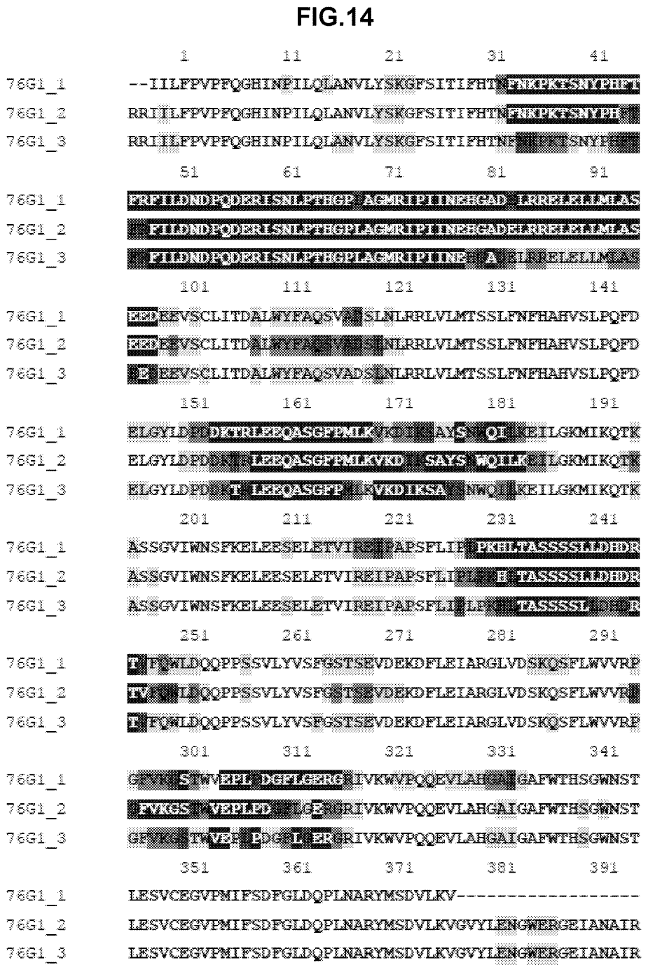

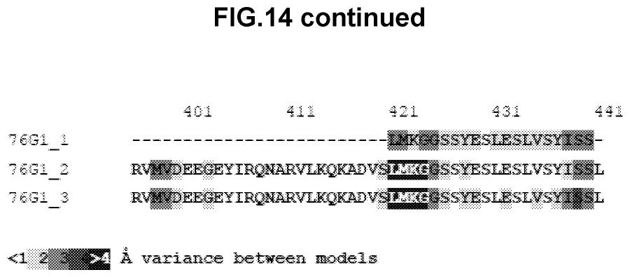

As a means for identifying UGT76G1 variants with increased activity and regioselectivity towards RebM or RebD, homology modeling and docking studies were performed. Three homology models were generated using standard setting in the SybyIX program with a combination of the following PDB-files 2PQ6 (% ID=31), 2C1X (% ID=28), 3HBF (% ID=28), 2VCE (% ID=35) as templates. The ligands present in PDB2VCE were used during the generation of main- and side-chains but removed prior to energy minimization. To yield the highest quality structures, models were energy minimized using an AMBER FF99 forcefield with either the standard settings or a gradient termination with a threshold of 0.1 kJ, a cutoff radius of 10 Å, and a maximum iteration of 5000 cycles. Statistics for the models are shown in Table 17, and variance between the models can be found in FIG. 14.

TABLE 17
Summary of UGT76G1 homology models.

| Statistic | Model 1 | Model 2 | Model 3 | Goal |
|---|---|---|---|---|
| Clashscore, | 1.1 | 0.42 | 3.38 | |
| all atoms | (99^{th} | (99^{th} | (97^{th} | |
| | percentile) | percentile) | percentile) | |
| Poor rotamers | 16 | 6 | 8 | <1% |
| | (4.47%) | (1.52%) | (2.02%) | |
| Ramachandran | 5 | 12 | 6 | <0.05% |
| outliers | (1.26%) | (2.70%) | (1.35%) | |
| Ramachandran | 354 | 375 | 404 | >98% |
| favored | (88.94%) | (84.27%) | (90.79%) | |
| MolProbity score | 1.89 | 1.46 | 1.89 | |
| | (81^{st} | (96^{th} | (81^{st} | |
| | percentile) | percentile) | percentile) | |
| Deviations > 0.25 Å | 3 | 1 | 5 | 0 |
| | (0.80%) | (0.24%) | (1.20%) | |
| Bad backbone bonds | 0/1599 | 0/1780 | 0/1780 | 0% |
| | (0%) | (0%) | (0%) | |
| Bad backbone | 0/1996 | 1/2224 | 0/2224 | <0.1% |
| angles | (0%) | (0.04%) | (0%) | |

After model generation, substrates were docked into the active site of the enzyme using the Surflex Dock suite in SybyIX to predict the amino acids forming the binding pocket. The UDPG portion of the UGT76G1 binding groove was located by aligning the 76G1 models with PDB2VCE and importing the ligand, UDPF2G, directly from the template. To dock the acceptor substrates, a protocol was generated using standard values covering the remaining part of the binding site. The dockings were performed using the GeomX settings on a ligand library containing steviol glycosides allowed with protein flexibility (model 1) or no flexibility (model 2). The docking results were analyzed using a combination of the scoring functions in SybyIX using top 3 docking results in base mode and top 1 docking result with protein flexibility.

All UGT76G1 amino acids are shown in Table 18 below. The sites for the saturation library were determined by selecting all residues found to be within 5 Å of RebD and RebM in the docking analysis on two or more models (shown as bold “x”). Furthermore, all residues found to be within 5 Å of the RebM and RebD 19-O-glucose moiety, which were positioned in the binding site for the RebD4→RebM reaction, were selected. Residues completely conserved between similar enzymes, which are shown in bold and with a “!,” were omitted from the screen.

TABLE 18
Prediction of amino acids involved in RebM and RebD binding in UGT76G1.

| Enzyme UGT76G1 | | | | | | | | | | |
|---|---|---|---|---|---|---|---|---|---|---|
| | | Top3 | Top3 | Top1 | Top1 | Top1 | | | | |
| | | (base) | (Base) | (PF) | (PF) | (Base) | | | | |
| | | Model no. | | | | | Model | | | |
| | | 2 | 1 | 1 | 1 | 1 | 1 + 2 | Unique | | |
| | | Substrate: | | | | | minus | 5 Å: | Unique | Total |
| | | | | | RebM | RebD | cons. | RebM | RebD | screened |
| | | RebM | RebM | RebM | 19Glcs | 19Glcs | AAs: | 19Glc: | 19Glcs: | residues: |
| | | Å: | | | | | | | | |
| | | 5 | 5 | 5 | 5 | 5 | 5 | 5 | 5 | 5 |
| | | No. of res in group: | | | | | | | | |
| Residue | | 73 | 58 | 42 | 15 | 11 | 23 | 12 | 3 | 38 |
| VAL | 20 | 20 | 20 | 20 | | | X | | | x |
| PRO | 21! | 21 | 21 | 21 | 21 | 21 | | | | |
| PHE | 22 | 22 | 22 | 22 | 22 | 22 | X | | | x |
| GLN | 23 | | 23 | 23 | 23 | 23 | | x | | x |
| GLY | 24 | 24 | 24 | 24 | | 24 | X | | | x |
| HIS | 25! | 25 | 25 | 25 | | | | | | |
| ILE | 26 | | 26 | 26 | | 26 | | | x | x |
| ASN | 27 | | 27 | 27 | | | | | | |
| THR | 48 | | 48 | | | | | | | |
| ASN | 49 | | 49 | 49 | 49 | 49 | | x | | x |
| PHE | 50 | | 50 | 50 | 50 | 50 | | x | | x |
| ASN | 51 | | 51 | 51 | 51 | 51 | | x | | x |
| PRO | 53 | | 53 | 53 | 53 | | | x | | x |
| LYS | 54 | | 54 | 54 | 54 | 54 | | x | | x |
| THR | 55 | | 55 | 55 | 55 | 55 | | x | | x |
| SER | 56 | | 56 | 56 | 56 | 56 | | x | | x |
| PRO | 80 | 80 | | | | | | | | |
| THR | 81 | 81 | | | | | | | | |
| HIS | 82 | 82 | | | | | | | | |
| GLY | 83 | 83 | | | | | | | | |
| PRO | 84 | 84 | | | | | | | | |
| LEU | 85 | 85 | 85 | | | | X | | | x |
| MET | 88 | 88 | | | | | | | | |
| ARG | 89 | 89 | | | | | | | | |
| ILE | 92 | | 92 | | | | | | | |
| GLU | 95 | | 95 | 95 | | | | | | |
| HIS | 96 | | 96 | 96 | | | | | | |
| ASP | 99 | | 99 | 99 | | | | | | |
| ARG | 103 | | 103 | 103 | | | | | | |
| THR | 123 | 123 | | | | | | | | |
| ASP | 124! | 124 | 124 | | | | | | | |
| ALA | 125 | 125 | | | | | | | | |
| LEU | 126 | 126 | 126 | 126 | | | X | | | x |
| TRP | 127 | 127 | 127 | 127 | | | X | | | x |
| TYR | 128 | 128 | 128 | | | | X | | | x |
| VAL | 143 | 143 | | | | | | | | |
| LEU | 144 | 144 | | | | | | | | |
| MET | 145 | 145 | 145 | | | | X | | | x |
| THR | 146 | 146 | 146 | 146 | | | X | | | x |
| SER | 147 | 147 | 147 | 147 | | | X | | | x |
| SER | 148 | 148 | | | | | | | | |
| PHE | 150 | 150 | | | | | | | | |
| ASN | 151 | 151 | 151 | 151 | | | X | | | x |
| PHE | 152 | 152 | | | | | | | | |
| ALA | 154 | 154 | | | | | | | | |
| HIS | 155 | 155 | 155 | 155 | | | X | | | x |
| VAL | 156 | 156 | | | | | | | | |
| SER | 157 | 157 | | | | | | | | |
| LEU | 158 | 158 | | | | | | | | |
| PRO | 159 | 159 | | | | | | | | |
| GLN | 160 | 160 | | | | | | | | |
| PHE | 161 | 161 | | | | | | | | |
| ASP | 162 | 162 | | | | | | | | |
| GLU | 163 | 163 | | | | | | | | |
| GLY | 165 | 165 | | | | | | | | |
| TYR | 166 | 166 | | | | | | | | |
| LEU | 167 | 167 | | | | | | | | |
| ASP | 168 | 168 | | | | | | | | |
| ASP | 189 | 189 | | | | | | | | |
| ILE | 190 | 190 | | | | | | | | |
| LYS | 191 | 191 | 191 | 191 | | | X | | | x |
| SER | 192 | 192 | | | | | | | | |
| ALA | 193 | 193 | | | | | | | | |
| TYR | 194 | 194 | | | | | | | | |
| SER | 195 | 195 | 195 | 195 | | | X | | | x |
| ASN | 196 | 196 | | | | | | | | |
| TRP | 197 | 197 | | | | | | | | |
| GLN | 198 | 198 | 198 | 198 | | | X | | | x |
| ILE | 199 | 199 | 199 | | | | X | | | x |
| LEU | 200 | 200 | 200 | 200 | | | X | | | x |
| LYS | 201 | 201 | | | | | | | | |
| GLU | 202 | 202 | | | | | | | | |
| ILE | 203 | 203 | 203 | 203 | | | X | | | x |
| LEU | 204 | 204 | 204 | 204 | | | X | | | x |
| GLY | 205 | 205 | | | | | | | | |
| LYS | 206 | 206 | | | | | | | | |
| MET | 207 | 207 | 207 | | | | | | | |
| ILE | 208 | 208 | | | | | | | | |
| LYS | 209 | 209 | | | | | | | | |
| SER | 253 | | 253 | 253 | 253 | 253 | | x | | x |
| LEU | 257 | | 257 | 257 | 257 | | | x | | x |
| PHE | 281! | | | | | 281 | | | | |
| GLY | 282! | | 282 | 282 | | 282 | | | | |
| SER | 283 | | 283 | 283 | 283 | 283 | | x | | x |
| THR | 284 | 284 | 284 | 284 | | 284 | X | | | x |
| SER | 285 | | 285 | 285 | | 285 | | | x | x |
| GLU | 286 | | 286 | | | | | | | |
| VAL | 309 | | 309 | | | | | | | |
| ARG | 311! | | 311 | | | | | | | |
| PHE | 314 | | 314 | 314 | 314 | 314 | | x | | x |
| LYS | 337 | | 337 | | | 337 | | | x | x |
| TRP | 338! | | 338 | 338 | 338 | 338 | | | | |
| HIS | 356! | | | | | 356 | | | | |
| GLY | 358! | 358 | | | | | | | | |
| TRP | 359! | 359 | 359 | | | | | | | |
| ASN | 360! | | 360 | | | | | | | |
| PHE | 377 | | 377 | | | | | | | |
| GLY | 378 | 378 | 378 | | | | X | | | x |
| LEU | 379 | 379 | 379 | 379 | | | X | | | x |
| ASP | 380 | 380 | 380 | 380 | | | X | | | x |
| GLN | 381! | 381 | 381 | 381 | | | | | | |
| PRO | 382 | 382 | | | | | | | | |
| LEU | 383 | 383 | | | | | | | | |
| ASN | 384 | 384 | | | | | | | | |

Bold indicates complete conservation in amino acid.
Bold “!” indicates amino acid residues that are completely conserved between similar enzymes and were omitted from the screen.

### Example 12: UGT76G1 Site Saturation Library Prescreen

Prior to performing the UGT76G1 site saturation library screening as described herein, culture growth and production of RebM and RebD were monitored in 96 and 4×24 deep-well plates. Using the standard lithium acetate protocol, the EFSC 3385 strain was transformed with p416GPD containing WT-76G1, and the transformants were plated on SC-URA plates. EFSC 3385 is a strain that is deficient in UGT76G1 and thus will make RebE until transformed with a plasmid containing an active UGT76G1. The strain contained a disruption in the UGT76G1 coding region, which was replaced with the spHIS5 marker, and also contained integrated copies of the UGT91 D2e-2X mutant, UGT74G1, ATR2, UGT85C2, S. rebaudiana CPR8 (2 copies), A. thaliana KS5 (2 copies), Synechococcus GGPPS7, codon optimized S. rebaudiana KAHe1 (2 copies), S. rebaudiana KO (two copies), the truncated Zea mays CDPS5 (2 copies), and EUGT11.

For the 96 deep-well plate condition, 96 colonies were transferred to a plate containing 1 mL SC-URA, and the plate was incubated at 30 °C and 400 RPM for 96 h. For the 4×24 deep-well plate condition, 96 colonies were transferred to a plate containing 3 mL SC-URA. The plate was incubated at 30 °C and 320 RPM for 96 h, and 200 μL were then transferred from each well to a 96 deep-well plate.

50 μL of the cultures from each plate were transferred to 96 well polymerase chain reaction (PCR) plates and diluted 1:1 with 100% dimethyl sulfoxide (DMSO). The plates were heat sealed, incubated at 80 °C for 10 min, and subsequently cooled to 25 °C. The plates were spun at 4000 RPM for 10 min, and 50 μL of the culture mixtures were transferred to a new plate for LC-MS analysis.

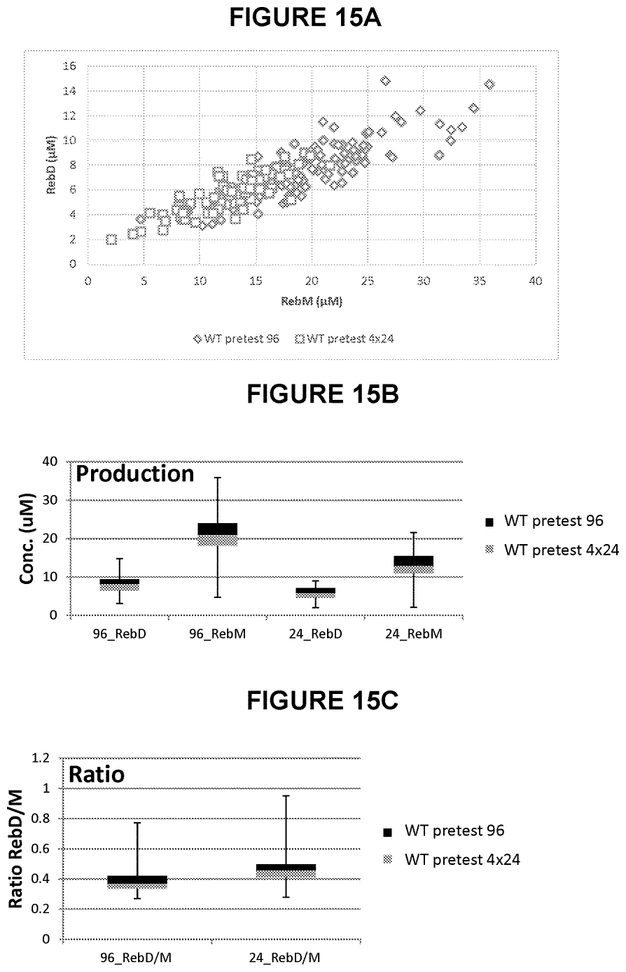

Results of the UGT76G1 site saturation library prescreen can be found in FIG. 15. Variance in RebD and RebM production can be explained by evaporation, particularly in the wells located at the edges of the plate, over the course of the 96 h incubation period. The higher concentrations of RebM and RebD produced by colonies grown in 96 deep-well plates suggest that these plates are better suited for LC-MS analysis, as compared to 4×24 deep-well plate, and were thus selected for use in the UGT76G1 site saturation library screen.

### Example 13: UGT76G1 Site Saturation Library Screen

Through the company, Baseclear, UGT76G1 was subcloned from EPSC2060 (p423GPD) to EPSB492 (p416GPD) using the Spel and Xhol restriction sites, and the site saturation libraries were created using degenerate NNS-primers. Using the standard lithium acetate protocol, the EFSC3385 strain was transformed with the library or with control plasmid containing WT-76G1, and the transformants were plated on SC-URA plates.

1 mL of SC-URA media was added to 96 deep-well plates, and colonies from each of the 38 site saturation library residues identified in Example 9 were picked and incubated in the 96 deep-well plates at 30 °C and 400 RPM for 96 h. 50 μL of each culture samples were then transferred to 96 well PCR plates containing 50 μL 100% DMSO. The plates were then heat sealed, incubated at 80 °C for 10 min, subsequently cooled to 12 °C, and spun at 4000 RPM for 10 min. 70 μL of each supernatant were transferred to a new plate for LC-MS analysis.

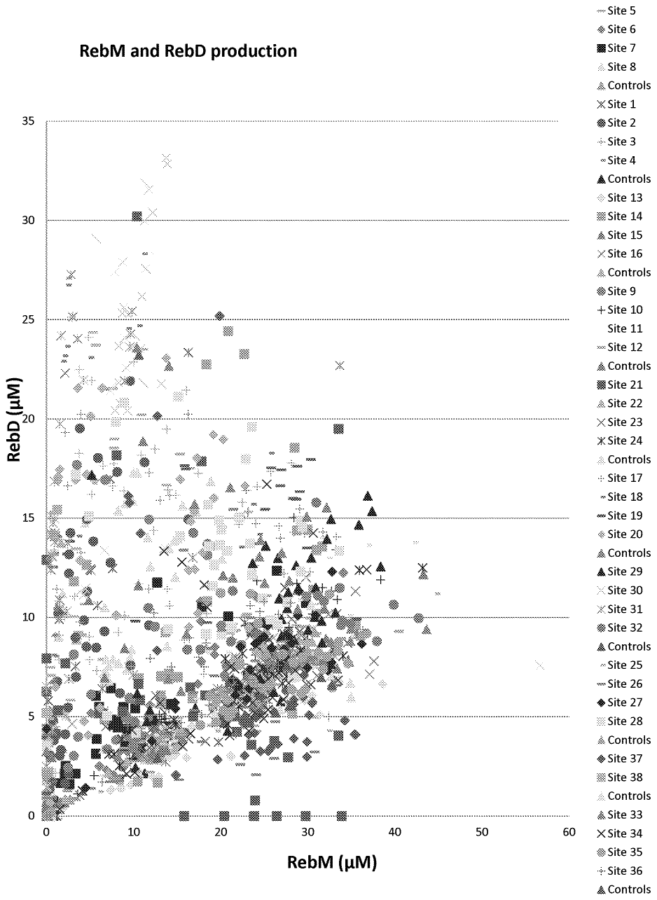

FIG. 16 shows all data points of the UGT76G1 site saturation library screen, with wild type production depicted with black triangles. The variant numbering system can be found in Table 19.

TABLE 19
Numbering for UGT76G1 site saturation library variants.

| Number | Residue | |
|---|---|---|
| 1 | VAL | 20 |
| 2 | PHE | 22 |
| 3 | GLN | 23 |
| 4 | GLY | 24 |
| 5 | ILE | 26 |
| 6 | ASN | 49 |
| 7 | PHE | 50 |
| 8 | ASN | 51 |
| 9 | PRO | 53 |
| 10 | LYS | 54 |
| 11 | THR | 55 |
| 12 | SER | 56 |
| 13 | LEU | 85 |
| 14 | LEU | 126 |
| 15 | TRP | 127 |
| 16 | TYR | 128 |
| 17 | MET | 145 |
| 18 | THR | 146 |
| 19 | SER | 147 |
| 20 | ASN | 151 |
| 21 | HIS | 155 |
| 22 | LYS | 191 |
| 23 | SER | 195 |
| 24 | GLN | 198 |
| 25 | ILE | 199 |
| 26 | LEU | 200 |
| 27 | GLU | 202 |
| 28 | ILE | 203 |
| 29 | SER | 253 |
| 30 | LEU | 257 |
| 31 | SER | 283 |
| 32 | THR | 284 |
| 33 | SER | 285 |
| 34 | PHE | 314 |
| 35 | LYS | 337 |
| 36 | GLY | 378 |
| 37 | LEU | 379 |
| 38 | ASP | 380 |

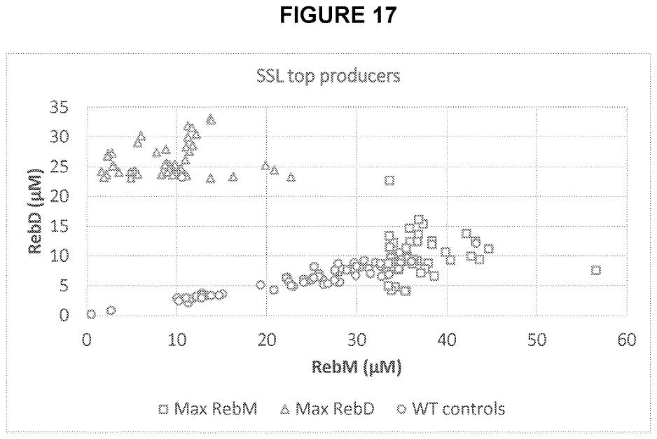

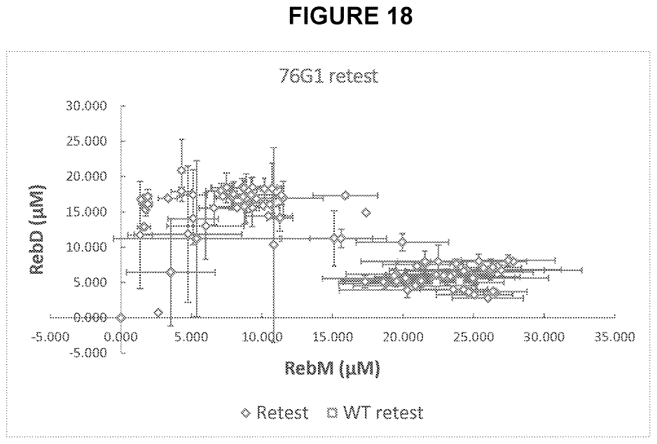

Table 20 and FIG. 17 show the UGT76G1 variant colonies with the highest selectivity towards production of either RebM or RebD, which were selected for further study. In FIG. 17, all data points with the “WT” prefix indicate RebM and RebD production of the wild type enzyme. It is shown that selected enzyme variants exhibited an inhibited RebD to RebM activity or an increased production of RebM, as compared to wild type controls.

TABLE 20
Top RebM- and RebD-producing colonies.

| Colony | RebM (μM) | RebD (μM) |
|---|---|---|
| A1 | 16.28 | 23.35 |
| A2 | 4.83 | 24.12 |
| A3 | 2.92 | 25.13 |
| A4 | 34.07 | 12.24 |
| A5 | 5.66 | 23.72 |
| A6 | 11.08 | 23.50 |
| A7 | 5.33 | 24.35 |
| A8 | 38.36 | 11.90 |
| A9 | 42.18 | 13.76 |
| A10 | 33.80 | 8.95 |
| A11 | 34.66 | 10.13 |
| A12 | 40.44 | 9.30 |
| B1 | 36.86 | 13.64 |
| B2 | 34.88 | 9.05 |
| B3 | 38.61 | 6.64 |
| B4 | 20.84 | 24.42 |
| B5 | 22.70 | 23.28 |
| B6 | 35.49 | 9.63 |
| B7 | 34.55 | 8.71 |
| B8 | 35.47 | 11.31 |
| B9 | 35.62 | 9.17 |
| B10 | 37.59 | 7.80 |
| B11 | 36.76 | 12.41 |
| B12 | 2.31 | 26.75 |
| C1 | 2.21 | 23.66 |
| C2 | 9.24 | 24.56 |
| C3 | 1.93 | 23.18 |
| C4 | 10.47 | 24.70 |
| C5 | 4.91 | 23.09 |
| C6 | 2.38 | 27.11 |
| C7 | 11.06 | 28.32 |
| C8 | 13.77 | 23.07 |
| C9 | 35.58 | 9.92 |
| C10 | 33.51 | 5.02 |
| C11 | 33.87 | 4.24 |
| C12 | 6.04 | 30.20 |
| D1 | 33.51 | 4.93 |
| D2 | 43.18 | 12.50 |
| D3 | 34.04 | 8.06 |
| D4 | 35.92 | 12.38 |
| D5 | 37.11 | 7.14 |
| D6 | 44.66 | 11.19 |
| D7 | 33.61 | 13.36 |
| D8 | 36.19 | 8.68 |
| D9 | 36.88 | 16.12 |
| D10 | 11.25 | 30.00 |
| D11 | 11.68 | 31.55 |
| E1 | 56.60 | 7.58 |
| E2 | 12.18 | 30.41 |
| E3 | 13.75 | 33.16 |
| E4 | 8.79 | 27.90 |
| E5 | 8.69 | 25.33 |
| E6 | 11.78 | 28.56 |
| E7 | 8.31 | 23.67 |
| E8 | 9.19 | 25.35 |
| E9 | 7.77 | 27.41 |
| E10 | 8.96 | 24.06 |
| E11 | 11.25 | 31.88 |
| E12 | 10.18 | 24.20 |
| F1 | 9.94 | 23.65 |
| F2 | 38.36 | 12.57 |
| F3 | 37.37 | 15.35 |
| F4 | 8.89 | 25.59 |
| F5 | 10.78 | 23.51 |
| F6 | 11.41 | 27.57 |
| F7 | 10.96 | 26.18 |
| F8 | 35.86 | 14.67 |
| F9 | 5.69 | 29.07 |
| F10 | 13.84 | 32.85 |
| F11 | 2.81 | 27.27 |
| F12 | 39.86 | 10.64 |
| G1 | 37.94 | 8.80 |
| G2 | 9.56 | 23.64 |
| G3 | 33.80 | 9.62 |
| G4 | 3.01 | 25.15 |
| G5 | 9.81 | 25.41 |
| G6 | 42.71 | 9.96 |
| G7 | 1.65 | 24.20 |
| G8 | 9.63 | 24.28 |
| G9 | 33.65 | 22.69 |
| G10 | 34.87 | 9.69 |
| G11 | 33.66 | 11.49 |
| G12 | 3.58 | 24.04 |
| H1 | 33.94 | 9.84 |
| H2 | 10.39 | 23.59 |
| H3 | 43.64 | 9.40 |
| H4 | 33.50 | 8.73 |
| H5 | 36.32 | 9.39 |
| H6 | 34.61 | 7.69 |
| H7 | 36.73 | 9.20 |
| H8 | 35.49 | 4.09 |
| H9 | 34.25 | 4.82 |
| H10 | 35.36 | 4.10 |
| H11 | 19.87 | 25.19 |

### Example 14: UGT76G1 Site Saturation Library Rescreen and Variant Sequencing

A rescreen of the 47 UGT76G1 variant colonies producing either the highest amounts of RebD or RebM was done in triplicate and showed the same trends as the initial screen (FIG. 19). The colonies to be sequenced were selected by compiling the results from the screen and rescreen. As the production levels of the screen and rescreen could not be directly compared, the colonies were ranked from highest producers to lowest producers of RebD and RebM, respectively, and the ranks from the screen and rescreen were averaged. From the averages, the top 16 RebD-, top 16 RebM-, and top 16 RebD/M-producing colonies (a total of 48 colonies) were identified. As some of the top RebD and top RebD/M producers were found to be the same colonies, duplicate colonies were counted only once, and additional colonies were chosen to reach the 48 total colonies to be sequenced. These colonies were then sequenced in duplicate with the GPDseq_fwd and CYC1seq-rev primers, shown below.

GPDseq_fwd primer seq:
(SEQ ID NO: 88)
CGG TAG GTA TTG ATT GTA ATT
CYC1seq_rev primer seq:
(SEQ ID NO: 89)
CTT TTC GGT TAG AGC GGA TGT

Tables 21-23 show the amounts and rankings of RebD, RebM, and RebD/RebM produced by the indicated variants, and Table 24 summarizes the mutations that selectively increase either RebD or RebM production. Amounts of RebM, RebD, RebA, Rubusoside, and RebB produced by wild type and UGT76G1 variant colonies are shown in Table 25.

TABLE 21
Identities of top RebD-producing UGT76G1 variants.

| | | | Rescreen | | | | | | | | |
|---|---|---|---|---|---|---|---|---|---|---|---|
| | Screen | | Sample 1 | | Sample 2 | | Sample 3 | | Average | | |
| | RebD | | RebD | | RebD | | RebD | | RebD | | |
| Colony | (μM) | Rank | (μM) | Rank | (μM) | Rank | (μM) | Rank | (μM) | RANK | Mutation |
| E2 | 25.33 | 21 | 17.00 | 21 | 18.99 | 7 | 19.35 | 3 | 20.17 | 13 | L257A |
| E5 | 33.16 | 1 | 17.51 | 15 | 15.34 | 29 | 18.49 | 8 | 21.13 | 13 | L257G |
| F9 | 29.07 | 8 | 21.05 | 3 | 17.23 | 15 | 13.89 | 28 | 20.31 | 14 | L257T |
| G5 | 25.15 | 23 | 21.53 | 2 | 19.07 | 4 | 14.50 | 25 | 20.06 | 14 | S283G |
| E3 | 28.56 | 9 | 16.42 | 22 | — | — | 18.28 | 10 | 21.09 | 14 | L257W |
| C7 | 27.11 | 15 | 18.64 | 8 | 14.64 | 32 | 18.97 | 4 | 19.84 | 15 | T146A |
| F6 | 25.59 | 18 | 18.17 | 11 | 15.55 | 25 | 18.87 | 5 | 19.55 | 15 | L257R, |
| | | | | | | | | | | | (S389F) |
| C5 | 28.32 | 10 | — | — | — | — | 16.03 | 20 | 22.18 | 15 | T146A |
| D11 | 30.00 | 7 | — | — | 15.58 | 24 | — | — | 22.79 | 16 | L257R |
| A5 | 23.72 | 35 | 18.52 | 10 | 16.76 | 19 | 18.66 | 6 | 19.42 | 18 | I26F |
| F1 | 24.06 | 33 | 19.91 | 4 | 16.90 | 17 | — | — | 20.29 | 18 | L257G |
| B12 | 24.56 | 26 | 18.11 | 12 | 17.29 | 14 | 15.98 | 21 | 18.99 | 18 | T146G |
| E4 | 30.41 | 5 | 16.30 | 23 | 17.46 | 11 | 12.88 | 34 | 19.26 | 18 | L257P |
| E10 | 23.65 | 38 | — | — | 19.31 | 3 | 17.12 | 15 | 20.03 | 19 | L257G |
| F4 | 12.57 | 55 | 19.36 | 5 | 17.43 | 13 | 18.55 | 7 | 16.98 | 20 | L257E |
| E9 | 24.20 | 31 | 17.49 | 16 | 16.93 | 16 | — | — | 19.54 | 21 | L257G |
| E7 | 23.67 | 36 | 19.24 | 6 | 15.51 | 26 | 16.74 | 17 | 18.79 | 21 | L257G |
| H2 | 23.59 | 40 | 15.85 | 26 | 17.95 | 8 | 17.63 | 12 | 18.75 | 22 | S285R |
| G7 | 24.20 | 30 | — | — | 16.34 | 21 | 17.22 | 14 | 19.26 | 22 | S283N |
| C12 | 30.20 | 6 | — | — | 14.50 | 33 | 14.36 | 27 | 19.68 | 22 | H155R |
| C1 | 26.75 | 16 | 15.32 | 27 | 16.30 | 22 | 14.43 | 26 | 18.20 | 23 | T146G |
| A6 | 23.50 | 42 | 14.96 | 29 | 19.59 | 2 | 16.25 | 19 | 18.57 | 23 | I26W |
| C4 | 24.70 | 25 | — | — | — | — | 15.94 | 22 | 20.32 | 24 | T146P |

TABLE 22
Identities of the top RebM-producing UGT76G1 variants.

| | | | Rescreen | | | | | | | | |
|---|---|---|---|---|---|---|---|---|---|---|---|
| | Screen | | Sample 1 | | Sample 2 | | Sample 3 | | Average | | |
| | RebD | | RebD | | RebD | | RebD | | RebD | | |
| Colony | (μM) | Rank | (μM) | Rank | (μM) | Rank | (μM) | Rank | (μM) | RANK | Mutation |
| G1 | 37.94 | 12 | — | — | 26.68 | 7 | 26.77 | 6 | 30.46 | 8 | T284G |
| H7 | 36.73 | 19 | — | — | 29.81 | 1 | 25.11 | 12 | 30.55 | 11 | K337P |
| B1 | 36.86 | 17 | — | — | — | — | 27.82 | 5 | 32.34 | 11 | T55K |
| D2 | 43.18 | 4 | 21.39 | 30 | 27.28 | 4 | — | — | 30.62 | 13 | Q198R |
| H3 | 43.64 | 3 | 23.32 | 21 | 24.67 | 14 | 22.03 | 23 | 28.42 | 15 | S285T |
| C11 | 33.87 | 39 | 28.92 | 3 | 26.07 | 8 | 24.21 | 14 | 28.27 | 16 | H155L |
| A10 | 33.80 | 41 | 25.52 | 10 | 23.48 | 15 | 29.50 | 1 | 28.08 | 17 | S56A |
| B11 | 36.76 | 18 | 25.28 | 12 | 20.88 | 28 | 24.69 | 13 | 26.90 | 18 | Y128S |
| H04 | 33.50 | 47 | 25.34 | 11 | 24.98 | 13 | 28.71 | 3 | 28.13 | 19 | K337E |
| B02 | 34.88 | 30 | 28.40 | 4 | — | — | 20.65 | 27 | 27.98 | 20 | T55E |
| D09 | 56.60 | 1 | 21.14 | 32 | 20.84 | 29 | — | — | 32.86 | 21 | S253G |
| D01 | 33.51 | 45 | 23.48 | 20 | 25.27 | 11 | 25.33 | 10 | 26.89 | 22 | H155L |
| H09 | 34.25 | 35 | — | — | 21.90 | 21 | 25.28 | 11 | 27.14 | 22 | L379V |
| G11 | 33.65 | 43 | 22.95 | 22 | 27.77 | 3 | — | — | 28.12 | 23 | T284R |
| B8 | 35.47 | 28 | 25.66 | 9 | 21.01 | 26 | 20.13 | 28 | 25.57 | 23 | Y128E |
| F2 | 11.41 | 58 | 31.01 | 1 | 21.06 | 25 | 26.62 | 7 | 22.53 | 23 | S253W |
| C10 | 33.51 | 46 | 22.04 | 28 | 25.54 | 10 | 25.42 | 8 | 26.63 | 23 | H155L |

TABLE 23
Identities of the top RebD/M-producing UGT76G1 variants.

| | | | | | Rank | |
|---|---|---|---|---|---|---|
| | Average | Average | Average | Average | RebD + | |
| | RebD | Rank | RebM | Rank | Rank | |
| Colony | (μM) | RebD | (μM) | RebM | (96-M) | Mutation |
| G7 | 19.26 | 22 | 1.51 | 84 | 34 | S283N |
| F9 | 20.31 | 14 | 5.27 | 71 | 38 | L257T |
| C1 | 18.20 | 23 | 1.90 | 79 | 40 | T146G |
| B12 | 18.99 | 18 | 3.74 | 74 | 41 | T146G |
| A5 | 19.42 | 18 | 6.76 | 68 | 46 | I26F |
| F6 | 19.55 | 15 | 8.23 | 63 | 48 | L257R + |
| | | | | | | S389F |
| C6 | 17.74 | 29 | 2.59 | 77 | 48 | T146G |
| F1 | 20.29 | 18 | 7.98 | 65 | 49 | L257G |
| E2 | 20.17 | 13 | 8.64 | 60 | 49 | L257A |
| C7 | 19.84 | 15 | 7.52 | 61 | 50 | T146A |
| C3 | 16.30 | 37 | 1.74 | 83 | 50 | T146G |
| E11 | 17.80 | 25 | 6.48 | 69 | 52 | L257G |
| A2 | 15.34 | 32 | 4.48 | 76 | 52 | Q23H |
| E5 | 21.13 | 13 | 9.32 | 57 | 52 | L257G |
| E4 | 19.26 | 18 | 8.00 | 61 | 54 | L257P |
| G5 | 20.06 | 14 | 8.79 | 56 | 54 | S283G |
| D11 | 22.79 | 16 | 9.75 | 58 | 54 | L257R |
| C5 | 22.18 | 15 | 10.81 | 57 | 54 | T146A |
| E9 | 19.54 | 21 | 8.21 | 62 | 55 | L257G |
| E7 | 18.79 | 21 | 8.00 | 62 | 55 | L257G |
| C2 | 18.29 | 26 | 6.95 | 67 | 55 | T146A |
| A3 | 15.25 | 35 | 3.19 | 76 | 56 | Q23G |
| E12 | 17.28 | 25 | 7.26 | 65 | 56 | L257W |
| E10 | 20.03 | 19 | 10.10 | 57 | 57 | L257G |

TABLE 24
Summary of UGT76G1 variants for RebD production and
RebM production.

| | |
|---|---|
| RebD | Q23G, Q23H, I26F, I26W, T146A, T146G, T146P, H155R, |
| | L257P, L257W, L257T, L257G, L257A, L257R, L257E, |
| | S283G and S283N |
| RebM | T55K, T55E, S56A, Y128S, Y128E, H155L, H155R, Q198R, |
| | S285R, S285T, S253W, S253G, T284R, T284G, S285G, |
| | K337E, K337P and L379V |

TABLE 25
Production of steviol glycosides by UGT76G1 variants.

| | RebM | RebD | RebA | Rubu | RebB | Total |
|---|---|---|---|---|---|---|
| | AVG | | | | | RebA-->RebM |
| Mutation | (μM) | | | | | SUM |
| WT | 22.35 | 4.98 | 7.03 | 0.65 | 2.54 | 34.37 |
| WT | 24.01 | 5.14 | 5.68 | 0.77 | 2.43 | 34.83 |

RebD-optimizing mutations

| | | | | | | |
|---|---|---|---|---|---|---|
| L257W | 9.35 | 18.46 | 4.70 | | 2.03 | 32.52 |
| L257A | 8.62 | 18.45 | 4.75 | | 1.97 | 31.82 |
| L257E | 8.72 | 18.44 | 3.70 | | 1.54 | 30.87 |
| L257G | 7.49 | 18.40 | 4.44 | 0.18 | 1.80 | 30.34 |
| S283G | 10.71 | 18.37 | 3.77 | | 1.58 | 32.85 |
| L257G | 10.18 | 18.22 | 4.47 | 0.20 | 1.87 | 32.86 |
| I26F | 7.13 | 17.98 | 3.81 | | 1.64 | 28.92 |
| Q23H | 4.30 | 17.88 | 3.04 | | 1.38 | 25.22 |
| L257R, | 8.01 | 17.53 | 4.21 | | 1.92 | 29.75 |
| S389F | | | | | | |
| T146A | 9.24 | 17.41 | 4.16 | 0.18 | 1.72 | 30.82 |
| L257T | 5.13 | 17.39 | 2.73 | 0.25 | 1.29 | 25.24 |
| L257W | 11.40 | 17.35 | 4.28 | | 1.94 | 33.04 |
| L257G | 7.23 | 17.21 | 4.19 | 0.45 | 1.81 | 28.62 |
| L257G | 7.90 | 17.16 | 4.16 | | 1.73 | 29.22 |
| S285R | 8.66 | 17.14 | 3.44 | | 1.70 | 29.25 |
| T146G | 1.91 | 17.13 | 1.69 | | 0.74 | 20.73 |
| L257G | 7.84 | 17.12 | 3.93 | | 1.80 | 28.88 |
| I26W | 11.51 | 16.93 | 5.22 | | 1.93 | 33.67 |
| S283N | 1.45 | 16.78 | 1.48 | | 0.67 | 19.70 |
| T146A | 10.57 | 16.03 | 4.07 | | 1.86 | 30.68 |
| T146G | 1.82 | 15.95 | 1.75 | 0.22 | 0.72 | 19.52 |
| T146P | 10.12 | 15.94 | 4.05 | | 1.96 | 30.11 |
| T146A | 9.31 | 15.60 | 4.34 | | 1.91 | 29.25 |
| L257R | 8.26 | 15.58 | 3.60 | 0.17 | 1.88 | 27.44 |
| L257P | 6.61 | 15.55 | 3.62 | 0.15 | 1.59 | 25.78 |
| T146G | 1.76 | 15.35 | 1.81 | 0.24 | 0.68 | 18.92 |
| H155R | 10.49 | 14.43 | 4.46 | 0.25 | 1.78 | 29.38 |
| L257G | 5.12 | 14.03 | 3.63 | | 0.95 | 22.78 |
| T146G | 1.64 | 12.86 | 1.65 | 0.21 | 0.67 | 16.14 |
| Q23G | 3.33 | 11.96 | 1.71 | 0.16 | 0.80 | 17.00 |

RebM-optimizing mutations

| | | | | | | |
|---|---|---|---|---|---|---|
| T55K | 27.82 | 7.96 | 6.28 | 0.40 | 2.31 | 42.06 |
| K337P | 27.46 | 8.14 | 5.83 | 0.41 | 2.18 | 41.43 |
| T284G | 26.72 | 7.29 | 7.27 | 0.34 | 2.72 | 41.28 |
| H155L | 26.40 | 3.70 | 6.79 | 0.30 | 2.50 | 36.89 |
| K337E | 26.34 | 7.14 | 6.35 | 0.39 | 2.66 | 39.83 |
| S253W | 26.23 | 6.83 | 5.82 | 0.37 | 2.26 | 38.89 |
| S56A | 26.17 | 6.50 | 6.94 | 0.66 | 2.46 | 39.61 |
| T284R | 25.36 | 7.99 | 6.24 | 0.24 | 2.56 | 39.58 |
| H155L | 24.69 | 3.65 | 7.38 | 0.39 | 2.66 | 35.72 |
| T55E | 24.52 | 6.73 | 5.84 | 0.46 | 2.09 | 37.09 |
| Q198R | 24.33 | 6.61 | 6.37 | 0.60 | 2.33 | 37.32 |
| H155L | 24.33 | 4.00 | 7.43 | 0.29 | 2.53 | 35.76 |
| Y128S | 23.62 | 6.20 | 6.48 | 0.43 | 2.29 | 36.29 |
| L379V | 23.59 | 4.01 | 7.09 | 0.28 | 2.72 | 34.69 |
| S285T | 23.34 | 5.90 | 5.73 | 0.44 | 2.30 | 34.97 |
| Y128E | 22.27 | 5.04 | 5.78 | 0.41 | 2.17 | 33.09 |
| S253G | 20.99 | 7.26 | 5.78 | 0.27 | 2.42 | 34.04 |

### Example 15: Determination of Relative Rates of UGT76G1 Glycosylation Reactions

UGT76G1 was only known in the literature to catalyze the 1,3-glycosylation of 1,2-stevioside to convert it into RebA and converting 1,2-bioside to RebB. The inventors have newly discovered that the reactions shown in Table 26 are catalyzed by UGT76G1.

TABLE 26
Newly discovered UGT76G1 reactions.

| Substrate | Product |
|---|---|
| Rebaudioside D | Rebaudioside M |
| Rubusoside | “Rebaudioside Q” (1,3-O-glycoside linkages |
| | on both the 13- and 19-O-glucose position) |
| Steviol 1,2 bioside | Rebaudioside B isomer (19-O) |
| isomer (19-O) | |

Similar to Example 9, p416GPD containing WT-76G1 was expressed in the protease deficient yeast strain DSY6 for 48 h in SC-ura media. 100 μL of cells were then reinoculated in 3 mL of SC-ura media for 16 h. The cells were lysed with 200 μL CelLytic™ Y according to manufacturer's description. 6 μL of the lysate were added to 24 μL of the reaction mixture consisting of 20 mM Tris-buffer (pH 8.0), 0.3 μMUDPG, and either 0.1 μM rubusoside, 0.2 μM 1,2-bioside, 0.2 μM 1,2-stevioside, 0.2 μM RebA, or 0.1 μM RebE. The reactions were incubated at 30 °C and stopped at 0, 1, 2, and 18 h by transferring 25 μL of the 30 μL reaction mixture to 25 μL DMSO. Amounts of steviol glycosides were analyzed by LC-MS and assessed by peak integration during data processing as “area under the curve.”

In FIG. 19, it is shown that an approximately 50% decrease in the “area under the curve” for rubusoside resulted in considerable production of 1,3-stevioside (RebG) over 18 h. RebQ, newly discovered by the inventors, was first detected at 18 h. Additionally, FIG. 19 shows that 1,2-stevioside was not completely consumed over the 18 h period for the production of RebA as 1,2-bioside was for the production of RebB.

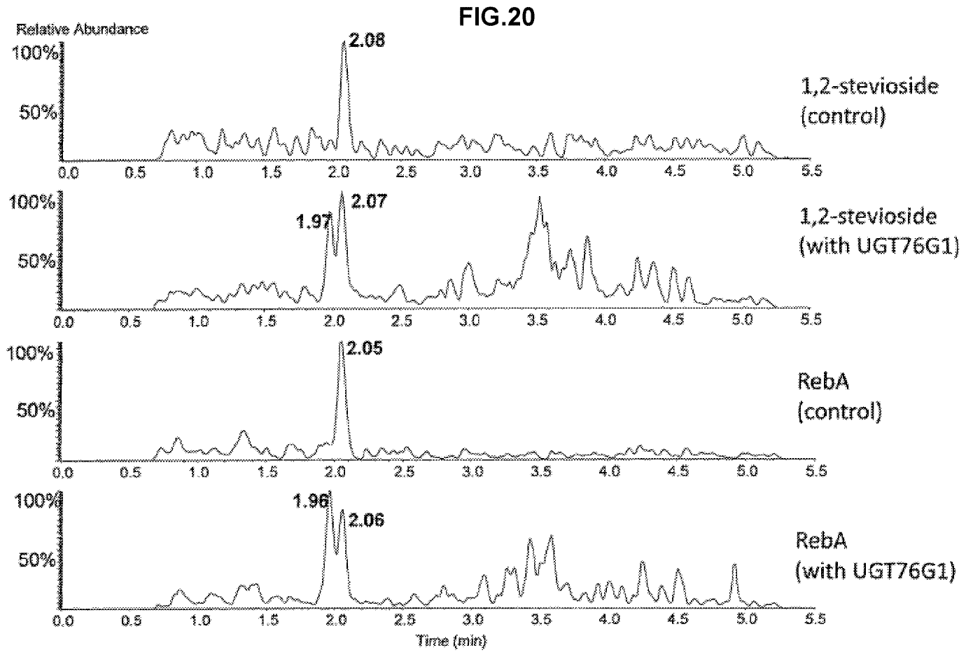

Furthermore, using either 1,2-stevioside or RebA as a substrate, a peak eluting at 1.96 min on the steviol+5 glucose chromatogram appeared (FIG. 20). Because RebD elutes at 1.11 min and UGT76G1 only catalyzes 1,3-glycosylation reactions, the peak eluting at 1.96 min appeared to be RebI. However, it was not possible to integrate the RebI peak because it was situated in a substrate artifact peak (FIG. 20).

These results collectively indicated that UGT76G1 preferentially catalyzed glycosylation of steviol glycoside substrates that are 1,2-di-glycosylated on the 13-O-position, followed by steviol glycoside substrates that are mono-glycosylated at the 13-O-position. There appeared to be little preference arising from the glycosylation state of the 19-O-position. FIG. 1 summarizes steviol glycoside glycosylation reactions and the enzymes by which they are known to be catalyzed.

### Example 16: Production of Steviol Glycosides by UGT76G1 Variants

Quantitative standards are not commercially available for each steviol glycoside, which prevented some concentration measurements. Therefore, production of additional steviol glycosides by the enzyme variants, as compared to the wild type enzyme, was assessed by peak integration during data processing. The “area under the curve” data for each variant was normalized to the wild type UGT76G1 and is shown in Table 27. These data were in agreement with previous examples but also showed that some of the variants did not produce 1,3-stevioside (RebG), rubusoside, “Reb Q,” and/or a steviol-tetraglycoside eluting at 1.43 min, as the wild type controls did. Increases in RebM and RebD production can be explained by this observation.

TABLE 27
“Area under the curve” data as a measure of the production of steviol glycosides by UGT76G1 variants relative
to wildtype UGT76G1 production.

| | | | | | | | | | | | | | 0.95 min | | 1,3 at 13 |
|---|---|---|---|---|---|---|---|---|---|---|---|---|---|---|---|
| | | | | | | | | | | | | | Stv4Glc | | and |
| | | | | 1,2 | 1,3 | | | 1,2- | 1,3- | 19- | 13- | | (suspect | 1.43 min | 19Pos |
| Variant | Reb M | Reb D | RebA | Stev | Stev | RebB | Rubu | Bios | Bios | SMG | SMG | Stev | RebE) | Stv4Glc | “Reb Q” |
| Q23H | 0.2 | 2.6 | 0.4 | 2.5 | — | 0.5 | — | — | — | — | 1.0 | — | 3.7 | 0.5 | — |
| Q23G | 0.2 | 2.5 | 0.3 | 2.0 | — | 0.3 | 0.3 | — | — | — | 0.6 | — | 2.8 | 0.4 | — |
| I26F | 0.3 | 3.8 | 0.6 | 2.2 | — | 0.7 | — | — | — | — | 1.1 | — | 3.0 | 0.5 | — |
| I26W | 0.5 | 3.6 | 0.9 | 1.8 | — | 0.8 | — | — | — | — | 0.6 | — | 2.5 | 0.4 | — |
| S56A | 1.2 | 1.4 | 1.2 | 1.2 | 0.7 | 1.0 | 1.0 | — | — | — | 1.2 | — | 1.4 | 0.3 | 0.7 |
| T55K | 1.3 | 1.7 | 1.0 | 1.0 | — | 1.0 | 0.6 | — | — | — | 1.0 | — | 1.1 | 0.9 | — |
| T55E | 1.2 | 1.4 | 1.0 | 1.3 | 0.8 | 0.9 | 0.7 | — | — | — | 0.9 | — | 1.7 | 0.3 | 0.9 |
| Y128E | 1.1 | 1.1 | 1.0 | 1.1 | — | 0.9 | 0.6 | — | — | — | 1.0 | — | 1.0 | 0.7 | 0.4 |
| Y128S | 1.1 | 1.3 | 1.1 | 1.2 | 0.6 | 1.0 | 0.7 | — | — | — | 1.0 | — | 1.3 | 1.0 | 0.6 |
| T146G | 0.1 | 3.6 | 0.3 | 3.7 | — | 0.3 | — | — | — | — | 1.0 | — | 7.6 | 0.9 | — |
| T146G | 0.1 | 3.3 | 0.3 | 4.0 | — | 0.3 | 0.4 | — | — | — | 1.4 | — | 7.3 | 1.0 | — |
| T146A | 0.4 | 3.3 | 0.7 | 2.1 | — | 0.8 | — | — | — | — | 0.9 | — | 3.2 | 0.5 | — |
| T146G | 0.1 | 2.7 | 0.3 | 3.8 | — | 0.3 | 0.3 | — | — | — | 1.2 | — | 6.2 | 0.9 | — |
| T146P | 0.5 | 3.4 | 0.7 | 2.2 | — | 0.8 | — | — | — | — | 0.9 | — | 3.0 | 0.5 | — |
| T146A | 0.5 | 3.4 | 0.7 | 2.2 | — | 0.8 | — | — | — | — | 0.8 | — | 3.3 | 0.5 | — |
| T146G | 0.1 | 3.4 | 0.3 | 3.7 | — | 0.3 | 0.3 | — | — | — | 1.2 | — | 6.6 | 0.9 | — |
| T146A | 0.4 | 3.7 | 0.7 | 2.2 | — | 0.7 | 0.3 | — | — | — | 1.1 | — | 3.8 | 0.5 | — |
| H155L | 1.2 | 0.8 | 1.2 | 1.0 | — | 1.1 | 0.4 | — | — | — | 1.0 | — | 0.6 | — | 0.4 |
| H155L | 1.3 | 0.8 | 1.1 | 0.8 | — | 1.1 | 0.5 | — | — | — | 1.0 | — | 0.5 | — | 0.3 |
| H155R | 0.5 | 3.1 | 0.7 | 2.2 | — | 0.8 | 0.4 | — | — | — | 0.9 | — | 2.8 | 0.6 | — |
| H155L | 1.2 | 0.8 | 1.2 | 1.0 | — | 1.1 | 0.6 | — | — | — | 1.1 | — | 0.8 | 0.5 | 0.5 |
| Q198R | 1.2 | 1.4 | 1.1 | 1.3 | 0.8 | 1.0 | 0.9 | — | — | — | 1.1 | — | 1.6 | — | 0.6 |
| S253G | 1.0 | 1.5 | 1.0 | 1.1 | — | 1.0 | 0.4 | — | — | — | 1.2 | — | 1.2 | 0.2 | — |
| L257R | 0.4 | 3.3 | 0.6 | 2.4 | — | 0.8 | 0.3 | — | — | — | 1.1 | — | 2.8 | 0.6 | — |
| L257A | 0.4 | 3.9 | 0.8 | 2.8 | — | 0.8 | — | — | — | — | 1.3 | — | 4.3 | 0.7 | — |
| L257W | 0.5 | 3.7 | 0.7 | 2.5 | — | 0.8 | — | — | — | — | 0.9 | — | 3.8 | 0.6 | — |
| L257P | 0.3 | 3.3 | 0.6 | 2.3 | — | 0.7 | 0.2 | — | — | — | 0.9 | — | 3.1 | 0.5 | — |
| L257G | 0.4 | 3.6 | 0.7 | 2.4 | — | 0.8 | — | — | — | — | 1.2 | — | 3.3 | 0.5 | — |
| L257G | 0.4 | 3.6 | 0.7 | 2.4 | — | 0.7 | — | — | — | — | 1.2 | — | 3.6 | 0.6 | — |
| L257G | 0.3 | 3.6 | 0.7 | 3.7 | — | 0.8 | 0.7 | — | — | — | 1.2 | — | 7.9 | 0.9 | — |
| L257G | 0.5 | 3.9 | 0.7 | 2.6 | — | 0.8 | 0.3 | — | — | — | 1.1 | — | 3.2 | 0.7 | — |
| L257G | 0.2 | 3.0 | 0.6 | 2.2 | — | 0.4 | — | — | — | — | 1.0 | — | 3.0 | 0.5 | — |
| L257W | 0.3 | 2.9 | 1.8 | 4.1 | — | 1.8 | 0.8 | — | — | — | 1.3 | — | 4.0 | 0.5 | — |
| L257G | 0.4 | 3.9 | 0.7 | 2.6 | — | 0.8 | 0.3 | — | — | — | 1.4 | — | 4.1 | 0.7 | — |
| S353W | 1.3 | 1.4 | 1.0 | 1.3 | — | 1.0 | 0.6 | — | — | — | 0.9 | — | 1.4 | 0.7 | 0.4 |
| L257E | 0.4 | 3.9 | 0.6 | 2.4 | — | 0.7 | — | — | — | — | 0.9 | — | 3.9 | 0.6 | — |
| L257R, | 0.4 | 3.7 | 0.7 | 2.5 | — | 0.8 | — | — | — | — | 1.1 | — | 3.5 | 0.5 | — |
| S389F | | | | | | | | | | | | | | | |
| L257T | 0.2 | 3.7 | 0.5 | 2.6 | — | 0.5 | 0.4 | — | — | — | 0.8 | — | 4.0 | 0.6 | — |
| T284G | 1.3 | 1.5 | 1.2 | 1.3 | — | 1.2 | 0.5 | — | — | — | 1.1 | — | 2.2 | 0.7 | — |
| S283G | 0.5 | 3.9 | 0.6 | 1.9 | — | 0.7 | — | — | — | — | 1.1 | — | 3.5 | 0.4 | — |
| S283N | 0.1 | 3.6 | 0.2 | 3.9 | — | 0.3 | — | — | — | — | 1.1 | — | 6.8 | 1.0 | — |
| T284R | 1.2 | 1.7 | 1.0 | 1.4 | — | 1.1 | 0.4 | — | — | — | 1.0 | — | 2.2 | 0.4 | — |
| S285R | 0.4 | 3.6 | 0.6 | 2.6 | — | 0.7 | — | — | — | — | 1.3 | — | 4.4 | 0.6 | — |
| S285T | 1.1 | 1.3 | 1.0 | 1.0 | — | 1.0 | 0.7 | — | — | — | 1.0 | — | 1.2 | 0.9 | 0.3 |
| K337E | 1.3 | 1.5 | 1.1 | 1.1 | — | 1.1 | 0.6 | — | — | — | 1.2 | — | 1.2 | 0.7 | — |
| K337P | 1.3 | 1.7 | 1.0 | 1.3 | — | 0.9 | 0.6 | — | — | — | 1.1 | — | 1.6 | 1.1 | 0.4 |
| L379V | 1.1 | 0.9 | 1.2 | 1.2 | — | 1.2 | 0.4 | — | — | — | 1.1 | — | 1.1 | 0.4 | — |

Table 28 summarizes trends in steviol glycoside production through RebD-optimizing and RebM-optimizing mutations, compared to the normalized production of wild type controls. The variants with increased RebD production appeared to primarily be the result of inhibiting the RebD→RebM reaction. Additional RebD production can stem from inhibition of the Rubu→RebG→RebQ steviol glycosylation branch as well as a reduction in RebB and of a tetraglucoside eluting at 1.43 min. The four-fold increase in 1,2-Stevioside and seven-fold increase in RebE were unexpected, but the RebE increase could be a seven-fold increase in a very small amount of sideproduct found in the wild type controls. Nevertheless, this finding indicated that the Stevioside→RebA reaction had also been partially inhibited by the RebD-optimizing mutations, which was seen as a reduction in RebA intermediate.

TABLE 28
Steviol glycoside production by the UGT76G1 wild type or RebD and RebM-optimizing mutations.

| | | | | | | | | | | | | | 0.95 | 1.43 | 1,3 at 13 |
|---|---|---|---|---|---|---|---|---|---|---|---|---|---|---|---|
| | | | | | | | | | | | | | min | min | and |
| | | | | 1,2 | 1,3 | | | 1,2 | 1,3 | 19- | 13- | | Stv4Glc | Stv4Glc | 19 Pos |
| | RebM | RebD | RebA | Stev | Stev | RebB | Rubu | Bios | Bios | SMG | SMG | Stev | (RebE) | (UNK) | (“RebQ”) |
| RebD | 0.1- | 2.5- | 0.3- | 1.8- | 0.0x | 0.3- | 0.0- | — | — | — | 0.9- | — | 3.0-7.0x | 0.4-0.9x | 0.0x |
| | 0.5x | 3.9x | 0.8x | 4.0x | | 0.8x | 0.4x | | | | 1.3x | | | | |
| RebM | 1.0- | 0.8- | 1.0- | 0.8- | 0.0- | 0.9- | 0.4- | — | — | — | 0.9- | — | 0.5-2.0x | 0.0-0.9x | 0.0-0.8x |
| | 1.3x | 1.7x | 1.2x | 1.3x | 0.8x | 1.1x | 1.0x | | | | 1.2x | | | | |
| WT | 23.2 | 5.1 | 6.4 | AUC | AUC | 2.5 | 0.7 | — | — | — | AUC | — | AUC | AUC | AUC |
| | μM | μM | μM | | | μM | μM | | | | | | | | |

The mutation resulting in the highest RebD levels L257G produced nearly four fold the RebD of the wild type and was found in six colonies sequenced u Other L257 mutations demonstrated nearly the same productivity d Mutants I26W and S283G showed the highest Reb6/stevioside ratio, indicating that these mutations lead to the greatest inhibition of the RebD→M reaction without mitigating the Stev→RebA reaction. These two mutations also completely abolished the Rubu→RebG→RebQ pathway and reduced the amount of the tetraglucoside eluting at 1.43 min while minimally affecting RebB production. The best RebD/RebM ratios were found with T146G and S283N mutants, which showed a 40-50-fold increase over the wild type. The S389Fmutation found with L257R demonstrated higher RebD production than L257R alone.

Generally, increases in RebM also resulted in increases in RebD, while decreasing or completely blocking the Rubu→RebG→RebQ glycosylation pathway and the tetraglucoside eluting at 1.43 min. Yet, the remaining steviol glycosides studied appeared unaffected. The top RebM producers, T55K and K337P, each increased RebM by 1.3-fold and decreased rubusoside by 0.6-fold, compared to the wild type. Since rubusoside was only present at 0.7 μM in the wild type, this observed decrease was insufficient to explain the increase in RebM. The Rubu→RebG→RebQ pathway was almost removed, with no 1,3-stevioside (RebG) produced by these mutants. As well, the variant with the K337P mutation produced 0.6-fold the levels of RebQ with the wild type. The mutations H155L and L379V each produced more RebM to RebD, with the wild type UGT76G1 producing approximately 4.58 RebM per RebD, and H155L and L379 producing approximately 6.66 and 5.88 RebM per RebD, respectively. Through the data uncovered by this study, UGT76G1 enzymes can be screened to identify species having improved kinetics towards RebD and RebM and that minimize side products, thereby increasing the flux towards the desired steviol glycosides.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

### REFERENCES

1. Critical Reviews in Food Science and Nutrition, 52:11, 988-998, DOI:10.1080/10408398.2010.519447.
2. J Nat Prod. 2013 Jun. 28; 76(6):1201-28. doi: 10.1021/np400203b. Epub 2013 May 28.
3. Plant Physiology and Biochemistry 63 (2013) 245e253.
4. Praveen Guleria and Sudesh Kumar Yadav, 2013. Insights into Steviol Glycoside Biosynthesis Pathway Enzymes Through Structural Homology Modelling. American Journal of Biochemistry and Molecular Biology, 3: 1-19.
5. The Plant Journal (2005) 41, 56-67 doi: 10.1111/j.1365-313X.2004.02275.
6. Madhav et al., “Functional and structural variation of uridine diphosphate glycosyltransferase (UGT) gene of Stevia rebaudianaeUGTSr involved in the synthesis of rebaudioside A” Plant Physiology and Biochemistry 63 (2013) 245e253.
7. Jewett M C, et al., Molecular Systems Biology, Vol. 4, article 220 (2008).
8. Masada S et al., FEBS Letters, Vol. 581, 2562-2566 (2007).

1. A recombinant host cell, comprising a recombinant gene encoding a uridine 5′-diphospho (UDP) glycosyl transferase polypeptide or a catalytically active portion thereof capable of beta 1,3 glycosylation of the C3′ of the 13-O-glucose, 19-O-glucose, or both 13-O-glucose and 19-O-glucose of a steviol glycoside having at least 50% sequence identity to the amino acid sequence set forth in SEQ ID NO:2;
wherein the recombinant host cell is capable of producing one or more steviol glycosides.

2. The recombinant host cell of claim 1, wherein the 5′-UDP glycosyl transferase polypeptide or a catalytically active portion thereof capable of beta 1,3 glycosylation of the C3′ of the 13-O-glucose, 19-O-glucose, or both 13-O-glucose and 19-O-glucose of a steviol glycoside comprises one or more amino acid substitutions of residues 23, 26, 55, 56, 128, 146, 155, 198, 253, 257, 283, 284, 285, 337, or 379 of SEQ ID NO:2.

3. The recombinant host cell of claim 1, wherein the 5′-UDP glycosyl transferase polypeptide or a catalytically active portion thereof capable of beta 1,3 glycosylation of the C3′ of the 13-O-glucose, 19-O-glucose, or both 13-O-glucose and 19-O-glucose of a steviol glycoside comprises at least one amino acid substitution of SEQ ID NO:2 that is Q23G, Q23H, I26F, I26W, T55E, T55K, S56A, Y128E, Y128S, T146A, T146G, T146P, H155L, H155R, Q198R, S253G, S253W, L257A, L257E, L257G, L257P, L257R, L257T, L257W, T284G, S283N, T284R, S285G, S285R, S285T, K337E, K337P, L379A, L379G, or L379V.

4. The recombinant host cell of claim 1, wherein the recombinant host cell further comprises:
(a) a gene encoding a polypeptide capable of synthesizing geranylgeranyl pyrophosphate (GGPP) from farnesyl diphosphate (FPP) and isopentenyl diphosphate (IPP);
wherein the polypeptide capable of synthesizing geranylgeranyl pyrophosphate (GGPP) from farnesyl diphosphate (FPP) and isopentenyl diphosphate (IPP) comprises a polypeptide having 70% or greater sequence identity to the amino acid sequence set forth in SEQ ID NO:24;
(b) a gene encoding a polypeptide capable of synthesizing ent-copalyl diphosphate from GGPP;
wherein the polypeptide capable of synthesizing ent-copalyl diphosphate from GGPP comprises a polypeptide having 70% or greater sequence identity to the amino acid sequence set forth in SEQ ID NO:13;
(c) a gene encoding a polypeptide capable of synthesizing ent-kaurene from ent-copalyl pyrophosphate;
wherein the polypeptide capable of synthesizing ent-kaurene from ent-copalyl pyrophosphate comprises a polypeptide having 70% or greater sequence identity to the amino acid sequence set forth in SEQ ID NO:21;
(d) a gene encoding a polypeptide capable of synthesizing ent-kaurenoic acid from ent-kaurene;
wherein the polypeptide capable of synthesizing ent-kaurenoic acid from ent-kaurene comprises a polypeptide having 70% or greater sequence identity to the amino acid sequence set forth in SEQ ID NO:25;
(e) a gene encoding a polypeptide capable of synthesizing steviol from ent-kaurenoic acid;
wherein the polypeptide capable of synthesizing steviol from ent-kaurenoic acid comprises a polypeptide having 70% or greater sequence identity to the amino acid sequence set forth in SEQ ID NO:11; and/or
(f) a gene encoding a polypeptide capable of reducing cytochrome P450 complex;
wherein the polypeptide capable of reducing cytochrome P450 complex comprises a polypeptide having 70% or greater sequence identity to the amino acid sequence set forth in any one of SEQ ID NO:4 or 9
wherein at least one of the genes in (e)-(j) is a recombinant gene.

5. A recombinant host cell capable of producing Rebaudioside M (RebM) in a whole-cell bioconversion of a plant-derived or a synthetic steviol glycoside in a cell culture of the host cell, the host cell comprising:
(a) a gene encoding a uridine 5′-diphospho (UDP) glycosyl transferase polypeptide capable of beta 1,3 glycosylation of the C3′ of the 13-O-glucose, 19-O-glucose, or both 13-O-glucose and 19-O-glucose of a steviol glycoside having at least 50% sequence identity to the amino acid sequence set forth in SEQ ID NO:2 and having:
(i) one or more amino acid substitutions of residues 23, 26, 55, 56, 128, 146, 155, 198, 253, 257, 283, 284, 285, 337, or 379; and/or
(ii) at least one amino acid substitution of SEQ ID NO:2 that is Q23G, Q23H, I26F, I26W, T55E, T55K, S56A, Y128E, Y128S, T146A, T146G, T146P, H155L, H155R, Q198R, S253G, S253W, L257A, L257E, L257G, L257P, L257R, L257T, L257W, T284G, S283N, T284R, S285G, S285R, S285T, K337E, K337P, L379A, L379G, or L379V; and
(b) a first recombinant gene encoding a first 5′-UDP glycosyl transferase polypeptide capable of beta 1,2 glycosylation of the C2′ of the 13-O-glucose, 19-O-glucose, or both 13-O-glucose and 19-O-glucose of the steviol glycoside
wherein the first 5′-UDP glycosyl transferase polypeptide comprises a polypeptide having 70% or greater sequence identity to the amino acid sequence set forth in SEQ ID NO:16; and/or
a second recombinant gene encoding a second 5′-UDP glycosyl transferase polypeptide capable of beta 1,2 glycosylation of the C2′ of the 13-O-glucose, 19-O-glucose, or both 13-O-glucose and 19-O-glucose of a steviol glycoside;
wherein the second 5′-UDP glycosyl transferase polypeptide comprises a polypeptide having 70% or greater sequence identity to the amino acid sequence set forth in any one of SEQ ID NOs:15 or 86
wherein at least one of the polypeptides is a recombinant polypeptide expressed in the host cell;
wherein the steviol glycoside is rubusoside, stevioside, Rebaudioside A (RebA), Rebaudioside E (RebE), or a combination thereof; and
wherein the recombinant host cell is capable of producing at least 600 mg/L of RebM when grown in a cell culture.

6. The recombinant host cell of claim 5, wherein the gene encoding the 5′-UDP glycosyl transferase polypeptide capable of beta 1,3 glycosylation of the C3′ of the 13-O-glucose, 19-O-glucose, or both 13-O-glucose and 19-O-glucose of the steviol glycoside and the first gene encoding the first 5′-UDP glycosyl transferase polypeptide capable of beta 1,2 glycosylation of the C2′ of the 13-O-glucose, 19-O-glucose, or both 13-O-glucose and 19-O-glucose of a steviol glycoside has a copy number of 2 or more.

7. The recombinant host cell of claim 6, wherein the recombinant host cell is capable of producing RebM enriched steviol glycoside compositions that have greater than at least 3% RebM by weight total steviol glycosides.

8. The recombinant host cell of claim 7, wherein the recombinant host cell is further capable of producing Rebaudioside D (RebD).

9. The recombinant host cell of claim 8, wherein the recombinant host cell is capable of producing at least 1000 mg/L of RebM when grown in a cell culture.

10. The recombinant host cell of claim 8 wherein the recombinant host cell is capable of producing at least 1000 mg/L of RebD when grown in a cell culture.

11. The recombinant host cell of claim 8, wherein the recombinant host cell is capable of producing RebM and RebD that are produced by the recombinant host cell at a ratio of at least 1:0.1 in a cell culture.

12. The recombinant host cell of claim 8, wherein the recombinant host cell is capable of producing RebD and RebM that are produced by the recombinant host cell at a ratio of at least 1:0.1 in a cell culture.

13. The recombinant host cell of claim 5, wherein the cell culture comprises:
(a) RebM that is produced in the whole-cell bioconversion in the cell culture of the host cell,
(b) glucose, uridine diphosphate (UDP)-glucose, UDP-rhamnose, UDP-xylose, fructose, and/or N-acetyl-glucosamine; and
(c) supplemental nutrients comprising trace metals, vitamins, salts, yeast nitrogen base (YNB) and/or amino acids.

14. The recombinant host cell of claim 5, wherein the recombinant host cell is permeabilized using a permeabilizing agent, wherein the permeabilizing agent is a solvent, a detergent, or a surfactant, by a mechanical shock, an electroporation, or an osmotic shock.

15. The recombinant host cell of claim 5, wherein the host cell has a reduced ability to degrade external sucrose.

16. The recombinant host cell of claim 5, wherein the host cell is:
(a) in suspension or immobilized;
(b) entrapped in a calcium or sodium alginate bead;
(c) linked to a hollow fiber tube reactor system;
(d) concentrated and entrapped within a membrane reactor system; or
(e) in fermentation broth or in a reaction buffer.

17. The recombinant host cell of claim 1, wherein the recombinant host cell is a fungal cell, an algal cell, or a bacterial cell.

18. A method of producing one or more steviol glycosides in a cell culture, comprising culturing the recombinant host cell of claim 1 in the cell culture, under conditions in which the genes are expressed; and wherein the one or more steviol glycosides is produced by the recombinant host cell.

19. The method of claim 18, wherein the genes are constitutively expressed or the expression of the genes is induced.

20. A method for producing one or more steviol glycosides, comprising whole-cell bioconversion of a plant-derived or synthetic steviol and/or steviol glycosides in a cell culture of a recombinant host cell using a 5′-UDP glycosyl transferase polypeptide capable of beta 1,3 glycosylation of the C3′ of the 13-O-glucose, 19-O-glucose, or both 13-O-glucose and 19-O-glucose of a steviol glycoside having at least 50% sequence identity to the amino acid sequence set forth in SEQ ID NO:2 and having:
(a) one or more amino acid substitutions of residues 23, 26, 55, 56, 128, 146, 155, 198, 253, 257, 283, 284, 285, 337, or 379; and/or
(b) at least one amino acid substitution of SEQ ID NO:2 that is Q23G, Q23H, I26F, I26W, T55E, T55K, S56A, Y128E, Y128S, T146A, T146G, T146P, H155L, H155R, Q198R, S253G, S253W, L257A, L257E, L257G, L257P, L257R, L257T, L257W, T284G, S283N, T284R, S285G, S285R, S285T, K337E, K337P, L379A, L379G, or L379V;
and one or more of:
(c) a first 5′-UDP glycosyl transferase polypeptide capable of beta 1,2 glycosylation of the C2′ of the 13-O-glucose, 19-O-glucose, or both 13-O-glucose and 19-O-glucose of a steviol glycoside;
wherein the first 5′-UDP glycosyl transferase polypeptide comprises a polypeptide having 70% or greater sequence identity to the amino acid sequence set forth in SEQ ID NO:16; and/or
a second 5′-UDP glycosyl transferase polypeptide capable of beta 1,2 glycosylation of the C2′ of the 13-O-glucose, 19-O-glucose, or both 13-O-glucose and 19-O-glucose of a steviol glycoside;
wherein the second 5′-UDP glycosyl transferase polypeptide comprises a polypeptide having 70% or greater sequence identity to the amino acid sequence set forth in any one of SEQ ID NOs:15 or 86, a polypeptide having a substitution at residues 211 and 286 of SEQ ID NO:15, or a combination thereof; and one or more of:
(d) a 5′-UDP glycosyl transferase polypeptide capable of glycosylating steviol or the steviol glycoside at its C-13 hydroxyl group;
wherein the 5′-UDP glycosyl transferase polypeptide comprises a polypeptide having 70% or greater sequence identity to the amino acid sequence set forth in SEQ ID NO:26; and
(e) a 5′-UDP glycosyl transferase polypeptide capable of glycosylating steviol or the steviol glycoside at its C-19 carboxyl group;
wherein the 5′-UDP glycosyl transferase polypeptide comprises a polypeptide having 70% or greater sequence identity to the amino acid sequence set forth in SEQ ID NO:19;
and a plant-derived or synthetic steviol precursor, a steviol, a steviol glycoside precursor, and/or a steviol glycoside to a reaction mixture;
wherein at least one of the polypeptides is a recombinant polypeptide; and producing the steviol glycoside composition thereby;
wherein the one or more steviol glycosides comprises RebM that is produced at a concentration of at least about 600 mg/L of the reaction mixture.

21. The method of any one of claim 20, wherein the recombinant host cell is a fungal cell, an algal cell, or a bacterial cell.

22. An in vitro method of producing one or more steviol glycosides, comprising adding:
(a) a 5′-UDP glycosyl transferase polypeptide capable of beta 1,3 glycosylation of the C3′ of the 13-O-glucose, 19-O-glucose, or both 13-O-glucose and 19-O-glucose of a steviol glycoside having at least 50% sequence identity to the amino acid sequence set forth in SEQ ID NO:2 and having:
(i) one or more amino acid substitutions of residues 23, 26, 55, 56, 128, 146, 155, 198, 253, 257, 283, 284, 285, 337, or 379; and/or
(ii) at least one amino acid substitution of SEQ ID NO:2 that is Q23G, Q23H, I26F, I26W, T55E, T55K, S56A, Y128E, Y128S, T146A, T146G, T146P, H155L, H155R, Q198R, S253G, S253W, L257A, L257E, L257G, L257P, L257R, L257T, L257W, T284G, S283N, T284R, S285G, S285R, S285T, K337E, K337P, L379A, L379G, or L379V; and
(b) a first 5′-UDP glycosyl transferase polypeptide capable of beta 1,2 glycosylation of the C2′ of the 13-O-glucose, 19-O-glucose, or both 13-O-glucose and 19-O-glucose of a steviol glycoside;
wherein the first 5′-UDP glycosyl transferase polypeptide comprises a polypeptide having 70% or greater sequence identity to the amino acid sequence set forth in SEQ ID NO:16; and/or
a second 5′-UDP glycosyl transferase polypeptide capable of beta 1,2 glycosylation of the C2′ of the 13-O-glucose, 19-O-glucose, or both 13-O-glucose and 19-O-glucose of a steviol glycoside;
wherein the second 5′-UDP glycosyl transferase polypeptide comprises a polypeptide having 70% or greater sequence identity to the amino acid sequence set forth in any one of SEQ ID NOs:15 or 86;
and one or more of:
(c) a 5′-UDP glycosyl transferase polypeptide capable of glycosylating steviol or the steviol glycoside at its C-13 hydroxyl group;
wherein the 5′-UDP glycosyl transferase polypeptide comprises a polypeptide having 70% or greater sequence identity to the amino acid sequence set forth in SEQ ID NO:26; and
(d) a 5′-UDP glycosyl transferase polypeptide capable of glycosylating steviol or the steviol glycoside at its C-19 carboxyl group;
wherein the 5′-UDP glycosyl transferase polypeptide comprises a polypeptide having 70% or greater sequence identity to the amino acid sequence set forth in SEQ ID NO:19;
and a plant-derived or synthetic steviol precursor, a steviol, a steviol glycoside precursor, and/or a steviol glycoside to a reaction mixture;
wherein at least one of the polypeptides is a recombinant polypeptide; and producing the steviol glycoside composition thereby;
wherein the one or more steviol glycosides comprises RebM that is produced at a concentration of at least about 600 mg/L of the reaction mixture.

23. The reaction mixture of claim 22, comprising:
(a) one or more steviol glycosides, comprising RebM;
(b) a 5′-UDP glycosyl transferase polypeptide capable of beta 1,3 glycosylation of the C3′ of the 13-O-glucose, 19-O-glucose, or both 13-O-glucose and 19-O-glucose of a steviol glycoside having at least 50% sequence identity to the amino acid sequence set forth in SEQ ID NO:2 and having:
(i) one or more amino acid substitutions of residues 23, 26, 55, 56, 128, 146, 155, 198, 253, 257, 283, 284, 285, 337, or 379; and/or
(ii) at least one amino acid substitution of SEQ ID NO:2 that is Q23G, Q23H, I26F, I26W, T55E, T55K, S56A, Y128E, Y128S, T146A, T146G, T146P, H155L, H155R, Q198R, S253G, S253W, L257A, L257E, L257G, L257P, L257R, L257T, L257W, T284G, S283N, T284R, S285G, S285R, S285T, K337E, K337P, L379A, L379G, or L379V;
(c) glucose, fructose, sucrose, xylose, rhamnose, uridine diphosphate (UDP)-glucose, UDP-rhamnose, UDP-xylose, and/or N-acetyl-glucosamine; and
(d) reaction buffer and/or salts.

24. A cell culture, comprising the recombinant host cell of claim 1, the cell culture further comprising:
(a) RebM that is produced by the recombinant host cell or in the whole-cell bioconversion in the cell culture of the recombinant host cell,
wherein RebM is produced at a concentration of at least about 600 mg/L of the cell culture;
(b) rubusoside, stevioside, Rebaudioside A, Rebaudioside E, or a combination thereof;
(c) glucose, fructose, sucrose, xylose, rhamnose, uridine diphosphate (UDP)-glucose, UDP-rhamnose, UDP-xylose, and/or N-acetyl-glucosamine; and
(d) supplemental nutrients comprising trace metals, vitamins, salts, YNB, and/or amino acids;
wherein the cell culture is enriched for RebM relative to a steviol glycoside composition from a Stevia plant and has a reduced level of Stevia plant-derived components relative to a plant-derived Stevia extract.

25. A cell culture lysate from the host cell of claim 1, comprising:
(a) RebM that is produced by the recombinant host cell or in the whole-cell bioconversion in the cell culture of the recombinant host cell,
wherein Rebaudioside M is produced at a concentration of at least about 600 mg/L of the cell culture;
(b) rubusoside, stevioside, RebA, RebE, or a combination thereof;
(c) glucose, fructose, sucrose, xylose, rhamnose, UDP-glucose, UDP-rhamnose, UDP-xylose, and/or N-acetyl-glucosamine; and
(d) supplemental nutrients comprising trace metals, vitamins, salts, YNB, and/or amino acids;
wherein the cell culture is enriched for RebM relative to a steviol glycoside composition from a Stevia plant and has a reduced level of Stevia plant-derived components relative to a plant-derived Stevia extract.


SEQUENCE LISTING (136 sequences)

SEQ ID NO: 1 - DNA, 1616 bp, synthetic construct
  misc_feature 1..1616: note = Synthetic oligonucleotide
  source 1..1616: mol_type = other DNA
cttgcgtgtaaacgtcagtcaaacccaatggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaaataacacgatgattaatcaagcacttggattgcatgctagctgagtagctggtaatttgagttattagaagcaaagactacttggtttaaattaaataaaggatggttgttggttatgtgagctagtttatgttatgttttgtaggctataaaagccttcatatgtttcttattgtttctgtttctaaggtgaaaaaaatgctcgtttttat

SEQ ID NO: 2 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 3 - DNA, 1446 bp, synthetic construct
  misc_feature 1..1446: note = Synthetic oligonucleotide
  source 1..1446: mol_type = other DNA
atggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 4 - protein, 707 aa, Stevia rebaudiana
MQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYSSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 5 - DNA, 2124 bp, synthetic construct
  misc_feature 1..2124: note = Synthetic oligonucleotide
  source 1..2124: mol_type = other DNA
atgcaatctaactccgtgaagatttcgccgcttgatctggtaactgcgctgtttagcggcaaggttttggacacatcgaacgcatcggaatcgggagaatctgctatgctgccgactatagcgatgattatggagaatcgtgagctgttgatgatactcacaacgtcggttgctgtattgatcggatgcgttgtcgttttggtgtggcggagatcgtctacgaagaagtcggcgttggagccaccggtgattgtggttccgaagagagtgcaagaggaggaagttgatgatggtaagaagaaagttacggttttcttcggcacccaaactggaacagctgaaggcttcgctaaggcacttgttgaggaagctaaagctcgatatgaaaaggctgtctttaaagtaattgatttggatgattatgctgctgatgacgatgagtatgaggagaaactaaagaaagaatctttggcctttttctttttggctacgtatggagatggtgagccaacagataatgctgccagattttataaatggtttactgagggagatgcgaaaggagaatggcttaataagcttcaatatggagtatttggtttgggtaacagacaatatgaacattttaacaagatcgcaaaagtggttgatgatggtcttgtagaacagggtgcaaagcgtcttgttcctgttggacttggagatgatgatcaatgtattgaagatgacttcaccgcatggaaagagttagtatggccggagttggatcaattacttcgtgatgaggatgacacaactgttgctactccatacacagctgctgttgcagaatatcgcgttgtttttcatgaaaaaccagacgcgctttctgaagattatagttatacaaatggccatgctgttcatgatgctcaacatccatgcagatccaacgtggctgtcaaaaaggaacttcatagtcctgaatctgaccggtcttgcactcatcttgaatttgacatctcgaacaccggactatcatatgaaactggggaccatgttggagtttactgtgaaaacttgagtgaagttgtgaatgatgctgaaagattagtaggattaccaccagacacttactcctccatccacactgatagtgaagacgggtcgccacttggcggagcctcattgccgcctcctttcccgccatgcactttaaggaaagcattgacgtgttatgctgatgttttgagttctcccaagaagtcggctttgcttgcactagctgctcatgccaccgatcccagtgaagctgatagattgaaatttcttgcatcccccgccggaaaggatgaatattctcaatggatagttgcaagccaaagaagtctccttgaagtcatggaagcattcccgtcagctaagccttcacttggtgttttctttgcatctgttgccccgcgcttacaaccaagatactactctatttcttcctcacccaagatggcaccggataggattcatgttacatgtgcattagtctatgagaaaacacctgcaggccgcatccacaaaggagtttgttcaacttggatgaagaacgcagtgcctatgaccgagagtcaagattgcagttgggccccaatatacgtccgaacatccaatttcagactaccatctgaccctaaggtcccggttatcatgattggacctggcactggtttggctccttttagaggtttccttcaagagcggttagctttaaaggaagccggaactgacctcggtttatccattttattcttcggatgtaggaatcgcaaagtggatttcatatatgaaaacgagcttaacaactttgtggagactggtgctctttctgagcttattgttgctttctcccgtgaaggcccgactaaggaatatgtgcaacacaagatgagtgagaaggcttcggatatctggaacttgctttctgaaggagcatatttatacgtatgtggtgatgccaaaggcatggccaaagatgtacatcgaaccctccacacaattgtgcaagaacagggatctcttgactcgtcaaaggcagaactctacgtgaagaatctacaaatgtcaggaagatacctccgtgacgtttggtaa

SEQ ID NO: 6 - DNA, 2358 bp, synthetic construct
  misc_feature 1..2358: note = Synthetic oligonucleotide
  source 1..2358: mol_type = other DNA
atgtctattaatttgagatcttccggttgtagctccccaataagcgcaactttggaaaggggtctagactctgaagttcaaacaagagcaaacaatgtatcttttgagcagaccaaagagaagatcaggaaaatgcttgagaaggtcgagttgagcgtgagtgcctatgacactagttgggtagctatggtcccatcaccatccagtcaaaacgcacctcttttcccacagtgcgtcaaatggctacttgataatcaacatgaggacggctcttggggattggataaccacgaccatcagagcttaaagaaagatgtgttgtcatccacattagcctctatcctagctcttaagaaatggggaataggcgaaagacagatcaataagggtctacagttcattgaattaaactctgcactagttaccgatgaaactatacaaaaacctacaggtttcgacatcatttttccaggaatgattaagtacgccagggaccttaatttgaccatacctcttggctcagaagtagtcgacgatatgatcaggaaaagagatctagacttaaagtgtgatagcgagaaattcagcaaaggtagagaggcttatcttgcctatgttcttgaaggaactaggaacttgaaggactgggacttaattgtgaaatatcagagaaagaacggtagtctatttgatagtccagctacaaccgccgcagctttcactcaatttggcaatgacggttgcttgaggtacttatgttcacttttacagaaattcgaggccgcagtgcctagtgtatatccatttgatcaatacgctagattaagcataatcgtcactttagaatcattgggaattgacagagatttcaagactgagataaaaagcatattggatgagacctataggtactggcttagaggtgacgaagaaatttgcctagatttggccacatgtgcacttgcttttaggttgcttttagcccacggctatgacgtgtcatacgatcctctaaagccatttgcagaggaatctggtttcagcgatacccttgagggatatgttaaaaacaccttttccgtattagagcttttcaaggctgcccaaagttaccctcatgagagtgctttgaaaaagcagtgttgctggacaaaacaatatctagaaatggaactaagttcatgggttaaaacaagcgttagggacaagtacttgaaaaaggaagtggaggatgctttggcatttccatcatatgcctctttagaaagaagtgaccacagaaggaaaattcttaatggctcagcagttgaaaacacaagagtaaccaagacctcttacaggttgcataatatatgtacatcagatatcttaaaacttgctgtcgacgatttcaacttttgccaatctattcatagagaggaaatggaaagattggatagatggatagtggagaatagactacaggaattaaagttcgccagacaaaaattggcttactgttactttagtggcgctgccacactattctctccagaattgtctgacgcaaggatctcatgggctaagggaggtgttctaaccacagtagtcgatgacttttttgatgttggcggtagtaaagaagagcttgagaacttaattcacttggtggaaaagtgggatcttaatggagttcctgaatactcttcagagcatgtagaaataattttctctgtcctaagagacactatcttagaaaccggtgataaagcctttacatatcagggcagaaacgttactcaccatattgtgaaaatatggttggacttacttaagagcatgctaagggaggctgaatggtccagtgacaaatcaaccccatctttggaagattacatggagaatgcctatatcagcttcgcattaggtcctattgtattgccagctacataccttataggacctccactacctgaaaagactgtcgactcccaccaatataatcaattatacaaattggttagtaccatgggtagactattaaacgatatccagggctttaagagggaatcagccgagggaaaacttaatgcagtgtctctacatatgaagcatgaaagagacaacagaagcaaagaggttattatagaatccatgaaaggattggctgaaaggaaaagagaggaattacacaaacttgtactagaagagaaaggtagtgtcgttccaagagaatgcaaggaagccttcttaaaaatgtcaaaagtgttgaaccttttttataggaaggatgatggcttcacatctaacgacttgatgagccttgtgaaatccgtcatctacgagcctgtttcacttcaaaaggagagtctaacttga

SEQ ID NO: 7 - protein, 785 aa, Arabidopsis thaliana
MSINLRSSGCSSPISATLERRLDSEVQTRANNVSFEQTKEKIRKMLEKVELSVSAYDTSWVAMVPSPSSQNAPLFPQCVKWLLDNQHEDGSWGLDNHDHQSLKKDVLSSTLASILALKKWGIGERQINKGLQFIELNSALVTDETIQKPTGFDIIFPGMIKYARDLNLTIPLGSEVVDDMIRKRDLDLKCDSEKFSKGREAYLAYVLEGTRNLKDWDLIVKYQRKNGSLFDSPATTAAAFTQFGNDGCLRYLCSLLQKFEAAVPSVYPFDQYARLSIIVTLESLGIDRDFKTEIKSILDETYRYWLRGDEEICLDLATCALAFRLLLAHGYDVSYDPLKPFAEESGFSDTLEGYVKNTFSVLELFKAAQSYPHESALKKQCCWTKQYLEMELSSWVKTSVRDKYLKKEVEDALAFPSYASLERSDHRRKILNGSAVENTRVTKTSYRLHNICTSDILKLAVDDFNFCQSIHREEMERLDRWIVENRLQELKFARQKLAYCYFSGAATLFSPELSDARISWAKGGVLTTVVDDFFDVGGSKEELENLIHLVEKWDLNGVPEYSSEHVEIIFSVLRDTILETGDKAFTYQGRNVTHHIVKIWLDLLKSMLREAEWSSDKSTPSLEDYMENAYISFALGPIVLPATYLIGPPLPEKTVDSHQYNQLYKLVSTMGRLLNDIQGFKRESAEGKLNAVSLHMKHERDNRSKEVIIESMKGLAERKREELHKLVLEEKGSVVPRECKEAFLKMSKVLNLFYRKDDGFTSNDLMSLVKSVIYEPVSLQEESLT

SEQ ID NO: 8 - DNA, 1503 bp, synthetic construct
  misc_feature 1..1503: note = Synthetic oligonucleotide
  source 1..1503: mol_type = other DNA
atggaagcctcttacctatacatttctattttgcttttactggcatcatacctgttcaccactcaacttagaaggaagagcgctaatctaccaccaaccgtgtttccatcaataccaatcattggacacttatacttactcaaaaagcctctttatagaactttagcaaaaattgccgctaagtacggaccaatactgcaattacaactcggctacagacgtgttctggtgatttcctcaccatcagcagcagaagagtgctttaccaataacgatgtaatcttcgcaaatagacctaagacattgtttggcaaaatagtgggtggaacatcccttggcagtttatcctacggcgatcaatggcgtaatctaaggagagtagcttctatcgaaatcctatcagttcataggttgaacgaatttcatgatatcagagtggatgagaacagattgttaattagaaaacttagaagttcatcttctcctgttactcttataacagtcttttatgctctaacattgaacgtcattatgagaatgatctctggcaaaagatatttcgacagtggggatagagaattggaggaggaaggtaagagatttcgagaaatcttagacgaaacgttgcttctagccggtgcttctaatgttggcgactacttaccaatattgaactggttgggagttaagtctcttgaaaagaaattgatcgctttgcagaaaaagagagatgactttttccagggtttgattgaacaggttagaaaatctcgtggtgctaaagtaggcaaaggtagaaaaacgatgatcgaactcttattatctttgcaagagtcagaacctgagtactatacagatgctatgataagatcttttgtcctaggtctgctggctgcaggtagtgatacttcagcgggcactatggaatgggccatgagcttactggtcaatcacccacatgtattgaagaaagctcaagctgaaatcgatagagttatcggtaataacagattgattgacgagtcagacattggaaatatcccttacatcgggtgtattatcaatgaaactctaagactctatccagcagggccattgttgttcccacatgaaagttctgccgactgcgttatttccggttacaatatacctagaggtacaatgttaatcgtaaaccaatgggcgattcatcacgatcctaaagtctgggatgatcctgaaacctttaaacctgaaagatttcaaggattagaaggaactagagatggtttcaaacttatgccattcggttctgggagaagaggatgtccaggtgaaggtttggcaataaggctgttagggatgacactaggctcagtgatccaatgttttgattgggagagagtaggagatgagatggttgacatgacagaaggtttgggtgtcacacttcctaaggccgttccattagttgccaaatgtaagccacgttccgaaatgactaatctcctatccgaactttaa

SEQ ID NO: 9 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 10 - DNA, 2139 bp, synthetic construct
  misc_feature 1..2139: note = Synthetic oligonucleotide
  source 1..2139: mol_type = other DNA
atgtcttcctcttcctcttccagtacctctatgattgatttgatggctgctattattaaaggtgaaccagttatcgtctccgacccagcaaatgcctctgcttatgaatcagttgctgcagaattgtcttcaatgttgatcgaaaacagacaattcgccatgatcgtaactacatcaatcgctgttttgatcggttgtattgtcatgttggtatggagaagatccggtagtggtaattctaaaagagtcgaacctttgaaaccattagtaattaagccaagagaagaagaaatagatgacggtagaaagaaagttacaatatttttcggtacccaaactggtacagctgaaggttttgcaaaagccttaggtgaagaagctaaggcaagatacgaaaagactagattcaagatagtcgatttggatgactatgccgctgatgacgatgaatacgaagaaaagttgaagaaagaagatgttgcatttttctttttggcaacctatggtgacggtgaaccaactgacaatgcagccagattctacaaatggtttacagagggtaatgatcgtggtgaatggttgaaaaacttaaagtacggtgttttcggtttgggtaacagacaatacgaacatttcaacaaagttgcaaaggttgtcgacgatattttggtcgaacaaggtgctcaaagattagtccaagtaggtttgggtgacgatgaccaatgtatagaagatgactttactgcctggagagaagctttgtggcctgaattagacacaatcttgagagaagaaggtgacaccgccgttgctaccccatatactgctgcagtattagaatacagagtttccatccatgatagtgaagacgcaaagtttaatgatatcactttggccaatggtaacggttatacagttttcgatgcacaacacccttacaaagctaacgttgcagtcaagagagaattacatacaccagaatccgacagaagttgtatacacttggaatttgatatcgctggttccggtttaaccatgaagttgggtgaccatgtaggtgttttatgcgacaatttgtctgaaactgttgatgaagcattgagattgttggatatgtcccctgacacttattttagtttgcacgctgaaaaagaagatggtacaccaatttccagttctttaccacctccattccctccatgtaacttaagaacagccttgaccagatacgcttgcttgttatcatcccctaaaaagtccgccttggttgctttagccgctcatgctagtgatcctactgaagcagaaagattgaaacacttagcatctccagccggtaaagatgaatattcaaagtgggtagttgaatctcaaagatcattgttagaagttatggcagaatttccatctgccaagcctccattaggtgtcttctttgctggtgtagcacctagattgcaaccaagattctactcaatcagttcttcacctaagatcgctgaaactagaattcatgttacatgtgcattagtctacgaaaagatgccaaccggtagaattcacaagggtgtatgctctacttggatgaaaaatgctgttccttacgaaaaatcagaaaagttgttcttaggtagaccaatcttcgtaagacaatcaaacttcaagttgccttctgattcaaaggttccaataatcatgataggtcctggtacaggtttagccccattcagaggtttcttgcaagaaagattggctttagttgaatctggtgtcgaattaggtccttcagttttgttctttggttgtagaaacagaagaatggatttcatctatgaagaagaattgcaaagattcgtcgaatctggtgcattggccgaattatctgtagctttttcaagagaaggtccaactaaggaatacgttcaacataagatgatggataaggcatccgacatatggaacatgatcagtcaaggtgcttatttgtacgtttgcggtgacgcaaagggtatggccagagatgtccatagatctttgcacacaattgctcaagaacaaggttccatggatagtaccaaagctgaaggtttcgtaaagaacttacaaacttccggtagatacttgagagatgtctggtga

SEQ ID NO: 11 - protein, 500 aa, Stevia rebaudiana
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 12 - DNA, 2490 bp, synthetic construct
  misc_feature 1..2490: note = Synthetic oligonucleotide
  source 1..2490: mol_type = other DNA
atggttttgtcttcttcttgtactacagtaccacacttatcttcattagctgtcgtgcaacttggtccttggagcagtaggattaaaaagaaaaccgatactgttgcagtaccagccgctgcaggaaggtggagaagggccttggctagagcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaaggacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 13 - protein, 827 aa, Zea mays
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 14 - DNA, 1377 bp, synthetic construct
  misc_feature 1..1377: note = Synthetic oligonucleotide
  source 1..1377: mol_type = other DNA
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 15 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 16 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 17 - DNA, 1389 bp, synthetic construct
  misc_feature 1..1389: note = Synthetic oligonucleotide
  source 1..1389: mol_type = other DNA
atggactccggctactcctcctcctacgccgccgccgccgggatgcacgtcgtgatctgcccgtggctcgccttcggccacctgctcccgtgcctcgacctcgcccagcgcctcgcgtcgcggggccaccgcgtgtcgttcgtctccacgccgcggaacatatcccgcctcccgccggtgcgccccgcgctcgcgccgctcgtcgccttcgtggcgctgccgctcccgcgcgtcgaggggctccccgacggcgccgagtccaccaacgacgtcccccacgacaggccggacatggtcgagctccaccggagggccttcgacgggctcgccgcgcccttctcggagttcttgggcaccgcgtgcgccgactgggtcatcgtcgacgtcttccaccactgggccgcagccgccgctctcgagcacaaggtgccatgtgcaatgatgttgttgggctctgcacatatgatcgcttccatagcagacagacggctcgagcgcgcggagacagagtcgcctgcggctgccgggcagggacgcccagcggcggcgccaacgttcgaggtggcgaggatgaagttgatacgaaccaaaggctcatcgggaatgtccctcgccgagcgcttctccttgacgctctcgaggagcagcctcgtcgtcgggcggagctgcgtggagttcgagccggagaccgtcccgctcctgtcgacgctccgcggtaagcctattaccttccttggccttatgccgccgttgcatgaaggccgccgcgaggacggcgaggatgccaccgtccgctggctcgacgcgcagccggccaagtccgtcgtgtacgtcgcgctaggcagcgaggtgccactgggagtggagaaggtccacgagctcgcgctcgggctggagctcgccgggacgcgcttcctctgggctcttaggaagcccactggcgtctccgacgccgacctcctccccgccggcttcgaggagcgcacgcgcggccgcggcgtcgtggcgacgagatgggttcctcagatgagcatactggcgcacgccgccgtgggcgcgttcctgacccactgcggctggaactcgaccatcgaggggctcatgttcggccacccgcttatcatgctgccgatcttcggcgaccagggaccgaacgcgcggctaatcgaggcgaagaacgccggattgcaggtggcaagaaacgacggcgatggatcgttcgaccgagaaggcgtcgcggcggcgattcgtgcagtcgcggtggaggaagaaagcagcaaagtgtttcaagccaaagccaagaagctgcaggagatcgtcgcggacatggcctgccatgagaggtacatcgacggattcattcagcaattgagatcttacaaggattga

SEQ ID NO: 18 - DNA, 1389 bp, synthetic construct
  misc_feature 1..1389: note = Synthetic oligonucleotide
  source 1..1389: mol_type = other DNA
atggatagtggctactcctcatcttatgctgctgccgctggtatgcacgttgtgatctgcccttggttggcctttggtcacctgttaccatgtctggatttagcccaaagactggcctcaagaggccatagagtatcatttgtgtctactcctagaaatatctctcgtttaccaccagtcagacctgctctagctcctctagttgcattcgttgctcttccacttccaagagtagaaggattgccagacggcgctgaatctactaatgacgtaccacatgatagacctgacatggtcgaattgcatagaagagcctttgatggattggcagctccattttctgagttcctgggcacagcatgtgcagactgggttatagtcgatgtatttcatcactgggctgctgcagccgcattggaacataaggtgccttgtgctatgatgttgttagggtcagcacacatgatcgcatccatagctgatagaagattggaaagagctgaaacagaatccccagccgcagcaggacaaggtaggccagctgccgccccaacctttgaagtggctagaatgaaattgattcgtactaaaggtagttcagggatgagtcttgctgaaaggttttctctgacattatctagatcatcattagttgtaggtagatcctgcgtcgagttcgaacctgaaacagtacctttactatctactttgagaggcaaacctattactttccttggtctaatgcctccattacatgaaggaaggagagaagatggtgaagatgctactgttaggtggttagatgcccaacctgctaagtctgttgtttacgttgcattgggttctgaggtaccactaggggtggaaaaggtgcatgaattagcattaggacttgagctggccggaacaagattcctttgggctttgagaaaaccaaccggtgtttctgacgccgacttgctaccagctgggttcgaagagagaacaagaggccgtggtgtcgttgctactagatgggtcccacaaatgagtattctagctcatgcagctgtaggggcctttctaacccattgcggttggaactcaacaatagaaggactgatgtttggtcatccacttattatgttaccaatctttggcgatcagggacctaacgcaagattgattgaggcaaagaacgcaggtctgcaggttgcacgtaatgatggtgatggttcctttgatagagaaggcgttgcagctgccatcagagcagtcgccgttgaggaagagtcatctaaagttttccaagctaaggccaaaaaattacaagagattgtggctgacatggcttgtcacgaaagatacatcgatggtttcatccaacaattgagaagttataaagactaa

SEQ ID NO: 19 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 20 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 21 - protein, 785 aa, Arabidopsis thaliana
MSINLRSSGCSSPISATLERGLDSEVQTRANNVSFEQTKEKIRKMLEKVELSVSAYDTSWVAMVPSPSSQNAPLFPQCVKWLLDNQHEDGSWGLDNHDHQSLKKDVLSSTLASILALKKWGIGERQINKGLQFIELNSALVTDETIQKPTGFDIIFPGMIKYARDLNLTIPLGSEVVDDMIRKRDLDLKCDSEKFSKGREAYLAYVLEGTRNLKDWDLIVKYQRKNGSLFDSPATTAAAFTQFGNDGCLRYLCSLLQKFEAAVPSVYPFDQYARLSIIVTLESLGIDRDFKTEIKSILDETYRYWLRGDEEICLDLATCALAFRLLLAHGYDVSYDPLKPFAEESGFSDTLEGYVKNTFSVLELFKAAQSYPHESALKKQCCWTKQYLEMELSSWVKTSVRDKYLKKEVEDALAFPSYASLERSDHRRKILNGSAVENTRVTKTSYRLHNICTSDILKLAVDDFNFCQSIHREEMERLDRWIVENRLQELKFARQKLAYCYFSGAATLFSPELSDARISWAKGGVLTTVVDDFFDVGGSKEELENLIHLVEKWDLNGVPEYSSEHVEIIFSVLRDTILETGDKAFTYQGRNVTHHIVKIWLDLLKSMLREAEWSSDKSTPSLEDYMENAYISFALGPIVLPATYLIGPPLPEKTVDSHQYNQLYKLVSTMGRLLNDIQGFKRESAEGKLNAVSLHMKHERDNRSKEVIIESMKGLAERKREELHKLVLEEKGSVVPRECKEAFLKMSKVLNLFYRKDDGFTSNDLMSLVKSVIYEPVSLQKESLT

SEQ ID NO: 22 - DNA, 894 bp, synthetic construct
  misc_feature 1..894: note = Synthetic oligonucleotide
  source 1..894: mol_type = other DNA
atggtcgcacaaactttcaacctggatacctacttatcccaaagacaacaacaagttgaagaggccctaagtgctgctcttgtgccagcttatcctgagagaatatacgaagctatgagatactccctcctggcaggtggcaaaagattaagacctatcttatgtttagctgcttgcgaattggcaggtggttctgttgaacaagccatgccaactgcgtgtgcacttgaaatgatccatacaatgtcactaattcatgatgacctgccagccatggataacgatgatttcagaagaggaaagccaactaatcacaaggtgttcggggaagatatagccatcttagcgggtgatgcgcttttagcttacgcttttgaacatattgcttctcaaacaagaggagtaccacctcaattggtgctacaagttattgctagaatcggacacgccgttgctgcaacaggcctcgttggaggccaagtcgtagaccttgaatctgaaggtaaagctatttccttagaaacattggagtatattcactcacataagactggagccttgctggaagcatcagttgtctcaggcggtattctcgcaggggcagatgaagagcttttggccagattgtctcattacgctagagatataggcttggcttttcaaatcgtcgatgatatcctggatgttactgctacatctgaacagttggggaaaaccgctggtaaagaccaggcagccgcaaaggcaacttatccaagtctattgggtttagaagcctctagacagaaagcggaagagttgattcaatctgctaaggaagccttaagaccttacggttcacaagcagagccactcctagcgctggcagacttcatcacacgtcgtcagcattaa

SEQ ID NO: 23 - DNA, 1542 bp, synthetic construct
  misc_feature 1..1542: note = Synthetic oligonucleotide
  source 1..1542: mol_type = other DNA
atggatgctgtgacgggtttgttaactgtcccagcaaccgctataactattggtggaactgctgtagcattggcggtagcgctaatcttttggtacctgaaatcctacacatcagctagaagatcccaatcaaatcatcttccaagagtgcctgaagtcccaggtgttccattgttaggaaatctgttacaattgaaggagaaaaagccatacatgacttttacgagatgggcagcgacatatggacctatctatagtatcaaaactggggctacaagtatggttgtggtatcatctaatgagatagccaaggaggcattggtgaccagattccaatccatatctacaaggaacttatctaaagccctgaaagtacttacagcagataagacaatggtcgcaatgtcagattatgatgattatcataaaacagttaagagacacatactgaccgccgtcttgggtcctaatgcacagaaaaagcatagaattcacagagatatcatgatggataacatatctactcaacttcatgaattcgtgaaaaacaacccagaacaggaagaggtagaccttagaaaaatctttcaatctgagttattcggcttagctatgagacaagccttaggaaaggatgttgaaagtttgtacgttgaagacctgaaaatcactatgaatagagacgaaatctttcaagtccttgttgttgatccaatgatgggagcaatcgatgttgattggagagacttctttccatacctaaagtgggtcccaaacaaaaagttcgaaaatactattcaacaaatgtacatcagaagagaagctgttatgaaatctttaatcaaagagcacaaaaagagaatagcgtcaggcgaaaagctaaatagttatatcgattaccttttatctgaagctcaaactttaaccgatcagcaactattgatgtccttgtgggaaccaatcattgaatcttcagatacaacaatggtcacaacagaatgggcaatgtacgaattagctaaaaaccctaaattgcaagataggttgtacagagacattaagtccgtctgtggatctgaaaagataaccgaagagcatctatcacagctgccttacattacagctattttccacgaaacactgagaagacactcaccagttcctatcattcctctaagacatgtacatgaagataccgttctaggcggctaccatgttcctgctggcacagaacttgccgttaacatctacggttgcaacatggacaaaaacgtttgggaaaatccagaggaatggaacccagaaagattcatgaaagagaatgagacaattgattttcaaaagacgatggccttcggtggtggtaagagagtttgtgctggttccttgcaagcccttttaactgcatctattgggattgggagaatggttcaagagttcgaatggaaactgaaggatatgactcaagaggaagtgaacacgataggcctaactacacaaatgttaagaccattgagagctattatcaaacctaggatctaa

SEQ ID NO: 24 - protein, 297 aa, Synechococcus sp.
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

SEQ ID NO: 25 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 26 - protein, 480 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLAR

SEQ ID NO: 27 - protein, 784 aa, Stevia rebaudiana
MNLSLCIASPLLTKSNRPAALSAIHTASTSHGGQTNPTNLIIDTTKERIQKQFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATEKSQPSPIGFDIIFPGLLEYAKNLDINLLSKQTDFSLMLHKRELEQKRCHSNEMDGYLAYISEGLGNLYDWNMVKKYQMKNGSVFNSPSATAAAFINHQNPGCLNYLNSLLDKFGNAVPTVYPHDLFIRLSMVDTIERLGISHHFRVEIKNVLDETYRCWVERDEQIFMDVVTCALAFRLLRINGYEVSPDPLAEITNELALKDEYAALETYHASHILYQEDLSSGKQILKSADFLKEIISTDSNRLSKLIHKEVENALKFPINTGLERINTRRNIQLYNVDNTRILKTTYHSSNISNTDYLRLAVEDFYTCQSIYREELKGLERWVVENKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 28 - protein, 784 aa, Stevia rebaudiana
MNLSLCIASPLLTKSSRPTALSAIHTASTSHGGQTNPTNLIIDTTKERIQKLFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATDKSQPSPIGFDIIFPGLLEYAKNLDINLLSKQTDFSLMLHKRELEQKRCHSNEIDGYLAYISEGLGNLYDWNMVKKYQMKNGSVFNSPSATAAAFINHQNPGCLNYLNSLLDKFGNAVPTVYPLDLYIRLSMVDTIERLGISHHFRVEIKNVLDETYRCWVERDEQIFMDVVTCALAFRLLRIHGYKVSPDQLAEITNELAFKDEYAALETYHASQILYQEDLSSGKQILKSADFLKGILSTDSNRLSKLIHKEVENALKFPINTGLERINTRRNIQLYNVDNTRILKTTYHSSNISNTYYLRLAVEDFYTCQSIYREELKGLERWVVQNKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 29 - protein, 590 aa, Zea mays
MAMPVKLTPASLSLKAVCCRFSSGGHALRFGSSLPCWRRTPTQRSTSSSTTRPAAEVSSGKSKQHDQEASEATIRQQLQLVDVLENMGISRHFAAEIKCILDRTYRSWLQRHEEIMLDTMTCAMAFRILRLNGYNVSSDELYHVVEASGLHNSLGGYLNDTRTLLELHKASTVSISEDESILDSIGSRSRTLLREQLESGGALRKPSLFKEVEHALDGPFYTTLDRLHHRWNIENFNIIEQHMLETPYLSNQHTSRDILALSIRDFSSSQFTYQQELQHLESWVKECRLDQLQFARQKLAYFYLSAAGTMFSPELSDARTLWAKNGVLTTIVDDFFDVAGSKEELENLVMLVEMWDEHHKVEFYSEQVEIIFSSIYDSVNQLGEKASLVQDRSITKHLVEIWLDLLKSMMTEVEWRLSKYVPTEKEYMINASLIFGLGPIVLPALYFVGPKISESIVKDPEYDELFKLMSTCGRLLNDVQTFEREYNEGKLNSVSLLVLHGGPMSISDAKRKLQKPIDTCRRDLLSLVLREESVVPRPCKELFWKMCKVCYFFYSTTDGFSSQVERAKEVDAVINEPLKLQGSHTLVSDV

SEQ ID NO: 30 - protein, 775 aa, Populus trichocarpa
MSCIRPWFCPSSISATLTDPASKLVTGEFKTTSLNFHGTKERIKKMFDKIELSVSSYDTAWVAMVPSPDCPETPCFPECTKWILENQLGDGSWSLPHGNPLLVKDALSSTLACILALKRWGIGEEQINKGLRFIELNSASVTDNEQHKPIGFDIIFPGMIEYAKDLDLNLPLKPTDINSMLHRRALELTSGGGKNLEGRRAYLAYVSEGIGKLQDWEMAMKYQRKNGSLFNSPSTTAAAFIHIQDAECLHYIRSLLQKFGNAVPTIYPLDIYARLSMVDALERLGIDRHFRKERKFVLDETYRFWLQGEEEIFSDNATCALAFRILRLNGYDVSLEDHFSNSLGGYLKDSGAALELYRALQLSYPDESLLEKQNSRTSYFLKQGLSNVSLCGDRLRKNIIGEVHDALNFPDHANLQRLAIRRRIKHYATDDTRILKTSYRCSTIGNQDFLKLAVEDFNICQSIQREEFKHIERWVVERRLDKLKFARQKEAYCYFSAAATLFAPELSDARMSWAKNGVLTTVVDDFFDVGGSEEELVNLIELIERWDVNGSADFCSEEVEIIYSAIHSTISEIGDKSFGWQGRDVKSHVIKIWLDLLKSMLTEAQWSSNKSVPTLDEYMTTAHVSFALGPIVLPALYFVGPKLSEEVAGHPELLNLYKVMSTCGRLLNDWRSFKRESEEGKLNAISLYMIHSGGASTEEETIEHFKGLIDSQRRQLLQLVLQEKDSIIPRPCKDLFWNMIKLLHTFYMKDDGFTSNEMRNVVKAIINEPISLDEL

SEQ ID NO: 31 - DNA, 2358 bp, synthetic construct
  misc_feature 1..2358: note = Synthetic oligonucleotide
  source 1..2358: mol_type = other DNA
atgtctatcaaccttcgctcctccggttgttcgtctccgatctcagctactttggaacgaggattggactcagaagtacagacaagagctaacaatgtgagctttgagcaaacaaaggagaagattaggaagatgttggagaaagtggagctttctgtttcggcctacgatactagttgggtagcaatggttccatcaccgagctcccaaaatgctccacttttcccacagtgtgtgaaatggttattggataatcaacatgaagatggatcttggggacttgataaccatgaccatcaatctcttaagaaggatgtgttatcatctacactggctagtatcctcgcgttaaagaagtggggaattggtgaaagacaaataaacaagggtctccagtttattgagctgaattctgcattagtcactgatgaaaccatacagaaaccaacagggtttgatattatatttcctgggatgattaaatatgctagagatttgaatctgacgattccattgggctcagaagtggtggatgacatgatacgaaaaagagatctggatcttaaatgtgatagtgaaaagttttcaaagggaagagaagcatatctggcctatgttttagaggggacaagaaacctaaaagattgggatttgatagtcaaatatcaaaggaaaaatgggtcactgtttgattctccagccacaacagcagctgcttttactcagtttgggaatgatggttgtctccgttatctctgttctctccttcagaaattcgaggctgcagttccttcagtttatccatttgatcaatatgcacgccttagtataattgtcactcttgaaagcttaggaattgatagagatttcaaaaccgaaatcaaaagcatattggatgaaacctatagatattggcttcgtggggatgaagaaatatgtttggacttggccacttgtgctttggctttccgattattgcttgctcatggctatgatgtgtcttacgatccgctaaaaccatttgcagaagaatctggtttctctgatactttggaaggatatgttaagaatacgttttctgtgttagaattatttaaggctgctcaaagttatccacatgaatcagctttgaagaagcagtgttgttggactaaacaatatctggagatggaattgtccagctgggttaagacctctgttcgagataaatacctcaagaaagaggtcgaggatgctcttgcttttccctcctatgcaagcctagaaagatcagatcacaggagaaaaatactcaatggttctgctgtggaaaacaccagagttacaaaaacctcatatcgtttgcacaatatttgcacctctgatatcctgaagttagctgtggatgacttcaatttctgccagtccatacaccgtgaagaaatggaacgtcttgataggtggattgtggagaatagattgcaggaactgaaatttgccagacagaagctggcttactgttatttctctggggctgcaactttattttctccagaactatctgatgctcgtatatcgtgggccaaaggtggagtacttacaacggttgtagacgacttctttgatgttggagggtccaaagaagaactggaaaacctcatacacttggtcgaaaagtgggatttgaacggtgttcctgagtacagctcagaacatgttgagatcatattctcagttctaagggacaccattctcgaaacaggagacaaagcattcacctatcaaggacgcaatgtgacacaccacattgtgaaaatttggttggatctgctcaagtctatgttgagagaagccgagtggtccagtgacaagtcaacaccaagcttggaggattacatggaaaatgcgtacatatcatttgcattaggaccaattgtcctcccagctacctatctgatcggacctccacttccagagaagacagtcgatagccaccaatataatcagctctacaagctcgtgagcactatgggtcgtcttctaaatgacatacaaggttttaagagagaaagcgcggaagggaagctgaatgcggtttcattgcacatgaaacacgagagagacaatcgcagcaaagaagtgatcatagaatcgatgaaaggtttagcagagagaaagagggaagaattgcataagctagttttggaggagaaaggaagtgtggttccaagggaatgcaaagaagcgttcttgaaaatgagcaaagtgttgaacttattttacaggaaggacgatggattcacatcaaatgatctgatgagtcttgttaaatcagtgatctacgagcctgttagcttacagaaagaatctttaacttga

SEQ ID NO: 32 - protein, 785 aa, Arabidopsis thaliana; identical to SEQ ID NO: 21

SEQ ID NO: 33 - protein, 513 aa, Stevia rebaudiana; identical to SEQ ID NO: 25

SEQ ID NO: 34 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 35 - protein, 525 aa, Fusarium fujikuroi
MSKSNSMNSTSHETLFQQLVLGLDRMPLMDVHWLIYVAFGAWLCSYVIHVLSSSSTVKVPVVGYRSVFEPTWLLRLRFVWEGGSIIGQGYNKFKDSIFQVRKLGTDIVIIPPNYIDEVRKLSQDKTRSVEPFINDFAGQYTRGMVFLQSDLQNRVIQQRLTPKLVSLTKVMKEELDYALTKEMPDMKNDEWVEVDISSIMVRLISRISARVFLGPEHCRNQEWLTTTAEYSESLFITGFILRVVPHILRPFIAPLLPSYRTLLRNVSSGRRVIGDIIRSQQGDGNEDILSWMRDAATGEEKQIDNIAQRMLILSLASIHTTAMTMTHAMYDLCACPEYIEPLRDEVKSVVGASGWDKTALNRFHKLDSFLKESQRFNPVFLLTFNRIYHQSMTLSDGTNIPSGTRIAVPSHAMLQDSAHVPGPTPPTEFDGFRYSKIRSDSNYAQKYLFSMTDSSNMAFGYGKYACPGRFYASNEMKLTLAILLLQFEFKLPDGKGRPRNITIDSDMIPDPRARLCVRKRSLRDE

SEQ ID NO: 36 - protein, 499 aa, Trametes versicolor
MEDPTVLYACLAIAVATFVVRWYRDPLRSIPTVGGSDLPILSYIGALRWTRRGREILQEGYDGYRGSTFKIAMLDRWIVIANGPKLADEVRRRPDEELNFMDGLGAFVQTKYTLGEAIHNDPYHVDIIREKLTRGLPAVLPDVIEELTLAVRQYIPTEGDEWVSVNCSKAARDIVARASNRVFVGLPACRNQGYLDLAIDFTLSVVKDRAIINMFPELLKPIVGRVVGNATRNVRRAVPFVAPLVEERRRLMEEYGEDWSEKPNDMLQWIMDEAASRDSSVKAIAERLLMVNFAAIHTSSNTITHALYHLAEMPETLQPLREEIEPLVKEEGWTKAAMGKMWWLDSFLRESQRYNGINIVSLTRMADKDITLSDGTFLPKGTLVAVPAYSTHRDDAVYADALVFDPFRFSRMRAREGEGTKHQFVNTSVEYVPFGHGKHACPGRFFAANELKAMLAYIVLNYDVKLPGDGKRPLNMYWGPTVLPAPAGQVLFRKRQVSL

SEQ ID NO: 37 - DNA, 1678 bp, synthetic construct
  misc_feature 1..1678: note = Synthetic oligonucleotide
  source 1..1678: mol_type = other DNA
aaacaaagaatgattcaagttctaacaccgatccttctcttcctcattttcttcgttttctggaaggtttacaagcaccagaaaaccaaaatcaatcttccaccgggaagcttcggatggccatttctgggcgaaactctggcactcctacgtgcaggttgggactcagagccggagagatttgttcgtgaacggatcaagaaacacggaagtcctctagtgtttaagacgtcgttgtttggcgaccgttttgcggtgttgtgtggacctgccggaaacaagttcctgttctgcaacgagaacaagctggtggcgtcgtggtggccggttccggtgaggaagcttttcggcaagtctctgctcacgattcgtggtgatgaagctaagtggatgaggaagatgttgttatcgtatctcggtcctgatgctttcgcaactcattatgccgtcaccatggacgtcgtcacccgtcggcatatcgacgttcattggcgagggaaggaagaggtgaacgtattccaaaccgttaagttatatgcctttgagcttgcatgtcgtttattcatgaacctagacgacccaaaccacattgcaaaactcggttccttgttcaacattttcttgaaaggcatcattgagcttccaatcgacgtcccagggacacgattttatagctccaaaaaagcagcagcagctatcaggattgaactaaaaaaattgattaaagcaagaaaactggaactgaaagaagggaaggcatcatcttcacaagacctcttatcacatttgcttacatctccagatgaaaatggtatgtttctaaccgaagaagagattgtagacaacatcttgttactactctttgcgggtcatgatacctcggctctttcaatcactttgctcatgaagactcttggcgaacattctgatgtttatgacaaggtgttaaaagagcaactagagatatcgaagacgaaagaagcatgggagtccctgaaatgggaggacatacaaaagatgaaatactcctggagtgttatatgtgaagtcatgagactaaatccacctgttataggaacctatagagaggcccttgtggatattgattatgcgggttataccatccccaaaggatggaagctgcactggagtgctgtatcgacacaaagggacgaggctaactttgaagacgtaacacgttttgacccatcacggtttgaaggcgcaggaccgactccattcacctttgttccgtttggaggggggcctagaatgtgtttagggaaagaatttgctcgattggaagtacttgcgtttcttcacaatattgtcaccaatttcaaatgggacctgttgatacctgatgagaaaatagaatatgatcccatggctaccccagcaaaggggcttccaattcgtcttcatccccatcaagtttgattacttcaagcatgaatcagtgatgtgaaggtaaaccataatggatcttattggtagttacagattatgtgtttttatggcatgaagaagttatgataaataaaattgtgttattctacaacttatgtaatttgtgcctgtaagtaactgaatctattaatgttttatgtgacatgaaacataaatgtataattagtaaattttctgctcaaaaaaaaaaaaaaaaaaaaaaaaaaaa

SEQ ID NO: 38 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 39 - protein, 476 aa, Stevia rebaudiana; identical to SEQ ID NO: 38

SEQ ID NO: 40 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 41 - protein, 526 aa, Vitis vinifera
MYFLLQYLNITTVGVFATLFLSYCLLLWRSRAGNKKIAPEAAAAWPIIGHLHLLAGGSHQLPHITLGNMADKYGPVFTIRIGLHRAVVVSSWEMAKECSTANDQVSSSRPELLASKLLGYNYAMFGFSPYGSYWREMRKIISLELLSNSRLELLKDVRASEVVTSIKELYKLWAEKKNESGLVSVEMKQWFGDLTLNVILRMVAGKRYFSASDASENKQAQRCRRVFREFFHLSGLFVVADAIPFLGWLDWGRHEKTLKKTAIEMDSIAQEWLEEHRRRKDSGDDNSTQDFMDVMQSVLDGKNLGGYDADTINKATCLTLISGGSDTTVVSLTWALSLVLNNRDTLKKAQEELDIQVGKERLVNEQDISKLVYLQAIVKETLRLYPPGPLGGLRQFTEDCTLGGYHVSKGTRLIMNLSKIQKDPRIWSDPTEFQPERFLTTHKDVDPRGKHFEFIPFGAGRRACPGITFGLQVLHLTLASFLHAFEFSTPSNEQVNMRESLGLTNMKSTPLEVLISPRLSSCSLYN

SEQ ID NO: 42 - protein, 479 aa, Medicago truncatula
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 43 - protein, 522 aa, Stevia rebaudiana
MGLFPLEDSYALVFEGLAITLALYYLLSFIYKTSKKTCTPPKASGEHPITGHLNLLSGSSGLPHLALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 44 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 45 - protein, 692 aa, Arabidopsis thaliana [sequence omitted]

SEQ ID NO: 46 - protein, 713 aa, Fusarium fujikuroi
MAELDTLDIVVLGVIFLGTVAYFTKGKLWGVTKDPYANGFAAGGASKPGRTRNIVEAMEESGKNCVVFYGSQTGTAEDYASRLAKEGKSRFGLNTMIADLEDYDFDNLDTVPSDNIVMFVLATYGEGEPTDNAVDFYEFITGEDASFNEGNDPPLGNLNYVAFGLGNNTYEHYNSMVRNVNKALEKLGAHRIGEAGEGDDGAGTMEEDFLAWKDPMWEALAKKMGLEEREAVYEPIFAINERDDLTPEANEVYLGEPNKLHLEGTAKGPFNSHNPYIAPIAESYELFSAKDRNCLHMEIDISGSNLKYETGDHIAIWPTNPGEEVNKFLDILDLSGKQHSVVTVKALEPTAKVPFPNPTTYDAILRYHLEICAPVSRQFVSTLAAFAPNDDIKAEMNRLGSDKDYFHEKTGPHYYNIARFLASVSKGEKWTKIPFSAFIEGLTKLQPRYYSISSSSLVQPKKISITAVVESQQIPGRDDPFRGVATNYLFALKQKQNGDPNPAPFGQSYELTGPRNKYDGIHVPVHVRHSNFKLPSDPGKPIIMIGPGTGVAPFRGFVQERAKQARDGVEVGKTLLFFGCRKSTEDFMYQKEWQEYKEALGDKFEMITAFSREGSKKVYVQHRLKERSKEVSDLLSQKAYFYVCGDAAHMAREVNTVLAQIIAEGRGVSEAKGEEIVKNMRSANQYQVCSDFVTLHCKETTYANSELQEDVWS

SEQ ID NO: 47 - protein, 480 aa, Stevia rebaudiana; identical to SEQ ID NO: 26

SEQ ID NO: 48 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 49 - protein, 527 aa, Streptomyces clavuligerus
MPDAHDAPPPQIRQRTLVDEATQLLTESAEDAWGEVSVSEYETARLVAHATWLGGHATRVAFLLERQHEDGSWGPPGGYRLVPTLSAVHALLTCLASPAQDHGVPHDRLLRAVDAGLTALRRLGTSDSPPDTIAVELVIPSLLEGIQHLLDPAHPHSRPAFSQHRGSLVCPGGLDGRTLGALRSHAAAGTPVPGKVWHASETLGLSTEAASHLQPAQGIIGGSAAATATWLTRVAPSQQSDSARRYLEELQHRYSGPVPSITPITYFERAWLLNNFAAAGVPCEAPAALLDSLEAALTPQGAPAGAGLPPDADDTAAVLLALATHGRGRRPEVLMDYRTDGYFQCFIGERTPSISTNAHVLETLGHHVAQHPQDRARYGSAMDTASAWLLAAQKQDGSWLDKWHASPYYATVCCTQALAAHASPATAPARQRAVRWVLATQRSDGGWGLWHSTVEETAYALQILAPPSGGGNIPVQQALTRGRARLCGALPLTPLWHDKDLYTPVRVVRAARAAALYTTRDLLLPPL

SEQ ID NO: 50 - protein, 516 aa, Bradyrhizobium diazoefficiens
MNALSEHILSELRRLLSEMSDGGSVGPSVYDTAQALRFHGNVTGRQDAYAWLIAQQQADGGWGSADFPLFRHAPTWAALLALQRADPLPGAADAVQTATRFLQRQPDPYAHAVPEDAPIGAELILPQFCGEAAWLLGGVAFPRHPALLPLRQACLVKLGAVAMLPSGHPLLHSWEAWGTSPTTACPDDDGSIGISPAATAAWRAQAVTRGSTPQVGRADAYLQMASRATRSGIEGVFPNVWPINVFEPCWSLYTLHLAGLFAHPALAEAVRVIVAQLEARLGVHGLGPALHFAADADDTAVALCVLHLAGRDPAVDALRHFEIGELFVTFPGERNASVSTNIHALHALRLLGKPAAGASAYVEANRNPHGLWDNEKWHVSWLYPTAHAVAALAQGKPQWRDERALAALLQAQRDDGGWGAGRGSTFEETAYALFALHVMDGSEEATGRRRIAQVVARALEWMLARHAAHGLPQTPLWIGKELYCPTRVVRVAELAGLWLALRWGRRVLAEGAGAAP

SEQ ID NO: 51 - DNA, 2490 bp, synthetic construct; identical to SEQ ID NO: 12
  misc_feature 1..2490: note = Synthetic oligonucleotide
  source 1..2490: mol_type = other DNA

SEQ ID NO: 52 - protein, 827 aa, Zea mays; identical to SEQ ID NO: 13

SEQ ID NO: 53 - DNA, 2570 bp, synthetic construct
  misc_feature 1..2570: note = Synthetic oligonucleotide
  source 1..2570: mol_type = other DNA
cttcttcactaaatacttagacagagaaaacagagctttttaaagccatgtctcttcagtatcatgttctaaactccattccaagtacaacctttctcagttctactaaaacaacaatatcttcttctttccttaccatctcaggatctcctctcaatgtcgctagagacaaatccagaagcggttccatacattgttcaaagcttcgaactcaagaatacattaattctcaagaggttcaacatgatttgcctctaatacatgagtggcaacagcttcaaggagaagatgctcctcagattagtgttggaagtaatagtaatgcattcaaagaagcagtgaagagtgtgaaaacgatcttgagaaacctaacggacggggaaattacgatatcggcttacgatacagcttgggttgcattgatcgatgccggagataaaactccggcgtttccctccgccgtgaaatggatcgccgagaaccaactttccgatggttcttggggagatgcgtatctcttctcttatcatgatcgtctcatcaatacccttgcatgcgtcgttgctctaagatcatggaatctctttcctcatcaatgcaacaaaggaatcacgtttttccgggaaaatattgggaagctagaagacgaaaatgatgagcatatgccaatcggattcgaagtagcattcccatcgttgcttgagatagctcgaggaataaacattgatgtaccgtacgattctccggtcttaaaagatatatacgccaagaaagagctaaagcttacaaggataccaaaagagataatgcacaagataccaacaacattgttgcatagtttggaggggatgcgtgatttagattgggaaaagctcttgaaacttcaatctcaagacggatctttcctcttctctccttcctctaccgcttttgcattcatgcagacccgagacagtaactgcctcgagtatttgcgaaatgccgtcaaacgtttcaatggaggagttcccaatgtctttcccgtggatcttttcgagcacatatggatagtggatcggttacaacgtttagggatatcgagatactttgaagaagagattaaagagtgtcttgactatgtccacagatattggaccgacaatggcatatgttgggctagatgttcccatgtccaagacatcgatgatacagccatggcatttaggctcttaagacaacatggataccaagtgtccgcagatgtattcaagaactttgagaaagagggagagtttttctgctttgtggggcaatcaaaccaagcagtaaccggtatgttcaacctataccgggcatcacaattggcgtttccaagggaagagatattgaaaaacgccaaagagttttcttataattatctgctagaaaaacgggagagagaggagttgattgataagtggattataatgaaagacttacctggcgagattgggtttgcgttagagattccatggtacgcaagcttgcctcgagtagagacgagattctatattgatcaatatggtggagaaaacgacgtttggattggcaagactctttataggatgccatacgtgaacaataatggatatctggaattagcaaaacaagattacaacaattgccaagctcagcatcagctcgaatgggacatattccaaaagtggtatgaagaaaataggttaagtgagtggggtgtgcgcagaagtgagcttctcgagtgttactacttagcggctgcaactatatttgaatcagaaaggtcacatgagagaatggtttgggctaagtcaagtgtattggttaaagccatttcttcttcttttggggaatcctctgactccagaagaagcttctccgatcagtttcatgaatacattgccaatgctcgacgaagtgatcatcactttaatgacaggaacatgagattggaccgaccaggatcggttcaggccagtcggcttgccggagtgttaatcgggactttgaatcaaatgtcttttgaccttttcatgtctcatggccgtgacgttaacaatctcctctatctatcgtggggagattggatggaaaaatggaaactatatggagatgaaggagaaggagagctcatggtgaagatgataattctaatgaagaacaatgacctaactaacttcttcacccacactcacttcgttcgtctcgcggaaatcatcaatcgaatctgtcttcctcgccaatacttaaaggcaaggagaaacgatgagaaggagaagacaataaagagtatggagaaggagatggggaaaatggttgagttagcattgtcggagagtgacacatttcgtgacgtcagcatcacgtttcttgatgtagcaaaagcattttactactttgctttatgtggcgatcatctccaaactcacatctccaaagtcttgtttcaaaaagtctagtaacctcatcatcatcatcgatccattaacaatcagtggatcgatgtatccatagatgcgtgaataatatttcatgtagagaaggagaacaaattagatcatgtagggttatca

SEQ ID NO: 54 - protein, 802 aa, Arabidopsis thaliana
MSLQYHVLNSIPSTTFLSSTKTTISSSFLTISGSPLNVARDKSRSGSIHCSKLRTQEYINSQEVQHDLPLIHEWQQLQGEDAPQISVGSNSNAFKEAVKSVKTILRNLTDGEITISAYDTAWVALIDAGDKTPAFPSAVKWIAENQLSDGSWGDAYLFSYHDRLINTLACVVALRSWNLFPHQCNKGITFFRENIGKLEDENDEHMPIGFEVAFPSLLEIARGINIDVPYDSPVLKDIYAKKELKLTRIPKEIMHKIPTTLLHSLEGMRDLDWEKLLKLQSQDGSFLFSPSSTAFAFMQTRDSNCLEYLRNAVKRFNGGVPNVFPVDLFEHIWIVDRLQRLGISRYFEEEIKECLDYVHRYWTDNGICWARCSHVQDIDDTAMAFRLLRQHGYQVSADVFKNFEKEGEFFCFVGQSNQAVTGMFNLYRASQLAFPREEILKNAKEFSYNYLLEKREREELIDKWIIMKDLPGEIGFALEIPWYASLPRVETRFYIDQYGGENDVWIGKTLYRMPYVNNNGYLELAKQDYNNCQAQHQLEWDIFQKWYEENRLSEWGVRRSELLECYYLAAATIFESERSHERMVWAKSSVLVKAISSSFGESSDSRRSFSDQFHEYIANARRSDHHFNDRNMRLDRPGSVQASRLAGVLIGTLNQMSFDLFMSHGRDVNNLLYLSWGDWMEKWKLYGDEGEGELMVKMIILMKNNDLTNFFTHTHFVRLAEIINRICLPRQYLKARRNDEKEKTIKSMEKEMGKMVELALSESDTFRDVSITFLDVAKAFYYFALCGDHLQTHISKVLFQKV

SEQ ID NO: 55 - protein, 983 aa, Diaporthe amygdali
MEFDEPLVDEARSLVQRTLQDYDDRYGFGTMSCAAYDTAWVSLVTKTVDGRKQWLFPECFEFLLETQSDAGGWEIGNSAPIDGILNTAASLLALKRHVQTEQIIQPQHDHKDLAGRAERAAASLRAQLAALDVSTTEHVGFEIIVPAMLDPLEAEDPSLVFDFPARKPLMKIHDAKMSRFRPEYLYGKQPMTALHSLEAFIGKIDFDKVRHHRTHGSMMGSPSSTAAYLMHASQWDGDSEAYLRHVIKHAAGQGTGAVPSAFPSTHFESSWILTTLFRAGFSASHLACDELNKLVEILEGSFEKEGGAIGYAPGFQADVDDTAKTISTLAVLGRDATPRQMIKVFEANTHFRTYPGERDPSLTANCNALSALLHQPDAAMYGSQIQKITKFVCDYWWKSDGKIKDKWNTCYLYPSVLLVEVLVDLVSLLEQGKLPDVLDQELQYRVAITLFQACLRPLLDQDAEGSWNKSIEATAYGILILTEARRVCFFDRLSEPLNEAIRRGIAFADSMSGTEAQLNYIWIEKVSYAPALLTKSYLLAARWAAKSPLGASVGSSLWTPPREGLDKHVRLFHQAELFRSLPEWELRASMIEAALFTPLLRAHRLDVFPRQDVGEDKYLDVVPFFWTAANNRDRTYASTLFLYDMCFIAMLNFQLDEFMEATAGILFRDHMDDLRQLIHDLLAEKTSPKSSGRSSQGTKDADSGIEEDVSMSDSASDSQDRSPEYDLVFSALSTFTKHVLQHPSIQSASVWDRKLLAREMKAYLLAHIQQAEDSTPLSELKDVPQKTDVTRVSTSTTTFFNWVRTTSADHISCPYSFHFVACHLGAALSPKGSNGDCYPSAGEKFLAAAVCRHLATMCRMYNDLGSAERDSDEGNLNSLDFPEFADSAGNGGIEIQKAALLRLAEFERDSYLEAFRRLQDESNRVHGPAGGDEARLSRRRMAILEFFAQQVDLYGQVYVIRDISARIPKNEVEKKRKLDDAFN

SEQ ID NO: 56 - protein, 881 aa, Physcomitrella patens
MASSTLIQNRSCGVTSSMSSFQIFRGQPLRFPGTRTPAAVQCLKKRRCLRPTESVLESSPGSGSYRIVTGPSGINPSSNGHLQEGSLTHRLPIPMEKSIDNFQSTLYVSDIWSETLQRTECLLQVTENVQMNEWIEEIRMYFRNMTLGEISMSPYDTAWVARVPALDGSHGPQFHRSLQWIIDNQLPDGDWGEPSLFLGYDRVCNTLACVIALKTWGVGAQNVERGIQFLQSNIYKMEEDDANHMPIGFEIVFPAMMEDAKALGLDLPYDATILQQISAEREKKMKKIPMAMVYKYPTTLLHSLEGLHREVDWNKLLQLQSENGSFLYSPASTACALMYTKDVKCFDYLNQLLIKFDHACPNVYPVDLFERLWMVDRLQRLGISRYFEREIRDCLQYVYRYWKDCGIGWASNSSVQDVDDTAMAFRLLRTHGFDVKEDCFRQFFKDGEFFCFAGQSSQAVTGMFNLSRASQTLFPGESLLKKARTFSRNFLRTKHENNECFDKWIITKDLAGEVEYNLTFPWYASLPRLEHRTYLDQYGIDDIWIGKSLYKMPAVTNEVFLKLAKADFNMCQALHKKELEQVIKWNASCQFRDLEFARQKSVECYFAGAATMFEPEMVQARLVWARCCVLTTVLDDYFDHGTPVEELRVFVQAVRTWNPELINGLPEQAKILFMGLYKTVNTIAEEAFMAQKRDVHHHLKHYWDKLITSALKEAEWAESGYVPTFDEYMEVAEISVALEPIVCSTLFFAGHRLDEDVLDSYDYHLVMHLVNRVGRILNDIQGMKREASQGKISSVQIYMEEHPSVPSEAMAIAHLQELVDNSMQQLTYEVLRFTAVPKSCKRIHLNMAKIMHAFYKDTDGFSSLTAMTGFVKKVLFEPVPE

SEQ ID NO: 57 - protein, 952 aa, Gibberella fujikuroi
MPGKIENGTPKDLKTGNDFVSAAKSLLDRAFKSHHSYYGLCSTSCQVYDTAWVAMIPKTRDNVKQWLFPECFHYLLKTQAADGSWGSLPTTQTAGILDTASAVLALLCHAQEPLQILDVSPDEMGLRIEHGVTSLKRQLAVWNDVEDTNHIGVEFIIPALLSMLEKELDVPSFEFPCRSILERMHGEKLGHFDLEQVYGKPSSLLHSLEAFLGKLDFDRLSHHLYHGSMMASPSSTAAYLIGATKWDDEAEDYLRHVMRNGAGHGNGGISGTFPTTHFECSWIIATLLKVGFTLKQIDGDGLRGLSTILLEALRDENGVIGFAPRTADVDDTAKALLALSLVNQPVSPDIMIKVFEGKDHFTTFGSERDPSLTSNLHVLLSLLKQSNLSQYHPQILKTTLFTCRWWWGSDHCVKDKWNLSHLYPTMLLVEAFTEVLHLIDGGELSSLFDESFKCKIGLSIFQAVLRIILTQDNDGSWRGYREQTCYAILALVQARHVCFFTHMVDRLQSCVDRGFSWLKSCSFHSQDLTWTSKTAYEVGFVAEAYKLAALQSASLEVPAATIGHSVTSAVPSSDLEKYMRLVRKTALFSPLDEWGLMASIIESSFFVPLLQAQRVEIYPRDNIKVDEDKYLSIIPFTWVGCNNRSRTFASNRWLYDMMYLSLLGYQTDEYMEAVAGPVFGDVSLLHQTIDKVIDNTMGNLARANGTVHSGNGHQHESPNIGQVEDTLTRFTNSVLNHKDVLNSSSSDQDTLRREFRTFMHAHITQIEDNSRFSKQASSDAFSSPEQSYFQWVNSTGGSHVACAYSFAFSNCLMSANLLQGKDAFPSGTQKYLISSVMRHATNMCRMYNDFGSIARDNAERNVNSIHFPEFTLCNGTSQNLDERKERLLKIATYEQGYLDRALEALERQSRDDAGDRAGSKDMRKLKIVKLFCDVTDLYDQLYVIKDLSSSMK

SEQ ID NO: 58 - protein, 361 aa, Stevia rebaudiana
MALVNPTALFYGTSIRTRPTNLLNPTQKLRPVSSSSLPSFSSVSAILTEKHQSNPSENNNLQTHLETPFNFDSYMLEKVNMVNEALDASVPLKDPIKIHESMRYSLLAGGKRIRPMMCIAACEIVGGNILNAMPAACAVEMIHTMSLVHDDLPCMDNDDFRRGKPISHKVYGEEMAVLTGDALLSLSFEHIATATKGVSKDRIVRAIGELARSVGSEGLVAGQVVDILSEGADVGLDHLEYIHIHKTAMLLESSVVIGAIMGGGSDQQIEKLRKFARSIGLLFQVVDDILDVTKSTEELGKTAGKDLLTDKTTYPKLLGIEKSREFAEKLNKEAQEQLSGFDRRKAAPLIALANYNAYRQN

SEQ ID NO: 59 - protein, 342 aa, Fusarium fujikuroi
MAEQQISNLLSMFDASHASQKLEITVQMMDTYHYRETPPDSSSSEGGSLSRYDERRVSLPLSHNAASPDIVSQLCFSTAMSSELNHRWKSQRLKVADSPYNYILTLPSKGIRGAFIDSLNVWLEVPEDETSVIKEVIGMLHNSSLIIDDFQDNSPLRRGKPSTHTVFGPAQAINTATYVIVKAIEKIQDIVGHDALADVTGTITTIFQGQAMDLWWTANAIVPSIQEYLLMVNDKTGALFRLSLELLALNSEASISDSALESLSSAVSLLGQYFQIRDDYMNLIDNKYTDQKGFCEDLDEGKYSLTLIHALQTDSSDLLTNILSMRRVQGKLTAQKRCWFWK

SEQ ID NO: 60 - protein, 300 aa, Mus musculus
MEKTKEKAERILLEPYRYLLQLPGKQVRSKLSQAFNHWLKVPEDKLQIIIEVTEMLHNASLLIDDIEDSSKLRRGFPVAHSIYGVPSVINSANYVYFLGLEKVLTLDHPDAVKLFTRQLLELHQGQGLDIYWRDTYTCPTEEEYKAMVLQKTGGLFGLAVGLMQLFSDYKEDLKPLLDTLGLFFQIRDDYANLHSKEYSENKSFCEDLTEGKFSFPTIHAIWSRPESTQVQNILRQRTENIDIKKYCVQYLEDVGSFAYTRHTLRELEAKAYKQIEACGGNPSLVALVKHLSKMFTEENK

SEQ ID NO: 61 - protein, 339 aa, Thalassiosira pseudonana
MARFYFLNALLMVISLQSTTAFTPAKLAYPTTTTALNVASAETSFSLDEYLASKIGPIESALEASVKSRIPQTDKICESMAYSLMAGGKRIRPVLCIAACEMFGGSQDVAMPTAVALEMIHTMSLIHDDLPSMDNDDLRRGKPTNHVVFGEDVAILAGDSLLSTSFEHVARETKGVSAEKIVDVIARLGKSVGAEGLAGGQVMDLECEAKPGTTLDDLKWIHIHKTATLLQVAVASGAVLGGATPEEVAACELFAMNIGLAFQVADDILDVTASSEDLGKTAGKDEATDKTTYPKLLGLEESKAYARQLIDEAKESLAPFGDRAAPLLAIADFIIDRKN

SEQ ID NO: 62 - protein, 355 aa, Streptomyces clavuligerus
MHLAPRRVPRGRRSPPDRVPERQGALGRRRGAGSTGCARAAAGVHRRRGGGEADPSAAVHRGWQAGGGTGLPDEVVSTAAALEMFHAFALIHDDIMDDSATRRGSPTVHRALADRLGAALDPDQAGQLGVSTAILVGDLALTWSDELLYAPLTPHRLAAVLPLVTAMRAETVHGQYLDITSARRPGTDTSLALRIARYKTAAYTMERPLHIGAALAGARPELLAGLSAYALPAGEAFQLADDLLGVFGDPRRTGKPDLDDLRGGKHTVLVALAREHATPEQRHTLDTLLGTPGLDRQGASRLRCVLVATGARAEAERLITERRDQALTALNALTLPPPLAEALARLTLGSTAHPA

SEQ ID NO: 63 - protein, 330 aa, Sulfolobus acidocaldarius
MSYFDNYFNEIVNSVNDIIKSYISGDVPKLYEASYHLFTSGGKRLRPLILTISSDLFGGQRERAYYAGAAIEVLHTFTLVHDDIMDQDNIRRGLPTVHVKYGLPLAILAGDLLHAKAFQLLTQALRGLPSETIIKAFDIFTRSIIIISEGQAVDMEFEDRIDIKEQEYLDMISRKTAALFSASSSIGALIAGANDNDVRLMSDFGTNLGIAFQIVDDILGLTADEKELGKPVFSDIREGKKTILVIKTLELCKEDEKKIVLKALGNKSASKEELMSSADIIKKYSLDYAYNLAEKYYKNAIDSLNQVSSKSDIPGKALKYLAEFTIRRRK

SEQ ID NO: 64 - protein, 297 aa, Synechococcus sp.; identical to SEQ ID NO: 24

SEQ ID NO: 65 - protein, 371 aa, Arabidopsis thaliana
MASVTLGSWIVVHHHNHHHPSSILTKSRSRSCPITLTKPISFRSKRTVSSSSSIVSSSVVTKEDNLRQSEPSSFDFMSYIITKAELVNKALDSAVPLREPLKIHEAMRYSLLAGGKRVRPVLCIAACELVGGEESTAMPAACAVEMIHTMSLIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 66 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 67 - protein, 459 aa, Ipomoea purpurea
MGSQATTYHMAMYPWFGVGHLTGFFRLANKLAGKGHRISFLIPKNTQSKLESFNLHPHLISFVPIVVPSIPGLPPGAETTSDVPFPSTHLLMEAMDKTQNDIEIILKDLKVDVVFYDFTHWLPSLARKIGIKSVFYSTISPLMHGYALSPERRVVGKQLTEADMMKAPASFPDPSIKLHAHEARGFTARTVMKFGGDITFFDRIFTAVSESDGLAYSTCREIEGQFCDYIETQFQKPVLLAGPALPVPSKSTMEQKWSDWLGKFKEGSVIYCAFGSECTLRKDKFQELLWGLELTGMPFFAALKPPFETESVEAAIPEELKEKIQGRGIVHGEWVQQQLFLQHPSVGCFVSHCGWASLSEALVNDCQIVLLPQVGDQIINARIMSVSLKVGVEVEKGEEDGVFSRESVCKAVKAVMDEKSEIGREVRGNHDKLRGFLMNADLDSKYMDSFNQKLQDLLG

SEQ ID NO: 68 - DNA, 1380 bp, synthetic construct
  misc_feature 1..1380: note = Synthetic oligonucleotide
  source 1..1380: mol_type = other DNA
atgggttctcaagctacaacttaccatatggccatgtatccatggtttggggttggacatttgactggtttcttccgtttggcaaacaaattagctggcaaaggacatagaatctcatttctaattcctaaaaacactcaatctaagttagaatctttcaaccttcatccacacttaatctcttttgtgcctatcgttgtcccaagtataccaggcctgccacctggtgcagagactacatcagatgttcctttcccaagtacacatttgctaatggaagcaatggacaagactcaaaacgatatagagattatcctgaaggatcttaaagtagatgttgttttctatgattttactcactggttgccttctctggccagaaagattggcattaagagtgtcttttactccaccatttctcctttaatgcatggatatgctttatcaccagaaagacgtgtagttggtaagcaattgacagaggcagatatgatgaaggccccagcttctttcccagacccatccattaagctacatgcacatgaagctaggggttttacagccagaaccgttatgaaattcggtggtgacatcacctttttcgatagaatattcacagcagtttccgaaagtgatggcctggcctactctacttgtagagagatcgagggacaattctgtgattacattgaaacacaattccagaagccagtcttgttagccggtccagctttgccagtcccatccaaatccactatggaacaaaagtggtcagattggttggggaaattcaaggaaggctccgtcatctactgtgctttcgggtctgaatgtacattgagaaaggacaaatttcaggaacttttatggggtttggaattgacaggaatgcctttcttcgctgctctgaagccaccttttgagactgagtctgttgaggctgctatccctgaggaactaaaggaaaagattcagggaagaggtatagtacatggagaatgggtacaacaacaattgtttcttcaacacccatctgtcgggtgcttcgtttctcactgcggctgggcaagtttatctgaagcccttgttaatgattgtcaaatcgtgttacttccacaagttggcgatcagattatcaacgccagaataatgtcagtatcacttaaagtgggcgtggaagttgaaaagggtgaggaggacggtgtcttttcaagagaatctgtgtgcaaggctgttaaagcagtaatggatgaaaaatctgaaatcggtagagaagtcagaggtaatcatgataaactgaggggtttcttgatgaatgcagacttagattcaaagtacatggattcattcaatcaaaagctacaagatttgctaggttaa

SEQ ID NO: 69 - protein, 438 aa, Bellis perennis
MDSKIDSKTFRVVMLPWLAYSHISSFLVFAKRLTNHNFHIYICSSQTNMQYLKNNLTSQYSKSIQLIELNLPSSSELPLQYHTTHGLPPHLTKTLSDDYQKSGPDFETILIKLNPHLVIYDFNQLWAPEVASTLHIPSIQLLSGCVALYALDAHLYTKPLDENLAKFPFPEIYPKNRDIPKGGSKYIERFVDCMRRSCEIILVRSTMELEGKYIDYLSKTLGKKVLPVGPLVQEASLLQDDHIWIMKWLDKKEESSVVFVCFGSEYILSDNEIEDIAYGLELSQVSFVWAIRAKTSALNGFIDRVGDKGLVIDKWVPQANILSHSSTGGFISHCGWSSTMESIRYGVPIIAMPMQFDQPYNARLMETVGAGIEVGRDGEGRLKREEIAAVVRKVVVEDSGESIREKAKELGEIMKKNMEAEVDGIVIENLVKLCEMNN

SEQ ID NO: 70 - DNA, 1317 bp, synthetic construct
  misc_feature 1..1317: note = Synthetic oligonucleotide
  source 1..1317: mol_type = other DNA
atggattctaaaatcgattcaaagacattcagagtcgttatgttgccttggcttgcatactcacacatttcatcattcctagtgtttgccaagagactaacaaatcataacttccacatctacatttgttcctctcaaacaaatatgcaatacctgaaaaacaacttgacgtctcagtattcaaaatctatacaactgattgagttgaatcttccatctagttccgaattgcctctgcagtatcatactactcacggactaccaccacaccttacgaaaacattgtctgatgattatcaaaagtccggacctgactttgaaaccattttgatcaaattgaacccacatctggtaatctacgactttaatcaactttgggctccagaggttgctagtacacttcatattccatccatacagttactgtctggttgcgtcgccttatatgccttagacgcccatctgtacacaaagccactagacgaaaacttggctaagtttcctttcccagaaatctatcctaaaaacagagatattcctaagggaggtagtaaatacatcgaaaggttcgtagactgtatgagaagatcttgtgaaatcatattagtcagaagtaccatggaacttgaaggaaaatacattgattacttgtctaagacattagggaaaaaggtgttgccagtagggcctctggtgcaagaggcttctttgttgcaagatgatcatatatggattatgaagtggttagacaaaaaggaggagtcatccgtcgtgtttgtttgttttggttctgagtacatcttatcagacaacgaaatagaagatattgcttatggcctagagttgtcccaagtaagtttcgtttgggcaataagagctaagacttctgccttaaatggcttcattgatagagtgggtgataaaggcttagtcatcgataaatgggttccacaggctaacatcttatctcactcttctactggtggattcattagtcattgcggttggtcatcaacaatggaatctattagatatggggttcctattatcgccatgccaatgcaattcgatcaaccttacaatgctaggttgatggaaactgttggtgcaggtatcgaagttggcagagatggcgaaggtagattgaaaagagaagagattgctgccgtggttagaaaggtcgttgttgaagattctggggaatccataagggagaaggcaaaggaattgggagaaatcatgaaaaaaaacatggaggccgaagtagatggtatagtgattgaaaatctagttaagctatgtgagatgaacaattaa

SEQ ID NO: 71 - protein, 477 aa, Arabidopsis thaliana
MMTLNSLSPAESKAISFLDTSRFNPIPKLSGGFSLRRRNQGRGFGKGVKCSVKVQQQQQPPPAWPGRAVPEAPRQSWDGPKPISIVGSTGSIGTQTLDIVAENPDKFRVVALAAGSNVTLLADQVRRFKPALVAVRNESLINELKEALADLDYKLEIIPGEQGVIEVARHPEAVTVVTGIVGCAGLKPTVAAIEAGKDIALANKETLIAGGPFVLPLANKHNVKILPADSEHSAIFQCIQGLPEGALRKIILTASGGAFRDWPVEKLKEVKVADALKHPNWNMGKKITVDSATLFNKGLEVIEAHYLFGAEYDDIEIVIHPQSIIHSMIETQDSSVLAQLGWPDMRLPILYTMSWPDRVPCSEVTWPRLDLCKLGSLTFKKPDNVKYPSMDLAYAAGRAGGTMTGVLSAANEKAVEMFIDEKISYLDIFKVVELTCDKHRNELVTSPSLEEIVHYDLWAREYAANVQLSSGARPVHA

SEQ ID NO: 72 - protein, 440 aa, Leishmania infantum
MHSTRHILRQRAVLVTGARTPFVKSFGALMKADTLELASASVAGLLNKTSLDPRDIDHIVWGNVVLQGSAHNCAREIVIDLNMPKKIIGNLTSMACASGLSSLSQACMLIEGGHADVVIAGGSDSVSNTEVPLPRSVTYGLMMAQRKGVMGFFKEAGYNPFKWFPGGIALTERSTGKTMGWHGDLIAELNSISRDDQEALAVASHANAARAEKAGYFKEEIVPVTIDKKGKKTEVTCDDVMQRDTEKMKAKMPSLKPVFRKEGGTITAATSSTLTDGGSAMLVMSEEKAKKLGYPTDVCVKSWYFSGIDPYPQLLLAPVLGWGPALKKAGLTPKDIDLYEIHEAFAAQVLATIKCLKSQEFFDRYANGAKPVLTEDIDLSKLNVNGGSLALGHPFAATGGRIVISLANELRRSGKRHGLVSICAAGGLGGVAILEHTASK

SEQ ID NO: 73 - protein, 527 aa, Saccharomyces cerevisiae
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 74 - protein, 1226 aa, Ganoderma lucidum
MRAVLRLLSTHTVFSPIETIVSVFVLATLAYFHILSGIKHSSFFASSHPPAIRPAFAHLTNGEWVAVSQHDWTEAWKHPGGSLDALELQQVVFTLDDKTQPSAVLDASAISQHLVSNVPALSGKAYSSLCHHPNVSGTSCFTSVSGPGASPILTLSFKPGTRDDWLGSLRKEKTITLDGVKYDVGAGKRQESIGDMESSKWVAYALSALVLRFWELTKADSLDILVVLTGYILMHVTFMRLFLASRALGSNFWLSAGIFSSATISFLFTLPMCRSMDIPLDPIALTEALPFLVCTVGFDKPLRLARAVMAHPNILKPQDDGRMKAAGDVILEALDRVGNMILRDYALEIAVLFVGVNSRVGGLKEFCAVAAALLAMDRLMTFTLYTAVLTIMVEVRRIKKVRDMTKARSRSSSITAVTANGTAIRGVLSRKSSKQSVTEPETTKNLRQRATDSAIGVKGSLLKDGGRLQEAEENPMARLKLLLIASFLTLHILNFCTTLTSATANARHQRHPFRTVQEVVPIPRVDITTPAIANILSHLAVAQEPMFTVVGSEPIELLVKVAAPVYVHALPLAPALRASNTNTGEAIENFMSSWSSLVGDPVVSKWIVALLAVSVALNGYLLKGIAAGSGLAAMRAVRSQGVRFRSRARSIVKISDEPEPEPEHSIDPAPVVFFASAAPAVEAPAPAPAPEPEPPVNRPPPLTIFSRPLNLETVDKKLQDALPIRSPPPVEPITPESREVEPTQVEVRSLAECVDVFENGPRPVSVALKTLNDEEVILLCQTGKIAPYALVKMLADFDRAVRVRRALISRASRTKTLENSLVPMKDYDYARVMGACCENVIGYMPLPLGIAGPLKIDGLMYPIPMATAEGTLVASTSRGCKALNAGGGVTTVLTADGMTRGPAIDFPSIVRAAEAKAFIESEDGYATIREAFESTSRFAKLQKIKCALAGRTLFVRFATRTGDAMGMNMISKATEKALDVLSHEFPEMVVLALSGNYCTDKKPAAISWIEGRGKSIVAEAVIPGKVVKSVLKTTVESLCNVNTKKNLIGSAMAGSVGGFNAHAANILTAVFLATGQDPAQNVESSNCMTLMEPTNGGEDLLMTISMPCIEVGTVGGGTILEPQGAVLDLLGVRGAHPTNPGQNAQQLARIIASAVMAGELSLISALAAGHLVRAHLAHNRSQLNTPMPSRPHTPGPEDVSHVQQLPTPSASDDKGVTAQGYVVEAK

SEQ ID NO: 75 - protein, 888 aa, Bos taurus
MLSRLFRMHGLFVASHPWEVIVGTVTLTICMMSMNMFTGNNKICGWNYECPKLEEDVLSSDIIILTITRCIAILYIYFQFQNLRQLGSKYILGIAGLFTIFSSFVFSTVVIHFLDKELTGLNEALPFFLLLVDLSRASALAKFALSSNSQDEVRENIARGMAILGPTFTLDALVECLVIGVGTMSGVRQLEIMCCFGCMSVLANYFVFMTFFPACVSLVLELSRESREGRPIWQLSHFARVLEEEENKPNPVTQRVKMIMSLGLVLVHAHSRWIADPSPQNSTADNSKVSLGLDENVSKRIEPSVSLWQFYLSKMISMDIEQVITLSLALLLAVKYIFFEQAETESTLSLKNPITSPVVTQKKITDDCCRRDPVLVRNDQKFHAMEEETRKNRERKVEVIKPLLAENDTSHRATFVVGNSSLLGTSLELETQEPEMELPVEPRPNEECLQILENAEKGAKFLSDAEIIQLVNAKHIPAYKLETLMETHERGVSIRRQLLSKKLPEPSSLQYLPYRDYNYSLVMGACCENVIGYMPIPVGVAGPLCLDGKEFQVPMATTEGCLVASTNRGCRAIGLGGGASSRVLADGMTRGPVVRFPRACDSAEVKAWLETPEGFTVIKEAFDSTSRVARLQKLHMSVAGRNLYIRFQSRSGDAMGMNMISKGTEKALSKLQEYFPEMQILAVSGNYCTDKKPAAINWIEGRGKSVVCEAVIPAKVVREVLKTTTEAMIEVNINKNLVGSAMAGSIGGYNAHAANIVTAIYIACGQDAAQNVGSSNCITLMEASGPTNEDLYISCTMPSIEIGTVGGGTNLLPQQACLQMLGVQGACRDNPGENARQLARIVCGTVMAGELSLMAALAAGHLVRSHMIHNRSKINLQDLQGTCTKKAA

SEQ ID NO: 76 - protein, 567 aa, Artemisia annua
MDLRRKLPPKPPSSTTTKQPSHRSHSPTPIPKASDALPLPLYLTNTFFFTLFFSVAYYLLHRWRDKIRSGTPLHVVTLTELSAIVLLIASFIYLLGFFGIDFVQSFTSRENEQLNNDDHNVVSTNNVLSDRRLVYDYGFDVTGDNDNDNDDDVIVKSVVSGEVNSYSLEASLGDCYRAAKIRKRAVERIVGREVLGLGFEGFDYESILGQCCEMPIGYVQVPVGVAGPLLLNGGEFMVPMATTEGCLVASTNRGCKAICLSGGATAILLKDGMTRAPVVRFATAERASQLKFYLEDGVNFDTLSVVFNKSSRFARLQNIQCSIAGKNLYIRFTCSTGDAMGMNMVSKGVQNVLDFLQNDFPDMDVIGISWKFCSDKKPTAVNWIEGRGKSVVFQAVITKKVVRKSALNPQTCTCRTLTCLRPLLVLLLLVLLVDLMHMLHIVSAVFIATGQDPAQNIESSHCITMMEAVNNGKDLHVNVTMPSIEVGTVGGGTQLASQSACLNLLGVKGACIESPGSNAQLLARIVAGSVLAGELSLMSAISAGQLVKSHMKYNRSSRDMSAIASKV

SEQ ID NO: 77 - protein, 435 aa, Trypanosoma cruzi
MFRRAILLGCSAAKTPWSECSNAQLVDAVKSRKISFYGLEQALEPDYRRAIEVRREVVSEIASQQPEAKKKQSALHTIPFENYDWNKVVGQNCENIIGYVPIPLGVAGPILIDGKEYPIPMATTEGALVASTHRGARAITRSGGCKTLLLGEGMTRAPVVELPSLEEAGRLHKYCNENFLSLKEAFESTTQYGKLNSLKCVLAGRKAYLRFRATTGDAMGMNMITKGVDKALSVLQQHFPSMEILALSGNYCTDKKPSAVNWIDGRGKSVVAEATLLADVVEDTLKCTVDSLVSLNIDKNLVGSAMAGSVGGFNAQAANAVAAIFIATGQDPAQVVESSMCITTMSKVGNDLLISVTMPSIEVGVVGGGTGLAAQRGCLELIGCGGPSKESPGTNAQLLSRVVAAGVLSAELSLMSGLAAGHLLSAHMRLNRKKK

SEQ ID NO: 78 - protein, 426 aa, Staphylococcus aureus
MQSLDKNFRHLSRQQKLQQLVDKQWLSEEQFNILLNHPLIDEEVANSLIENVIAQGALPVGLLPNIIVDDKAYVVPMMVEEPSVVAAASYGAKLVNQTGGFKTVSSERIMIGQIVFDGVDDTEKLSADIKALEKQIHQIADEAYPSIKARGGGYQRIAIDTFPEQQLLSLKVFVDTKDAMGANMLNTILEAITAFLKNEFPQSDILMSILSNHATASVVKVQGEIDVKDLARGERTGEEVAKRMERASVLAQVDIHRAATHNKGVMNGIHAVVLATGNDTRGAEASAHAYASKDGQYRGIATWRYDQERQRLIGTIEVPMTLAIVGGGTKVLPIAKASLELLNVESAQELGHVVAAVGLAQNFAACRALVSEGIQQGHMSLQYKSLAIVVGAKGDEIAQVAEALKQEPRANTQVAERILQDLRSQQ

SEQ ID NO: 79 - protein, 481 aa, Arabidopsis thaliana
MHITKPHAAMFSSPGMGHVIPVIELGKRLSANNGFHVTVFVLETDAASAQSKFLNSTGVDIVKLPSPDIYGLVDPDDHVVTKIGVIMRAAVPALRSKIAAMHQKPTALIVDLFGTDALCLAKEFNMLSYVFIPTNARFLGVSIYYPNLDKDIKEEHTVQRNPLAIPGCEPVRFEDTLDAYLVPDEPVYRDFVRHGLAYPKADGILVNTWEEMEPKSLKSLLNPKLLGRVARVPVYPIGPLCRPIQSSETDHPVLDWLNEQPNESVLYISFGSGGCLSAKQLTELAWGLEQSQQRFVWVVRPPVDGSCCSEYVSANGGGTEDNTPEYLPEGFVSRTSDRGFVVPSWAPQAEILSHRAVGGFLTHCGWSSTLESVVGGVPMIAWPLFAEQNMNAALLSDELGIAVRLDDPKEDISRWKIEALVRKVMTEKEGEAMRRKVKKLRDSAEMSLSIDGGGLAHESLCRVTKECQRFLERVVDLSRGA

SEQ ID NO: 80 - protein, 513 aa, Arabidopsis thaliana
MGAYETEKPTKDAAALETQSPEDFDQPSPLRKIISVASIAAGVQFGWALQLSLLTPYVQLLGIPHKWSSLIWLCGPVSGMIVQPIVGFHSDRCRSKFGRRRPFIATGAALVAVAVFLIGYAADFGYKMGDKLEEKVKVRAIGIFALGFWILDVANNTLQGPCRAFLADLAAGDAKRTRVANAFFSFFMAVGNVLGYAAGSYTNLHKMFPFTMTKACDIYCANLKTCFFLSITLLLIVTVTSLWYVNDKQWSPPPRNADDDEKTSSVPLFGEIFGAFKVMKRPMWMLLIVTALNWIAWFPFLLFDTDWMGREVFGGDSDGNERSKKLYSLGVQSGAMGLMFNSIVLGFMSLGVEWIGRKLGGAKRLWGIVNFILAAGLAMTVLVTKFAEDHRKTAGDLAGPSASVKAGALSLFAVLGIPLAITFSTPFALASIFSSCSGAGQGLSLGVLNLAIVIPQMIVSLGGGPFDALFGGGNLPAFIVAAIAAAISGVLALTVLPSPPPDAPKATTMGGFH

SEQ ID NO: 81 - protein, 806 aa, Coffea arabica
MAERVLTRVHSLRERLDATLAAHRNDVLLFMSRLETHGKGILKPHQLLAEFEEINKDGKQKIHDHAFEEVLKSTQEAIVLPPWVALAIRLRPGVWEYVRVNVHALVVEELTVPEYLHFKEELVDGSKNGNFVLELDFEPFTASFPKPTLTKYIGDGVEFLNRHLSAKMFHDKESMAPLLDFLRVHQYKGKTMMLNDRIKDLNTLQAVLRKAEEYLTTLSADTPYSEFEHKFQEIGLERGWGDTAERVLEMICMLLDLLGAPDSCTLEKFLGRIPMVFNVVILSPHGYFAQENVLGYPDTGGQVVYILDQVPALEREMLKRIKEQGLDVKPRILIITRLLPDAPGTTCGQRLEKVYGSEYSHILRVPFRTEKGVVRKWISRFEVWPYMETFTEDVAKEVTAELQAKPDLVIGNYSEGNLVASLLAHKLGVTQCTIAHALEKTKYPDSDIYLSKFDEKYHFSCQFTADLIAMNHTDFIITSTFQEIAGSKDTVGQYESHMAFTMPGLYRVVHGIDVFDPKFNIVSPGADTNLYFPHTEKEKRLTSFHPEIEELLFSDVENEEHLCVLKDKKKPILFTMARLDRVKNLTGLVELYAKNPKLRELVNLVVVGGDRRKESKDLEEQAEMKKMYSLIETYNLNGQFRWISSQMNRVRNGELYRYIADTRGAFVQPAFYEAFGLTVVEAMTCGLPTFATNHGGPAEIIIHGKSGFHIDPYHGEQVSELLANFFERCKKEPSYWDTISAGGLKRIQEKYTWQIYSDRLLTLAGVYGFWKCVSKLDRQEIRRYLEMFYALKYRKLAEAVPLAVDQ

SEQ ID NO: 82 - DNA, 19 bp, synthetic construct
  misc_feature 1..19: note = Synthetic oligonucleotide
  source 1..19: mol_type = other DNA
tgaattcgttaacgaattc

SEQ ID NO: 83 - DNA, 19 bp, synthetic construct
  misc_feature 1..19: note = Synthetic oligonucleotide
  source 1..19: mol_type = other DNA
tgaattcgttaacgaactc

SEQ ID NO: 84 - DNA, 19 bp, synthetic construct
  misc_feature 1..19: note = Synthetic oligonucleotide
  source 1..19: mol_type = other DNA
tgaattcgttaacgaagtc

SEQ ID NO: 85 - DNA, 19 bp, synthetic construct
  misc_feature 1..19: note = Synthetic oligonucleotide
  source 1..19: mol_type = other DNA
tgaattcgttaacgaaatt

SEQ ID NO: 86 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 87 - DNA, 21 bp, synthetic construct
  misc_feature 1..21: note = Synthetic oligonucleotide
  source 1..21: mol_type = other DNA
cggtaggtattgattgtaatt

SEQ ID NO: 88 - DNA, 21 bp, synthetic construct
  misc_feature 1..21: note = Synthetic oligonucleotide
  source 1..21: mol_type = other DNA
cttttcggttagagcggatgt

SEQ ID NO: 89 - DNA, 1422 bp, synthetic construct
  misc_feature 1..1422: note = Synthetic oligonucleotide
  source 1..1422: mol_type = other DNA
atggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaa

SEQ ID NO: 90 - DNA, 1422 bp, synthetic construct
  misc_feature 1..1422: note = Synthetic oligonucleotide
  source 1..1422: mol_type = other DNA
atggctacttctgattccatcgttgacgatagaaagcaattgcatgttgctacttttccatggttggctttcggtcatattttgccatacttgcaattgtccaagttgattgctgaaaagggtcacaaggtttcattcttgtctaccaccagaaacatccaaagattgtcctctcatatctccccattgatcaacgttgttcaattgactttgccaagagtccaagaattgccagaagatgctgaagctactactgatgttcatccagaagatatcccttacttgaaaaaggcttccgatggtttacaaccagaagttactagattcttggaacaacattccccagattggatcatctacgattatactcattactggttgccatccattgctgcttcattgggtatttctagagcccatttctctgttactactccatgggctattgcttatatgggtccatctgctgatgctatgattaacggttctgatggtagaactaccgttgaagatttgactactccaccaaagtggtttccatttccaacaaaagtctgttggagaaaacacgatttggctagattggttccatacaaagctccaggtatttctgatggttacagaatgggtatggttttgaaaggttccgattgcttgttgtctaagtgctatcatgaattcggtactcaatggttgcctttgttggaaacattgcatcaagttccagttgttccagtaggtttgttgccaccagaaattccaggtgacgaaaaagacgaaacttgggtttccatcaaaaagtggttggatggtaagcaaaagggttctgttgtttatgttgctttgggttccgaagctttggtttctcaaaccgaagttgttgaattggctttgggtttggaattgtctggtttgccatttgtttgggcttacagaaaacctaaaggtccagctaagtctgattctgttgaattgccagatggtttcgttgaaagaactagagatagaggtttggtttggacttcttgggctccacaattgagaattttgtctcatgaatccgtctgtggtttcttgactcattgtggttctggttctatcgttgaaggtttgatgtttggtcacccattgattatgttgccaatctttggtgaccaaccattgaacgctagattattggaagataagcaagtcggtatcgaaatcccaagaaatgaagaagatggttgcttgaccaaagaatctgttgctagatctttgagatccgttgtcgttgaaaaagaaggtgaaatctacaaggctaacgctagagaattgtccaagatctacaacgataccaaggtcgaaaaagaatacgtttcccaattcgttgactacttggaaaagaatgctagagctgttgccattgatcatgaatcttga

SEQ ID NO: 91 - DNA, 1503 bp, Stevia rebaudiana
  source 1..1503: mol_type = other DNA
atggaggcttcatatctatacatttccattcttctgcttctagcttcgtacctcttcaccacccaacttcgtcgtaaatccgccaatctaccgccgacggtgttcccatccatccccataatcggtcatctctacctcctcaaaaaaccactctatagaacactagccaaaattgccgccaaatacggccctatcctccaactccaactagggtaccgccgtgtcctcgtaatctcctccccttccgccgccgaagaatgcttcaccaacaacgacgttatcttcgccaaccgtccgaagacgctattcggaaaaattgtagggggtaccagcctcgggtcgctgtcgtacggcgaccagtggcgcaacctccgccgcgttgcatccatcgagattctatcagtccaccggctcaacgagtttcacgacatacgtgttgacgaaaaccggcttctgatccgtaaactaagatccagttcttctccggtgactctgataacggtgttttacgcactaacgttaaatgtgattatgagaatgatctccggaaagaggtatttcgactcgggtgatcgggaattggaggaggaagggaagcgattccgggagatactcgatgagacacttttgctcgcgggtgcttctaatgttggggattacttgccgattctgaattggttgggggtgaagagcttggagaagaagctaatcgcattgcagaaaaagagagatgatttctttcagggactgatcgagcaagttcggaaatctagaggggctaaagtgggaaaaggaaggaagacgatgatcgagttgttgttatccctacaagaatctgaacctgagtattacactgacgccatgatccgatcatttgtgctgggtttattagcagcagggagtgatacatcggctggaactatggaatgggcgatgtctcttttggtaaaccacccgcacgtattaaaaaaggcacaggctgaaattgatcgagtcatcggcaacaaccgtctaattgatgagtccgacatagggaatataccttacattggttgcatcataaacgagacgctcagattgtaccctgcgggcccgttgctatttccccatgagtcatcagcggactgtgttatcagcgggtacaacatccctcgtgggacgatgcttattgtcaaccaatgggcgatacatcatgacccaaaggtgtgggacgaccctgagacattcaaaccggaaagattccaagggcttgaagggacacgagacgggtttaagctgatgccttttgggtccggaaggaggggttgtccaggggagggattggcgattcgtttgcttgggatgactctcgggtcggttatccaatgctttgattgggaacgagtcggtgacgagatggttgatatgaccgaaggtcttggggtcacgttgcctaaagctgtaccattagtcgccaagtgcaagccgcgttccgaaatgacgaatctactctctgagctatga

SEQ ID NO: 92 - protein, 436 aa, Archaeoglobus fulgidus
MQVLRLDRRHYKSGKIRRAMSSRIPGFYKLSVEERLKKVAEFAGLSDEEVKAVLSQGLPLDVADRMIENVIGTFELPLGIATNFLIDGKDYLIPMAIEEPSVVAAASNAARMARESGGFTTDYTGSLMIGQIQVTKLLNPNAAKFEVLRQKDEIIERANECDPMLVNLGGGCKDIEARVIDTIMGKMLIVHLIVDVKDAMGANAVNTMCEKVAPFIERITGGKVYLRIISNLAAYRLARAKAVFDKDVIGGEEVVEGIMLAYAFAAADPFRCATHNKGIMNGISALMIATGNDFRAIEAGAHSYAAIGGYKPLTTYEVDRKGNLVGTIEIPMAVGVIGGATKVNPLAKISLKILGVNTAEELARVAAALGLAQNFAALRALATEGIQRGHMELHARNLAIMAGATGDEVDRVVEIMVRDGKIRLDYAKEVLERLRS

SEQ ID NO: 93 - protein, 428 aa, Pseudomonas mevalonii
MSLDSRLPAFRNLSPAARLDHIGQLLGLSHDDVSLLANAGALPMDIANGMIENVIGTFELPYAVASNFQINGRDVLVPLVVEEPSIVAAASYMAKLARANGGFTTSSSAPLMHAQVQIVGIQDPLNARLSLLRRKDEIIELANRKDQLLNSLGGGCRDIEVHTFADTPRGPMLVAHLIVDVRDAMGANTVNTMAEAVAPLMEAITGGQVRLRILSNLADLRLARAQVRITPQQLETAEFSGEAVIEGILDAYAFAAVDPYRAATHNKGIMNGIDPLIVATGNDWRAVEAGAHAYACRSGHYGSLTTWEKDNNGHLVGTLEMPMPVGLVGGATKTHPLAQLSLRILGVKTAQALAEIAVAVGLAQNLGAMRALATEGIQRGHMALHARNIAVVAGARGDEVDWVARQLVEYHDVRADRAVALLKQKRGQ

SEQ ID NO: 94 - DNA, 1323 bp, synthetic construct
  misc_feature 1..1323: note = Synthetic oligonucleotide
  source 1..1323: mol_type = other DNA
atgcattctaccagacatatcttaagacaaagggccgtcctagttacaggcgctagaacaccattcgtgaaatcatttggggctcttatgaaagcagataccttggaattggcatcagcatcagtcgctgggttgctgaacaagacctcactggaccctagagatatcgatcatatcgtttggggtaatgttgtacttcaaggatcagctcataactgcgccagagaaatagttatcgaccttaacatgcctaaaaagatcatcggtaatttgacatctatggcctgtgcttcaggcttatcttctttgtcacaagcctgtatgctaatagagggtggtcatgccgatgtcgtcattgctggcggttctgattcagtctccaacactgaagtgcctttgccaagatccgtcacttacggtctaatgatggcccaaaggaagggtgttatgggcttctttaaggaagcaggatacaacccattcaaatggtttccaggcggtattgctttaaccgaacgtagtacaggaaaaactatgggttggcatggagacttaattgctgagttaaactctatatctagagatgaccaggaagccctggctgtggcttctcatgcaaatgctgctagagcagaaaaagctgggtactttaaggaggaaattgtacctgtgacaatcgacaaaaagggcaaaaagactgaagtaacatgtgatgatgttatgcaaagagatacagaaaagatgaaggccaagatgccatcattgaagcctgttttcagaaaagagggaggtacaataacagcagccacttccagtactctgactgatggtggctctgcaatgttggttatgtcagaggaaaaggccaaaaagttgggttatccaactgatgtctgcgtgaagtcttggtatttcagtggtatcgatccttacccacaacttttgttagcaccagttctaggttggggtccagctttgaaaaaggccggattaacccctaaagatatcgatttgtacgaaattcacgaagcatttgctgcacaagttctagccacaattaagtgtttgaagtctcaggaattcttcgataggtacgctaacggtgcaaagccagtattaactgaggatattgatctttctaaactaaatgttaatggcggttccttagcacttggccacccattcgccgctacaggaggtagaatcgtaatctctctagcaaatgagttgagaagatccggaaagagacacgggctggtcagtatttgtgcagctggagggttaggcggagtagctatacttgagcatacagcaagtaagtaa

SEQ ID NO: 95 - DNA, 1584 bp, synthetic construct
  misc_feature 1..1584: note = Synthetic oligonucleotide
  source 1..1584: mol_type = other DNA
atggcagctgaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 96 - DNA, 3681 bp, synthetic construct
  misc_feature 1..3681: note = Synthetic oligonucleotide
  source 1..3681: mol_type = other DNA
atgagagctgtccttagattgttatcaacacatactgttttctctcctattgaaacaattgtatctgttttcgtgttagctacattagcttacttccacatcttgtccggaatcaagcactcaagtttctttgcatcttctcatcctcctgctatcagacctgcttttgcacatctgaccaacggggaatgggttgccgtctcccaacatgattggactgaagcatggaagcatcctggcggttcacttgatgcattagaacttcaacaagtagttttcactttagatgacaagactcaaccatctgctgtgctagatgcatccgcaattagtcagcacttagtttccaatgttcctgcattatctggaaaagcctactcttcattgtgccaccatccaaatgtatcaggcacctcctgttttacatcagtttctggtccaggagcttcaccaatcttgacactgagttttaagcctggaactagagacgattggttaggatcattaaggaaggagaaaactatcacactagatggggttaagtacgacgttggagccggaaaaagacaagagtcaatcggcgatatggaatcatctaagtgggttgcttatgcattatcagctttggtacttagattttgggaattaacaaaggcagattccttagatatactagtggttctaactgggtacatcctaatgcacgtaacattcatgagattgttcttggcatccagagcacttggcagtaacttttggttatcagctggcatattctcctccgcaacaatttctttcctattcactttaccaatgtgtagatctatggatattccacttgatccaattgccttgacagaagccctgccattcttggtgtgtaccgtaggttttgacaaaccacttagattggcaagagctgtgatggctcatcctaatatccttaaacctcaagatgatggtaggatgaaagctgccggagatgtcattcttgaggcactggacagagttggtaacatgatattgagagattacgctttagagatcgcagttctattcgttggcgttaactccagagttggcggtcttaaggaattttgtgctgtagctgcagcattacttgctatggacagattaatgacattcacactttatacagcagtgttaaccatcatggttgaggtaaggcgtatcaaaaaggtcagagatatgactaaggctagatctagaagttcttctattaccgccgttacagccaacggcaccgccataagaggcgttttgagtagaaaatcttcaaaacaatctgtgacagaaccagagacaactaaaaacctaagacaaagagccactgattcagccatcggtgttaagggttcattgctgaaagatggaggcagattgcaggaagccgaggagaatccaatggcaagattaaagctattgttaatcgcttccttcttaacactacacatcttgaacttttgtactactttgacttcagccacagctaacgcaagacatcaaagacatccttttagaaccgttcaagaggtagtaccaattcctagagttgacattactaccccagccatagccaatatcttgtctcatctagctgtggctcaggaacctatgttcactgttgttggcagtgaacctatcgaacttcttgttaaagtcgctgctccagtctacgtccatgctctaccattggcccctgctttaagagcttcaaacactaatactggagaagctattgaaaactttatgagttcatggtctagtctggtaggtgacccagttgttagtaagtggatcgtagcattgctagctgtctctgttgcattgaatggatacttgttaaagggtatagccgcaggttccgggttggctgccatgagagctgttagatctcaaggtgttcgtttcagatctagagctagaagtatcgtaaagatatctgatgaacctgagccagagccagaacactctatcgacccagcaccagtagtgttcttcgcttccgcagcaccagctgtagaggcccctgctccagctcctgcacctgaaccagaaccaccagtcaacagaccaccaccattgactattttctcaagaccactgaacttagaaacagtggacaaaaagttacaagatgctctgccaataagatccccaccacctgttgaaccaatcactccagaatctagagaagtggaaccaacccaagtagaagtaagatctctagctgaatgtgtggatgtgttcgagaatgggccaagaccagtctcagtggctttaaagactctgaatgatgaggaagttatcctgctttgccaaacaggtaagatagctccatatgcattggttaagatgttggctgatttcgatagggccgtacgtgtcagaagagcacttattagtagagcttcacgtacaaaaactttagaaaactcactggttcctatgaaagattatgattacgccagagtcatgggtgcctgttgtgaaaacgttatcggatacatgccattaccactagggattgcaggtccattgaagattgatggcttgatgtatcctataccaatggcaaccgcagaaggtaccttggttgcatctacttctaggggctgtaaggccttaaatgctggtggaggggtcacaactgtcttgacagcagatggcatgacaagagggccagctatagactttccttccatcgtcagagctgcagaggctaaggccttcattgaatcagaagatggatacgctacaatcagggaggctttcgagtctacttctagatttgccaagttgcaaaagatcaagtgtgcactagctggtcgtactctttttgtcagatttgctactagaacaggagatgccatgggtatgaacatgatttctaaggctaccgaaaaggcacttgatgtcctgagtcacgagttccctgaaatggtcgtccttgctttgtctggtaactactgcacagacaaaaagcctgcagctatttcatggatcgaaggtaggggaaaatctattgtagcagaagcagttattcctggtaaggtcgttaagtcagtcctgaaaacaacagtcgagtctctttgcaatgtcaacactaagaaaaacctgattggttcagccatggcaggttctgttggtggtttcaacgctcatgccgccaacatcctaacagctgtgttcctagccacaggtcaggatcctgctcaaaatgtcgaatcttctaattgcatgactttaatggaaccaacaaacggcggtgaggatttgctaatgacaatttcaatgccatgtatagaggtaggaaccgttggtggagggacaattctggaaccacaaggtgcagttttggatttgttgggcgttagaggggctcaccctactaatcctggtcaaaacgctcaacagttagccagaattatcgcatcagctgtaatggcaggcgaattgtctttgataagtgccttagccgcaggtcatttggttagagctcatcttgcccacaatcgttctcaattgaatacaccaatgccatccagaccacatactcctggccctgaggatgtctcacatgtgcagcagctacctacaccatctgcatctgatgataaaggtgttacagctcaaggttacgttgtcgaagcaaaataa

SEQ ID NO: 97 - DNA, 2667 bp, synthetic construct
  misc_feature 1..2667: note = Synthetic oligonucleotide
  source 1..2667: mol_type = other DNA
atgttatcaagattgttcagaatgcatggtctatttgttgcttctcacccttgggaagtaatagttggtactgtaacattaacgatctgtatgatgtctatgaacatgtttaccggaaacaacaagatttgtggttggaattatgagtgtcctaagctggaagaggatgtgttgagttcagacatcatcatacttactataacaagatgcattgcaatattgtatatctacttccaatttcaaaaccttagacaattgggtagtaaatacatcctaggcatcgccggattgttcactattttctctagttttgttttctcaaccgtcgttattcactttttggacaaagagttaactggtttgaacgaagctctaccattcttcttgctgctggtagatttgtccagagcttccgctttagctaaattcgctctgtcctctaattctcaagatgaagttagagagaatatagcaaggggaatggccatacttggacctactttcacacttgatgcccttgtcgaatgtttggttattggggttggcacaatgtccggcgttagacagttagaaatcatgtgttgttttggctgtatgagtgtcttggctaactactttgtctttatgacattctttccagcttgcgtttctttggtattggagctgtcaagagaatcaagagaaggcagaccaatatggcaactatcacatttcgccagagtgttagaagaggaggaaaacaaacctaatcctgtcacacagagagtgaaaatgatcatgtctttgggtttagtcctagtgcatgctcattctagatggatcgcagatccatcccctcagaattctacagctgataactctaaagttagtttaggtttagatgaaaatgtaagtaagaggattgaaccttccgtgtctttgtggcaattctacttatcaaaaatgatttccatggatattgaacaagtgataacgttgtctttggctttattgttagccgttaagtacattttctttgagcaagccgaaacggaatctacattatcactgaaaaacccaattacatccccagtcgttacccagaaaaagataactgatgattgctgtagaagagatccagtgttggtcaggaatgatcaaaagttccacgccatggaggaggaaactaggaaaaacagagaaaggaaagttgaagttatcaagcctctattagcagaaaatgacacttcacatagggccactttcgttgtcggcaattcatctcttttaggtacgtcattggagctggaaacacaggaaccagaaatggaactaccagttgaaccaagaccaaatgaggaatgtttgcaaatactagagaacgctgaaaagggagccaagttcctatctgatgccgagattatccagctggtcaatgccaagcacattcctgcctacaagttggaaacccttatggagacacatgagagaggtgtgtctattaggagacaattactatctaaaaagttacctgaaccaagttccctacaatacctgccttatagagattacaattactccttggtaatgggagcttgttgtgaaaatgtcattgggtacatgccaattccagtgggtgtcgccggtccactatgtttggacggtaaggaatttcaagtacctatggcaacgactgaaggctgcttagttgcatctacaaacagaggttgtagagccattggattaggtggcggtgcttcttcaagagtcttggctgacggtatgactagaggtcctgttgtgagatttcctagggcctgtgactctgcagaagttaaggcttggttggaaactccagaaggtttcaccgtaatcaaagaggcctttgattccacatcaagggtggccagattacaaaaactacacatgtctgtcgctgggagaaatctgtatatcagatttcaatccagatccggcgacgcaatgggtatgaatatgatttcaaaagggacagaaaaggctttgtcaaagctgcaggagtatttcccagagatgcaaatcttggccgtatctggcaactattgcacagacaaaaagcctgccgccatcaactggattgaaggaagaggcaaatctgtggtttgtgaagctgtaattccagccaaagttgttagagaagtgttaaagaccacaacagaagctatgattgaagtaaacataaacaaaaacttagtagggtctgccatggctggttcaattggaggatacaacgctcatgctgccaatattgtaaccgctatctacatcgcatgtggacaagatgctgcccaaaatgtcggttcctcaaattgcatcacattgatggaagcatctggccctacaaacgaggatttgtatatcagttgcacaatgccatctatagaaatagggactgtgggaggaggaactaacttacttccacagcaagcctgcttacaaatgctgggtgtacaaggagcctgtagagataatccaggggagaacgctagacaacttgccagaattgtttgtgggacagttatggctggtgaacttagtctaatggcagctttggctgctgggcacctggtgagatctcatatgattcataatagaagtaagattaaccttcaagatttgcaaggtacgtgtacgaaaaaggctgcctaa

SEQ ID NO: 98 - DNA, 1704 bp, synthetic construct
  misc_feature 1..1704: note = Synthetic oligonucleotide
  source 1..1704: mol_type = other DNA
atggatttgagaaggaaattaccacctaagcctccatcttcaacaacaacaaaacagccaagtcataggtcccattctcctacgccaattccaaaggcttcagatgcattgcctcttccattgtacctgaccaatacgtttttcttcactcttttcttttccgtagcatattacctgttgcataggtggagagacaagattagatccggaacacctttacacgttgtgacactgactgaactatccgcaattgtactgctgattgcttccttcatctatcttttaggctttttcggtattgattttgtgcaatctttcacatcaagagaaaatgagcaactaaacaacgatgatcacaacgtcgtgtcaacaaacaatgttttatctgatagaaggttagtttacgactatggattcgatgtgacaggagacaacgataacgataatgatgacgatgttattgtgaaaagtgtcgtttctggggaagttaattcttatagtttggaggcttccctaggagattgttacagagccgcaaagattagaaagagagccgtcgagagaattgtcgggagagaagtattaggcttgggtttcgagggatttgattatgaatctatcctggggcaatgttgtgaaatgcctatcgggtacgtccaagtgccagtaggtgtcgctggacctttattgttaaatggtggggaattcatggttccaatggctacaactgaaggctgtcttgtagcttccactaatagaggttgtaaagccatatgcttatcaggtggtgccactgccatattgctaaaagatggtatgacaagagccccagtagtgagattcgccacagctgagagagcttcacaactaaagttttacttggaagatggtgtcaatttcgatacattgtctgttgtctttaacaaaagttcaagatttgccagattgcaaaacatccaatgctcaattgccggtaaaaacttgtacattaggtttacttgctccacaggcgacgccatgggtatgaacatggtttcaaaaggagtacaaaatgtattagactttttacaaaatgattttcctgatatggacgtaattgggatctcttggaagttctgctctgacaaaaagccaacagctgtcaactggattgagggcagaggaaagtctgtcgttttccaggccgtaattaccaaaaaggtggttagaaagtctgcactgaaccctcaaacttgcacatgtagaactttgacctgtttaagaccattattggttctgctacttctggttttgctagtggacttaatgcatatgcttcatatcgtgtctgccgtgttcatcgctaccggtcaagatccagctcagaatatcgaatctagtcactgtatcactatgatggaggctgtcaacaatggtaaggatttgcacgttaatgttacgatgccatctatagaagttggcacggtgggaggtggcactcagctagcctctcaatcagcctgtttgaacttgcttggtgtaaagggtgcctgtatagaatccccaggatcaaacgcccagttgttagctagaatcgttgctggttctgttctggcaggcgaattaagtttgatgtcagctataagtgctgggcaactagttaaatctcatatgaaatacaataggtctagtagagatatgtcagcaatagcttctaaggtctaa

SEQ ID NO: 99 - DNA, 1308 bp, synthetic construct
  misc_feature 1..1308: note = Synthetic oligonucleotide
  source 1..1308: mol_type = other DNA
atgtttagaagagctatactgttaggatgctctgctgccaagacaccatggtctgagtgttctaacgctcaattagttgatgcagttaagtctagaaagatctcattctacggtcttgaacaagccttggaaccagattatagaagggctatcgaagtaaggagagaggttgtctctgaaatcgcctcacaacagccagaagcaaaaaagaagcaatccgcattgcacacaataccatttgagaattatgattggaataaggtcgttggccaaaactgtgaaaacattattggatacgtcccaataccactgggcgttgctggccctattttgattgatggtaaagagtacccaataccaatggctacaacagaaggcgctttggtcgctagtactcatagaggtgctagagctattacaagatccggaggttgtaagacattgttattaggtgaaggtatgacaagagcaccagtggttgaattgccttcattagaggaagctgggcgtttgcacaagtactgtaatgagaacttcttatctttaaaggaagcatttgaatcaactacccaatatggaaaacttaattctttaaagtgcgtactagctggtagaaaagcataccttagattcagagccactacaggcgatgctatgggcatgaacatgataacaaagggtgtagacaaagcactgtctgttctacagcaacatttcccttcaatggaaatcctagccctaagtggtaattactgtaccgacaaaaagccatctgctgtaaattggattgatggcagaggtaaatcagtggttgcagaagccactttattggctgatgttgtcgaagatactctgaaatgtacagtcgattctttggtatccttgaatatcgacaaaaaccttgttgggtcagctatggctggttctgttggaggttttaacgcccaggctgcaaacgctgtggcagccattttcattgcaaccggtcaagatcctgctcaagtggtagaaagttcaatgtgtatcactacaatgtccaaggtaggtaacgatctattgatctctgtgaccatgccttctatcgaggtcggggtcgtgggaggagggactggtcttgctgcccaaagaggatgcttagagttaatagggtgcggaggcccatctaaggagtctcctggtactaatgcccaacttctaagtagagttgttgcagctggcgttttatcagccgaactttccttgatgtccggactggcagcaggtcatctattgtcagcacatatgagattgaacagaaagaagaaataa

SEQ ID NO: 100 - DNA, 1281 bp, synthetic construct
  misc_feature 1..1281: note = Synthetic oligonucleotide
  source 1..1281: mol_type = other DNA
atgcaatccctggacaaaaactttagacacttatcaagacaacagaagttacaacagctagttgataaacaatggctatcagaggaacaattcaatattctacttaaccacccacttattgatgaagaggtagcaaactcattgatagaaaatgtcatcgcacagggcgcactgcctgttggtttactaccaaatatcatcgttgatgacaaagcatacgtcgtgcctatgatggtggaagagccatctgttgttgccgctgcttcatacggcgctaaattggtgaaccaaacaggtggtttcaaaaccgtgtcctcagaacgtatcatgataggtcaaatagtatttgatggagtcgatgataccgagaaactgtctgcagatatcaaggctcttgaaaaacaaatccatcagattgcagatgaggcttacccttctattaaggccagaggtggaggctatcaaaggatcgccatcgatacattcccagaacaacagttgctttcattgaaggttttcgttgatactaaggatgctatgggcgctaatatgttaaacacaatcctagaagcaatcacagcctttttgaaaaacgaattcccacaatctgatatcttgatgtctatcctttccaaccacgcaacagccagtgttgtcaaggtccagggtgaaatagacgttaaggatttggcaagaggagaacgtactggagaagaggtcgctaagagaatggaaagagcatctgtgttagctcaagtggacattcatagagcagcaacacacaataagggtgttatgaatggcattcatgctgtagtcttggctacaggtaatgatactagaggtgcagaagcctctgctcacgcttacgcttccaaagacggtcaatatagagggatagctacatggagatacgatcaagagagacaaaggttaataggaactatagaagttccaatgactctggccattgttggtggcggtaccaaggtactgcctattgctaaggcctctttagaactgttaaacgtagaaagtgcccaagagttgggacatgttgtcgctgccgttggactagctcaaaacttcgctgcatgtagagctttggtttccgaaggtattcaacaagggcatatgtctttgcaatacaagtctttagccatcgtagtcggggctaagggcgatgaaattgctcaggtagccgaagcactaaagcaagagccaagagcaaacactcaagttgcagagagaattttgcaagatttgagaagtcaacaataa

SEQ ID NO: 101 - DNA, 1311 bp, synthetic construct
  misc_feature 1..1311: note = Synthetic oligonucleotide
  source 1..1311: mol_type = other DNA
atgcaggtcttaagattggataggagacattacaaaagtggcaagattagaagagcaatgagttctagaattcctggtttctacaaattgtcagtcgaggaaagactgaaaaaggttgctgaatttgcagggttatctgatgaggaagtgaaagctgttttgtcacaaggtttacctttggacgtagctgatagaatgatcgaaaatgtgatcggtacatttgaattaccacttggtatagcaaccaatttccttattgatggcaaggattatctaatccctatggctatagaggaaccatcagtagttgcagctgcttctaacgcagctagaatggccagagagtctggcgggtttacaactgattacacagggtccctgatgattggtcaaattcaagtcacaaaactgttgaatccaaatgcagctaagttcgaagttctacgtcaaaaagacgaaatcatagaaagagcaaatgagtgtgatccaatgttggtgaatttgggcggtggatgtaaagatatagaagcaagggtgatcgatacaatcatgggtaagatgctaattgttcatctgatcgttgatgttaaagacgctatgggtgcaaatgctgtcaacactatgtgtgaaaaagttgctcctttcatcgaacgtattactgggggaaaggtctatcttagaatcatttccaacttggctgcatatagacttgctagagcaaaggccgtttttgacaaagacgttattggcggagaggaggttgtagaagggatcatgcttgcatacgccttcgctgccgctgacccatttcgttgcgccacccacaataagggtatcatgaatggcatatcagccttaatgatcgctacaggaaacgactttagagccattgaagcaggagctcattcctatgctgcaataggtggatacaaaccactaactacctacgaagttgatagaaaaggtaatctagtaggcacaattgaaatacctatggcagtaggcgtgattggtggtgcaaccaaagtcaacccactagccaagatctctcttaagatactaggagtgaacactgctgaagagttagccagagtcgcagccgctctaggtttggctcaaaactttgctgccttaagagccttggccacagaaggtatccaaagaggtcacatggaattacatgccaggaacttagcaatcatggctggagctactggagatgaggttgacagagttgtagagattatggtgagagatggcaaaatcagattggactacgctaaggaagtattggagagactgcgttcctaa

SEQ ID NO: 102 - DNA, 1287 bp, synthetic construct
  misc_feature 1..1287: note = Synthetic oligonucleotide
  source 1..1287: mol_type = other DNA
atgtccttagattcaagactgccagctttcagaaatctgtctccagctgcaagactagatcacattggccaacttttgggactaagtcatgacgacgtttcccttttagcaaacgccggtgctttaccaatggatatcgctaatggtatgattgaaaatgtaatcgggacctttgaactgccatatgcagtggccagtaactttcagatcaatggccgtgacgtcttagtaccattagttgtggaggaacctagtatcgttgctgcagcctcttacatggcaaagttagctagagccaatggtgggttcactacatcttcatctgctccactaatgcatgcacaagtacaaattgtcggcattcaggatccactaaacgcaagattgtctttactgcgtagaaaggatgagatcatagaattagccaataggaaggaccaacttctgaattcattgggcggtggttgcagagacatagaggtgcatacatttgccgatactccaagaggaccaatgcttgtagcacaccttattgtcgatgtgcgtgatgccatgggagctaatactgttaacactatggctgaagcagtagcacctctgatggaagccataacaggtggccaggtaagattgagaatcctttccaatttggctgatcttagattggccagagcccaagtgagaatcactcctcagcaattggaaactgccgaattctcaggtgaggcagtaattgagggtatcttggacgcatatgcttttgccgctgtggacccttacagagccgctacccacaacaaaggcataatgaacggtatcgatcctttgatcgtcgctacaggaaatgattggagagctgttgaggcaggagctcatgcatacgcttgtagatccggacattacggttcattaacaacatgggaaaaagataacaatggacacttggtcgggacattggaaatgcctatgccagttggtttagttgggggtgctacaaaaacccatcctcttgctcaattgtctttgaggatacttggtgtcaaaactgctcaagcactagccgaaattgccgttgctgttggtttggcacaaaacttgggtgcaatgcgtgctttagctacagaaggcatccaaagaggacatatggctctacacgctagaaacattgcagttgttgcaggagccagaggtgatgaggttgattgggtggctagacaacttgtcgaatatcatgatgtcagagcagacagggctgtggcattactgaaacagaagagaggtcaataa

SEQ ID NO: 103 - DNA, 2355 bp, synthetic construct
  misc_feature 1..2355: note = Synthetic oligonucleotide
  source 1..2355: mol_type = other DNA
atgaatttgagtttgtgtatagcatctccactattgaccaaatctaatagaccagctgctttatcagcaattcatacagctagtacatcccatggtggccaaaccaaccctacgaatctgataatcgatacgaccaaggagagaatacaaaaacaattcaaaaatgttgaaatttcagtttcttcttatgatactgcgtgggttgccatggttccatcacctaattctccaaagtctccatgtttcccagaatgtttgaattggctgattaacaaccagttgaatgatggatcttggggtttagtcaatcacacgcacaatcacaaccatccacttttgaaagattctttatcctcaactttggcttgcatcgtggccctaaagagatggaacgtaggtgaggatcagattaacaaggggcttagtttcattgaatctaacttggcttccgcgactgaaaaatctcaaccatctccaataggattcgatatcatctttccaggtctgttagagtacgccaaaaatctagatatcaacttactgtctaagcaaactgatttctcactaatgttacacaagagagaattagaacaaaagagatgtcattcaaacgaaatggatggttacctagcttatatctctgaaggtcttggtaatctttacgattggaatatggtgaaaaagtaccagatgaaaaatggctcagttttcaattccccttctgcaactgcggcagcattcattaaccatcaaaatccaggatgcctgaactatttgaattcactactagacaaattcggcaacgcagttccaactgtataccctcacgatttgtttatcagattgagtatggtggatacaattgaaagacttggtatatcccaccactttagagtcgagatcaaaaatgttttggatgagacataccgttgttgggtggagagagatgaacaaatctttatggatgttgtgacgtgcgcgttggcctttagattgttgcgtattaacggttacgaagttagtccagatccacttgccgaaattacaaacgaattagctttaaaggatgaatacgccgctcttgaaacatatcatgcgtcacatatcctttaccaagaggacttatcatctggaaaacaaattcttaaatctgctgatttcctgaaggaaatcatatccactgatagtaatagactgtccaaactgatccataaagaggttgaaaatgcacttaagttccctattaacaccggcttagaacgtattaacacaagacgtaacatccagctttacaacgtagacaatactagaatcttgaaaaccacttaccattcttccaacatatcaaacactgattacctaagattagctgttgaagatttctacacatgtcagtctatctatagagaagagctgaaaggattagagagatgggtcgttgagaataagctagatcaattgaaatttgccagacaaaagacagcttattgttacttctcagttgccgccactttatcaagtccagaattgtcagatgcacgtatttcttgggctaaaaacggaattttgacaactgttgttgatgatttctttgatattggcgggacaatcgacgaattgacaaacctgattcaatgcgttgaaaagtggaatgtcgatgtcgataaagactgttgctcagaacatgttagaatactgttcttggctctgaaagatgctatctgttggatcggggatgaggctttcaaatggcaagctagagatgtgacgtctcacgtcattcaaacctggctagaactgatgaactctatgttgagagaagcaatttggactagagatgcatacgttcctacattaaacgagtatatggaaaacgcttatgtctcctttgctttgggtcctatcgttaagcctgccatatactttgtaggaccaaagctatccgaggaaatcgtcgaatcatcagaataccataacttgttcaagttaatgtccacacaaggcagattacttaatgatattcattctttcaaaagagagtttaaggaaggaaagttaaatgctgttgctctgcatctttctaatggcgaaagtggtaaagtcgaagaggaagtagttgaggaaatgatgatgatgatcaaaaacaagagaaaggagttgatgaaactaatcttcgaagagaacggttcaattgttcctagagcatgtaaggatgcattttggaacatgtgtcatgtgctaaactttttctacgcaaacgacgatggttttactgggaacacaatactagatacagtaaaagacatcatatacaaccctttggtcttagtaaacgaaaacgaggagcaaagataa

SEQ ID NO: 104 - DNA, 2355 bp, synthetic construct
  misc_feature 1..2355: note = Synthetic oligonucleotide
  source 1..2355: mol_type = other DNA
atgaatctgtccctttgtatagctagtccactgttgacaaaatcttctagaccaactgctctttctgcaattcatactgccagtactagtcatggaggtcaaacaaacccaacaaatttgataatcgatactactaaggagagaatccaaaagctattcaaaaatgttgaaatctcagtatcatcttatgacaccgcatgggttgcaatggtgccatcacctaattccccaaaaagtccatgttttccagagtgcttgaattggttaatcaataatcagttaaacgatggttcttggggtttagtcaaccacactcataaccacaatcatccattattgaaggactctttatcatcaacattagcctgtattgttgcattgaaaagatggaatgtaggtgaagatcaaatcaacaagggtttatcattcatagaatccaatctagcttctgctaccgacaaatcacaaccatctccaatcgggttcgacataatcttccctggtttgctggagtatgccaaaaaccttgatatcaacttactgtctaaacaaacagatttctctttgatgctacacaaaagagagttagagcagaaaagatgccattctaacgaaattgacgggtacttagcatatatctcagaaggtttgggtaatttgtatgactggaacatggtcaaaaagtatcagatgaaaaatggatccgtattcaattctccttctgcaactgccgcagcattcattaatcatcaaaaccctgggtgtcttaactacttgaactcactattagataagtttggaaatgcagttccaacagtctatcctttggacttgtacatcagattatctatggttgacactatagagagattaggtatttctcatcatttcagagttgagatcaaaaatgttttggacgagacatacagatgttgggtcgaaagagatgagcaaatctttatggatgtcgtgacctgcgctctggcttttagattgctaaggatacacggatacaaagtatctcctgatcaactggctgagattacaaacgaactggctttcaaagacgaatacgccgcattagaaacataccatgcatcccaaatactttaccaggaagacctaagttcaggaaaacaaatcttgaagtctgcagatttcctgaaaggcattctgtctacagatagtaataggttgtctaaattgatacacaaggaagtagaaaacgcactaaagtttcctattaacactggtttagagagaatcaatactaggagaaacattcagctgtacaacgtagataatacaaggattcttaagaccacctaccatagttcaaacatttccaacacctattacttaagattagctgtcgaagacttttacacttgtcaatcaatctacagagaggagttaaagggcctagaaagatgggtagttcaaaacaagttggatcaactgaagtttgctagacagaagacagcatactgttatttctctgttgctgctaccctttcatccccagaattgtctgatgccagaataagttgggccaaaaatggtattcttacaactgtagtcgatgatttctttgatattggaggtactattgatgaactgacaaatcttattcaatgtgttgaaaagtggaacgtggatgtagataaggattgctgcagtgaacatgtgagaatacttttcctggctctaaaagatgcaatatgttggattggcgacgaggccttcaagtggcaagctagagatgttacatctcatgtcatccaaacttggcttgaactgatgaactcaatgctaagagaagcaatctggacaagagatgcatacgttccaacattgaacgaatacatggaaaacgcttacgtctcatttgccttgggtcctattgttaagccagccatatactttgttgggccaaagttatccgaagagattgttgagtcttccgaatatcataacctattcaagttaatgtcaacacaaggcagacttctgaacgatatccactccttcaaaagagaattcaaggaaggtaagctaaacgctgttgctttgcacttgtctaatggtgaatctggcaaagtggaagaggaagtcgttgaggaaatgatgatgatgatcaaaaacaagagaaaggaattgatgaaattgattttcgaggaaaatggttcaatcgtacctagagcttgtaaagatgctttttggaatatgtgccatgttcttaacttcttttacgctaatgatgatggcttcactggaaatacaatattggatacagttaaagatatcatctacaacccacttgttttggtcaatgagaacgaggaacaaagataa

SEQ ID NO: 105 - DNA, 1773 bp, synthetic construct
  misc_feature 1..1773: note = Synthetic oligonucleotide
  source 1..1773: mol_type = other DNA
atggctatgccagtgaagctaacacctgcgtcattatccttaaaagctgtgtgctgcagattctcatccggtggccatgctttgagattcgggagtagtctgccatgttggagaaggacccctacccaaagatctacttcttcctctactactagaccagctgccgaagtgtcatcaggtaagagtaaacaacatgatcaggaagctagtgaagcgactatcagacaacaattacaacttgtggatgtcctggagaatatgggaatatccagacattttgctgcagagataaagtgcatactagacagaacttacagatcttggttacaaagacacgaggaaatcatgctggacactatgacatgtgctatggcttttagaatcctaagattgaacggatacaacgtttcatcagatgaactataccacgttgtagaggcatctggtctgcataattctttgggtgggtatcttaacgataccagaacactacttgaattacacaaggcttcaacagttagtatctctgaggatgaatctatcttagattcaattggctctagatccagaacattgcttagagaacaattggagtctggtggcgcactgagaaagccttctttattcaaagaggttgaacatgcactggatggacctttttacaccacacttgatagacttcatcataggtggaatattgaaaacttcaacattattgagcaacacatgttggagactccatacttatctaaccagcatacatcaagggatatcctagcattgtcaattagagatttttcctcctcacaattcacttatcaacaagagctacagcatctggagagttgggttaaggaatgtagattagatcaactacagttcgcaagacagaaattagcgtacttttacctatcagccgcaggcaccatgttttctcctgagctttctgatgcgagaacattatgggccaaaaacggggtgttgacaactattgttgatgatttctttgatgttgccggttctaaagaggaattggaaaacttagtcatgctggtcgaaatgtgggatgaacatcacaaagttgaattctattctgagcaggtcgaaatcatcttctcttccatctacgattctgtcaaccaattgggtgagaaggcctctttggttcaagacagatcaattacaaaacaccttgttgaaatatggttagacttgttaaagtccatgatgacggaagttgaatggagactgtcaaaatacgtgcctacagaaaaggaatacatgattaatgcctctcttatcttcggcctaggtccaatcgttttaccagctttgtatttcgttggtccaaagatttcagaaagtatagtaaaggacccagaatatgatgaattgttcaaactaatgtcaacatgtggtagattgttgaatgacgtgcaaacgttcgaaagagaatacaatgagggtaaactgaattctgtcagtctattggttcttcacggaggcccaatgtctatttcagacgcaaagaggaaattacaaaagcctattgatacgtgtagaagagatcttctttctttggtccttagagaagagtctgtagtaccaagaccatgtaaggaactattctggaaaatgtgtaaagtgtgctatttcttttactcaacaactgatgggttttctagtcaagtcgaaagagcaaaagaggtagacgctgtcataaatgagccactgaagttgcaaggttctcatacactggtatctgatgtttaa

SEQ ID NO: 106 - DNA, 2232 bp, synthetic construct
  misc_feature 1..2232: note = Synthetic oligonucleotide
  source 1..2232: mol_type = other DNA
atgcagaacttccatggtacaaaggaaaggatcaaaaagatgtttgacaagattgaattgtccgtttcttcttatgatacagcctgggttgcaatggtcccatcccctgattgcccagaaacaccttgttttccagaatgtactaaatggatcctagaaaatcagttgggtgatggtagttggtcacttcctcatggcaatccacttctagttaaagatgcattatcttccactcttgcttgtattctggctcttaaaagatggggaatcggtgaggaacagattaacaaaggactgagattcatagaactcaactctgctagtgtaaccgataacgaacaacacaaaccaattggatttgacattatctttccaggtatgattgaatacgctatagacttagacctgaatctaccactaaaaccaactgacattaactccatgttgcatcgtagagcccttgaattgacatcaggtggaggcaaaaatctagaaggtagaagagcttacttggcctacgtctctgaaggaatcggtaagctgcaagattgggaaatggctatgaaataccaacgtaaaaacggatctctgttcaatagtccatcaacaactgcagctgcattcatccatatacaagatgctgaatgcctccactatattcgttctcttctccagaaatttggaaacgcagtccctacaatataccctctcgatatctatgccagactttcaatggtagatgccctggaacgtcttggtattgatagacatttcagaaaggagagaaagttcgttctggatgaaacatacagattttggttgcaaggagaagaggagattttctccgataacgcaacctgtgctttggccttcagaatattgagacttaatggttacgatgtctctcttgaagatcacttctctaactctctgggcggttacttaaaggactcaggagcagctttagaactgtacagagccctccaattgtcttacccagacgagtccctcctggaaaagcaaaattctagaacttcttacttcttaaaacaaggtttatccaatgtctccctctgtggtgacagattgcgtaaaaacataattggagaggtgcatgatgctttaaacttttccgaccacgctaacttacaaagattagctattcgtagaaggattaagcattacgctactgacgatacaaggattctaaaaacttcctacagatgctcaacaatcggtaaccaagattttctaaaacttgcagtggaagatttcaatatctgtcaatcaatacaaagagaggaattcaagcatattgaaagatgggtcgttgaaagacgtctagacaagttaaagttcgctagacaaaaagaggcctattgctatttctcagccgcagcaacattgtttgcccctgaattgtctgatgctagaatgtcttgggccaaaaatggtgtattgacaactgtggttgatgatttcttcgatgtcggaggctctgaagaggaattagttaacttgatagaattgatcgagcgttgggatgtgaatggcagtgcagatttttgtagtgaggaagttgagattatctattctgctatccactcaactatctctgaaataggtgataagtcatttggctggcaaggtagagatgtaaagtctcaagttatcaagatctggctggacttattgaaatcaatgttaactgaagctcaatggtcttcaaacaagtctgttcctaccctagatgagtatatgacaaccgcccatgtttcattcgcacttggtccaattgtacttccagccttatacttcgttggcccaaagttgtcagaagaggttgcaggtcatcctgaactactaaacctctacaaagtcacatctacttgtggcagactactgaatgattggagaagttttaagagagaatccgaggaaggtaagctcaacgctattagtttatacatgatccactccggtggtgcttctacagaagaggaaacaatcgaacatttcaaaggtttgattgattctcagagaaggcaactgttacaattggtgttgcaagagaaggatagtatcatacctagaccatgtaaagatctattttggaatatgattaagttattacacactttctacatgaaagatgatggcttcacctcaaatgagatgaggaatgtagttaaggcaatcattaacgaaccaatctcactggatgaattatga

SEQ ID NO: 107 - DNA, 1542 bp, synthetic construct; identical to SEQ ID NO: 23
  misc_feature 1..1542: note = Synthetic oligonucleotide
  source 1..1542: mol_type = other DNA

SEQ ID NO: 108 - DNA, 1530 bp, synthetic construct
  misc_feature 1..1530: note = Synthetic oligonucleotide
  source 1..1530: mol_type = other DNA
atggcatttttctctatgatttcaattttgttgggatttgttatttcttctttcatcttcatctttttcttcaaaaagttacttagttttagtaggaaaaacatgtcagaagtttctactttgccaagtgttccagtagtgcctggttttccagttattgggaatttgttgcaactaaaggagaaaaagcctcataaaactttcactagatggtcagagatatatggacctatctactctataaagatgggttcttcatctcttattgtattgaacagtacagaaactgctaaggaagcaatggtcactagattttcatcaatatctaccagaaaattgtcaaacgccctaacagttctaacctgcgataagtctatggtcgccacttctgattatgatgacttccacaaattagttaagagatgtttgctaaatggacttcttggtgctaatgctcaaaagagaaaaagacactacagagatgctttgattgaaaatgtgagttccaagctacatgcacacgctagagatcatccacaagagccagttaactttagagcaattttcgaacacgaattgtttggtgtagcattaaagcaagccttcggtaaagacgtagaatccatatacgtcaaggagttaggcgtaacattatcaaaagatgaaatctttaaggtgcttgtacatgatatgatggagggtgcaattgatgtagattggagagatttcttcccatatttgaaatggatccctaataagtcttttgaagctaggatacaacaaaagcacaagagaagactagctgttatgaacgcacttatacaggacagattgaagcaaaatgggtctgaatcagatgatgattgttaccttaacttcttaatgtctgaggctaaaacattgactaaggaacagatcgcaatccttgtctgggaaacaatcattgaaacagcagatactaccttagtcacaactgaatgggccatatacgagctagccaaacatccatctgtgcaagataggttgtgtaaggagatccagaacgtgtgtggtggagagaaattcaaggaagagcagttgtcacaagttccttaccttaacggcgttttccatgaaaccttgagaaaatactcacctgcaccattagttcctattagatacgcccacgaagatacacaaatcggtggctaccatgttccagctgggtccgaaattgctataaacatctacgggtgcaacatggacaaaaagagatgggaaagaccagaagattggtggccagaaagattcttagatgatggcaaatatgaaacatctgatttgcataaaacaatggctttcggagctggcaaaagagtgtgtgccggtgctctacaagcctccctaatggctggtatcgctattggtagattggtccaagagttcgaatggaaacttagagatggtgaagaggaaaatgtcgatacttatgggttaacatctcaaaagttatacccactaatggcaatcatcaatcctagaagatcctaa

SEQ ID NO: 109 - DNA, 1578 bp, synthetic construct
  misc_feature 1..1578: note = Synthetic oligonucleotide
  source 1..1578: mol_type = other DNA
atgagtaagtctaatagtatgaattctacatcacacgaaaccctttttcaacaattggtcttgggtttggaccgtatgccattgatggatgttcactggttgatctacgttgctttcggcgcatggttatgttcttatgtgatacatgttttatcatcttcctctacagtaaaagtgccagttgttggatacaggtctgtattcgaacctacatggttgcttagacttagattcgtctgggaaggtggctctatcataggtcaagggtacaataagtttaaagactctattttccaagttaggaaattgggaactgatattgtcattataccacctaactatattgatgaagtgagaaaattgtcacaggacaagactagatcagttgaacctttcattaatgattttgcaggtcaatacacaagaggcatggttttcttgcaatctgacttacaaaaccgtgttatacaacaaagactaactccaaaattggtttccttgaccaaggtcatgaaggaagagttggattatgctttaacaaaagagatgcctgatatgaaaaatgacgaatgggtagaagtagatatcagtagtataatggtgagattgatttccaggatctccgccagagtctttctagggcctgaacactgtcgtaaccaggaatggttgactactacagcagaatattcagaatcacttttcattacagggtttatcttaagagttgtacctcatatcttaagaccattcatcgcccctctattaccttcatacaggactctacttagaaacgtttcaagtggtagaagagtcatcggtgacatcataagatctcagcaaggggatggtaacgaagatatactttcctggatgagagatgctgccacaggagaggaaaagcaaatcgataacattgctcagagaatgttaattctttctttagcatcaatccacactactgcgatgaccatgacacatgccatgtacgatctatgtgcttgccctgagtacattgaaccattaagagatgaagttaaatctgttgttggggcttctggctgggacaagacagcgttaaacagatttcataagttggactccttcctaaaagagtcacaaagattcaacccagtattcttattgacattcaatagaatctaccatcaatctatgaccttatcagatggcactaacattccatctggaacacgtattgctgttccatcacacgcaatgttgcaagattctgcacatgtcccaggtccaaccccacctactgaatttgatggattcagatatagtaagatacgttctgatagtaactacgcacaaaagtacctattctccatgaccgattcttcaaacatggctttcggatacggcaagtatgcttgtccaggtagattttacgcgtctaatgagatgaaactaacattagccattttgttgctacaatttgagttcaaactaccagatggtaaaggtcgtcctagaaatatcactatcgattctgatatgattccagacccaagagctagactttgcgtcagaaaaagatcacttagagatgaatga

SEQ ID NO: 110 - DNA, 1500 bp, synthetic construct
  misc_feature 1..1500: note = Synthetic oligonucleotide
  source 1..1500: mol_type = other DNA
atggaagatcctactgtcttatatgcttgtcttgccattgcagttgcaactttcgttgttagatggtacagagatccattgagatccatcccaacagttggtggttccgatttgcctattctatcttacatcggcgcactaagatggacaagacgtggcagagagatacttcaagagggatatgatggctacagaggatctacattcaaaatcgcgatgttagaccgttggatcgtgatcgcaaatggtcctaaactagctgatgaagtcagacgtagaccagatgaagagttaaactttatggacggattaggagcattcgtccaaactaagtacaccttaggtgaagctattcataacgatccataccatgtcgatatcataagagaaaaactaacaagaggccttccagccgtgcttcctgatgtcattgaagagttgacacttgcggttagacagtacattccaacagaaggtgatgaatgggtgtccgtaaactgttcaaaggccgcaagagatattgttgctagagcttctaatagagtctttgtaggtttgcctgcttgcagaaaccaaggttacttagatttggcaatagactttacattgtctgttgtcaaggatagagccatcatcaatatgtttccagaattgttgaagccaatagttggcagagttgtaggtaacgccaccagaaatgttcgtagagctgttccttttgttgctccattggtggaggaaagacgtagacttatggaagagtacggtgaagactggtctgaaaaacctaatgatatgttacagtggataatggatgaagctgcatccagagatagttcagtgaaggcaatcgcagagagattgttaatggtgaacttcgcggctattcatacctcatcaaacactatcactcatgctttgtaccaccttgccgaaatgcctgaaactttgcaaccacttagagaagagatcgaaccattagtcaaagaggagggctggaccaaggctgctatgggaaaaatgtggtggttagattcatttctaagagaatctcaaagatacaatggcattaacatcgtatctttaactagaatggctgacaaagatattacattgagtgatggcacatttttgccaaaaggtactctagtggccgttccagcgtattctactcatagagatgatgctgtctacgctgatgccttagtattcgatcctttcagattctcacgtatgagagcgagagaaggtgaaggtacaaagcaccagttcgttaatacttcagtcgagtacgttccatttggtcacggaaagcatgcttgtccaggaagattcttcgccgcaaacgaattgaaagcaatgttggcttacattgttctaaactatgatgtaaagttgcctggtgacggtaaacgtccattgaacatgtattggggtccaacagttttgcctgcaccagcaggccaagtattgttcagaaagagacaagttagtctataa

SEQ ID NO: 111 - DNA, 1578 bp, synthetic construct
  misc_feature 1..1578: note = Synthetic oligonucleotide
  source 1..1578: mol_type = other DNA
atgggtttgttcccattagaggattcctacgcgctggtctttgaaggactagcaataacactggctttgtactatctactgtctttcatctacaaaacatctaaaaagacatgtacacctcctaaagcatctggtgaaatcattccaattacaggaatcatattgaatctgctatctggctcaagtggtctacctattatcttagcacttgcctctttagcagacagatgtggtcctattttcaccattaggctgggtattaggagagtgctagtagtatcaaattgggaaatcgctaaggagattttcactacccacgatttgatagtttctaatagaccaaaatacttagccgctaagattcttggtttcaattatgtttcattctctttcgctccatacggcccatattgggtcggaatcagaaagattattgctacaaaactaatgtcttcttccagacttcagaagttgcaatttgtaagagtttttgaactagaaaactctatgaaatctatcagagaatcatggaaggagaaaaaggatgaagagggaaaggtattagttgagatgaaaaagtggttctgggaactgaatatgaacatagtgttaaggacagttgctggtaaacaatacactggtacagttgatgatgccgatgcaaagcgtatctccgagttattcagagaatggtttcactacactggcagatttgtcgttggagacgcttttccttttctaggttggttggacctgggcggatacaaaaagacaatggaattagttgctagtagattggactcaatggtcagtaaatggttagatgagcatcgtaaaaagcaagctaacgatgacaaaaaggaggatatggatttcatggatatcatgatctccatgacagaagcaaattcaccacttgaaggatacggcactgatactattatcaagaccacatgtatgactttgattgtttcaggagttgatacaacctcaatcgtacttacttgggccttatcacttttgttaaacaacagagatactttgaaaaaggcacaagaggaattagatatgtgcgtaggtaaaggaagacaagtcaacgagtctgatcttgttaacttgatatacttggaagcagtgcttaaagaggctttaagactttacccagcagcgttcttaggcggaccaagagcattcttggaagattgtactgttgctggttatagaattccaaagggcacctgcttgttgattaacatgtggaaactgcatagagatccaaacatttggagtgatccttgcgaattcaagccagaaagatttttgacacctaatcaaaaggatgttgatgtgatcggtatggatttcgaattgataccatttggtgccggcagaagatattgtccaggtactagattggctttacagatgttgcatatcgtattagcgacattgctgcaaaacttcgaaatgtcaacaccaaacgatgcgccagtcgatatgactgcttctgttggcatgacaaatgccaaagcatcacctttagaagtcttgctatcacctcgtgttaaatggtcctaa

SEQ ID NO: 112 - DNA, 1431 bp, synthetic construct
  misc_feature 1..1431: note = Synthetic oligonucleotide
  source 1..1431: mol_type = other DNA
atgatacaagttttaactccaattctactcttcctcatcttcttcgttttctggaaagtctacaaacatcaaaagactaaaatcaatctaccaccaggttccttcggctggccatttttgggtgaaaccttagccttacttagagcaggctgggattctgagccagaaagattcgtaagagagcgtatcaaaaagcatggatctccacttgttttcaagacatcactatttggagacagattcgctgttctttgcggtccagctggtaataagtttttgttctgcaacgaaaacaaattagtggcatcttggtggccagtccctgtaaggaagttgttcggtaaaagtttactcacaataagaggagatgaagcaaaatggatgagaaaaatgctattgtcttacttgggtccagatgcatttgccacacattatgccgttactatggatgttgtaacacgtagacatattgatgtccattggaggggcaaggaggaagttaatgtatttcaaacagttaagttgtacgcattcgaattagcttgtagattattcatgaacctagatgacccaaaccacatcgcgaaactcggtagtcttttcaacattttcctcaaagggatcatcgagcttcctatagacgttcctggaactagattttactccagtaaaaaggccgcagctgccattagaattgaattgaaaaagctcattaaagctagaaaactcgaattgaaggagggtaaggcgtcttcttcacaggacttgctttctcatctattaacatcacctgatgagaatgggatgttcttgacagaagaggaaatagtcgataacattctacttttgttattcgctggtcacgatacctctgcactatcaataacacttttgatgaaaaccttaggtgaacacagtgatgtgtacgacaaggttttgaaggaacaattagaaatttccaaaacaaaggaggcttgggaatcactaaagtgggaagatatccagaagatgaagtactcatggtcagtaatctgtgaagtcatgagattgaatcctcctgtcatagggacatacagagaggcgttggttgatatcgactatgctggttacactatcccaaaaggatggaagttgcattggtcagctgtttctactcaaagagacgaagccaatttcgaagatgtaactagattcgatccatccagatttgaaggggcaggccctactccattcacatttgtgcctttcggtggaggtcctagaatgtgtttaggcaaagagtttgccaggttagaagtgttagcatttctccacaacattgttaccaactttaagtgggatcttctaatccctgatgagaagatcgaatatgatccaatggctactccagctaagggcttgccaattagacttcatccacaccaagtctaa

SEQ ID NO: 113 - DNA, 1578 bp, synthetic construct
  misc_feature 1..1578: note = Synthetic oligonucleotide
  source 1..1578: mol_type = other DNA
atggagtctttagtggttcatacagtaaatgctatctggtgtattgtaatcgtcgggattttctcagttggttatcacgtttacggtagagctgtggtcgaacaatggagaatgagaagatcactgaagctacaaggtgttaaaggcccaccaccatccatcttcaatggtaacgtctcagaaatgcaacgtatccaatccgaagctaaacactgctctggcgataacattatctcacatgattattcttcttcattattcccacacttcgatcactggagaaaacagtacggcagaatctacacatactctactggattaaagcaacacttgtacatcaatcatccagaaatggtgaaggagctatctcagactaacacattgaacttgggtagaatcacccatataaccaaaagattgaatcctatcttaggtaacggaatcataacctctaatggtcctcattgggcccatcagcgtagaattatcgcctacgagtttactcatgataagatcaagggtatggttggtttgatggttgagtctgctatgcctatgttgaataagtgggaggagatggtaaagagaggcggagaaatgggatgcgacataagagttgatgaggacttgaaagatgtttcagcagatgtgattgcaaaagcctgtttcggatcctcattttctaaaggtaaggctattttctctatgataagagatttgcttacagctatcacaaagagaagtgttctattcagattcaacggattcactgatatggtctttgggagtaaaaagcatggtgacgttgatatagacgctttagaaatggaattggaatcatccatttgggaaactgtcaaggaacgtgaaatagaatgtaaagatactcacaaaaaggatctgatgcaattgattttggaaggggcaatgcgttcatgtgacggtaacctttgggataaatcagcatatagaagatttgttgtagataattgtaaatctatctacttcgcagggcatgatagtacagctgtctcagtgtcatggtgtttgatgttactggccctaaacccatcatggcaagttaagatccgtgatgaaattctgtcttcttgcaaaaatggtattccagatgccgaaagtatcccaaaccttaaaacagtgactatggttattcaagagacaatgagattataccctccagcaccaatcgtcgggagagaagcctctaaagatatcagattgggcgatctagttgttcctaaaggcgtctgtatatggacactaataccagctttacacagagatcctgagatttggggaccagatgcaaacgatttcaaaccagaaagattttctgaaggaatttcaaaggcttgtaagtatcctcaaagttacattccatttggtctgggtcctagaacatgcgttggtaaaaactttggcatgatggaagtaaaggttcttgtttccctgattgtctccaagttctctttcactctatctcctacctaccaacatagtcctagtcacaaacttttagtagaaccacaacatggggtggtaattagagtggtttaa

SEQ ID NO: 114 - DNA, 1590 bp, synthetic construct
  misc_feature 1..1590: note = Synthetic oligonucleotide
  source 1..1590: mol_type = other DNA
atgtacttcctactacaatacctcaacatcacaaccgttggtgtctttgccacattgtttctctcttattgtttacttctctggagaagtagagcgggtaacaaaaagattgccccagaagctgccgctgcatggcctattatcggccacctccacttacttgcaggtggatcccatcaactaccacatattacattgggtaacatggcagataagtacggtcctgtattcacaatcagaataggcttgcatagagctgtagttgtctcatcttgggaaatggcaaaggaatgttcaacagctaatgatcaagtgtcttcttcaagacctgaactattagcttctaagttgttgggttataactacgccatgtttggtttttcaccatacggttcatactggagagaaatgagaaagatcatctctctcgaattactatctaattccagattggaactattgaaagatgttagagcctcagaagttgtcacatctattaaggaactatacaaattgtgggcggaaaagaagaatgagtcaggattggtttctgtcgagatgaaacaatggttcggagatttgactttaaacgtgatcttgagaatggtggctggtaaaagatacttctccgcgagtgacgcttcagaaaacaaacaggcccagcgttgtagaagagtcttcagagaattcttccatctctccggcttgtttgtggttgctgatgctataccttttcttggatggctcgattggggaagacacgagaagaccttgaaaaagaccgccatagaaatggattccatcgcccaggagtggcttgaggaacatagacgtagaaaagattctggagatgataattctacccaagatttcatggacgttatgcaatctgtgctagatggcaaaaatctaggcggatacgatgctgatacgattaacaaggctacatgcttaactcttatatcaggtggcagtgatactactgtagtttctttgacatgggctcttagtcttgtgttaaacaatagagatactttgaaaaaggcacaggaagagttagacatccaagtcggtaaggaaagattggttaacgagcaagacatcagtaagttagtttacttgcaagcaatagtaaaagagacactcagactttatccaccaggtcctttgggtggtttgagacaattcactgaagattgtacactaggtggctatcacgtttcaaaaggaactagattaatcatgaacttatccaagattcaaaaagatccacgtatttggtctgatcctactgaattccaaccagagagattccttacgactcataaagatgtcgatccacgtggtaaacactttgaattcattccattcggtgcaggaagacgtgcatgtcctggtatcacattcggattacaagtactacatctaacattggcatctttcttgcatgcgtttgaattttcaacaccatcaaatgagcaggttaacatgagagaatcattaggtcttacgaatatgaaatctaccccattagaagttttgatttctccaagactatcccttaattgcttcaaccttatgaaaatttga

SEQ ID NO: 115 - DNA, 1440 bp, synthetic construct
  misc_feature 1..1440: note = Synthetic oligonucleotide
  source 1..1440: mol_type = other DNA
atggaacctaacttttacttgtcattactattgttgttcgtgaccttcatttctttaagtctgtttttcatcttttacaaacaaaagtccccattgaatttgccaccagggaaaatgggttaccctatcataggtgaaagtttagaattcctatccacaggctggaagggacatcctgaaaagttcatatttgatagaatgcgtaagtacagtagtgagttattcaagacttctattgtaggcgaatccacagttgtttgctgtggggcagctagtaacaaattcctattctctaacgaaaacaaactggtaactgcctggtggccagattctgttaacaaaatcttcccaacaacttcactggattctaatttgaaggaggaatctataaagatgagaaagttgctgccacagttcttcaaaccagaagcacttcaaagatacgtcggcgttatggatgtaatcgcacaaagacattttgtcactcactgggacaacaaaaatgagatcacagtttatccacttgctaaaagatacactttcttgcttgcgtgtagactgttcatgtctgttgaggatgaaaatcatgtggcgaaattctcagacccattccaactaatcgctgcaggcatcatttcacttcctatcgatcttcctggtactccattcaacaaggccataaaggcttcaaatttcattagaaaagagctgataaagattatcaaacaaagacgtgttgatctggcagagggtacagcatctccaacccaggatatcttgtcacatatgctattaacatctgatgaaaacggtaaatctatgaacgagttgaacattgccgacaagattcttggactattgataggaggccacgatacagcttcagtagcttgcacatttctagtgaagtacttaggagaattaccacatatctacgataaagtctaccaagagcaaatggaaattgccaagtccaaacctgctggggaattgttgaattgggatgacttgaaaaagatgaagtattcatggaatgtggcatgtgaggtaatgagattgtcaccacctttacaaggtggttttagagaggctataactgactttatgtttaacggtttctctattccaaaagggtggaagttatactggtccgccaactctacacacaaaaatgcagaatgtttcccaatgcctgagaaattcgatcctaccagatttgaaggtaatggtccagcgccttatacatttgtaccattcggtggaggccctagaatgtgtcctggaaaggaatacgctagattagaaatcttggttttcatgcataatctggtcaaacgttttaagtgggaaaaggttattccagacgaaaagattattgtcgatccattcccaatcccagctaaagatcttccaatccgtttgtatcctcacaaagcttaa

SEQ ID NO: 116 - DNA, 2133 bp, synthetic construct
  misc_feature 1..2133: note = Synthetic oligonucleotide
  source 1..2133: mol_type = other DNA
atgcaatcagattcagtcaaagtctctccatttgatttggtttccgctgctatgaatggcaaggcaatggaaaagttgaacgctagtgaatctgaagatccaacaacattgcctgcactaaagatgctagttgaaaatagagaattgttgacactgttcacaacttccttcgcagttcttattgggtgtcttgtatttctaatgtggagacgttcatcctctaaaaagctggtacaagatccagttccacaagttatcgttgtaaagaagaaagagaaggagtcagaggttgatgacgggaaaaagaaagtttctattttctacggcacacaaacaggaactgccgaaggttttgctaaagcattagtcgaggaagcaaaagtgagatatgaaaagacctctttcaaggttatcgatctagatgactacgctgcagatgatgatgaatatgaggaaaaactgaaaaaggaatccttagccttcttcttcttggccacatacggtgatggtgaacctactgataatgctgctaacttctacaagtggttcacagaaggcgacgataaaggtgaatggctgaaaaagttacaatacggagtatttggtttaggtaacagacaatatgaacatttcaacaagatcgctattgtagttgatgataaacttactgaaatgggagccaaaagattagtaccagtaggattaggggatgatgatcagtgtatagaagatgacttcaccgcctggaaggaattggtatggccagaattggatcaacttttaagggacgaagatgatacttctgtgactaccccatacactgcagccgtattggagtacagagtggtttaccatgataaaccagcagactcatatgctgaagatcaaacccatacaaacggtcatgttgttcatgatgcacagcatccttcaagatctaatgtggctttcaaaaaggaactacacacctctcaatcagataggtcttgtactcacttagaattcgatatttctcacacaggactgtcttacgaaactggcgatcacgttggcgtttattccgagaacttgtccgaagttgtcgatgaagcactaaaactgttagggttatcaccagacacatacttctcagtccatgctgataaggaggatgggacacctatcggtggtgcttcactaccaccaccttttcctccttgcacattgagagacgctctaaccagatacgcagatgtcttatcctcacctaaaaaggtagctttgctggcattggctgctcatgctagtgatcctagtgaagccgataggttaaagttcctggcttcaccagccggaaaagatgaatatgcacaatggatcgtcgccaaccaacgttctttgctagaagtgatgcaaagttttccatctgccaagcctccattaggtgtgttcttcgcagcagtagctccacgtttacaaccaagatactactctatcagttcatctcctaagatgtctcctaacagaatacatgttacatgtgctttggtgtacgagactactccagcaggcagaattcacagaggattgtgttcaacctggatgaaaaatgctgtccctttaacagagtcacctgattgctctcaagcatccattttcgttagaacatcaaatttcagacttccagtggatccaaaagttccagtcattatgataggaccaggcactggtcttgccccattcaggggctttcttcaagagagattggccttgaaggaatctggtacagaattgggttcttctatctttttctttggttgccgtaatagaaaagttgactttatctacgaggacgagcttaacaattttgttgagacaggagcattgtcagaattgatcgtcgcattttcaagagaagggactgccaaagagtacgttcagcacaagatgagtcaaaaagcctccgatatatggaaacttctaagtgaaggtgcctatctttatgtctgtggcgatgcaaagggcatggccaaggatgtccatagaactctgcatacaattgttcaggaacaagggagtctggattcttccaaggctgaattgtacgtcaaaaacttacagatgtctggaagatacttaagagatgtttggtaa

SEQ ID NO: 117 - DNA, 2079 bp, synthetic construct
  misc_feature 1..2079: note = Synthetic oligonucleotide
  source 1..2079: mol_type = other DNA
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 118 - DNA, 2142 bp, synthetic construct
  misc_feature 1..2142: note = Synthetic oligonucleotide
  source 1..2142: mol_type = other DNA
atggcagaattagatacacttgatatagtagtattaggtgttatctttttgggtactgtggcatactttactaagggtaaattgtggggtgttaccaaggatccatacgctaacggattcgctgcaggtggtgcttccaagcctggcagaactagaaacatcgtcgaagctatggaggaatcaggtaaaaactgtgttgttttctacggcagtcaaacaggtacagcggaggattacgcatcaagacttgcaaaggaaggaaagtccagattcggtttgaacactatgatcgccgatctagaagattatgacttcgataacttagacactgttccatctgataacatcgttatgtttgtattggctacttacggtgaaggcgaaccaacagataacgccgtggatttctatgagttcattactggcgaagatgcctctttcaatgagggcaacgatcctccactaggtaacttgaattacgttgcgttcggtctgggcaacaatacctacgaacactacaactcaatggtcaggaacgttaacaaggctctagaaaagttaggagctcatagaattggagaagcaggtgagggtgacgacggagctggaactatggaagaggactttttagcttggaaagatccaatgtgggaagccttggctaaaaagatgggcttggaggaaagagaagctgtatatgaacctattttcgctatcaatgagagagatgatttgacccctgaagcgaatgaggtatacttgggagaacctaataagctacacttggaaggtacagcgaaaggtccattcaactcccacaacccatatatcgcaccaattgcagaatcatacgaacttttctcagctaaggatagaaattgtctgcatatggaaattgatatttctggtagtaatctaaagtatgaaacaggcgaccatatcgcgatctggcctaccaacccaggtgaagaggtcaacaaatttcttgacattctagatctgtctggtaagcaacattccgtcgtaacagtgaaagccttagaacctacagccaaagttccttttccaaatccaactacctacgatgctatattgagataccatctggaaatatgcgctccagtttctagacagtttgtctcaactttagcagcattcgcccctaatgatgatatcaaagctgagatgaaccgtttgggatcagacaaagattacttccacgaaaagacaggaccacattactacaatatcgctagatttttggcctcagtctctaaaggtgaaaaatggacaaagataccattttctgctttcatagaaggccttacaaaactacaaccaagatactattctatctcttcctctagtttagttcagcctaaaaagattagtattactgctgttgtcgaatctcagcaaattccaggtagagatgacccattcagaggtgtagcgactaactacttgttcgctttgaagcagaaacaaaacggtgatccaaatccagctccttttggccaatcatacgagttgacaggaccaaggaataagtatgatggtatacatgttccagtccatgtaagacattctaactttaagctaccatctgatccaggcaaacctattatcatgatcggtccaggtaccggtgttgccccttttagaggcttcgtccaagagagggcaaaacaagccagagatggtgtagaagttggtaaaacactgctgttctttggatgtagaaagagtacagaagatttcatgtatcaaaaagagtggcaagagtacaaggaagctcttggcgacaaattcgaaatgattacagctttttcaagagaaggatctaaaaaggtttatgttcaacacagactgaaggaaagatcaaaggaagtttctgatcttctatcccaaaaagcatacttctacgtttgcggagacgccgcacatatggcacgtgaagtgaacactgtgttagcacagatcatagcagaaggccgtggtgtatcagaagccaagggtgaggaaattgtcaaaaacatgagatcagcaaatcaataccaagtgtgttctgatttcgtaactttacactgtaaagagacaacatacgcgaattcagaattgcaagaggatgtctggagttaa

SEQ ID NO: 119 - DNA, 2364 bp, synthetic construct
  misc_feature 1..2364: note = Synthetic oligonucleotide
  source 1..2364: mol_type = other DNA
atgaaaaccgggtttatctcaccagcaacagtatttcatcacagaatctcaccagcgaccactttcagacatcacttatcacctgctactacaaactctacaggcattgtcgccttaagagacatcaacttcagatgtaaagcagtttctaaagagtactctgatctgttgcagaaagatgaggcttctttcacaaaatgggacgatgacaaggtgaaagatcatcttgataccaacaaaaacttatacccaaatgatgagattaaggaatttgttgaatcagtaaaggctatgttcggtagtatgaatgacggggagataaacgtctctgcatacgatactgcatgggttgctttggttcaagatgtcgatggatcaggtagtcctcagttcccttcttctttagaatggattgccaacaatcaattgtcagatggatcatggggagatcatttgctgttctcagctcacgatagaatcatcaacacattagcatgcgttattgcacttacaagttggaatgttcatccttctaagtgtgaaaaaggtttgaattttctgagagaaaacatttgcaaattagaagatgaaaacgcagaacatatgccaattggttttgaagtaacattcccatcactaattgatatcgcgaaaaagttgaacattgaagtacctgaggatactccagcacttaaagagatctacgcacgtagagatatcaagttaactaagatcccaatggaagttcttcacaaggtacctactactttgttacattctttggaaggaatgcctgatttggagtgggaaaaactgttaaagctacaatgtaaagatggtagtttcttgttttccccatctagtaccgcattcgccctaatgcaaacaaaagatgagaaatgcttacagtatctaacaaatatcgtcactaagttcaacggtggcgtgcctaatgtgtacccagtcgatttgtttgaacatatttgggttgttgatagactgcagagattggggattgccagatacttcaaatcagagataaaagattgtgtagagtatatcaataagtactggaccaaaaatggaatttgttgggctagaaatactcacgttcaagatatcgatgatacagccatgggattcagagtgttgagagcgcacggttatgacgtcactccagatgtttttagacaatttgaaaaagatggtaaattcgtttgctttgcagggcaatcaacacaagccgtgacaggaatgtttaacgtttacagagcctctcaaatgttgttcccaggggagagaattttggaagatgccaaaaagttctcttacaattacttaaaggaaaagcaaagtaccaacgaattgctggataaatggataatcgctaaagatctacctggtgaagttggttatgctctggatatcccatggtatgcttccttaccaagattggaaactcgttattaccttgaacaatacggcggtgaagatgatgtctggataggcaagacattatacagaatgggttacgtgtccaataacacatatctagaaatggcaaagctggattacaataactatgttgcagtccttcaattagaatggtacacaatacaacaatggtacgtcgatattggtatagagaagttcgaatctgacaacatcaagtcagtcctggtttcttactacttggctgcggcttcaatattcgaacctgagagatctaaggagagaatcgcttgggcaaagacaacaatcttagtcgataagatcacatcaattttcgattcctctcagtcaagtaaggaagatattactgcctttattgacaagtttcgtaacaagtcctcctctaaaaagcactctatcaacggtgaaccatggcatgaagttatggtagctttgaaaaagaccttacacggctttgctctggatgctcttatgactcattctcaagatatacatccacagttacatcaagcctgggaaatgtggttgactaaactacaagacggcgtagatgttactgctgagctaatggtccaaatgatcaacatgactgctggcagatgggtatcaaaggaattacttactcatccacaatatcaaagattgtctactgtgacaaattctgtgtgtcacgatattaccaaacttcacaatttcaaggagaattccaccacagtggattcaaaggttcaggaactagtccagttggtttttagtgacacaccagatgatttggatcaagatatgaaacaaacattcctgacagtgatgaagacattctactacaaggcgtggtgtgatccaaacactataaacgatcatatatctaaagttttcgaaatcgtaatttga

SEQ ID NO: 120 - DNA, 1584 bp, synthetic construct
  misc_feature 1..1584: note = Synthetic oligonucleotide
  source 1..1584: mol_type = other DNA
atgcctgatgcacacgatgctccacctccacaaataagacagagaacactagtagatgaggctacccaactgctaactgagtccgcagaagatgcatggggtgaagtcagtgtgtcagaatacgaaacagcaaggctagttgcccatgctacatggttaggtggacacgccacaagagtggccttccttctggagagacaacacgaagacgggtcatggggtccaccaggtggatataggttagtccctacattatctgctgttcacgcattattgacatgtcttgcctctcctgctcaggatcatggcgttccacatgatagacttttaagagctgttgacgcaggcttgactgccttgagaagattggggacatctgactccccacctgatactatagcagttgagctggttatcccatctttgctagagggcattcaacacttactggaccctgctcatcctcatagtagaccagccttctctcaacatagaggctctcttgtttgtcctggtggactagatgggagaactctaggagctttgagatcacacgccgcagcaggtacaccagtaccaggaaaagtctggcacgcttccgagactttgggcttgagtaccgaagctgcttctcacttgcaaccagcccaaggtataatcggtggctctgctgctgccacagcaacatggctaaccagggttgcaccatctcaacagtcagattctgccagaagataccttgaggaattacaacacagatactctggcccagttccttccattacccctatcacatacttcgaaagagcatggttattgaacaattttgcagcagccggtgttccttgtgaggctccagctgctttgttggattccttagaagcagcacttacaccacaaggtgctcctgctggagcaggattgcctccagatgctgatgatacagccgctgtgttgcttgcattggcaacacatgggagaggtagaagaccagaagtactgatggattacaggactgacgggtatttccaatgctttattggggaaaggactccatcaatttcaacaaacgctcacgtattggaaacattagggcatcatgtggcccaacatccacaagatagagccagatacggatcagccatggataccgcatcagcttggctgctggcagctcaaaagcaagatggctcttggttagataaatggcatgcctcaccatactacgctactgtttgttgcacacaagccctagccgctcatgcaagtcctgcaactgcaccagctagacagagagctgtcagatgggttttagccacacaaagatccgatggcggttggggtctatggcattcaactgttgaagagactgcttatgccttacagatcttggccccaccttctggtggtggcaatatcccagtccaacaagcacttactagaggcagagcaagattgtgtggagccttgccactgactcctttatggcatgataaggatttgtatactccagtaagagtagtcagagctgccagagctgctgctctgtacactaccagagatctattgttaccaccattgtaa

SEQ ID NO: 121 - DNA, 1551 bp, synthetic construct
  misc_feature 1..1551: note = Synthetic oligonucleotide
  source 1..1551: mol_type = other DNA
atgaacgccctatccgaacacattttgtctgaattgagaagattattgtctgaaatgagtgatggcggatctgttggtccatctgtgtatgatacggcccaggccctaagattccacggtaacgtaacaggtagacaagatgcatatgcttggttgatcgcccagcaacaagcagatggaggttggggctctgccgactttccactctttagacatgctccaacatgggctgcacttctcgcattacaaagagctgatccacttcctggcgcagcagacgcagttcagaccgcaacaagattcttgcaaagacaaccagatccatacgctcatgccgttcctgaggatgcccctattggtgctgaactgatcttgcctcagttttgtggagaggctgcttggttgttgggaggtgtggccttccctagacacccagccctattaccattaagacaggcttgtttagtcaaactgggtgcagtcgccatgttgccttcaggacacccattgctccactcctgggaggcatggggtacttctccaacaacagcctgtccagacgatgatggttctataggtatctcaccagcagctacagccgcctggagagcccaggctgtgaccagaggctcaactcctcaagtgggcagagctgacgcatacttacaaatggcttcaagagcaacgagatcaggcatagaaggagtcttccctaatgtttggcctataaacgtattcgaaccatgctggtcactgtacactctccatcttgccggtctgttcgcccatccagcactggctgaggctgtaagagttatcgttgctcaacttgaagcaagattgggagtgcatggcctcggaccagctttacattttgctgccgacgctgatgatactgcagttgccttatgcgttctgcatttggctggcagagatcctgcagttgacgcattgagacattttgaaattggtgagctctttgttacattcccaggagagagaaatgctagtgtctctacgaacattcacgctcttcatgctttgagattgttaggtaaaccagctgccggagcaagtgcatacgtcgaagcaaatagaaatccacatggtttgtgggacaacgaaaaatggcacgtttcatggctttatccaactgcacacgccgttgcagctctagctcaaggcaagcctcaatggagagatgaaagagcactagccgctctactacaagctcaaagagatgatggtggttggggagctggtagaggatccactttcgaggaaaccgcctacgctcttttcgctttacacgttatggacggatctgaggaagccacaggcagaagaagaatcgctcaagtcgtcgcaagagccttagaatggatgctagctagacatgccgcacatggattaccacaaacaccactctggattggtaaggaattgtactgtcctactagagtcgtaagagtagctgagctagctggcctgtggttagcattaagatggggtagaagagtattagctgaaggtgctggtgctgcaccttaa

SEQ ID NO: 122 - DNA, 2952 bp, synthetic construct
  misc_feature 1..2952: note = Synthetic oligonucleotide
  source 1..2952: mol_type = other DNA
atggaatttgatgaaccattggttgacgaagcaagatctttagtgcagcgtactttacaagattatgatgacagatacggcttcggtactatgtcatgtgctgcttatgatacagcctgggtgtctttagttacaaaaacagtcgatgggagaaaacaatggcttttcccagagtgttttgaatttctactagaaacacaatctgatgccggaggatgggaaatcgggaattcagcaccaatcgacggtatattgaatacagctgcatccttacttgctctaaaacgtcacgttcaaactgagcaaatcatccaacctcaacatgaccataaggatctagcaggtagagctgaacgtgccgctgcatctttgagagcacaattggctgcattggatgtgtctacaactgaacacgtcggttttgagataattgttcctgcaatgctagacccattagaagccgaagatccatctctagttttcgattttccagctaggaaacctttgatgaagattcatgatgctaagatgagtagattcaggccagaatacttgtatggcaaacaaccaatgaccgccttacattcattagaggctttcataggcaaaatcgacttcgataaggtaagacaccaccgtacccatgggtctatgatgggttctccttcatctaccgcagcctacttaatgcacgcttcacaatgggatggtgactcagaggcttaccttagacacgtgattaaacacgcagcagggcagggaactggtgctgtaccatctgctttcccatcaacacattttgagtcatcttggattcttaccacattgtttagagctggattttcagcttctcatcttgcctgtgatgagttgaacaagttggtcgagatacttgagggctcattcgagaaggaaggtggggcaatcggttacgctccagggtttcaagcagatgttgatgatactgctaaaacaataagtacattagcagtccttggaagagatgctacaccaagacaaatgatcaaggtatttgaagctaatacacattttagaacataccctggtgaaagagatccttctttgacagctaattgtaatgctctatcagccttactacaccaaccagatgcagcaatgtatggatctcaaattcaaaagattaccaaatttgtctgtgactattggtggaagtctgatggtaagattaaagataagtggaacacttgctacttgtacccatctgtcttattagttgaggttttggttgatcttgttagtttattggagcagggtaaattgcctgatgttttggatcaagagcttcaatacagagtcgccatcacattgttccaagcatgtttaaggccattactagaccaagatgccgaaggatcatggaacaagtctatcgaagccacagcctacggcatccttatcctaactgaagctaggagagtttgtttcttcgacagattgtctgagccattgaatgaggcaatccgtagaggtatcgctttcgccgactctatgtctggaactgaagctcagttgaactacatttggatcgaaaaggttagttacgcacctgcattattgactaaatcctatttgttagcagcaagatgggctgctaagtctcctttaggcgcttccgtaggctcttctttgtggactccaccaagagaaggattggataagcatgtcagattattccatcaagctgagttattcagatcccttccagaatgggaattaagagcctccatgattgaagcagctttgttcacaccacttctaagagcacatagactagacgttttccctagacaagatgtaggtgaagacaaatatcttgatgtagttccattcttttggactgccgctaacaacagagatagaacttacgcttccactctattcctttacgatatgtgttttatcgcaatgttaaacttccagttagacgaattcatggaggccacagccggtatcttattcagagatcatatggatgatttgaggcaattgattcatgatcttttggcagagaaaacttccccaaagagttctggtagaagtagtcagggcacaaaagatgctgactcaggtatagaggaagacgtgtcaatgtccgattcagcttcagattcccaggatagaagtccagaatacgacttggttttcagtgcattgagtacctttacaaaacatgtcttgcaacacccatctatacaaagtgcctctgtatgggatagaaaactacttgctagagagatgaaggcttacttacttgctcatatccaacaagcagaagattcaactccattgtctgaattgaaagatgtgcctcaaaagactgatgtaacaagagtttctacatctactactaccttctttaactgggttagaacaacttccgcagaccatatatcctgcccatactccttccactttgtagcatgccatctaggcgcagcattgtcacctaaagggtctaacggtgattgctatccttcagctggtgagaagttcttggcagctgcagtctgcagacatttggccaccatgtgtagaatgtacaacgatcttggatcagctgaacgtgattctgatgaaggtaatttgaactccttggacttccctgaattcgccgattccgcaggaaacggagggatagaaattcagaaggccgctctattaaggttagctgagtttgagagagattcatacttagaggccttccgtcgtttacaagatgaatccaatagagttcacggtccagccggtggtgatgaagccagattgtccagaaggagaatggcaatccttgaattcttcgcccagcaggtagatttgtacggtcaagtatacgtcattagggatatttccgctcgtattcctaaaaacgaggttgagaaaaagagaaaattggatgatgctttcaattga

SEQ ID NO: 123 - DNA, 2646 bp, synthetic construct
  misc_feature 1..2646: note = Synthetic oligonucleotide
  source 1..2646: mol_type = other DNA
atggcttctagtacacttatccaaaacagatcatgtggcgtcacatcatctatgtcaagttttcaaatcttcagaggtcaaccactaagatttcctggcactagaaccccagctgcagttcaatgcttgaaaaagaggagatgccttaggccaaccgaatccgtactagaatcatctcctggctctggttcatatagaatagtaactggcccttctggaattaaccctagttctaacgggcacttgcaagagggttccttgactcacaggttaccaataccaatggaaaaatctatcgataacttccaatctactctatatgtgtcagatatttggtctgaaacactacagagaactgaatgtttgctacaagtaactgaaaacgtccagatgaatgagtggattgaggaaattagaatgtactttagaaatatgactttaggtgaaatttccatgtccccttacgacactgcttgggtggctagagttccagcgttggacggttctcatgggcctcaattccacagatctttgcaatggattatcgacaaccaattaccagatggggactggggcgaaccttctcttttcttgggttacgatagagtttgtaatactttagcctgtgtgattgcgttgaaaacatggggtgttggggcacaaaacgttgaaagaggaattcagttcctacaatctaacatatacaagatggaggaagatgacgctaatcatatgccaataggattcgaaatcgtattccctgctatgatggaagatgccaaagcattaggtttggatttgccatacgatgctactattttgcaacagatttcagccgaaagagagaaaaagatgaaaaagatcccaatggcaatggtgtacaaatacccaaccactttacttcactccttagaaggcttgcatagagaagttgattggaataagttgttacaattacaatctgaaaatggtagttttctttattcacctgcttcaaccgcatgcgccttaatgtacactaaggacgttaaatgttttgattacttaaaccagttgttgatcaagttcgaccacgcatgcccaaatgtatatccagtcgatctattcgaaagattatggatggttgacagattgcagagattagggatctccagatactttgaaagagagattagagattgtttacaatacgtctacagatattggaaagattgtggaatcggatgggcttctaactcttccgtacaagatgttgatgatacagccatggcgtttagacttttaaggactcatggtttcgacgtaaaggaagattgctttagacagtttttcaaggacggagaattcttctgcttcgcaggccaatcatctcaagcagttacaggcatgtttaatctttcaagagccagtcaaacattgtttccaggagaatctttattgaaaaaggctagaaccttctctagaaacttcttgagaacaaagcatgagaacaacgaatgtttcgataaatggatcattactaaagatttggctggtgaagtcgagtataacttgaccttcccatggtatgcctctttgcctagattagaacataggacatacttagatcaatatggaatcgatgatatctggataggcaaatctttatacaaaatgcctgctgttaccaacgaagttttcctaaagttggcaaaggcagactttaacatgtgtcaagctctacacaaaaaggaattggaacaagtgataaagtggaacgcgtcctgtcaattcagagatcttgaattcgccagacaaaaatcagtagaatgctattttgctggtgcagccacaatgttcgaaccagaaatggttcaagctagattagtctgggcaagatgttgtgtattgacaactgtcttagacgattactttgaccacgggacacctgttgaggaacttagagtgtttgttcaagctgtcagaacatggaatccagagttgatcaacggtttgccagagcaagctaaaatcttgtttatgggcttatacaaaacagttaacacaattgcagaggaagcattcatggcacagaaaagagacgtccatcatcatttgaaacactattgggacaagttgataacaagtgccctaaaggaggccgaatgggcagagtcaggttacgtcccaacatttgatgaatacatggaagtagctgaaatttctgttgctctagaaccaattgtctgtagtaccttgttctttgcgggtcatagactagatgaggatgttctagatagttacgattaccatctagttatgcatttggtaaacagagtcggtagaatcttgaatgatatacaaggcatgaagagggaggcttcacaaggtaagatctcatcagttcaaatctacatggaggaacatccatctgttccatctgaggccatggcgatcgctcatcttcaagagttagttgataattcaatgcagcaattgacatacgaagttcttaggttcactgcggttccaaaaagttgtaagagaatccacttgaatatggctaaaatcatgcatgccttctacaaggatactgatggattctcatcccttactgcaatgacaggattcgtcaaaaaggttcttttcgaacctgtgcctgagtaa

SEQ ID NO: 124 - DNA, 2859 bp, synthetic construct
  misc_feature 1..2859: note = Synthetic oligonucleotide
  source 1..2859: mol_type = other DNA
atgcctggtaaaattgaaaatggtaccccaaaggacctcaagactggaaatgattttgtttctgctgctaagagtttactagatcgagctttcaaaagtcatcattcctactacggattatgctcaacttcatgtcaagtttatgatacagcttgggttgcaatgattccaaaaacaagagataatgtaaaacagtggttgtttccagaatgtttccattacctcttaaaaacacaagccgcagatggctcatggggttcattgcctacaacacagacagcgggtatcctagatacagcctcagctgtgctggcattattgtgccacgcacaagagcctttacaaatattggatgtatctccagatgaaatggggttgagaatagaacacggtgtcacatccttgaaacgtcaattagcagtttggaatgatgtggaggacaccaaccatattggcgtcgagtttatcataccagccttactttccatgctagaaaaggaattagatgttccatcttttgaatttccatgtaggtccatcttagagagaatgcacggggagaaattaggtcatttcgacctggaacaagtttacggcaagccaagctcattgttgcactcattggaagcatttctcggtaagctagattttgatcgactatcacatcacctataccacggcagtatgatggcatctccatcttcaacggctgcttatcttattggggctacaaaatgggatgacgaagccgaagattacctaagacatgtaatgcgtaatggtgcaggacatgggaatggaggtatttctggtacatttccaactactcatttcgaatgtagctggattatagcaacgttgttaaaggttggctttactttgaagcaaattgacggcgatggcttaagaggtttatcaaccatcttacttgaggcgcttcgtgatgagaatggtgtcataggctttgcccctagaacagcagatgtagatgacacagccaaagctctattggccttgtcattggtaaaccagccagtgtcacctgatatcatgattaaggtctttgagggcaaagaccattttaccacttttggttcagaaagagatccatcattgacttccaacctgcacgtccttttatctttacttaaacaatctaacttgtctcaataccatcctcaaatcctcaaaacaacattattcacttgtagatggtggtggggttccgatcattgtgtcaaagacaaatggaatttgagtcacctatatccaactatgttgttggttgaagccttcactgaagtgctccatctcattgacggtggtgaattgtctagtctgtttgatgaatcctttaagtgtaagattggtcttagcatctttcaagcggtacttagaataatcctcacccaagacaacgacggctcttggagaggatacagagaacagacgtgttacgcaatattggctttagttcaagcgagacatgtatgctttttcactcacatggttgacagactgcaatcatgtgttgatcgaggtttctcatggttgaaatcttgctcttttcattctcaagacctgacttggacctctaaaacagcttatgaagtgggtttcgtagctgaagcatataaactagctgctttacaatctgcttccctggaggttcctgctgccaccattggacattctgtcacgtctgccgttccatcaagtgatcttgaaaaatacatgagattggtgagaaaaactgcgttattctctccactggatgagtggggtctaatggcttctatcatcgaatcttcatttttcgtaccattactgcaggcacaaagagttgaaatataccctagagataatatcaaggtggacgaagataagtacttgtctattatcccattcacatgggtcggatgcaataataggtctagaactttcgcaagtaacagatggctatacgatatgatgtacctttcattactcggctatcaaaccgacgagtacatggaagctgtagctgggccagtgtttggggatgtttccttgttacatcaaacaattgataaggtgattgataatacaatgggtaaccttgcgagagccaatggaacagtacacagtggtaatggacatcagcacgaatctcctaatataggtcaagtcgaggacaccttgactcgtttcacaaattcagtcttgaatcacaaagacgtccttaactctagctcatctgatcaagatactttgagaagagagtttagaacattcatgcacgctcatataacacaaatcgaagataactcacgattcagtaagcaagcctcatccgatgcgttttcctctcctgaacaatcttactttcaatgggtgaactcaactggtggctcacatgtcgcttgcgcctattcatttgccttctctaattgcctcatgtctgcaaatttgttgcagggtaaagacgcatttccaagcggaacgcaaaagtacttaatctcctctgttatgagacatgccacaaacatgtgtagaatgtataacgactttggctctattgccagagacaacgctgagagaaatgttaatagtattcattttcctgagtttactctctgtaacggaacttctcaaaacctagatgaaaggaaggaaagacttctgaaaatcgcaacttacgaacaagggtatttggatagagcactagaggccttggaaagacagagtagagatgatgccggagacagagctggatctaaagatatgagaaagttgaaaatcgttaagttattctgtgatgttacggacttatacgatcagctctacgttatcaaagatttgtcatcctctatgaagtaa

SEQ ID NO: 125 - DNA, 1086 bp, synthetic construct
  misc_feature 1..1086: note = Synthetic oligonucleotide
  source 1..1086: mol_type = other DNA
atggctttggtaaacccaaccgctcttttctatggtacctctatcagaacaagacctacaaacttactaaatccaactcaaaagctaagaccagtttcatcatcttccttaccttctttctcatcagttagtgcgattcttactgaaaaacatcaatctaatccttctgagaacaacaatttgcaaactcatctagaaactcctttcaactttgatagttatatgttggaaaaagtcaacatggttaacgaggcgcttgatgcatctgtcccactaaaagacccaatcaaaatccatgaatccatgagatactctttattggcaggcggtaagagaatcagaccaatgatgtgtattgcagcctgcgaaatagtcggaggtaatatccttaacgccatgccagccgcatgtgccgtggaaatgattcatactatgtctttggtgcatgacgatcttccatgtatggataatgatgacttcagaagaggtaaacctatttcacacaaggtctacggggaggaaatggcagtattgaccggcgatgctttactaagtttatctttcgaacatatagctactgctacaaagggtgtatcaaaggatagaatcgtcagagctataggggagttggcccgttcagttggctccgaaggtttagtggctggacaagttgtagatatcttgtcagagggtgctgatgttggattagatcacctagaatacattcacatccacaaaacagcaatgttgcttgagtcctcagtagttattggcgctatcatgggaggaggatctgatcagcagatcgaaaagttgagaaaattcgctagatctattggtctactattccaagttgtggatgacattttggatgttacaaaatctaccgaagagttggggaaaacagctggtaaggatttgttgacagataagacaacttacccaaagttgttaggtatagaaaagtccagagaatttgccgaaaaacttaacaaggaagcacaagagcaattaagtggctttgatagacgtaaggcagctcctttgatcgcgttagccaactacaatgcgtaccgtcaaaattga

SEQ ID NO: 126 - DNA, 1029 bp, synthetic construct
  misc_feature 1..1029: note = Synthetic oligonucleotide
  source 1..1029: mol_type = other DNA
atggctgagcaacaaatatctaacttgctgtctatgtttgatgcttcacatgctagtcagaaattagaaattactgtccaaatgatggacacataccattacagagaaacgcctccagattcctcatcttctgaaggcggttcattgtctagatacgacgagagaagagtctctttgcctctcagtcataatgctgcctctccagatattgtatcacaactatgtttttccactgcaatgtcttcagagttgaatcacagatggaaatctcaaagattaaaggtggccgattctccttacaactatatcctaacattaccatcaaaaggaattagaggtgcctttatcgattccctgaacgtatggttggaggttccagaggatgaaacatcagtcatcaaggaagttattggtatgctccacaactcttcattaatcattgatgacttccaagataattctccacttagaagaggaaagccatctacccatacagtcttcggccctgcccaggctatcaatactgctacttacgttatagttaaagcaatcgaaaagatacaagacatagtgggacacgatgcattggcagatgttacgggtactattacaactattttccaaggtcaggccatggacttgtggtggacagcaaatgcaatcgttccatcaatacaggaatacttacttatggtaaacgataaaaccggtgctctctttagactgagtttggagttgttagctctgaattccgaagccagtatttctgactctgctttagaaagtttatctagtgctgtttccttgctaggtcaatacttccaaatcagagacgactatatgaacttgatcgataacaagtatacagatcagaaaggcttctgcgaagatcttgatgaaggcaagtactcactaacacttattcatgccctccaaactgattcatccgatctactgaccaacatcctttcaatgagaagagtgcaaggaaagttaacggcacaaaagagatgttggttctggaaatga

SEQ ID NO: 127 - DNA, 903 bp, synthetic construct
  misc_feature 1..903: note = Synthetic oligonucleotide
  source 1..903: mol_type = other DNA
atggaaaagactaaggagaaagcagaacgtatcttgctggagccatacagatacttattacaactaccaggaaagcaagtccgttctaaactatcacaagcgttcaatcactggttaaaagttcctgaagataagttacaaatcattattgaagtcacagaaatgctacacaatgcttctttactgatcgatgatatagaggattcttccaaactgagaagaggttttcctgtcgctcattccatatacggggtaccaagtgtaatcaactcagctaattacgtctacttcttgggattggaaaaagtattgacattagatcatccagacgctgtaaagctattcaccagacaacttcttgaattgcatcaaggtcaaggtttggatatctattggagagacacttatacttgcccaacagaagaggagtacaaagcaatggttctacaaaagactggcggtttgttcggacttgccgttggtctgatgcaacttttctctgattacaaggaggacttaaagcctctgttggataccttgggcttgtttttccagattagagatgactacgctaacttacattcaaaggaatattcagaaaacaaatcattctgtgaagatttgactgaagggaagtttagttttccaacaatccacgccatttggtcaagaccagaatctactcaagtgcaaaacattctgcgtcagagaacagagaatattgacatcaaaaagtattgtgttcagtacttggaagatgttggttcttttgcttacacaagacatacacttagagaattagaggcaaaagcatacaagcaaatagaagcctgtggaggcaatccttctctagtggcattggttaaacatttgtccaaaatgttcaccgaggaaaacaagtaa

SEQ ID NO: 128 - DNA, 1020 bp, synthetic construct
  misc_feature 1..1020: note = Synthetic oligonucleotide
  source 1..1020: mol_type = other DNA
atggcaagattctattttcttaacgcactattgatggttatctcattacaatcaactacagccttcactccagctaaacttgcttatccaacaacaacaacagctctaaatgtcgcctccgccgaaacttctttcagtctagatgaatacttggcctctaagataggacctatagagtctgccttggaagcatcagtcaaatccagaattccacagaccgataagatctgcgaatctatggcctactctttgatggcaggaggcaagagaattagaccagtgttgtgtatcgctgcatgtgagatgttcggtggatcccaagatgtcgctatgcctactgctgtggcattagaaatgatacacacaatgtctttgattcatgatgatttgccatccatggataacgatgacttgagaagaggtaaaccaacaaaccatgtcgttttcggcgaagatgtagctattcttgcaggtgactctttattgtcaacttccttcgagcacgtcgctagagaaacaaaaggagtgtcagcagaaaagatcgtggatgttatcgctagattaggcaaatctgttggtgccgagggccttgctggcggtcaagttatggacttagaatgtgaagctaaaccaggtaccacattagacgacttgaaatggattcatatccataaaaccgctacattgttacaagttgctgtagcttctggtgcagttctaggtggtgcaactcctgaagaggttgctgcatgcgagttgtttgctatgaatataggtcttgcctttcaagttgccgacgatatccttgatgtaaccgcttcatcagaagatttgggtaaaactgcaggcaaagatgaagctactgataagacaacttacccaaagttattaggattagaagagagtaaggcatacgcaagacaactaatcgatgaagccaaggaaagtttggctccttttggagatagagctgcccctttattggccattgcagatttcattattgatagaaagaattga

SEQ ID NO: 129 - DNA, 1068 bp, synthetic construct
  misc_feature 1..1068: note = Synthetic oligonucleotide
  source 1..1068: mol_type = other DNA
atgcacttagcaccacgtagagtccctagaggtagaagatcaccacctgacagagttcctgaaagacaaggtgccttgggtagaagacgtggagctggctctactggctgtgcccgtgctgctgctggtgttcaccgtagaagaggaggaggcgaggctgatccatcagctgctgtgcatagaggctggcaagccggtggtggcaccggtttgcctgatgaggtggtgtctaccgcagccgccttagaaatgtttcatgcttttgctttaatccatgatgatatcatggatgatagtgcaactagaagaggctccccaactgttcacagagccctagctgatcgtttaggcgctgctctggacccagatcaggccggtcaactaggagtttctactgctatcttggttggagatctggctttgacatggtccgatgaattgttatacgctccattgactccacatagactggcagcagtactaccattggtaacagctatgagagctgaaaccgttcatggccaatatcttgatataactagtgctagaagacctgggaccgatacttctcttgcattgagaatagccagatataagacagcagcttacacaatggaacgtccactgcacattggtgcagccctggctggggcaagaccagaactattagcagggctttcagcatacgccttgccagctggagaagccttccaattggcagatgacctgctaggcgtcttcggtgatccaagacgtacagggaaacctgacctagatgatcttagaggtggaaagcatactgtcttagtcgccttggcaagagaacatgccactccagaacagagacacacattggatacattattgggtacaccaggtcttgatagacaaggcgcttcaagactaagatgcgtattggtagcaactggtgcaagagccgaagccgaaagacttattacagagagaagagatcaagcattaactgcattgaacgcattaacactgccacctcctttagctgaggcattagcaagattgacattagggtctacagctcatcctgcctaa

SEQ ID NO: 130 - DNA, 993 bp, synthetic construct
  misc_feature 1..993: note = Synthetic oligonucleotide
  source 1..993: mol_type = other DNA
atgtcatatttcgataactacttcaatgagatagttaattccgtgaacgacatcattaagtcttacatctctggcgacgtaccaaaactatacgaagcctcctaccatttgtttacatcaggaggaaagagactaagaccattgatccttacaatttcttctgatcttttcggtggacagagagaaagagcatactatgctggcgcagcaatcgaagttttgcacacattcactttggttcacgatgatatcatggatcaagataacattcgtagaggtcttcctactgtacatgtcaagtatggcctacctttggccattttagctggtgacttattgcatgcaaaagcctttcaattgttgactcaggcattgagaggtctaccatctgaaactatcatcaaggcgtttgatatctttacaagatctatcattatcatatcagaaggtcaagctgtcgatatggaattcgaagatagaattgatatcaaggaacaagagtatttggatatgatatctcgtaaaaccgctgccttattctcagcttcttcttccattggggcgttgatagctggagctaatgataacgatgtgagattaatgtccgatttcggtacaaatcttgggatcgcatttcaaattgtagatgatatacttggtttaacagctgatgaaaaagagctaggaaaacctgttttcagtgatatcagagaaggtaaaaagaccatattagtcattaagactttagaattgtgtaaggaagacgagaaaaagattgtgttaaaagcgctaggcaacaagtcagcatcaaaggaagagttgatgagttctgctgacataatcaaaaagtactcattggattacgcctacaacttagctgagaaatactacaaaaacgccatcgattctctaaatcaagtttcaagtaaaagtgatattccagggaaggcattgaaatatcttgctgaattcaccatcagaagacgtaagtaa

SEQ ID NO: 131 - DNA, 894 bp, synthetic construct; identical to SEQ ID NO: 22
  misc_feature 1..894: note = Synthetic oligonucleotide
  source 1..894: mol_type = other DNA

SEQ ID NO: 132 - DNA, 1116 bp, synthetic construct
  misc_feature 1..1116: note = Synthetic oligonucleotide
  source 1..1116: mol_type = other DNA
atggcctctgttactttgggttcctggatcgtcgtccaccaccataaccatcaccatccatcatctatcctaactaaatctcgttcaagatcctgtcctattacactaaccaaaccaatctcttttcgttcaaagagaacagtttcctctagtagttctatcgtgtcctctagtgtcgtcactaaggaagacaatctgagacagtctgaaccttcttcctttgatttcatgtcatatatcattactaaggcagaactagtgaataaggctcttgattcagcagttccattaagagagccattgaaaatccatgaagcaatgagatactctcttctagctggcgggaagagagtcagacctgtactctgcatagcagcgtgcgaattagttggtggcgaggaatcaaccgctatgcctgccgcttgtgctgtagaaatgattcatacaatgtcactgatacacgatgatttgccatgtatggataacgatgatctgagaaggggtaagccaactaaccataaggttttcggcgaagatgttgccgtcttagctggtgatgctttgttatctttcgcgttcgaacatttggcatccgcaacatcaagtgatgttgtgtcaccagtaagagtagttagagcagttggagaactggctaaagctattggaactgagggtttagttgcaggtcaagtcgtcgatatctcttccgaaggtcttgatttgaatgatgtaggtcttgaacatctcgaattcatccatcttcacaagacagctgcacttttagaagccagtgcggttctcggcgcaattgttggcggagggagtgatgacgaaattgagagattgaggaagtttgctagatgtataggattactgttccaagtagtagacgatatactagatgtgacaaagtcttccaaagagttgggaaaaacagctggtaaagatttgattgccgacaaattgacctaccctaagattatggggctagaaaaatcaagagaatttgccgagaaactcaatagagaggcgcgtgatcaactgttgggtttcgattctgataaagttgcaccactcttagccttagccaactacatcgcttacagacaaaactaa

SEQ ID NO: 133 - DNA, 2340 bp, synthetic construct
  misc_feature 1..2340: note = Synthetic oligonucleotide
  source 1..2340: mol_type = other DNA
gcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaaggacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 134 - protein, 777 aa, Zea mays
AQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 135 - DNA, 1555 bp, synthetic construct
  misc_feature 1..1555: note = Synthetic oligonucleotide
  source 1..1555: mol_type = other DNA
atggcggaacaacaaaagatcaagaaatcaccacacgttctactcatcccattccctttacaaggccatataaaccctttcatccagtttggcaaacgattaatctccaaaggtgtcaaaacaacacttgttaccaccatccacaccttaaactcaaccctaaaccacagtaacaccaccaccacctccatcgaaatccaagcaatttccgatggttgtgatgaaggcggttttatgagtgcaggagaatcatatttggaaacattcaaacaagttgggtctaaatcactagctgacttaatcaagaagcttcaaagtgaaggaaccacaattgatgcaatcatttatgattctatgactgaatgggttttagatgttgcaattgagtttggaatcgatggtggttcgtttttcactcaagcttgtgttgtaaacagcttatattatcatgttcataagggtttgatttctttgccattgggtgaaactgtttcggttcctggatttccagtgcttcaacggtgggagacaccgttaattttgcagaatcatgagcaaatacagagcccttggtctcagatgttgtttggtcagtttgctaatattgatcaagcacgttgggtcttcacaaatagtttttacaagctcgaggaagaggtaatagagtggacgagaaagatatggaacttgaaggtaatcgggccaacacttccatccatgtaccttgacaaacgacttgatgatgataaagataacggatttaatctctacaaagcaaaccatcatgagtgcatgaactggttagacgataagccaaaggaatcagttgtttacgtagcatttggtagcctggtgaaacatggacccgaacaagtggaagaaatcacacgggctttaatagatagtgatgtcaacttcttgtgggttatcaaacataaagaagagggaaagctcccagaaaatctttcggaagtaataaaaaccggaaagggtttgattgtagcatggtgcaaacaattggatgtgttagcacacgaatcagtaggatgctttgttacacattgtgggttcaactcaactcttgaagcaataagtcttggagtccccgttgttgcaatgcctcaattttcggatcaaactacaaatgccaagcttctagatgaaattttgggtgttggagttagagttaaggctgatgagaatgggatagtgagaagaggaaatcttgcgtcatgtattaagatgattatggaggaggaaagaggagtaataatccgaaagaatgcggtaaaatggaaggatttggctaaagtagccgttcatgaaggtggtagctcagacaatgatattgtcgaatttgtaagtgagctaattaaggcttaaatttttgttgctttgtattttatgtgttatggttttttgatttagatgtattcaattaatattgaatcataactaaattcaagattattgtttgtaatattctttgtcctaaaattttgcgacttaaaacctttagtttataaaaagaaattagaaaatactattgcacgga

SEQ ID NO: 136 - DNA, 2409 bp, synthetic construct
  misc_feature 1..2409: note = Synthetic oligonucleotide
  source 1..2409: mol_type = other DNA
atgtctcttcagtatcatgttctaaactccattccaagtacaacctttctcagttctactaaaacaacaatatcttcttctttccttaccatctcaggatctcctctcaatgtcgctagagacaaatccagaagcggttccatacattgttcaaagcttcgaactcaagaatacattaattctcaagaggttcaacatgatttgcctctaatacatgagtggcaacagcttcaaggagaagatgctcctcagattagtgttggaagtaatagtaatgcattcaaagaagcagtgaagagtgtgaaaacgatcttgagaaacctaacggacggggaaattacgatatcggcttacgatacagcttgggttgcattgatcgatgccggagataaaactccggcgtttccctccgccgtgaaatggatcgccgagaaccaactttccgatggttcttggggagatgcgtatctcttctcttatcatgatcgtctcatcaatacccttgcatgcgtcgttgctctaagatcatggaatctctttcctcatcaatgcaacaaaggaatcacgtttttccgggaaaatattgggaagctagaagacgaaaatgatgagcatatgccaatcggattcgaagtagcattcccatcgttgcttgagatagctcgaggaataaacattgatgtaccgtacgattctccggtcttaaaagatatatacgccaagaaagagctaaagcttacaaggataccaaaagagataatgcacaagataccaacaacattgttgcatagtttggaggggatgcgtgatttagattgggaaaagctcttgaaacttcaatctcaagacggatctttcctcttctctccttcctctaccgcttttgcattcatgcagacccgagacagtaactgcctcgagtatttgcgaaatgccgtcaaacgtttcaatggaggagttcccaatgtctttcccgtggatcttttcgagcacatatggatagtggatcggttacaacgtttagggatatcgagatactttgaagaagagattaaagagtgtcttgactatgtccacagatattggaccgacaatggcatatgttgggctagatgttcccatgtccaagacatcgatgatacagccatggcatttaggctcttaagacaacatggataccaagtgtccgcagatgtattcaagaactttgagaaagagggagagtttttctgctttgtggggcaatcaaaccaagcagtaaccggtatgttcaacctataccgggcatcacaattggcgtttccaagggaagagatattgaaaaacgccaaagagttttcttataattatctgctagaaaaacgggagagagaggagttgattgataagtggattataatgaaagacttacctggcgagattgggtttgcgttagagattccatggtacgcaagcttgcctcgagtagagacgagattctatattgatcaatatggtggagaaaacgacgtttggattggcaagactctttataggatgccatacgtgaacaataatggatatctggaattagcaaaacaagattacaacaattgccaagctcagcatcagctcgaatgggacatattccaaaagtggtatgaagaaaataggttaagtgagtggggtgtgcgcagaagtgagcttctcgagtgttactacttagcggctgcaactatatttgaatcagaaaggtcacatgagagaatggtttgggctaagtcaagtgtattggttaaagccatttcttcttcttttggggaatcctctgactccagaagaagcttctccgatcagtttcatgaatacattgccaatgctcgacgaagtgatcatcactttaatgacaggaacatgagattggaccgaccaggatcggttcaggccagtcggcttgccggagtgttaatcgggactttgaatcaaatgtcttttgaccttttcatgtctcatggccgtgacgttaacaatctcctctatctatcgtggggagattggatggaaaaatggaaactatatggagatgaaggagaaggagagctcatggtgaagatgataattctaatgaagaacaatgacctaactaacttcttcacccacactcacttcgttcgtctcgcggaaatcatcaatcgaatctgtcttcctcgccaatacttaaaggcaaggagaaacgatgagaaggagaagacaataaagagtatggagaaggagatggggaaaatggttgagttagcattgtcggagagtgacacatttcgtgacgtcagcatcacgtttcttgatgtagcaaaagcattttactactttgctttatgtggcgatcatctccaaactcacatctccaaagtcttgtttcaaaaagtctag